(19) United States
(12) Patent Application Publication
(10) Pub. No.: US 2026/0297673 A1
(43) Pub. Date: Oct. 1, 2026

(54) DNA METHYLATION-BASED ALGORITHM TO DIAGNOSE IRRITABLE BOWEL SYNDROME AND OTHER GI CONDITIONS

(71) Applicant: THE REGENTS OF THE UNIVERSITY OF CALIFORNIA, OAKLAND, CA (US)

(72) Inventors: Lin Chang, MANHATTAN BEACH, CA (US); Swapna Joshi, CULVER CITY, CA (US)

(73) Assignee: THE REGENTS OF THE UNIVERSITY OF CALIFORNIA, OAKLAND, CA (US)

(21) Appl. No.: 19/478,560

(22) PCT Filed: Apr. 26, 2024
(86) PCT No.: PCT/US2024/026561

§ 371 Date: Oct. 24, 2025

Related U.S. Application Data
(60) Provisional application No. 63/499,017, filed on Apr. 28, 2023.

(51) Int. Cl. C12Q 1/6883; G16B 20/00

(52) U.S. Cl. CPC C12Q 1/6883; G16B 20/00; C12Q 2600/154

(57) ABSTRACT

Blood-based genome-wide DNA methylation profiles are utilized to develop a reliable test to diagnose irritable bowel syndrome (IBS) versus inflammatory bowel disease (IBD), celiac disease and other gastrointestinal diseases that mimic IBS. These methods provide a means to rule out IBS and rule-in active IBD or celiac disease, as well as to rule-out active IBD and celiac disease, and to rule-in IBS.

This application claims benefit of U.S. provisional patent application No. 63/499,017, filed Apr. 28, 2023, the entire contents of which are incorporated by reference into this application.

### STATEMENT REGARDING FEDERALLY SPONSORED RESEARCH

This invention was made with government support under Grant Numbers DK104078 and DK064539, awarded by the National Institutes of Health. The government has certain rights in the invention.

### BACKGROUND

Irritable bowel syndrome (IBS), inflammatory bowel disease (IBD) and celiac disease (CeD) present with similar gastrointestinal (GI) symptoms. While IBD and CeD are diagnosed by endoscopy with tissue confirmation, IBS is diagnosed by symptom criteria due to a lack of a reliable diagnostic test. Epigenetic modifications including DNA methylation have not been studied well in IBS, IBD and CeD.

There remains a need for blood-based diagnostic classifiers that can differentiate IBS, IBD, and CeD, as well as other GI diseases that mimic IBS.

### SUMMARY

The tools and methods described herein address these needs and more. Described herein are methods of classifying biological samples based on gastrointestinal disease. In some embodiments, the method comprises: (a) measuring the amount of DNA methylation at CpG sites in DNA in a biological sample obtained from the subject to generate a genome-wide DNA CpG methylation profile of the subject, wherein the measuring comprises probing at least 200,000 CpG sites; (b) performing a normalization of technical noise and bias correction on the profile; (c) filtering the profile to retain data from a first set of probed CpG sites that is the same as a first set of CpG sites from a first training data set trained on a first pairwise comparison between biological samples, wherein the first pairwise comparison is selected from: (i) irritable bowel syndrome (IBS) and inflammatory bowel disease (IBD); (ii) IBS and celiac disease (CeD); (ili) IBD and CeD; (iv) IBS and healthy control; (v) IBD and healthy control; and (vi) CeD and healthy control; and (d) classifying the CpG methylation profile of the biological samples as IBS, IBD, CeD, or healthy based on a probability cutoff derived from the first training data set. The classifying can be implemented by use of a computer. Also provided is a system for performing the methods described herein.

In some embodiments, the filtering of step (c) is repeated to retain data from a second set of probes that is the same as a second set of probes from a second training data set trained on a second pairwise comparison between biological samples, wherein the second pairwise comparison is selected from (i) to (vi), and wherein the second pairwise comparison differs from the first pairwise comparison.

In some embodiments, the filtering of step (c) is repeated to retain data from subsequent sets of probes corresponding to subsequent training data sets trained on subsequent pairwise comparisons between biological samples, wherein each of the remaining pairwise comparisons of (i) to (vi) is performed.

In some embodiments, the probing comprises contacting the biological samples with a methylation microarray. In some embodiments, the biological sample comprises blood, plasma, serum, or mucosal tissue. In some embodiments, the sample is peripheral blood mononuclear cells (PBMCs), peripheral blood lymphocytes (PBL), or whole blood.

In some embodiments, the method further comprises measuring the amount of fecal calprotectin (FCP) and/or blood C-reactive protein (CRP) in the biological sample. In some embodiments, 120 mcg/g FCP is the cut off for determining likelihood of IBD. In some embodiments, 150 mcg/g FCP is the cut off for determining likelihood of IBD. In some embodiments, the cutoff is 8-10 mg/l CRP for IBD. In some embodiments, the cutoff is 8 mg/l CRP for IBD. In some embodiments, an elevated (>4 U/ml) anti-tissue transglutaminase antibody is indicative of CeD.

In some embodiments, the training data set is trained using GLMNET algorithm. In some embodiments, the training data set is trained using regularization parameter value and elastic net mixing parameter. This parameter minimizes the misclassification error.

Also described is a method of screening for, diagnosing, or detecting irritable bowel syndrome (IBS), inflammatory bowel disease (IBD) and/or celiac disease (CeD) in a subject. In some embodiments, the method comprises performing the method of classifying described herein on a biological sample obtained from the subject, and identifying the subject as having IBS, IBD, or CeD based on the classifying of step (d). In some embodiments, the method further comprises treating the subject for IBS, IBD, or CeD.

Treatment for IBS can include fiber supplements, laxatives, anti-diarrheal medications, such as loperamide, a bile acid binder, such as cholestyramine, colestipol or colesevelam, anticholinergic medications, such as dicyclomine or hysocyamine, tricyclic antidepressants, such as amitriptyline, imipramine, desipramine or nortriptyline, serotonin and norepinephrine reuptake inhibitor (SNRI) antidepressants, such as duloxetine, SSRI antidepressants, such as fluoxetine or paroxetine, tetracyclic antidepressant, such as mirtazapine, pregabalin or gabapentin. Medications approved for certain people with IBS include: Alosetron, Eluxadoline, Rifaximin, Lubiprostone, Plecanatide, Tenapanor and Linaclotide.

Treatment for IBD can include anti-inflammatory drugs, which include aminosalicylates, such as mesalamine, balsalazide and olsalazine, and immune system suppressors. Time-limited courses of corticosteroids can be both anti-inflammatory and immunosuppressing. Some examples of immunosuppressant drugs include azathioprine, mercaptopurine and methotrexate. Small molecules can also be used for IBD treatment. These include tofacitinib, upadacitinib and ozanimod. Biologics for treating IBD include infliximab, adalimumab, golimumab, certolizumab, vedolizumab, ustekinumab, and risankizumab. In some cases, treatment can include antibiotics, anti-diarrheal medications, such as psyllium powder or methylcellulose, and/or loperamide, pain relievers, and vitamins and supplements.

Treatment for CeD can include gluten-free diet, vitamin and mineral supplements, steroids to control inflammation, and other drugs, such as azathioprine or budesonide.

In some embodiments, the normalization and correction comprise background correction and dye bias correction. In some embodiments, the resulting signal intensities are normalized using the ‘quantile normalization’ method. In some embodiments, CpGs associated with X and Y chromosomes, single nucleotide polymorphisms (SNPs) and repeats, non-specific or cross-reactive probes and probes showing low variability and extreme methylation values are removed.

### BRIEF DESCRIPTION OF THE DRAWINGS

FIG. 1. DNA methylation-based diagnostic algorithm for ruling-in IBS. Dx, diagnosis; IBS, irritable bowel syndrome; IBD, inflammatory bowel disease.

FIG. 2. DNA methylation-based diagnostic algorithm for ruling out IBS and/or ruling-in IBD based on fecal calprotectin/C-reactive protein (CRP) levels. Dx, diagnosis; IBS, irritable bowel syndrome; IBD, inflammatory bowel disease.

FIG. 3. DNA methylation-based diagnostic algorithm for ruling out IBS or ruling-in celiac disease based on celiac serologies. Dx, diagnosis; IBS, irritable bowel syndrome; IBD, inflammatory bowel diseases.

FIG. 4. Double cross validation process includes two nested cross-validation loops referred to as outer and inner loops. The entire data are divided into training and test sets (clear and hashed rectangles of “Outer loop”, respectively). The training set, which is further divided into tuning/calibration and validation sets (clear and hashed rectangles at right, respectively), is used as an inner loop. The calibration set is used for model building (tuning hyperparameters) and the validation set is used to estimate the errors. The model with the lowest prediction error within the inner loop is selected as the best model. Then the test set in the outer loop is used to assess the predictive performance of the model. Multiple splits (N=10) of inner and outer datasets avoids the bias with respect to variable selection resulting from use of a single training set.

FIG. 5. Receiver operating characteristic (ROC) curves and the area under the ROC curves (AUC) for IBS vs IBD, IBS vs celiac disease and IBD vs celiac disease comparisons. The x-axis represents the sensitivity and γ-axis represents the specificity, and each point on the ROC curve represents a sensitivity/specificity pair corresponding to a particular decision threshold. The AUC values for IBS vs IBD, IBS vs celiac disease and IBD vs celiac disease classifiers were 0.85, 0.82 and 0.78.

FIG. 6. Correlation between DNA methylation principal components and covariates shows correlation values (r) for correlations between six principal components of DNA methylation data in IBS and healthy control subjects and potential confounders. Age and chip/batch were used as covariates for all differential methylation analyses and cell-types were used as additional covariates for machine-learning models.

FIGS. 7A-7F. Differentially methylated CpG across chromosomal regions for IBS versus IBD (7A), IBS versus CeD (7B), IBD versus CeD (7C), IBS versus healthy controls (7D), IBD versus healthy controls (7E), and CeD versus healthy controls (7F). Manhattan plots are depicted to visualize distribution of differentially methylated CpG site-associated genes across chromosomal regions. Hashed circles are CpGs with a p-value corresponding to FDR<0.05 in IBS vs IBD, IBD vs healthy controls, IBD vs CeD and CeD vs healthy controls. For IBS vs healthy controls, hashed dots represent p<0.0005.

FIGS. 8A-8C. Gene ontology terms associated with classifiers. FIG. 8A shows the GO terms enriched in IBS classifier-associated genes in: IBS vs healthy control (first panel), IBS vs IBD (middle panel), and IBS vs Celiac disease (third panel) comparisons. Cell adhesion, Neuronal pathways and transport-associated genes were among the enriched GO terms represented in all the three comparisons. FIG. 8B shows the GO terms enriched in IBD classifier-associated genes in: IBD vs healthy control (first panel), IBD vs IBS (middle panel), and IBD vs Celiac disease (third panel) comparisons. Inflammation-related terms were represented in all the comparisons. FIG. 8C shows the GO terms enriched in Celiac disease classifier-associated genes in: Celiac disease vs healthy control (first panel), Celiac disease vs IBS (second panel), and Celiac disease vs IBD comparisons (third panel). Major histocompatibility complex (MHC) and antigen presentation pathways were among the top terms in all the three celiac-associated classifiers.

FIG. 9. ROC curve for testing IBD versus healthy control classifiers on external validation data.

FIG. 10. Permutation testing of classifiers associated with the pairs of diagnostic groups.

### DETAILED DESCRIPTION

Described herein are methods of utilizing blood-based genome-wide DNA methylation profiles to develop a reliable test to diagnose irritable bowel syndrome (IBS) versus inflammatory bowel disease (IBD), celiac disease and other gastrointestinal diseases that mimic IBS. These methods provide a means to rule out IBS and rule-in active IBD or celiac disease, as well as to rule-out active IBD and celiac disease, and to rule-in IBS.

### Definitions

All scientific and technical terms used in this application have meanings commonly used in the art unless otherwise specified. As used in this application, the following words or phrases have the meanings specified.

As used herein, a “control” or “reference” sample means a sample that is representative of normal measures of the respective marker, such as would be obtained from normal, healthy control subjects, or a baseline amount of marker to be used for comparison. Typically, a baseline will be a measurement taken from the same subject or patient. The sample can be an actual sample used for testing, or a reference level or range, based on known normal measurements of the corresponding marker.

As used herein, a “significant difference” means a difference that can be detected in a manner that is considered reliable by one skilled in the art, such as a statistically significant difference, or a difference that is of sufficient magnitude that, under the circumstances, can be detected with a reasonable level of reliability. In one example, an increase or decrease of 10% relative to a reference sample is a significant difference. In other examples, an increase or decrease of 20%, 30%, 40%, or 50% relative to the reference sample is considered a significant difference. In yet another example, an increase of two-fold relative to a reference sample is considered significant.

“Nucleotide sequence” refers to a heteropolymer of deoxyribonucleotides, ribonucleotides, or peptide-nucleic acid sequences that may be assembled from smaller fragments, isolated from larger fragments, or chemically synthesized de novo or partially synthesized by combining shorter oligonucleotide linkers, or from a series of oligonucleotides, to provide a sequence which is capable of expressing the encoded protein.

As used herein, the term “probe” refers to an oligonucleotide, naturally or synthetically produced, via recombinant methods or by PCR amplification, that hybridizes to at least part of another oligonucleotide of interest. A probe can be single-stranded or double-stranded. In the context of an array as described herein, a probe can be methylation-specific.

As used herein, “hybridizes,” “hybridizing,” and “hybridization” means that the oligonucleotide forms a noncovalent interaction with the target DNA molecule under standard conditions. Standard hybridizing conditions are those conditions that allow an oligonucleotide probe or primer to hybridize to a target DNA molecule. Such conditions are readily determined for an oligonucleotide probe or primer and the target DNA molecule using techniques well known to those skilled in the art. The nucleotide sequence of a target polynucleotide is generally a sequence complementary to the oligonucleotide primer or probe. The hybridizing oligonucleotide may contain nonhybridizing nucleotides that do not interfere with forming the noncovalent interaction. The nonhybridizing nucleotides of an oligonucleotide primer or probe may be located at an end of the hybridizing oligonucleotide or within the hybridizing oligonucleotide. Thus, an oligonucleotide probe or primer does not have to be complementary to all the nucleotides of the target sequence as long as there is hybridization under standard hybridization conditions.

The term “complement” and “complementary” as used herein, refers to the ability of two nucleic acid molecules to base pair with each other. For example, in DNA, adenine (A) is complementary to thymine (T). In RNA, adenine (A) is complementary to uracil (U). In some embodiments, complementarity refers to an antisense compound that is capable of base pairing with its target nucleic acid. For example, if a nucleobase at a certain position of an antisense compound is capable of hydrogen bonding with a nucleobase at a certain position of a target nucleic acid, then the position of hydrogen bonding between the oligonucleotide and the target nucleic acid is considered to be complementary at that nucleobase pair. Nucleobases comprising certain modifications may maintain the ability to pair with a counterpart nucleobase and thus, are still capable of nucleobase complementarity. Typically, two DNA molecules are complementary if they hybridize under the standard conditions referred to above. Typically, two DNA molecules are complementary if they have at least about 80% sequence identity, preferably at least about 90% sequence identity.

As used herein, the term “subject” includes any human or non-human animal. The term “non-human animal” includes all vertebrates, e.g., mammals and non-mammals, such as non-human primates, horses, sheep, dogs, cows, pigs, chickens, and other veterinary subjects. In a typical embodiment, the subject is a human.

As used herein, “a” or “an” means at least one, unless clearly indicated otherwise.

For use in the methods described herein, representative examples of the sample include, but are not limited to, blood, plasma or serum, saliva, urine, cerebral spinal fluid, milk, cervical secretions, semen, tissue, cell cultures, and other bodily fluids or tissue specimens.

### Methods

Described herein are methods of classifying biological samples based on gastrointestinal disease. In some embodiments, the method comprises: (a) measuring the amount of DNA methylation at CpG sites in DNA in a biological sample obtained from the subject to generate a genome-wide DNA CpG methylation profile of the subject, wherein the measuring comprises probing at least 200,000 CpG sites; (b) performing a normalization of technical noise and bias correction on the profile; (c) filtering the profile to retain data from a first set of probed CpG sites that is the same as a first set of CpG sites from a first training data set trained on a first pairwise comparison between biological samples, wherein the first pairwise comparison is selected from: (i) irritable bowel syndrome (IBS) and inflammatory bowel disease (IBD); (ii) IBS and celiac disease (CeD); (iii) IBD and CeD; (iv) IBS and healthy control; (v) IBD and healthy control; and (vi) CeD and healthy control; and (d) classifying the CpG methylation profile of the biological samples as IBS, IBD, CeD, or healthy based on a probability cutoff derived from the first training data set. The classifying can be implemented by use of a computer. Also provided is a system for performing the methods described herein.

In some embodiments, the filtering of step (c) is repeated to retain data from a second set of probes that is the same as a second set of probes from a second training data set trained on a second pairwise comparison between biological samples, wherein the second pairwise comparison is selected from (i) to (vi), and wherein the second pairwise comparison differs from the first pairwise comparison.

In some embodiments, the filtering of step (c) is repeated to retain data from subsequent sets of probes corresponding to subsequent training data sets trained on subsequent pairwise comparisons between biological samples, wherein each of the remaining pairwise comparisons of (i) to (vi) is performed.

In some embodiments, the probing comprises contacting the biological samples with a methylation microarray. In some embodiments, the biological sample comprises blood, plasma, serum, or mucosal tissue. In some embodiments, the sample is peripheral blood mononuclear cells (PBMCs), peripheral blood lymphocytes (PBL), or whole blood.

In some embodiments, the method further comprises measuring the amount of fecal calprotectin and/or blood C-reactive protein (CRP) in the biological sample. In some embodiments, 120 mcg/g FCP is the cut off for determining likelihood of IBD. In some embodiments, 150 mcg/g FCP is the cut off for determining likelihood of IBD. Values significantly higher, especially those over 250 μg/g, are strongly indicative of active IBD. In some embodiments, the cutoff is 8-10 mg/l CRP for IBD. In some embodiments, the cutoff is 8 mg/l CRP for IBD. In some embodiments, an elevated (>4 U/ml) anti-tissue transglutaminase antibody is indicative of CeD.

In some embodiments, the training data set is trained using GLMNET algorithm. In some embodiments, the training data set is trained using regularization parameter value and elastic net mixing parameter. This parameter minimizes the misclassification error.

In some embodiments, the normalization and correction comprise background correction and dye bias correction. In some embodiments, the resulting signal intensities are normalized using the ‘quantile normalization’ method. In some embodiments, CpGs associated with X and Y chromosomes, single nucleotide polymorphisms (SNPs) and repeats, non-specific or cross-reactive probes and probes showing low variability and extreme methylation values are removed.

Also described is a method of screening for, diagnosing, or detecting irritable bowel syndrome (IBS), inflammatory bowel disease (IBD) and/or celiac disease (CeD) in a subject. In some embodiments, the method comprises performing the method of classifying described herein on a biological sample obtained from the subject, and identifying the subject as having IBS, IBD, or CeD based on the classifying of step (d). In some embodiments, the method further comprises treating the subject for IBS, IBD, or CeD.

### Treatment for Gastrointestinal Conditions

Treatment for IBS can include fiber supplements, laxatives, anti-diarrheal medications, such as loperamide, a bile acid binder, such as cholestyramine, colestipol or colesevelam, anticholinergic medications, such as dicyclomine or hysocyamine, tricyclic antidepressants, such as amitriptyline, imipramine, desipramine or nortriptyline, serotonin and norepinephrine reuptake inhibitor (SNRI) antidepressants, such as duloxetine, selective serotonin reuptake inhibitor (SSRI) antidepressants, such as fluoxetine or paroxetine, tetracyclic antidepressant, such as mirtazapine, pregabalin or gabapentin. Medications approved for certain people with IBS include: Alosetron, Eluxadoline, Rifaximin, Lubiprostone, Plecanatide, Tenapanor and Linaclotide.

Treatment for IBD can include anti-inflammatory drugs, which include aminosalicylates, such as mesalamine, balsalazide and olsalazine, and immune system suppressors. Time-limited courses of corticosteroids can be both anti-inflammatory and immunosuppressing. Some examples of immunosuppressant drugs include azathioprine, mercaptopurine and methotrexate. Small molecules can also be used for IBD treatment. These include tofacitinib, upadacitinib and ozanimod. Biologics for treating IBD include infliximab, adalimumab, golimumab, certolizumab, vedolizumab, ustekinumab, and risankizumab. In some cases, treatment can include antibiotics, anti-diarrheal medications, such as psyllium powder or methylcellulose, and/or loperamide, pain relievers, and vitamins and supplements.

Treatment for CeD can include gluten-free diet, vitamin and mineral supplements, steroids to control inflammation, and other drugs, such as azathioprine or budesonide.

### EXAMPLES

The following examples are presented to illustrate the present invention and to assist one of ordinary skill in making and using the same. The examples are not intended in any way to otherwise limit the scope of the invention.

### Example 1: Development of Blood-Based Genome-Wide DNA Methylation Profiles

This Example demonstrates describe the analytic process of utilizing blood-based genome-wide DNA methylation profiles to develop a reliable test to diagnose irritable bowel syndrome (IBS) vs inflammatory bowel disease (IBD), celiac disease and other gastrointestinal diseases that mimic IBS.

### Methods

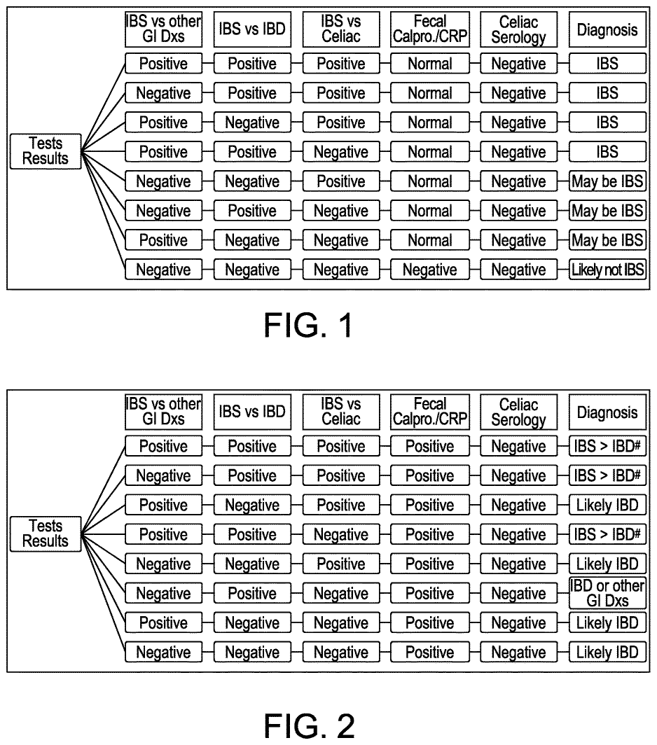

The methods described herein have been applied to Infinium MethylationEPIC BeadChip, Illumina™ DNA methylation array, which enables interrogation of over 850,000 methylation sites quantitatively at single nucleotide resolution. The analysis is performed in three steps, data preprocessing, classifier development and evaluation, and diagnostic algorithm development. Each step involves implementation of a set of codes written in R statistical language (1).
1. Data preprocessing (package names Italicized with no quotes, functions Italicized with single quotes and methods or object classes normal font with double quotes):
1.1. Data import and normalization: As outlined in FIG. 1, this part of analysis includes steps to preprocess the raw Illumina DNA methylation array (IDAT) files generated by the Illumina iScan scanner and output normalized and filtered data. Data preprocessing was performed using ENmix package (2). Quality Control (QC) metrics were generated using ‘QCinfo’ function. Samples that did not pass the QC threshold were eliminated. Using ‘preprocessENmix’ function, we used “oob” method for background correction and “RELIC” for dye bias correction. This resulted in signal intensity data on methylated and unmethylated channels of “MethylSet” class object. The data were quantile normalized using ‘norm.quantile’, separately for Methylated and Unmethylated intensities for Infinium I or Infinium II probes (“quantile1” method). Probe design type bias was corrected using Regression on Correlated Probes (RCP) method, followed by filtering on QC metrics. Detection P value threshold to identify low quality data point was set to 0.000001. “Impute” parameter was set to “True” (after removing rows or columns with too many missing values, default threshold of >5% missing values). All the steps were combined into a single function which can be applied to either single or multiple IDAT files. Cell types were estimated using ‘estimateCellCounts2’ function and “preprocessNoob” method for Epic platform in FlowSorted.Blood.EPIC package (3). Methylation age was predicted using “methyAge” function.
1.2. Probe filtration: Of 855,790 CpGs on the array, QC information-based filtering resulted in 846,790 probes. Additionally, a file with non-informative probes was created with unique probe IDs that were excluded (X and Y chromosome derived using IlluminaHumanMethylationEPICanno.ilm10b2.hg19 package, n=18,880; probes with SNPs and repeats, n=25,329; and non-specific or cross-reactive probes, n=29,233; a total of 778,660 unique probes). Data were analyzed with and without correction for batch effects (technical variability due to pipetting errors between batches of samples that were run together on a 96 well plate). For the batch effect adjusted analyses, the batch effects were removed using ‘removeBatchEffect’ function separately for training and test folds from the “methylSet” object.

### 1.3. Sample Subgrouping

Epic methylation data were available on 384 subjects. After normalization and initial filtering, data were split into PBMC and Blood. The breakdown of the number of samples per group for PBMCs and whole blood is shown in Table 1.

PBMC and blood data were further filtered separately to exclude probes with low variability (standard deviation <0.02) and those very high or very low average beta values (beta <0.1 or >0.9), which are likely to be skewed (4). This resulted in 282,250 probes which were used as an input for various analysis comparing groups. This number can change based on the nature and the number of samples included as well as the cutoff used for each of the above filtering steps. The PBMC and blood data were further subdivided into the following comparison groups which belonged to one of the two categories including 1. one vs one: comparing one diagnosis group (Dx) vs healthy control (HC) group or one Dx vs other Dx, and 2. one vs all other Dxs combined. Although the results presented below do not include HC in the 2nd category, it may be potentially included to improve model performance. The GI disease controls included celiac disease, inflammatory bowel disease (IBD; Crohn's disease and ulcerative colitis), and colon cancer. The resulting comparison groups included:

### Comparisons of IBS Vs Single Other GI Disease Control

IBS vs HC, IBS as disease and healthy control (HC) as control group
IBS vs IBD, IBS as disease and IBD as control group
IBS vs Celiac, IBS as disease and celiac as control group
IBD vs HC, IBD as disease and HC as control group
Celiac vs HC, Celiac as disease and HC as control group
IBD vs Celiac, IBD as disease and Celiac as control group One Dx vs all other Dxs
IBS vs Celiac+IBD+Colon cancer
IBD vs IBS+Celiac+Colon cancer
Celiac vs IBS+IBD+Colon cancer

### 2. Classifier Development and Evaluation

### 2.1. Generating Training and Test Fold Data

We implemented nested folds to split the data into 10 folds of outer training set and outer test set (90:10). The outer training set was further split into inner training and inner test set (90:10), for choosing model hyperparameters. To account for age, age was either added as a variable in both training and test datasets or the effect of age was regressed out using linear regression for training and test data separately.

### 2.2. Selection of Model Hyperparameters

For each dataset, models on training data were generated using generalized linear models (GLM) via penalized maximum likelihood using glmnet package. Regularization parameter (A) was selected using 10-fold cross-validation on inner training and test data. A model corresponding to A that minimizes the error of cross-validation (Amin) was chosen and prediction was made for each of the 10 outer training-test data splits. Separate models were generated for lasso or elastic net penalties (α=0, 0.1, 0.2, . . . , 1), and the model associated with a resulting in the best performance was chosen as the final model.

Glmnet is one of the >200 available methods to train a model using machine learning. A comprehensive list of methods is compiled here-https://topepo.github.jo/caret/available-models.html. Additionally, newer methods are constantly being developed which can fine-tune the hyperparameters to enhance model performance.

Additionally, an ensemble of methods can be utilized to test multiple models, which may perform better than individual models. For example, in our analysis, individual models were not successful at predicting patterns in the whole blood samples with a limited sample size, whereas, the ensemble method performed better and resulted in more significant predictions.

### 2.3. Evaluation of Model Performance

Various performance metrics were generated based on the 2×2 confusion matrix for each of the comparison, created using caret package and GLMs. These included p-values from linear regression of predicted and true values, accuracy (confidence intervals), accuracy p values, kappa, sensitivity, specificity, positive predictive value (PPV), negative predictive values (NPV), precision, recall, F1, prevalence and balanced accuracy. In these analyses, a model with a low p value (<0.05) and highest sensitivity, and specificity were chosen as the best models. However, all other metrics mentioned above help in addressing different aspects of model performance.

### Results

Accuracy of prediction models: Table 2 shows the performance metrics based on the number of correct and incorrect predictions on new (test) samples. The accuracy of discrimination between IBS vs HC was 0.6 (p<0.017), however, the specificity was low (0.43). Models developed for IBS vs IBD and IBS vs Celiac disease performed better with an accuracy of 0.76 and 0.77, respectively. The performance of IBD vs HC and Celiac vs HC models better, with accuracies 0.82 and 0.84, respectively.

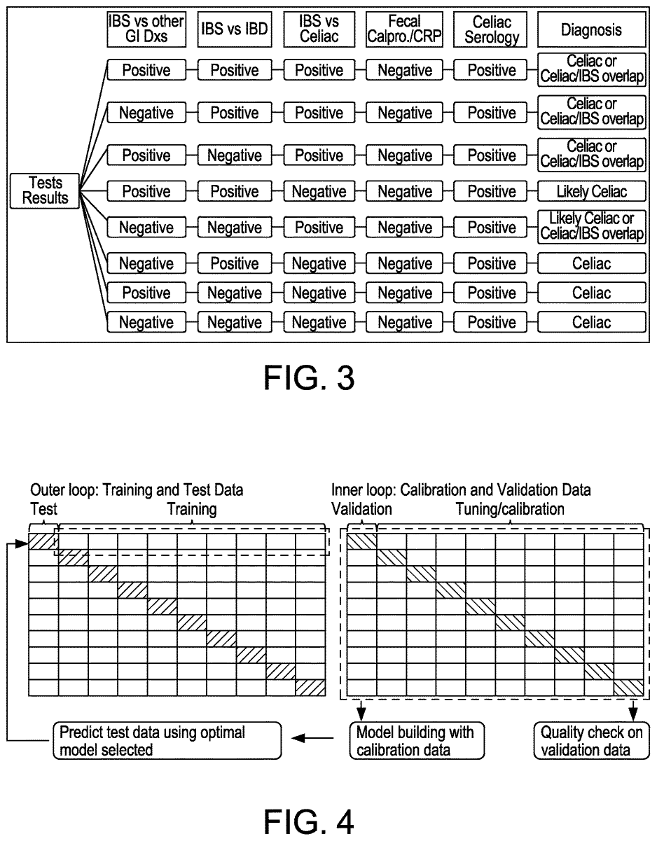

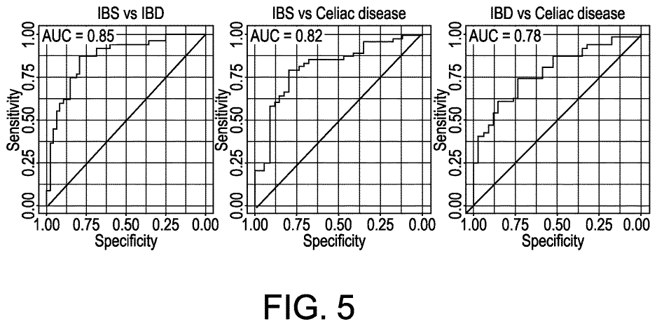

Diagnostic algorithm: Based on the probabilistic models described above, which identify linear and non-linear relationships between DNA methylation and GI diseases, we designed an algorithm that can be used to diagnose IBS. The first three tests involve DNA methylation pattern-based classification of incoming sample into IBS vs IBD, IBS vs celiac disease, and IBS vs other GI diseases (e.g., microscopic colitis, bile acid diarrhea, colon cancer) where three positive tests indicate a high likelihood of a positive IBS diagnosis. In the case of three negative tests, this would indicate that the diagnosis is very unlikely to be IBS. In the case of at least two positives out of the three tests, it suggests a probable diagnosis of IBS but may not confidently rule out other GI diseases that present with IBS-like symptoms. Additionally, a negative diagnosis of IBS on at least two of the three tests suggests a low likelihood of IBS. In these situations, IBD and celiac disease non-invasive markers including fecal calprotectin/C-reactive protein (CRP) and celiac serologies, respectively, can help to rule out IBS and rule-in active IBD (e.g., elevated fecal calprotectin and/or CRP) or celiac disease (positive celiac serologies) or rule-out active IBD and celiac disease (normal fecal calprotectin and CRP, and negative celiac serologies, respectively). Fecal calprotectin and/or C-reactive protein (CRP) and celiac serologies can further help with the diagnosis of IBS. For example, normal fecal calprotectin levels <50 has been shown to be associated with a <1% chance of having IBD (i.e., ruling out IBD) and those between 50 and 150 are still suggestive of IBS (if the DNA methylation-based comparisons of IBS vs IBD, IBS vs celiac, IBS vs other GI diseases are positive), mildly active IBD, gastrointestinal infection or microscopic colitis. A higher fecal calprotectin, e.g. >150, will be more suggestive of an inflammatory or infectious GI disorder, e.g., IBD (5). Similarly, abnormal celiac serologies would rule-out IBS alone and warrant diagnostic workup for celiac disease. In general, abnormal fecal calprotectin/C-reactive protein (CRP) or celiac serologies would require additional diagnostic workup for IBD, celiac disease and other organic GI diseases such as microscopic colitis. The specific steps involved in making diagnostic decisions are presented in Table 2 and a workflow is shown in FIG. 1. Some patients will have already undergone an upper endoscopy and colonoscopy with biopsies and small bowel imaging tests which show no evidence of celiac disease, IBD, GI cancer, microscopic colitis, which would increase the probability that the patient has IBS as long as they meet diagnostic symptom criteria for IBS. This would be valuable in clinical practice because the blood test would confirm the diagnosis of IBS, i.e., be a rule-in test for IBS. Additionally, similar algorithms can be implemented to diagnose pediatric IBS, IBD and celiac disease using PBMCs and whole blood samples (FIGS. 2 and 3).

The diagnostic performance of a ‘rule-in’ IBS test can be further improved by developing algorithms to diagnose IBS from symptomatic patients who have a diagnostic test that is negative for IBD, celiac and other GI diseases, e.g., colon cancer.

To make the diagnostic call, DNA methylation data from a new sample will be generated using the EPIC platform and processed through the preprocessing steps followed by the three DNA methylation-based classification models to output class labels for the most probable prediction. The votes for each class will be counted and information from fecal calprotectin/C-reactive protein (CRP) or celiac serologies will be used to make the final diagnostic calls.

### CONCLUSIONS

Although no single set of DNA methylation-based classifier was able to diagnose IBS with a high sensitivity and specificity, we developed an algorithm which utilizes the information based on models developed using GI disease controls in diagnosing IBS with a greater confidence.

### REFERENCES

Team RC. R: A language and environment for statistical computing. Vienna, Austria: R Foundation for Statistical Computing; 2020.
Xu Z, Niu L, Li L, Taylor J A. Nucleic Acids Res. 2016; 44 (3): e20.
Salas L A, Koestler D C. Illumina EPIC data on immunomagnetic sorted peripheral adult blood cells. 2020.
Mansell G, et al. BMC Genomics. 2019; 20 (1): 366.
Menees S B, et al. Am J Gastroenterol. 2015; 110 (3): 444-54.
American Gastroenterological A. Gastroenterology. 2019; 157 (3): 856-7.
Lacy B E, et al. Am J Gastroenterol. 2021; 116 (1): 17-44.

TABLE 1
Patient population

| | | | | | | Healthy | | |
|---|---|---|---|---|---|---|---|---|
| | | | | | Colon | Controls | Pediatric | Pediatric |
| | Crohn's | UC | IBS | Celiac | Cancer | (HCs) | HCs | IBS |
| PBMCs | 33 | 41 | 52 | 43 | 7 | 68 | 13 | 17 |
| Whole | 14 | 14 | 10 | 27 | 5 | 10 | 13 | 12 |
| Blood | | | | | | | | |

TABLE 2
Performance of model predictions

| | IBS VS | | IBS vs | | | IBS vs | IBD vs |
|---|---|---|---|---|---|---|---|
| | healthy | | Celiac (IBS | | | Disease | Celiac (IB |
| | control | IBS vs | as Dx | IBD vs | Celiac | controls (IBD + | as Dx |
| | (HC) | IBD | group) | HC | vs HC | Celiac + CC) | group) |
| AUC | 0.62 | 0.781 | 0.765 | 0.87 | 0.86 | 0.71 | 0.824 |
| Accuracy | 0.6 | 0.758 | 0.77 | 0.82 | 0.84 | 0.71 | 0.765 |
| Precision | 0.54 | 0.712 | 0.85 | 0.87 | 0.79 | 0.53 | 0.804 |
| (PPV) | | | | | | | |
| Sensitivity | 0.79 | 0.87 | 0.73 | 0.72 | 0.79 | 0.67 | 0.787 |
| (Recall) | | | | | | | |
| Specificity | 0.43 | 0.64 | 0.82 | 0.91 | 0.87 | 0.74 | 0.735 |
| NPV | 0.71 | 0.833 | 0.68 | 0.8 | 0.87 | 0.80 | 0.714 |
| P value | 0.017 | <0.001 | <0.001 | <0.001 | <0.001 | <0.001 | <0.001 |
| Confusion | TN = 24 | TN = 30 | TN = 28 | TN = 51 | TN = 49 | TN = 65 | TN = 25 |
| matrix | FN = 10 | FN = 6 | FN = 13 | FN = 13 | FN = 7 | FN = 16 | FN = 10 |
| | FP = 32 | FP = 17 | FP = 6 | FP = 5 | FP = 7 | FP = 23 | FP = 9 |
| | TP = 38 | TP = 42 | TP = 35 | TP = 34 | TP = 27 | TP = 32 | TP = 37 |

This table shows the performance metrics for classification models developed for each comparison mentioned in columns. IBD, inflammatory bowel diseases; IBS, irritable bowel syndrome.

FIG. 1 shows the possible combinations of DNA methylation tests and resulting diagnoses when the Fecal Calprotectin, C-reactive protein (CRP) and celiac serologies are negative. These non-invasive tests are currently recommended in gastroenterology society guidelines for IBS even in the setting without red flags (6, 7). The first column (IBS vs GI diseases other than IBD and celiac) represents the diagnosis based on an algorithm trained to classify IBS patients from other GI diagnoses such as non-celiac gluten sensitivity, microscopic colitis, bile acid diarrhea, colon cancer, etc., which may or may not present with red flags (weight loss, blood in stool, etc.). When all the tests are negative the patient is likely not IBS. However, they may still be IBD in remission or celiac disease on a gluten free diet.

FIG. 2 shows the possible combinations of DNA methylation tests and resulting diagnoses when the Fecal Calprotectin and/or C-reactive protein (CRP) are positive. The first column (IBS vs other GI diseases) represents diagnosis based on an algorithm trained to classify IBS patients from other GI diagnoses such as non-celiac gluten sensitivity, microscopic colitis, colon cancer, bile acid diarrhea, which may or may not present with red flags (weight loss, blood in stool, etc.). ^{#}The diagnosis is more likely IBS than IBD if the fecal calprotectin and/or CRP levels are only mildly elevated, e.g., fecal calprotectin level >50 but <150. If the fecal calprotectin and/or CRP levels are moderately or greatly increased, the probability of having IBS is very low and the probability of having IBD is high.

Additionally, more than one positive DNA methylation test for IBS diagnosis suggests a greater likelihood for IBS compared to IBD.

FIG. 3 shows the possible combinations of DNA methylation tests and resulting diagnoses when the celiac serologies are positive. The first column (IBS vs other GI diseases) represents diagnosis based on an algorithm trained to classify IBS patients from other GI diagnoses such as non-celiac gluten sensitivity, microscopic colitis, bile acid diarrhea, colon cancer, etc.

### Example 2: Representative Workflow

This Example illustrates the steps one can take to prepare, train, and analyze DNA methylation data using a machine learning model. Once a model has been trained, test samples can be evaluated using corresponding parameters. This approach can be used to provide a non-invasive system for guiding detection and diagnosis of gastrointestinal disorders and facilitate effective treatment.

### Input Data Preparation

The preprocessing steps are aimed at preparing DNA methylation data obtained as a raw scanner output to be used as an input into a machine learning models. There are 855,790 CpGs on Epic v1.0 array. The following filtering and preprocessing steps were standardized which use ENmix R package.
1. Background and dye bias correction performed using ‘preprocessENmix’
2. Quality control performed using ‘QCinfo’ with a detection P threshold of 1e-6 and data quantile normalized using ‘norm.quantile’ function
3. Probe design type bias correction using Regression on Correlated Probes (rcp function) and the resulting betas [Methylated/(Methylated+Unmethylated probes)] filtered based on QC metrics resulting in 846,790 probes.
4. Additional filtering by exclusion of X and Y chromosomal probes (n=18,880), probes with SNPs and repeats (n=25,329), and non-specific or cross-reactive probes (n=29,233) resulting in 78,660 unique probes.
5. Annotation of probes using IlluminaHumanMethylationEPICanno.ilm10b2.hg19 package.
6. Sample selection to include disease pairs.
7. Exclusion of probes with a low variance (standard deviation <0.02) and extreme beta values (beta <0.1 or >0.9) resulting in 180,000 to 250,000 probes depending on the diseases in question.
8. Preparation of metadata file with patient details including age, DNA methylation run or batch number, and cell-type composition.

### Training Process

After normalization and initial filtering, we prepared the data for input into the machine learning models. Table 3 shows all the comparison groups tested and the methylation platforms used to generate the data. While comparisons involving IBD and celiac disease including IBS vs IBD, IBS vs celiac and IBD vs celiac were performed on Epic array data, IBS vs HC was performed using three separate analyses. The first one included a larger set of IBS and HC samples that were run on Epic or 450K methylation array. This analysis used the platform type as the batch covariate. The second analysis included IBS and HC samples that were down-sampled to include balanced sample numbers for IBS and HCs to avoid over-fitting. The third analysis included IBS patient and HC samples that were run on Epic platform only. This step allowed for identification of differential methylation on probes that were not represented on 450K platform. All comparisons are shown in Table 3.

TABLE 3
Comparison groups and DNA methylation platforms

| Comparison groups | Platform | N Group 1 | N Group 2 |
|---|---|---|---|
| IBS vs IBD | Epic | 48 | 43 |
| IBS vs Celiac | Epic | 48 | 34 |
| IBD vs Celiac | Epic | 43 | 34 |
| IBS vs Healthy Control | 1. Epic only | 1. 48 | 1. 55 |
| | 2. Epic + 450K | 2. 148 | 2. 86 |
| | 3. Down-sampled | 3. 86 | 3. 86 |
| | Epic + 450K | | |
| IBD vs Healthy Control | Epic | 43 | 55 |
| Celiac vs Healthy Control | Epic | 34 | 55 |

### Covariates

Age, cell type proportions (six cell types including CD8-T cells, CD4-T cells, natural killer cells, B-cells, monocytes, neutrophils) and DNA methylation batch were included as covariates in the model along with the set of pre-filtered and normalized DNA methylation probes. IBS vs HC analysis for the larger dataset included platform (450K or Epic) as the batch covariate. For analyses involving IBD samples, sample collection site was used as an additional batch covariate.

### Structure of the Model

The unique features of the models include the use of GLMNET algorithm, and a combination of regularization parameter (A) value and elastic net mixing parameter (a, range 0 [ridge] to 1 [lasso]). To identify the best fitting model, we implemented a 10-fold cross validation with 10 repeats. Specifically,
1. Split the dataset into 10 parts using function ‘createFolds’ function from ‘caret’ package.
2. For each set:
   a. One in ten samples was used as a hold-out sample
   b. Model trained on the remaining samples
   c. Within the training data set, generalized linear models (GLM) via penalized maximum likelihood using glmnet package were fitted with model hyperparameters tuned to obtain regularization parameter (A) that minimizes the error (λmin) using ‘cv.glmnet’ function. Specific arguments included, type.measure=“deviance”, family=“binomial”, parallel=TRUE, nfolds=10
   d. One model for each elastic net mixing parameter (a, range 0 [ridge] to 1 [lasso]) was fitted
3. The step 2 resulted in one best performing model for every a. After repeating this on various training: test splits (from step 1), summarized output for all repeats was generated which included the following evaluation scores:
   a. Probability cutoff
   b. Alpha (a)
   c. AUC
   d. Accuracy
   e. Kappa
   f. Accuracy Lower Confidence Interval (CI)
   g. Accuracy Upper CI
   h. Accuracy Null
   i. Accurac P value
   j. Mcnemar P Value
   k. Sensitivity
   l. Specificity
   m. Pos Pred Value
   n. Neg Pred Value
   o. Precision
   p. Recall
   q. F1
   r. Probability cutoff
   s. Prevalence
   t. Detection Rate
   u. Detection Prevalence
   v. Balanced Accuracy
   w. True Negatives (TN)
   x. False Positives (FP)
   y. False Negatives (FN)
   z. True Positives (TP)
4. An accuracy p-value of <0.05 was considered significant and the best performing model was defined as a model with the lowest accuracy p-value and a high balanced accuracy value when multiple models have a low p-value. The model outputted a set of classifiers (one for each repeat) for each pair of the GI diseases in question.

TABLE 4A

| | IBS vs HC | | |
|---|---|---|---|
| | Epic + 450K | | IBS vs |
| | down-sampled | IBS vs IBD | Celiac |
| Probability Cutoff | 0.44 | 0.59 | 0.53 |
| Alpha (α) | 0.5 | 0.6 | 0.9 |
| AUC | 0.69 (0.61-0.77) | 0.85 (0.77-0.93) | 0.82 (.72-0.91) |
| Accuracy | 0.69 | 0.80 | 0.78 |
| Kappa | 0.37 | 0.59 | 0.55 |
| Accuracy Lower | 0.61 | 0.70 | 0.68 |
| Accuracy Upper | 0.75 | 0.87 | 0.86 |
| Accuracy Null | 0.5 | 0.52 | 0.59 |
| Accuracy P-Value | 5.94E−07 | 2.22E−08 | 1.60E−04 |
| Mcnemar P-Value | 0.13 | 1.00 | 1.00 |
| Sensitivity | 0.76 | 0.79 | 0.81 |
| Specificity | 0.62 | 0.8 | 0.74 |
| Pos Pred Value | 0.66 | 0.81 | 0.81 |
| Neg Pred Value | 0.72 | 0.78 | 0.74 |
| Precision | 0.66 | 0.81 | 0.81 |
| Recall | 0.76 | 0.79 | 0.81 |
| F1 | 0.71 | 0.80 | 0.81 |
| Prevalence | 0.5 | 0.52 | 0.59 |
| Detection Rate | 0.38 | 0.41 | 0.48 |
| Detection | 0.57 | 0.51 | 0.59 |
| Prevalence | | | |
| Balanced | 0.69 | 0.80 | 0.77 |
| Accuracy | | | |
| TN | 53 | 36 | 25.00 |
| FP | 33 | 9 | 9.00 |
| FN | 21 | 10 | 9.00 |
| TP | 65 | 38 | 39.00 |

TABLE 4B

| | IBD vs HC | Celiac vs HC | IBD vs Celiac |
|---|---|---|---|
| Probability Cutoff | 0.35 | 0.34 | 0.57 |
| Alpha (α) | 0.2 | 0.2 | 0.3 |
| AUC | 0.92 (0.88-0.97) | 0.85 | 0.78 (0.67-0.88) |
| Accuracy | 0.82 | 0.8 | 0.73 |
| Kappa | 0.65 | 0.58 | 0.46 |
| Accuracy Lower | 0.73 | 0.70 | 0.62 |
| Accuracy Upper | 0.89 | 0.88 | 0.83 |
| Accuracy Null | 0.55 | 0.62 | 0.57 |
| Accuracy P-Value | 1.33E−08 | 2.23E−04 | 1.84E−03 |
| Monemar P-Value | 0.10 | 0.81 | 0.66 |
| Sensitivity | 0.89 | 0.76 | 0.73 |
| Specificity | 0.77 | 0.82 | 0.74 |
| Pos Pred Value | 0.75 | 0.72 | 0.79 |
| Neg Pred Value | 0.90 | 0.85 | 0.68 |
| Precision | 0.75 | 0.72 | 0.79 |
| Recall | 0.89 | 0.76 | 0.73 |
| F1 | 0.82 | 0.74 | 0.76 |
| Prevalence | 0.45 | 0.38 | 0.57 |
| Detection Rate | 0.40 | 0.29 | 0.42 |
| Detection | 0.52 | 0.4 | 0.53 |
| Prevalence | | | |
| Balanced Accuracy | 0.83 | 0.79 | 0.73 |
| TN | 43 | 46 | 25 |
| FP | 13 | 10 | 9 |
| FN | 5 | 8 | 12 |
| TP | 40 | 26 | 33 |

Preparing and Running the New Sample Outside of this Study

Preprocessing steps of DNA methylation data generated using Illumina Epic array will include normalization of technical noise as described in the “Input Data Preparation” section and filtering the data to retain the same set of probes as the training data for a particular disease pair. This data can be directly input into “predict” function along with the training model to generate the summary. The class will be predicted based on the probability cutoff, as mentioned in the first row of all the model outputs. For example, if the probability associated with the new sample, when run on the IBS vs HC training model is predicted as 0.3, which is less than 0.44, it will be classified as a healthy control and if is predicted to be 0.5, it will be classified as IBS.

### Functional Significance of Selected Genes

To identify IBS-specific GO terms, common pathways between IBS vs HC, IBS vs IBD and IBS vs celiac classifiers with a p<0.05 were identified. To identify pathways that are well represented across the repeats, we performed gene ontology analyses on classifiers identified in all repeats and selected terms that were overrepresented (enrichment p<0.05) in >30 or >50% repeats for each comparison. These analyses were repeated for IBD and celiac-associated classifiers to identify disease-specific pathways.

### Example 3: Genome-Wide DNA Methylation Analysis of Irritable Bowel Syndrome, Inflammatory Bowel Diseases, Celiac Disease and Healthy Controls Identifies Disease-Specific Epigenetic Changes and Pathways

This Example demonstrates that blood-based DNA methylation biomarkers provide a non-invasive test to distinguish IBS, IBD and CeD. Gene ontology suggested functional significance of the classifiers in disease-specific pathology.

Irritable bowel syndrome (IBS), inflammatory bowel disease (IBD) and celiac disease (CeD) present with similar gastrointestinal (GI) symptoms. While IBD and CeD are diagnosed by endoscopy with histological confirmation, IBS is diagnosed by symptom criteria due to a lack of a reliable diagnostic test. DNA methylation-based diagnostic biomarkers have not been investigated in IBS, IBD and CeD. We aimed at building blood-derived DNA methylation-based diagnostic classifiers using machine learning to differentiate IBS, IBD, and CeD, subsequently identifying gene ontology (GO) terms associated with disease-specific classifiers.

Genome-wide DNA methylation of peripheral blood mononuclear cells from patients with IBS, IBD, CeD, and healthy controls was measured using Illumina's 450K or EPIC arrays. Differential DNA methylation between IBS, IBD, CeD and HCs was measured using general linear models (GLMs) in “limma”. Classifiers were developed using machine learning (ML) by training penalized generalized linear models using double cross-validation with diagnosis as an outcome, methylation as a predictor and age and other confounders as covariates. An external dataset on 304 IBD and HCs was used as an independent validation cohort. Methylation sites selected using GLMs and those selected by the prediction models were used to identify GO enrichment.

Results were based on 315 participants (148 IBS, 47 IBD, 34 CeD and 86 healthy controls) who had DNA methylation data. IBS vs IBD and IBD vs CeD showed the highest number of differentially methylated CpG sites followed by IBD vs HC, CeD vs HC and IBS vs HC. Classification accuracies based on the area under the receiver operating characteristic curves for IBS vs IBD, IBS vs CeD and IBD vs CeD were 0.80 (95% CI=0.7-0.87, p=6.75E-10), 0.78 (95% CI=0.68-0.86, p=4.57E-10) and 0.73 (95% CI=0.62-0.83, p=0.03), respectively. The performance of IBD vs HCs was successfully validated in the external validation dataset. IBS-associated differentially methylated genes and the ML-based classifiers were enriched in cell adhesion and neuronal pathways, while IBD and CeD classifiers were enriched in inflammation and MHC class II pathways, respectively (p<0.05).

Irritable bowel syndrome (IBS) is a highly prevalent stress-sensitive, chronic gastrointestinal (GI) disorder characterized by chronic abdominal pain associated with diarrhea and/or constipation. IBS has a worldwide prevalence of 4.5-11% (1) and is associated with a significant healthcare and economic burden (2). There is currently no valid diagnostic test that can reliably diagnose IBS and therefore symptom-based criteria are used. Most IBS patients have seen at least three physicians and undergo multiple expensive and invasive tests before a diagnosis of IBS, as IBS is often considered a diagnosis of exclusion (3,4). Other GI conditions, such as inflammatory bowel disease (IBD), celiac disease (CeD) and colon cancer, can present with similar GI symptoms but are diagnosed by confirmatory tissue histopathology and treated differently. Expensive and invasive diagnostic evaluations are performed to exclude these other conditions before a diagnosis of IBS is made.

IBD, comprised of Crohn's disease and ulcerative colitis (UC), are chronic, immune-mediated conditions manifesting as intestinal mucosal inflammation. IBD affects approximately 3 million Americans, evenly divided between Crohn's disease and UC. (5,6) In general, the diagnosis of IBD is made by gastrointestinal disease specialists and requires endoscopic and histological confirmation. The time between onset of symptoms and referral to a specialist can vary substantially. One of the major sources of delay in diagnosis is treatment of the patient under the assumption that they have IBS rather than IBD. (7) About 10% of IBD patients are misdiagnosed as IBS.7 Delays in diagnosis are associated with worse outcomes, including increased incidence of complications and need for early surgery (8-10).

CeD is a gluten-induced, immune-mediated enteropathy, with an estimated worldwide prevalence of approximately 1% (11-14). CeD can present with variable manifestations and can be misdiagnosed as IBS (15). GI society guidelines recommend screening for CeD with serologies in patients with IBS, chronic diarrhea, and anemia, for instance. However, up to 5% of patients are seronegative (14). Additionally, some patients self-initiate a gluten-free diet, which is not uncommon in clinical practice (16,17). Substantial delays in diagnosing CeD even up to 13 years can occur in clinical practice (13, 18, 19). Delays in diagnosis and treatment of CeD can lead to long-term complications such as osteoporosis, infertility, anemia, and lymphoma and increased health care utilization and pharmacotherapy (20,21).

Thus, there is a significant unmet need for an objective biomarker-based test that can distinguish IBS, IBD and CeD, allow an earlier diagnosis and appropriate treatment plan and reduce unnecessary medical costs and tests (e.g., computed tomography [CT] scans, abdominal ultrasounds, stool studies, breath tests, repeat endoscopies). The importance of a diagnostic test for patients was supported by a recent large scale survey of individuals with IBS who reported that a fast and accurate diagnostic test for IBS is one of the top ten research priorities (22). Such a discovery has the transformative potential of shifting the paradigm of diagnosing IBS, IBD and CeD.

Epigenetic modifications including DNA methylation are mechanisms that regulate gene expression and higher-order DNA structure. DNA methylation has emerged as a leading mechanism linking gene-environment interactions to long-term behavioral development, particularly in complex disorders (23,24). In normal mammalian somatic genomes, DNA methylation mainly occurs at Cytosines in a CpG dinucleotide context. CpG methylation is generally absent from short stretches of CpG-rich sequences known as CpG islands (CGIs) which typically occur at or near the transcription start site of genes (25). Hypermethylation of CGI promoters is tightly linked with transcriptional repression of the affected gene and therefore have been viewed as an epimutation causing the silencing of a gene. In contrast, recent studies show that gene body methylation is positively correlated with gene expression and can be potential therapeutic targets (26).

Aberrant DNA methylation has been associated with a variety of cancers and non-cancerous disorders, including psychiatric and neurodegenerative disorders. Our previous study on DNA methylation and targeted bisulphite sequencing in peripheral blood mononuclear cells (PBMCs) identified changes in DNA methylation in IBS patients compared to healthy controls (27). This study highlighted a role for neuronal and oxidative stress pathways in the pathophysiology of IBS. DNA methylation changes in blood have been associated with inflammation in patients with IBD (28), however, there is a lack of blood-based DNA methylation studies in CeD. Additionally, there are no studies investigating DNA methylation as a biomarker for the differential diagnosis of IBS from other diseases that mimic IBS.

Therefore, we hypothesized that DNA methylation marks in PBMCs can serve as a biomarker for diagnosing IBS and for distinguishing IBS from IBD and CeD. Similarly, DNA methylation-based biomarkers may be used for non-invasive diagnoses of IBD and CeD. This study sought to develop DNA methylation-based classifiers to discriminate IBS, IBD, CeD, and healthy controls, and also to investigate gene ontology (GO) terms and pathways associated with the classifiers.

### Materials and Methods

### Study Population

Male and female participants with IBS, IBD, and CeD, and healthy controls ages 18-55 were recruited by community advertisement or at GI clinics at the University of California, Los Angeles (UCLA). IBS and healthy control (HC) samples were collected between 2009 and 2020 (analyzed on 450K or EPIC platforms). IBD and CeD samples were collected between 2012 and 2020. In addition to the UCLA cohort, we included banked PBMC samples from patients with IBD obtained from the Crohn's and Colitis Foundation (CCF) Study of a Prospective Adult Research Cohort with IBD (SPARC IBD) with identical inclusion/exclusion criteria. The SPARC IBD cohort has been previously described (29). The diagnosis of IBS and bowel habit subtypes were based on Rome III (30) or IV (31) criteria depending on time of recruitment and confirmed by a clinician with expertise in IBS. Healthy controls had no personal or family history of IBS or other chronic pain conditions. Additional exclusion criteria for IBS and healthy controls included infectious or inflammatory disorders, active psychiatric illness over the past six months assessed by structured clinical interview for the DSM-IV (MINI) (30), use of corticosteroids in the past six months, use of narcotics in the past two months, and alcohol abuse. Questionnaires administered to the participants are described in detail in the Supplementary Methods. The Bowel Symptom Questionnaire (BSQ), Hospital Anxiety and Depression Scale (HAD) (32), and Adverse Childhood Experiences (ACE) (33) questionnaires were administered to all UCLA patients in this study including IBS, IBD and CeD. Additional questionnaires administered to IBS patients included Irritable Bowel Syndrome Severity Scoring System (IBS-SSS) (34).

The diagnosis of IBD was confirmed by endoscopy with pathologic tissue confirmation. Since treatments such as inflammation-reducing drugs used in IBD have been associated with changes in DNA methylation (35), we recruited patients that were treatment naïve or currently on no IBD treatment including biologic agents (anti-TNF therapy, ustekinumab, risankizumab or vedolizumab) or other agents (tofacitinib, upadicitinib, ozanimod, 6-mercaptopurine, azathioprine, methotrexate, or 5-aminosalicylic acid [5-ASA]). Additional exclusion criteria included Crohn's disease treated surgically without evidence of subsequent disease and history of coexistent IBS or CeD. Patients with IBD reported active GI symptoms, e.g., abdominal pain, bloating, diarrhea, and/or blood in stool. Disease activity was assessed with the following instruments: Simple Endoscopic Score for Crohn's Disease (36) (SES-CD, UCLA and SPARC IBD cohorts), and Crohn's Disease Activity Index (37) (CDAI, UCLA cohort), Short-CDAI (38) (SCDAI, SPARC IBD cohort), Simple Clinical Colitis Activity Index (39) (SCCAI, UCLA cohort), and Ulcerative Colitis Disease Activity Index (40) (UCDAI, SPARC IBD cohort) as described in the Supplementary Methods.

Patients with CeD reported active GI symptoms, e.g. abdominal pain, bloating, diarrhea and had their diagnosis confirmed by positive anti-tissue transglutaminase antibodies (anti-TTG) and/or anti-endomysial antibodies (anti-EMA) and the presence of at least Marsh II lesions on duodenal biopsies. Patients currently adherent to a gluten-free diet >2 weeks, history of co-existent diagnosis of IBD, IBS, other causes of malabsorption, or any other medical conditions that could explain their GI symptoms, were excluded. Questionnaires administered to CeD patients included the validated Celiac Symptom Index (CSI) (41) and Celiac Dietary Adherence Test (CDAT) (42).

All study participants who had a history of >½ pack per day of cigarettes were excluded. One Crohn's (4%) and three CeD patients (9%) reported as being former smokers. A small percentage of participants reported the use of drugs including selective serotonin reuptake inhibitors (SSRIs) and serotonin-norepinephrine reuptake inhibitors (SNRIs, 7-9%), tricyclic anti-depressants (TCAs) or benzodiazepines (3-9%), statins (1-9%), and beta blockers (0-3%) (Supplementary Table 1), which can affect DNA methylation (43). None of the healthy controls used these medications. Participants recruited at UCLA were compensated. The study was approved by the UCLA Institutional Review Board, and all subjects signed a written informed consent prior to the study.

### Statistical and Bioinformatic Analyses

### Clinical and Demographic Data

Group differences in demographic characteristics including age, sex, body mass index (BMI), race, ethnicity and smoking status were assessed using t-tests, ANOVA or Fisher's tests. Summary statistics were created for disease activity scores for IBS, IBD and CeD.

### DNA Methylation Data Processing

The methods used to process DNA methylation data and implementation of machine learning algorithms are presented in detail in Supplementary Methods. In short, raw Illumina DNA methylation array data (IDAT) files generated by the Illumina iScan scanner were processed using Enmix DNA methylation analysis pipeline (44).

DNA methylation data pre-processing: Details of the analysis are presented in Supplementary Methods. Briefly, Quality Control (QC) metrics were generated and samples that did not pass the QC threshold were eliminated. Background correction and dye bias correction were applied to the data and the resulting signal intensities were normalized using the ‘quantile normalization’ method. Cell types were estimated as described previously (45). QC information-based filtering was implemented to filter unwanted probes out of the 855,790 CpGs on the array.

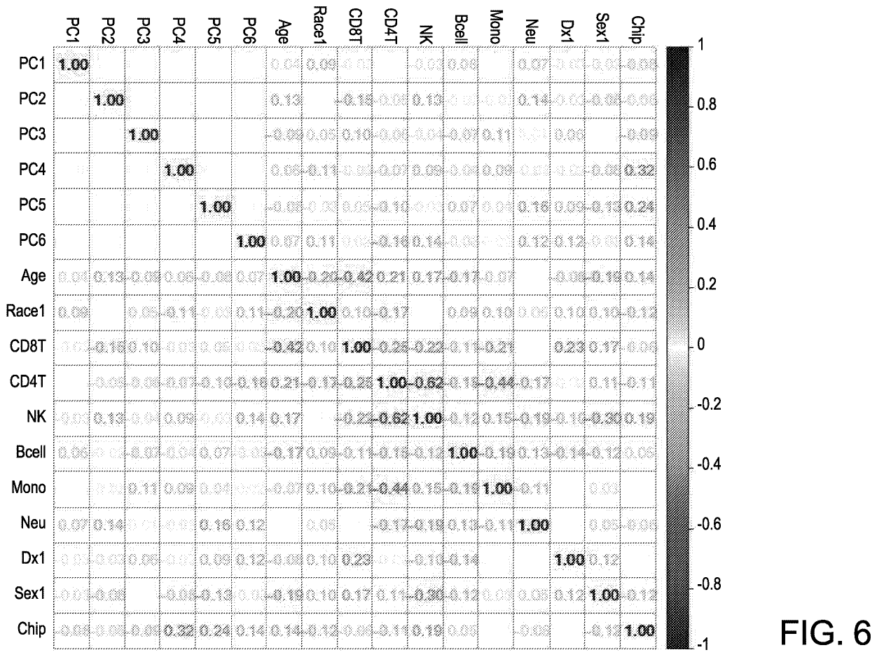

Additionally, CpGs on X and Y chromosomes, single nucleotide polymorphisms (SNPs) and repeats, non-specific or cross-reactive probes and probes showing low variability and extreme methylation values were removed. The remaining sites were used as input for classifier development. All the analyses were performed using R programming language (cran.r-project.org). To select the covariates that affected DNA methylation, we correlated the principal components derived from the methylation data, for example on IBS and HC data with potential confounders (FIG. 6).

Differential Methylation: Group differences between IBS, IBD, CeD and HC were analyzed using general linear models using ‘limma’ package using age and batch as covariates. P-values were adjusted for multiple tests using false FDR.

Classifier development and evaluation: Using the genome-wide DNA methylation data, we developed machine learning (ML)-based classifiers to test their performance as diagnostic biomarkers in classifying the GI diseases and HCs. Generalized linear models (GLM) were fit via penalized maximum likelihood using glmnet package, considering diagnosis as outcome, normalized and filtered DNA methylation probes (CpG sites) as predictors, and age, cell-type proportions, and technical batch as covariates. We used a double cross-validation method (46) to evaluate and test machine learning models, which is a preferred method since the models are trained and tested on independent datasets. Double cross-validation process includes two nested cross-validation loops referred to as outer and inner loops. The training dataset was further sub-divided into tuning/calibration and validation sets which form the inner loop. The calibration set was used for model building (hyperparameter tuning) and the validation set was used to estimate the errors. The model with the lowest prediction error within the inner loop was selected as the best model. This model was then applied to the test dataset and the class labels were predicted. Multiple splits (N=10) of inner and outer datasets were run in order to avoid the bias with respect to variable selection resulting from use of a single training set (FIG. 4). Lasso, ridge or elastic net regression models (penalty term representing shrinkage α=0 to 1) were fitted and the model associated with an α resulting in the best performance was chosen as the final model. Optimal cutoffs were chosen so as to have a minimum difference between the sensitivity and specificity and the values close to the area under the receiver operating characteristic curve (AUC) (47). Performance metrics including, accuracy (mean and 95% confidence interval [CI]), sensitivity, specificity, F1, and p values were generated based on the 2×2 confusion matrix constructed using the predicted and true test sample labels.

Gene ontology (GO) analysis: Enrichment of GO terms and or pathways associated with differentially methylated CpG and ML-based classifiers for each comparison was assessed using ‘missMethyl’ package in R (48) as described in Supplementary Methods.

Sensitivity analyses: FIG. 6 shows correlation between DNA methylation principal components (PCs) and potential confounders. We performed a sensitivity analysis to assess the performance of the classifiers excluding patients who consumed some medications that can potentially alter DNA methylation (43) including SSRIs, benzodiazepines, statins or beta blockers (Table 8). Since IBD PBMC samples were derived from two sites (UCLA and CCF), we repeated IBS vs IBD comparison and included the “site” variable as an additional batch covariate.

### Assessment of Classifier Performance

External validation set: We downloaded whole blood HM450K DNA methylation data on IBD vs healthy controls from the Gene Expression Omnibus (GEO) database (accession #GSE87648) for validating the performance of our IBD vs model. The processed dataset includes 460398 probes on 304 samples (204 IBD and 100 healthy controls). We filtered the external data to include probes that overlapped with the probes on Epic array to match the internal IBD and HC dataset. We trained our algorithm on our IBD and HC samples using GLMNET with 10-fold CV as described in the previous section and predicted the classification of external samples. The results were evaluated using AUC and CI.

Permutation testing: Permutation-based testing to assess classifier performance has been used extensively in classification problems in computational biology to test if there is a real class structure in the data (49). We generated 100 random permutations to generate AUCs from classifiers generated from shuffled labels to create null distributions. We compared the IBS vs IBD, IBS vs CeD, IBD vs CeD, and IBS, IBD, and CeD vs models generated on the actual class labels to the corresponding null distributions and calculated the p-values for differences in the distributions.

### Results

We analyzed DNA methylation profiles of 315 participants including IBS (N=148; 65% women, 45 constipation-predominant IBS [IBS-C], 54 IBS-D, 49 mixed or unsubtyped IBS [IBS-M or IBS-U]), CeD (N=34; 68% women), IBD (N=47; 22 UC and 25 Crohn's disease, 49% women], and healthy controls (N=86; 55% women). Table 5 shows demographic characteristics of study participants. There were significant overall group differences in mean age, race and ethnicity (p=5.97e-06, 5.0e-4, 1.0e-3, respectively). There were no significant group differences in the percentage of women or BMI (Fisher p=0.31 and ANOVA p=0.26, respectively).

TABLE 5
Demographic Characteristics of the Study Population

| | IBS | UC | Crohn's | CeD | Healthy | |
|---|---|---|---|---|---|---|
| | (N = 148) | (N = 22) | disease (N = 25) | (N = 35) | controls (N = 86) | P value |
| Age; Mean (SD) | 30.9 (10.9) | 45.8 | 36.6 | 36.1 | 30.5 | 1.22e−07^{#} |
| | | (14.5) | (14.7) | (13.4) | (10.2) | |
| Sex (% Female) | 96 (65%) | 12 (55%) | 12 (48%) | 23 (66%) | 47 (55%) | 0.31^{$} |
| BMI; Mean (SD) | 25.8 (5.6) | 25.0 (5.9) | 24.0 (5.2) | 24.9 (4.5) | 26.6 (4.2) | 0.31^{#} |
| Race | | | | | | 0.0005^{$} |
| Caucasian | 77 (52%) | 14 (64%) | 20 (80%) | 25 (71%) | 35 (41%) | |
| Black | 14 (9%) | 1 (5%) | 0 | 0 | 10 (12%) | |
| Asian | 27 (18%) | 1 (5%) | 1 (4%) | 2 (6%) | 22 (26%) | |
| Multiracial | 22 (15%) | 1 (5%) | 1 (4%) | 0 | 10 (12%) | |
| American Indian | 3 (2%) | 1 (5%) | 0 | 1 (3%) | 6 (7%) | |
| Unknown | 5 (3%) | 3 (14%) | 3 (12%) | 5 (14%) | 3 (3%) | |
| Ethnicity (%) | | | | | | 0.001^{$} |
| Hispanic | 34 (23%) | 3 (14%) | 1 (4%) | 5 (14%) | 23 (27%) | |
| Non-Hispanic | 109 (74%) | 15 (68%) | 21 (84%) | 25 (74%) | 63 (73%) | |
| Unknown | 5 (3%) | 4 (18%) | 3 (12%) | 4 (11%) | 0 (0%) | |

^{#}ANOVA p value;
^{$}Fisher test p value; IBS, irritable bowel syndrome; UC, ulcerative colitis; CeD, celiac disease; BMI, body mass index; SD, standard deviation

About 35% IBS, 28% IBD and 31% CeD patients used medications such as antidepressants, statins or NSAIDs (Table 8). Severity of IBS symptoms were moderate with mean (standard deviation [SD]) overall severity score of 9.45 (4.18; range 0-20) and IBS-SSS of 237 (89.70; range 0-500) which represents moderately severe symptoms. The mean (SD) ACE score in IBS patients was 1.91 (1.89), Table 9A. UC disease activity assessed by UCDAI and SCCAI (mean [SD]=4.29 [1.25] and 6.17 [4.84], respectively) indicated mild to moderate disease. Crohn's disease activity assessed by CDAI, SCDAI and SES-CD indicated mild to moderate disease (mean [SD]=146.36 [78.10], 243.82 [73.77] and 4.40 [5.30], respectively) Table 9B). CeD activity assessed by CSI scores indicated moderate disease (mean [SD]=38 [10.98]) and CDAT scores (Mean [SD]=14.94 [3.88]) indicated a poor adherence to a gluten-free diet (GFD) by the CeD patients in this study (>13 indicates non-adherence, Table 9C).

Based on the medical records, a history of anxiety and/or depression was the most common co-morbidity (28%) associated with IBD, followed by thyroid disease (8%) and gastroesophageal reflux disease (GERD, 4%) in the UCLA cohort, and IBD-associated arthropathy amongst SPARC IBD patients (5%). Patients with CeD also reported the diagnosis of thyroid disease (14%), anemia and GERD (9%) and anxiety or depression (6%).

Differentially Methylated CpG Sites Associated with Diagnostic Groups

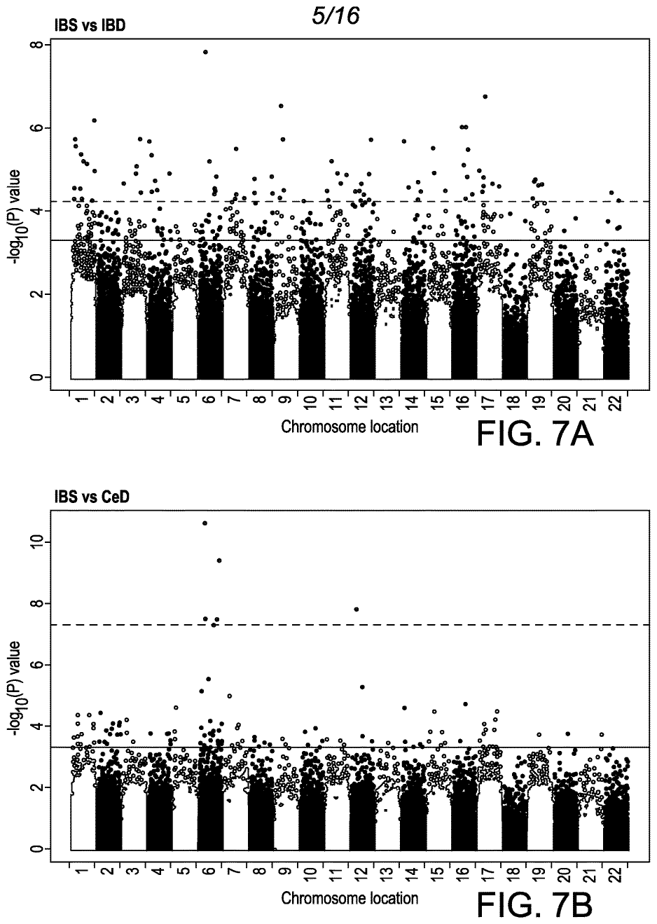

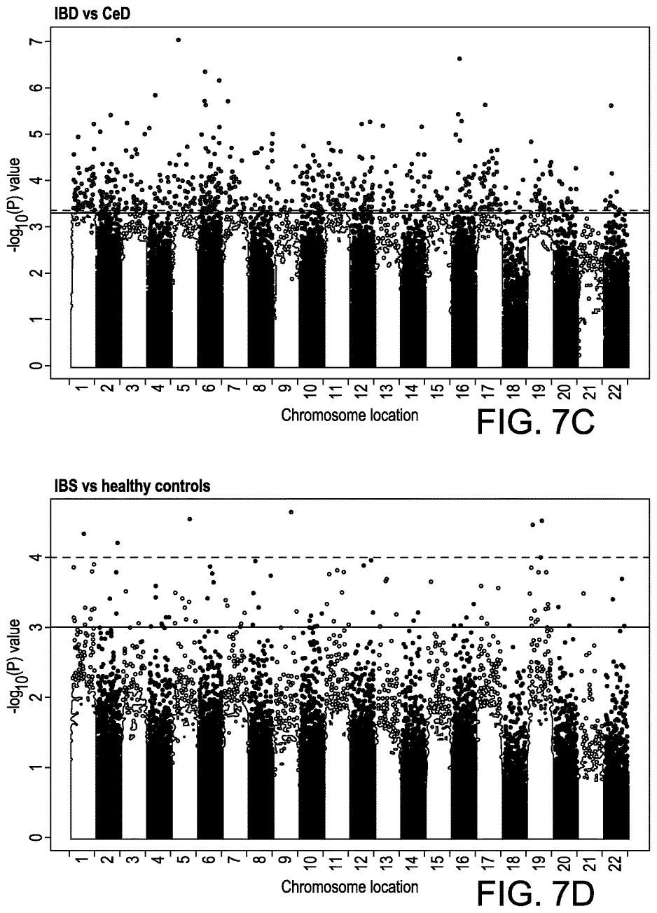

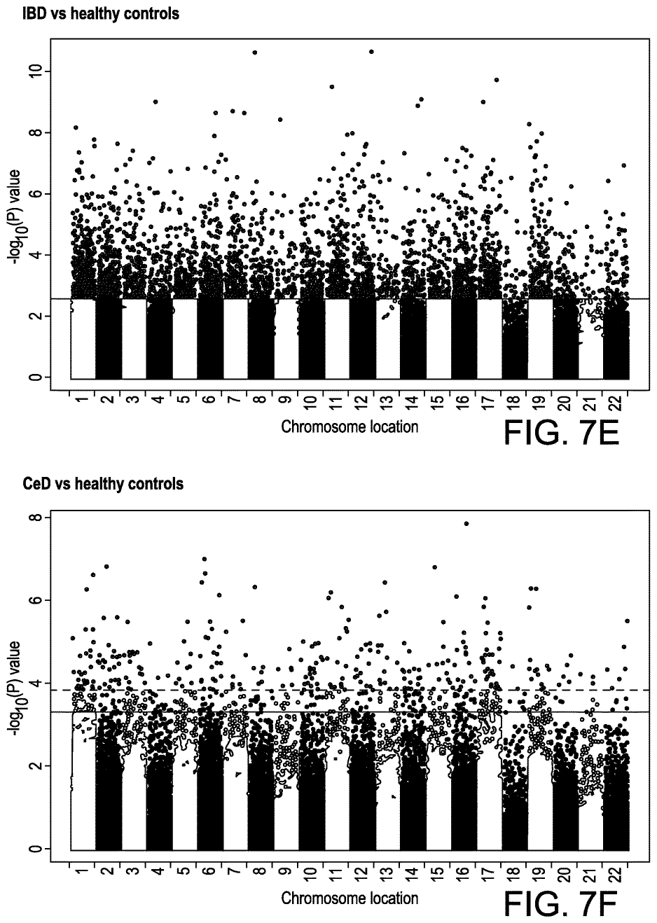

There were significant differences in CpG methylation between IBD vs HCs (FDR<0.05, N=4130), IBD vs CeD (FDR<0.05, N=655), CeD vs HCs (FDR<0.05, 311), IBS vs IBD (FDR<0.05, N=86) and IBS vs CeD (FDR<0.05, N=6, FDR<0.05). Between IBS and HCs, 48 CpGs were differentially methylated at p<0.0005, but none at FDR<0.05 (FIG. 7, Tables 10A-10F).

Gene Ontology Terms Associated with Differentially Methylated Genes

GO analyses on genes with differentially methylated CpG sites between disease groups suggested an association of terms including “humoral immune response” and “central nervous system maturation”, with IBS vs IBD; “MHC class II protein complex binding” and “immune system process” with IBS vs CeD; and “adaptive immune response” and “cytokine production” with IBD vs CeD. Disease groups vs HC comparisons resulted in terms including “ion transport activity” and “calcium-release channel with IBS vs HC; “immune response” and “leukocyte activation” with IBD vs HC; and “regulation of immune system process” and “T-cell activation” with CeD vs HC comparisons (Tables 11A-11F).

### DNA Methylation-Based Classifiers to Discriminate IBS, IBD, CeD and HCs

To identify disease-associated classifiers, penalized regression models were trained independently on pairs of diagnoses including, IBS, IBD, CeD and healthy controls (Table 7) and tested on a holdout test dataset resulted in disease specific classifiers.

### DNA Methylation-Based Classifiers for IBS, IBD and CeD

Our results showed that the selected classifiers showed high accuracy for discrimination between various patient groups. Table 6 shows the performance metrics for IBS compared to healthy controls, IBD and CeD. The AUC and accuracy for IBS vs IBD classifier were 0.85 and 0.80, respectively (p=6.75E-10) and those for IBS vs CeD were 0.82 and 0.78, respectively (p=4.57E-10). FIG. 4 shows the ROC curves for these comparisons. For IBD vs CeD, AUC and accuracy were 0.78 and 0.73 (p=0.002), respectively. When comparing IBS vs Crohn's disease and UC separately, the classifiers for IBS performed slightly better in discriminating against Crohn's disease compared to UC (Crohn's: Accuracy [95% CI]=0.80 [0.66-0.9]; UC: Accuracy [95% CI]=0.75 [0.60-0.87]).

TABLE 6
Performance Metrics for IBS, IBD and CeD Associated Classifiers

| | | | | IBS vs | IBD vs | CeD vs |
|---|---|---|---|---|---|---|
| | | IBS vs | IBD vs | healthy | healthy | healthy |
| | IBS vs IBD^{#} | CeD | CeD | controls* | controls^{#} | controls |
| AUC | 0.85 (0.77-0.93) | 0.82 (0.72-0.91) | 0.78 (0.67-0.88) | 0.69 (0.61-0.77) | 0.92 (0.88-0.97) | 0.85 (0.77-0.93) |
| Accuracy | 0.80 | 0.78 | 0.73 | 0.69 | 0.82 | 0.80 |
| (95% CI) | (0.7-0.87) | (0.68-0.86) | (0.62 -0.83) | (0.61-0.75) | (0.73-0.89) | (0.70-0.88) |
| Sensitivity | 0.79 | 0.81 | 0.73 | 0.76 | 0.89 | 0.76 |
| Specificity | 0.80 | 0.74 | 0.74 | 0.62 | 0.77 | 0.82 |
| F1 | 0.80 | 0.81 | 0.76 | 0.71 | 0.82 | 0.74 |
| Accuracy | 4.57E−10 | 2.21E−07 | 1.84E−073 | 3.06E−05 | 3.41E−16 | 7.11E−09 |
| P-Value | | | | | | |

*For IBS vs healthy control comparison, DNA methylation data from 450K and EPIC arrays was used, for all other comparisons only subjects with EPIC array data were used;
^{#}Covariates included age, cell-type proportions, technical batch effects for all columns however, IBD vs healthy control comparison included site of IBD sample collection as an additional covariate. F1 denotes weighted average of precision and recall; AUC, area under the receiver operating characteristic (ROC) curve; IBS, irritable bowel syndrome; IBD, inflammatory bowel diseases; CeD, celiac disease.

When comparing GI diseases to healthy controls, the AUC and accuracy for IBS vs healthy controls were 0.69 and 0.69, respectively (p=3.06-05). The classification accuracies between the different bowel habit subtypes within IBS including IBS-D, IBS-C and IBS-M vs healthy controls were similar to the overall IBS group (Table 12). AUC and accuracy of IBD vs healthy controls were 0.92 and 0.82 (p=1.33E-08), and those of CeD vs healthy controls were 0.85 and 0.80 (p=7.11E-09).

### Assessment of Classifier Performance

### External Validation Set

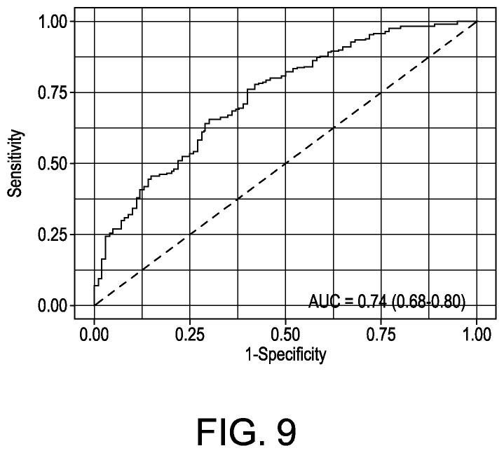

Classifiers developed based on IBD vs data from this study were used to predict the classes of samples from an independent IBD vs DNA methylation dataset as described in the Methods section. The classifier successfully classified a majority of subjects to correct classes (FIG. 9, AUC=0.74, p<0.0001).

### Permutation Testing

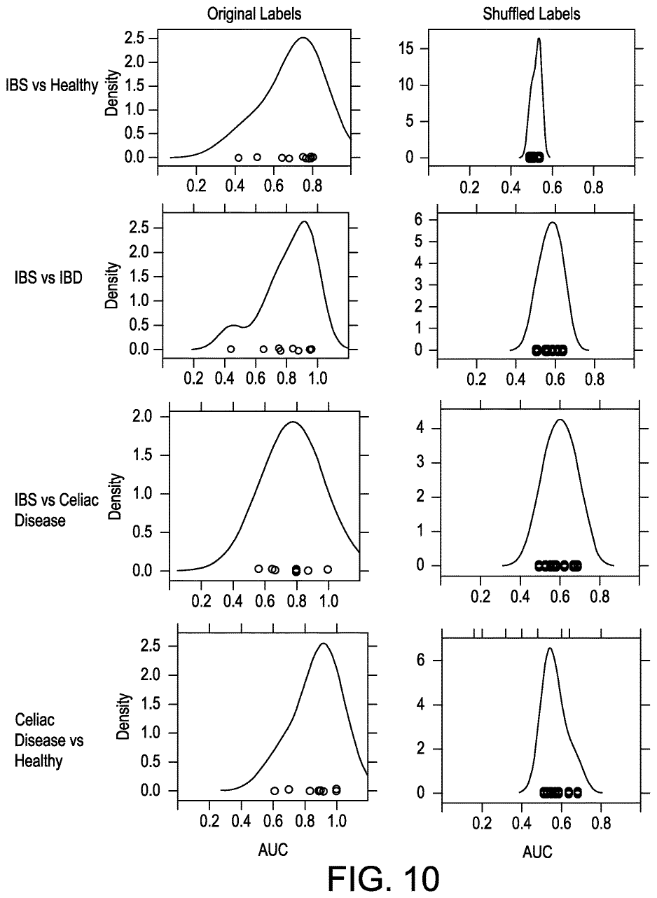

Using permutation testing, we demonstrated that the true labels for IBD vs HC, IBS vs HC, IBS vs IBD, IBS vs Celiac, and IBS vs HC performed significantly better than the permutated labels (P<0.05, FIG. 10).

GO Terms and Pathways Associated with Classifiers

### IBS vs HCs, IBD and CeD

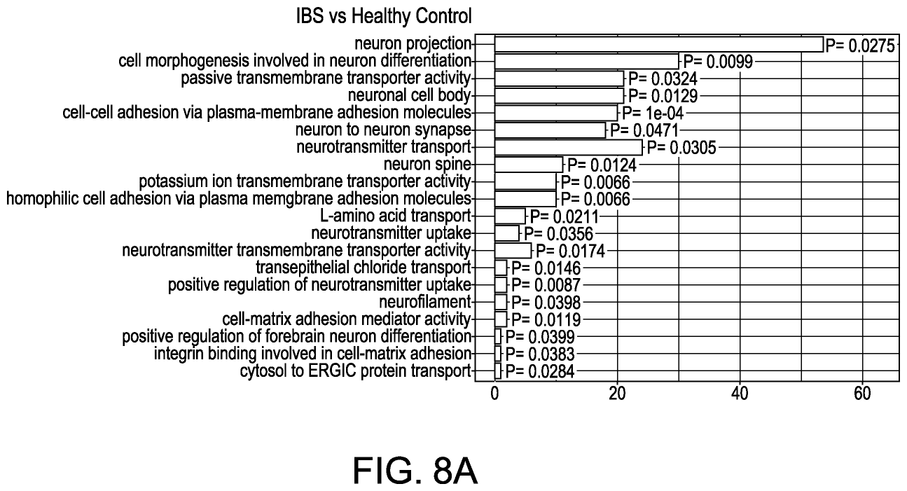

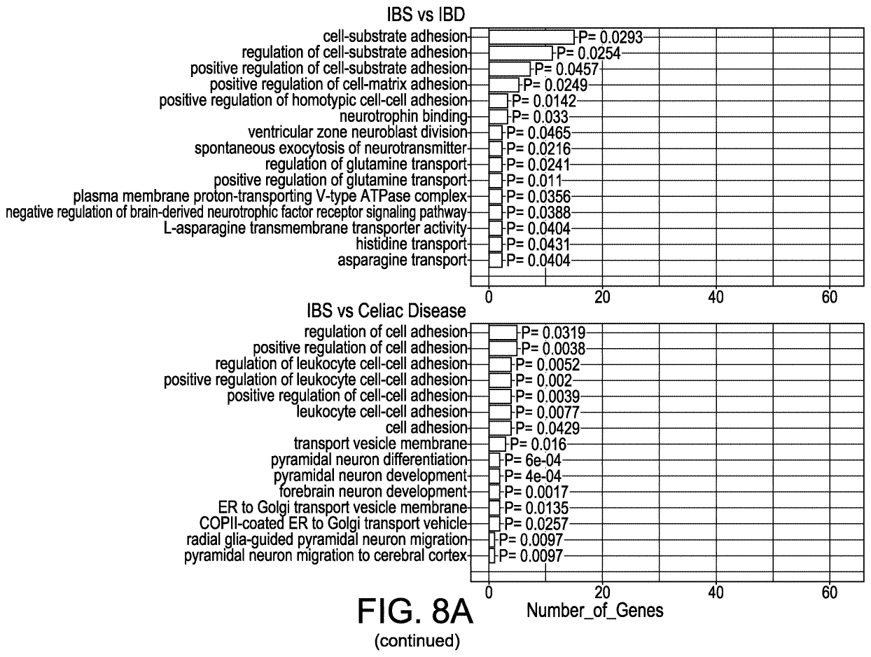

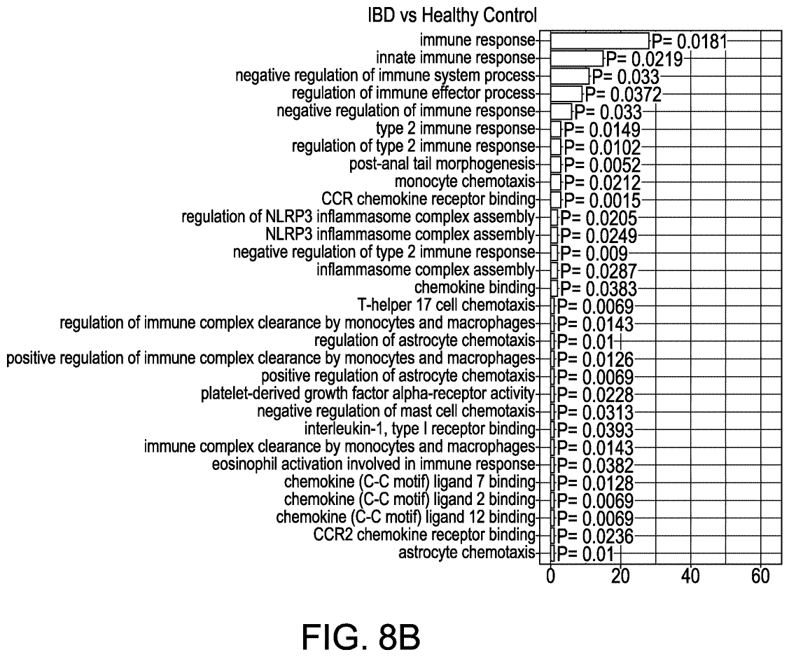

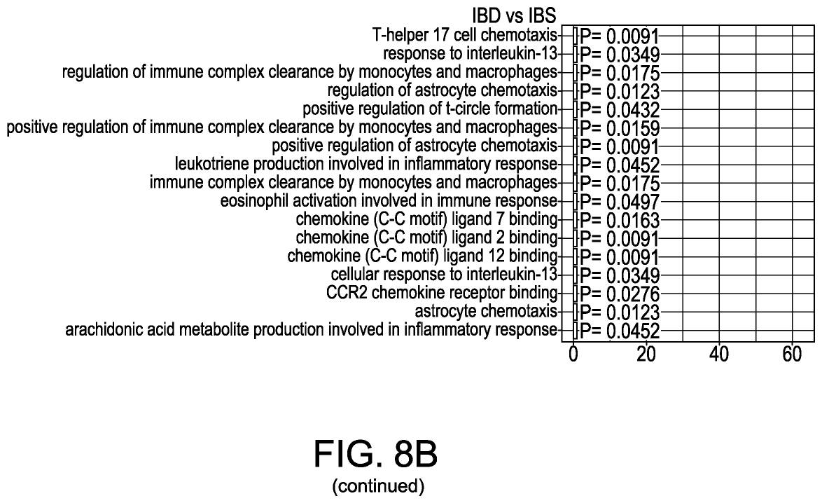

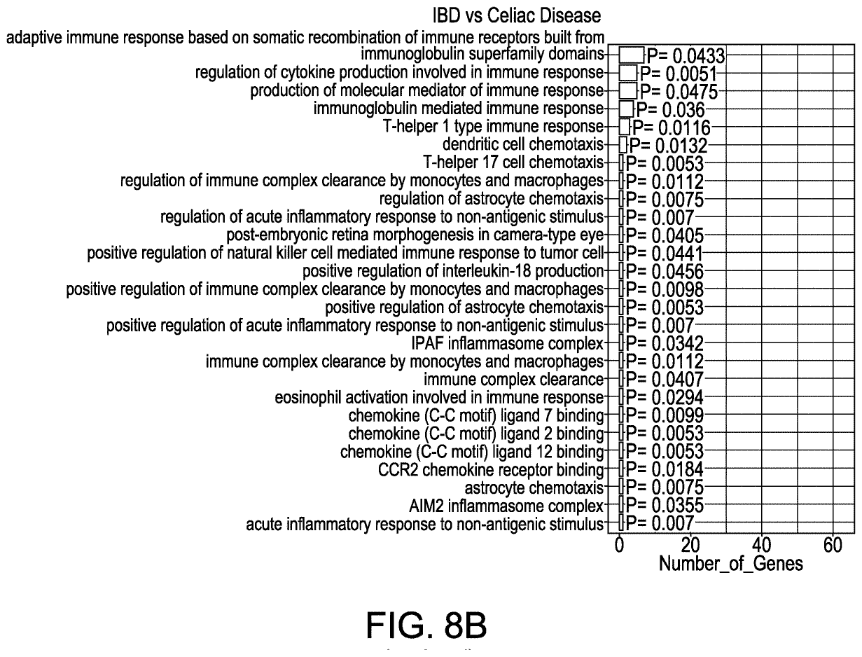

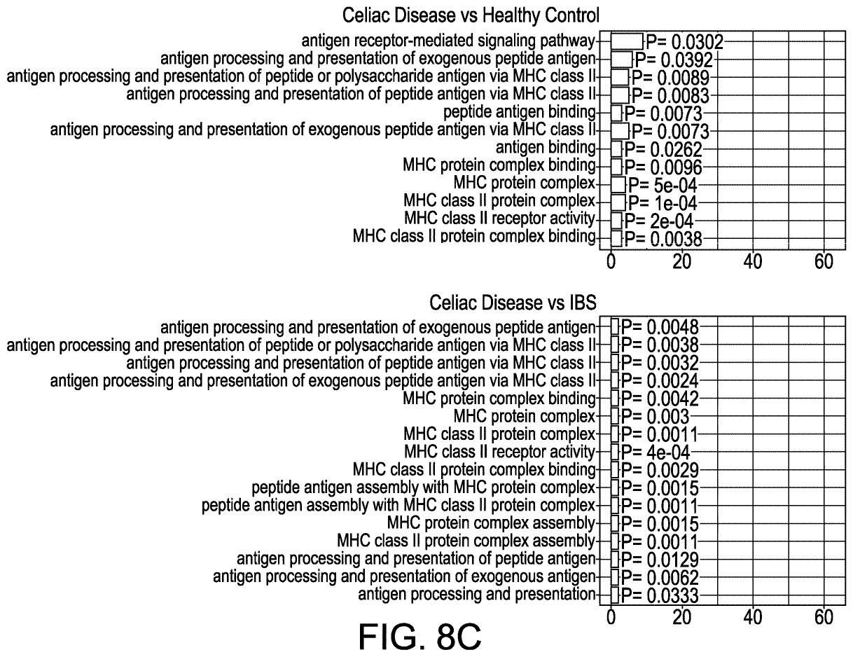

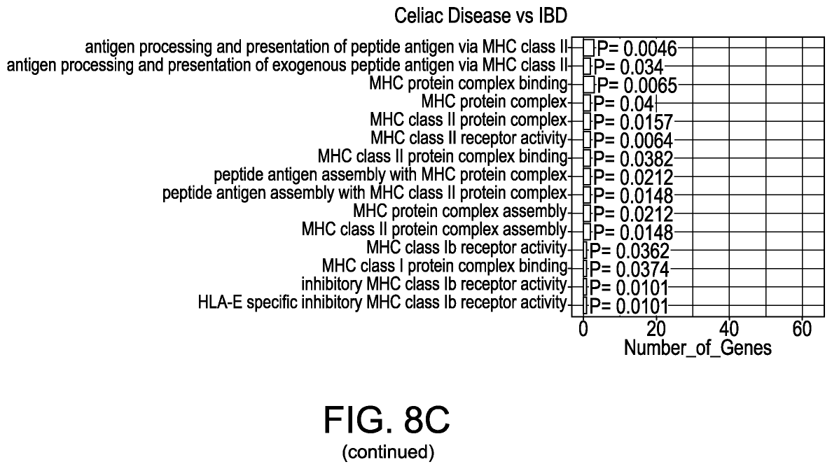

GO terms associated with IBS-related classifiers, i.e., overlapping terms between IBS vs HCs, IBS vs IBD and IBS vs CeD classifiers were enriched in cell adhesion and neuronal pathway-associated terms including “cell-adhesion”, “neurotransmitter uptake”, “forebrain neuron development” and “sensory perception of pain”, suggesting a role for IBS-associated genes in permeability and visceral pain (50) (FIG. 8A, Table 14A, p<0.05).

Analysis of GO terms associated with the differentially methylated genes as well as classifiers suggested that IBS was associated with epigenetic changes in neuronal and immune system pathways.

### IBD vs HCs, IBD and CeD

The IBD classifiers were primarily enriched in immune and inflammation-related pathways including “innate immune response”, “T-helper 1 type immune response” and “T-helper 17 cell chemotaxis” (p<0.05, FIG. 7B).

Analysis of GO terms associated with the differentially methylated genes as well as classifiers suggested that IBD was associated with epigenetic changes in inflammation and immune response pathways.

### CeD vs HCs, IBS and IBD

CeD classifiers were enriched in major histo-compatibility complex (MHC) pathways-related terms such as “MHC class II protein complex” and “antigen processing and presentation of exogenous peptide antigen” (p<0.05, FIG. 7C).

Analysis of GO terms associated with the differentially methylated genes as well as classifiers suggested that CeD was associated with epigenetic changes in MHC pathway.

### Sensitivity Analyses

DNA methylation is known to be sensitive to confounders. FIG. 6 shows correlation between DNA methylation principal components (PCs) and potential confounders.

We repeated IBS vs IBD and IBS vs CeD comparisons excluding patients who consumed medications including anti-depressants, NSAIDs and statins, which are reported to affect DNA methylation (43) and recalculated the performance metrics. The AUC for the new model was comparable to that of the original model in both comparisons (Table 13), suggesting that the models chosen were robust to the effects of these confounders.

### Discussion

We report, for the first-time, a comprehensive analysis of genome-wide DNA methylation data in most common GI disorders including IBS, IBD and CeD, and HCs. Here, we identify the differentially methylated CpG sites and potential blood-based diagnostic biomarkers based on DNA methylation profiles that can accurately distinguish IBS, IBD and CeD. The genes associated with the differentially methylated CpG sites corroborated with the pathophysiologic mechanisms of the disease and the biomarkers we report here showed a good to excellent (51) AUC for differentiating IBS, IBD and CeD. These biomarkers can potentially allow an earlier diagnosis and appropriate treatment plan and reduce unnecessary medical costs.

Smaller studies with a limited genomic coverage exist in IBD (23) and CeD (24) populations involving multiple univariate tests which can have limited power to detect smaller linear and non-linear changes. Using machine learning frameworks enabled us to not only develop predictive models, but to also select for multiple relevant CpG sites without having to conduct repetitive association tests and consequential stringent multiple hypothesis adjustment. Moreover, our thorough preprocessing steps (including filtering specific sites and individuals, accounting for confounding) as well as the use of a double cross validation methodology lay the framework for reduced risk of overfitting and therefore improved reproducibility (and out of sample prediction). Finally, our stringent inclusion criteria of symptomatic IBD and CeD patients diagnosed with standard diagnostic criteria but not taking treatment for their diseases helped make our model more amenable to replicability and applicability to the target population of undiagnosed patients with chronic bowel symptoms.

The GO analyses suggested that the disease-specific classifiers identified in this study were associated with pathways that are relevant to the pathophysiology of the corresponding diseases. For example, IBS-associated classifiers were associated with cell adhesion, neuronal signaling and pain pathways, which are the most widely studied pathways in IBS (52-55). Similarly, inflammatory and immune pathways and related terms were associated with the IBD classifiers further supporting the functional significance of associated classifiers (56). Additionally, the CeD classifier was associated with MHC class II receptor activity. MHC class II encoded by human leukocyte antigen (HLA) is a chief genetic determinant of CeD, and certain HLA-DQ allotypes like DQ2 and/or DQ8, which are allelic variants within the constant region of HLA genes are known to predispose to the disease by presenting posttranslationally modified gluten peptides to CD4+ T cells (57).

Although endoscopies with biopsies remain the gold standard for diagnosing IBD and CeD, these are invasive, costly procedures which are associated with a risk of complications (58,59). Existing noninvasive biomarkers such as C-reactive protein (CRP), fecal calprotectin and lactoferrin have limitations including statistical heterogeniety between studies, variable cut-off points, and overlapping values between active inflammatory disease and non-inflammatory disease such as IBS (59).

There is currently no reliable diagnostic test for all IBS bowel habit subtypes with acceptable test characteristics that are needed for widespread clinical use. However, a commercial blood test for IBS-D is currently available. Studies have demonstrated increased circulating antibodies to cytolethal distending toxin B and vinculin (anti-CdtB, anti-vinculin) in patients with IBS-D. (60) The development of these biomarkers is based on studies in animal models showing cross-reactivity of anti-CdtB, which is produced by bacteria that cause gastroenteritis, with host vinculin and the association of these antibodies with an IBS phenotype. A subsequent follow up study comparing test characteristics in IBS-D patients with a smaller number of patients with IBS-M (n=25) and IBS-C (n=30) showed highest plasma levels of anti-CdtB and anti-vinculin in patients with IBS-D, lowest levels in IBS-C and levels in between these two groups in IBS-M. (61) However, this anti-CdtB or anti-vinculin test, which utilized blood samples from IBS-D patients who participated in a clinical trial for rifaximin, had a low sensitivity (<50%) for diagnosing IBS but a high specificity (60). Thus, at least half of patients with IBS have a negative test. A study conducted in Australia using population and outpatient-based samples found that anti-CdtB and anti-vinculin levels had a poor ability to discriminate IBS from organic GI disease, including IBD. (62) Recent GI society guidelines for IBS did not issue recommendations for or against the use of this serologic test. (16,17)

The diagnostic performance of a blood-based DNA methylation test could potentially be further improved by developing algorithms utilizing other non-invasive tests that aid in distinguishing gut inflammation and CeD, e.g., CRP, fecal calprotectin, and celiac serologies. However, while these tests help to rule out IBD and CeD if normal, they are not helpful in ruling in IBS. There are significant overlaps in elevated CRP and mildly elevated fecal calprotectin in IBS and IBD. For example, a normal fecal calprotectin levels <40 microgram (ug)/gram (g) has been shown to be associated with a <1% chance of having IBD (63) (i.e., ruling out IBD) and those between 40 μg/g and 150 μg/g may be seen in IBS, mildly active IBD, GI infection, GI neoplasm, CeD, or microscopic colitis. A higher fecal calprotectin, e.g., >150, will be more suggestive of an active inflammatory or infectious GI disorder, e.g., IBD (63). Abnormal fecal calprotectin or CRP would prompt additional diagnostic workup such as endoscopy with biopsies for IBD, CeD, and other organic GI diseases. Similarly, elevated celiac serologies would suggest the diagnosis of CeD but endoscopy with tissue confirmation is required in North America. However, celiac serologies and intestinal histology can be normal in CeD patients adherent to a prolonged gluten-free diet which limits their use in these patients (12). A gluten challenge followed by repeat serologies and/or small intestinal biopsy would be typically needed in those with self-initiated GFD, which can be unappealing and difficult for symptomatic patients. An accurate non-invasive diagnostic test for CeD would be helpful in this patient population. Use of a DNA methylation-based diagnostic test in this patient population requires further study since we only evaluated patients with active CeD not on a GFD or consuming one for <2 weeks.

A non-invasive diagnostic test for IBS can be valuable in different ways depending on the type of clinical practice and patient population. For gastroenterologists, limited or more comprehensive diagnostic testing including upper and lower endoscopies may be done in the presence of no or positive alarm features, respectively. However, in the common scenario of a negative diagnostic evaluation, a rule in test for IBS will be beneficial, particularly for patients who want confirmation that they have IBS before starting treatment, and for health care providers who can avoid ordering additional or repeated costly diagnostic tests and instead, can institute appropriate treatment to relieve IBS symptoms. The sensitivity and specificity of a diagnostic test for IBS such as the one we propose in this paper would arguably be quite acceptable in this scenario. For primary care physicians, a diagnostic test for IBS would allow them to make a positive diagnosis, recommend IBS treatment and avoid ordering more diagnostic testing and referring to a gastroenterologist for an endoscopic procedure. Other clinical scenarios where a DNA methylation-based test for IBS could be helpful are the diagnosis of coexistent symptomatic IBS in patients with CeD, IBD or other GI disease that has been treated or in remission which is between 32-40% of patients (64) or in patients with non-celiac gluten or wheat sensitivity, but further studies are needed.

Although machine learning is a powerful tool that learns latent patterns in the data to make predictions, it is susceptible to overfitting by learning patterns that arise from known or unknown confounding variables (65,66). To avoid overfitting, we nested the hyperparameter optimization using double cross-validation or nested cross validation (FIG. 4). Since this method involves testing on a dataset which was independent of the training dataset, it provides almost unbiased estimates of the true errors (46). Although, the performance accuracy of our model was slightly better on internal dataset compared to the external data, it is known that external validations generally perform poorer than the development model (67).

We accounted for confounders by including covariates such as batch effects, cell counts and age in our models, by applying strict exclusionary criteria such as smoking and medication intake or by performing additional sensitivity analyses such as excluding patients taking anti-depressants and accounting for differences by collection sites. Although IBS vs HC classifiers showed a low overall accuracy, the utility of a diagnostic test in discriminating against HCs is unclear since HCs lack GI symptoms. Additionally, a positive test for IBS vs HCs is likely to diagnose IBS with 70% sensitivity, providing a rule-in test. Additionally, our GO analysis suggested that the biomarkers associated with IBS vs HC comparison were associated with pathways relevant to IBS pathophysiology.

In conclusion, using comprehensive data on DNA methylation in various GI conditions and healthy controls, this Example shows that blood-based DNA methylation changes provide non-invasive biomarkers to distinguish IBS, IBD and CeD, leading to a faster diagnosis and a rule-in test for IBS. GO analysis supports the functional significance of the classifiers in disease-specific pathology. Future analysis will be aimed at testing these markers further and assessing their utility in predicting response to treatment.

### REFERENCES

1. Lovell R M, Ford A C. Clin Gastroenterol Hepatol 2012; 10:712-721 e4.
2. Cash B, et al. Am J Manag Care 2005; 11: S7-16.
3. Ladabaum U, et al. Clin Gastroenterol Hepatol 2012; 10:37-45.
4. Spiegel B M, et al. Am J Gastroenterol 2010; 105:848-58.
5. Dahlhamer J M, et al. MMWR Morb Mortal Wkly Rep 2016; 65:1166-1169.
6. Kappelman M D, et al. Clin Gastroenterol Hepatol 2007; 5:1424-9.
7. Card T R, et al. United European Gastroenterol J 2014; 2:505-12.
8. Nahon S, et al. Dig Dis Sci 2016; 61:3278-3284.
9. Nguyen V Q, et al. Inflamm Bowel Dis 2017; 23:1825-1831.
10. Kang H S, et al. World J Gastroenterol 2019; 25:989-1001.
11. Mustalahti K, et al. Ann Med 2010; 42:587-95.
12. Lebwohl B, Rubio-Tapia A. Gastroenterology 2021; 160:63-75.
13. Therrien A, et al. J Clin Gastroenterol 2020; 54:8-21.
14. Diagnosis and Management of Gluten-Associated Disorders: Springer Cham, 2021.
15. Irvine A J, et al. Am J Gastroenterol 2017; 112:65-76.
16. Lacy B E, et al. Am J Gastroenterol 2021; 116:17-44.
17. Smalley W, et al. Gastroenterology 2019; 157:851-854.
18. Fuchs V, et al. United European Gastroenterol J 2018; 6:567-575.
19. Rubio-Tapia A, et al. Am J Gastroenterol 2012; 107:1538-44; quiz 1537, 1545.
20. Ukkola A, et al. BMC Gastroenterol 2012; 12:136.
21. Card T R, et al. Scand J Gastroenterol 2013; 48:801-7.
22. Black C J, et al. Lancet Gastroenterol Hepatol 2023; 8:499-501.
23. Roth T L. Dev Psychopathol 2013; 25:1279-91.
24. Bjornsson H T, et al. Trends Genet 2004; 20:350-8.
25. Deaton A M, Bird A. Genes Dev 2011; 25:1010-22.
26. Yang X, et al. Cancer Cell 2014; 26:577-90.
27. Mahurkar S, et al. Neurogastroenterol Motil 2016; 28:410-22.
28. Kalla R, et al. J Crohns Colitis 2022.
29. Raffals L E, et al. Inflamm Bowel Dis 2022; 28:192-199.
30. Longstreth G F, et al. Gastroenterology 2006; 130:1480-91.
31. Mearin F, et al. Gastroenterology 2016; 150:1393-1407.
32. Zigmond A S, Snaith R P. Acta Psychiatr Scand 1983; 67:361-70.
33. Park S H, et al. Neurogastroenterol Motil 2016; 28:1252-60.
34. Francis C Y, et al. Aliment Pharmacol Ther 1997; 11:395-402.
35. Lin S, et al. J Crohns Colitis. 2023 Aug. 8: jad133.
36. Daperno M, et al. Gastrointest Endosc 2004; 60:505-12.
37. Best W R, et al. Gastroenterology 1976; 70:439-44
38. Thia K, et al. Inflamm Bowel Dis 2011; 17:105-11.
39. Walmsley R S, et al. Gut 1998; 43:29-32.
40. Sutherland L R, et al. Gastroenterology 1987; 92:1894-8.
41. Leffler D A, et al. Clin Gastroenterol Hepatol 2009; 7:530-6, 536 e1-2.
42. Leffler D A, et al. Clin Gastroenterol Hepatol 2009; 7:1328-34, 1334 e1-3.
43. Thompson M, et al. NPJ Genom Med 2022; 7:50.
44. Xu Z, et al. Nucleic Acids Res 2016; 44: e20.
45. Salas L A, Koestler D C. Illumina EPIC data on immunomagnetic sorted peripheral adult blood cells, 2020. github.com/immunomethylomics/FlowSorted.Blood.EPIC
46. Varma S, Simon R. BMC Bioinformatics 2006; 7:91.
47. Unal I. Comput Math Methods Med 2017; 2017:3762651.
48. Phipson B, et al. Bioinformatics 2016; 32:286-8.
49. Ojala M. GGC. 2009 Ninth IEEE International Conference on Data Mining, Miami Beach, FL, USA, 2009, pp. 908-913 2009.
50. Zhou Q, et al. Pain 2009; 146:41-6.
51. Nahm F S. Korean J Anesthesiol 2022; 75:25-36.
52. Verne G N, et al. Pain 2003; 103:99-110.
53. Videlock E J, et al. Am J Physiol Gastrointest Liver Physiol 2018; 315: G140-G157.
54. Moloney R D, et al. Front Psychiatry 2015; 6:15.
55. Mahurkar-Joshi S, Chang L. Front Psychiatry 2020; 11:805.
56. Chen M L, Sundrud M S. Inflamm Bowel Dis 2016; 22:1157-67.
57. Sollid L M. Immunogenetics 2017; 69:605-616.
58. Cobb W S, et al. Am Surg 2004; 70:750-7; discussion 757-8.
59. Mosli M H, et al. Am J Gastroenterol 2015; 110:802-19; quiz 820.
60. Pimentel M, et al. PLOS One 2015; 10: e0126438.
61. Rezaie A, et al. Dig Dis Sci 2017; 62:1480-1485.
62. Talley N J, et al. Clin Transl Gastroenterol 2019; 10: e00064.
63. Menees S B, et al. Am J Gastroenterol 2015; 110:444-54.
64. Fairbrass K M, et al. Lancet Gastroenterol Hepatol 2020; 5:1053-1062.
65. Maksimovic J, et al. Nucleic Acids Res 2015; 43: e106.
66. Thompson M, et al. Genome Biol 2019; 20:138.
67. Ramspek C L, et al. Clin Kidney J 2021; 14:49-58.

### Supplementary Methods

### Questionnaires

### Bowel Symptom Questionnaire (BSQ)

The BSQ was administered to all irritable bowel syndrome (IBS) patients, inflammatory bowel disease (IBD) patients from UCLA cohort and all celiac disease (CeD) patients and the overall disease severity scores were reported. It is a self-report measure of GI symptoms. It includes multiple questions including the Rome diagnostic questions for IBS and bowel habit subtypes, overall GI symptom severity, and individual GI symptoms. IBS, IBD and CeD participants rated the current overall intensity of their GI symptoms over the past week on a 20-point ordinal scale from 0 meaning none to 20 meaning most severe overall GI symptoms. (1-3)

### Irritable Bowel Syndrome Severity Scoring System (IBS-SSS)

IBS-SSS (4) was administered to IBS patients. This validated instrument measures the frequency and severity of abdominal pain, severity of abdominal distension, dissatisfaction with bowel habits, and interference of IBS with daily life. It is scored on a scale from 0-100 in each of the 5 categories, and a total IBS-SSS score is the sum of these five with a range of 0-500 with remission <75, mild 75-175, moderate 175-300, severe >300.

### Hospital Anxiety and Depression Scale (HAD)

The HAD (5) is a validated self-assessment mood scale specifically designed for use in non-psychiatric settings for assessment of symptoms of anxiety and depression. The HAD provides two 7-item subscales: anxiety and depression, with scores ranging from 0-21, with 0-7 representing ‘non-cases’, 8-10 ‘doubtful cases’, and 11-21 representing ‘cases’.

### Adverse Childhood Experiences (ACE) Questionnaire

The ACE questionnaire assesses the presence of early adverse life events before age 18 with 18 questions in eight ACE domains (number of questions) of physical (1), emotional (2), and sexual abuse (4), and includes household substance abuse (2), parental separation or divorce (1), mental illness in household (2), incarcerated household member (1), and parent treated violently (2) (6). ACE score is calculated by assigning one point for each domain ‘Yes’=1 or ‘No’=0 (ACE score range is 0-8=Physical abuse+Emotional Abuse+Sexual Abuse+Household Substance Abuse+Parent Separation/Divorce+Household Mental Illness+Incarcerated Household Member+Parent Treated Violently). ACE can also be scored as presence (ACE total score ≥1) or absence (ACE total score=0) of an ACE.

### Crohn's Disease Activity Index (CDAI)

The CDAI scores the used to assess disease activity in the UCLA Crohn's disease (CD) patients (7). The CDAI instrument consists of eight variables, two of which are subjective, and each weighted according to its ability to be predictive of disease activity. The total score ranges from 0 to over 600. CDAI scores <150 represent clinical remission, 150-220 mild disease, 220-450 moderate disease, and >450 severe disease.

### Short Crohn's Disease Activity Index (SCDAI)

The SCDAI disease activity scores were available on the CCF CD patients. SCDAI is a short version of CDAI and can be used to reliably replace full CDAI score for assessment of disease activity in mildly to moderately active CD patients (8). The range of score is same as SCDAI.

### Simple Endoscopic Score for Crohn's Disease (SES-CD)

SES-CD score were available for both UCLA and CCF patients. This is a simple endoscopic scoring system for Crohn's disease which assesses the size of mucosal ulcers, the ulcerated surface, affected surface, and luminal narrowing (9). The calculated score is interpreted as remission (0-2), mild (3-6), moderate (7-15), severe (>15).

### Simple Clinical Colitis Activity Index (SCCAI)

SCCAI is used in initial assessment of patients with ulcerative colitis Ulcerative Colitis (UC) and correlates well with more complex scoring systems (10). The score ranges from 0 to 19, where a score of >5 indicates active disease.

### Ulcerative Colitis Disease Activity Score (UCDAI)

UCDAI provides an assessment of the disease severity in UC patients (11). The score can range from 0-9 with a score <2 indicating remission, 2-4 mild, 5-7 moderate and >7 severe disease.

### Coeliac Disease Adherence Test (CDAT Score)

CDAT is a gluten-free diet (GFD) adherence assessment method which consists of seven questions, and the score ranges from 7-35; score >13 indicates non-adherence (12).

### Celiac Symptom Index (CSI) Score

CSI score (13) ranges from 16-80 and <30 indicates high quality of life (QoL) and an excellent gluten-free diet adherence and >45 indicates relatively poor Qol and worse gluten-free diet adherence.

### DNA Methylation Arrays

Illumina's Human Methylation 450K array or Infinium MethylationEPIC BeadChip, Illumina™ were used to assess DNA methylation levels in PBMCs. The analysis was performed at three levels including, data preprocessing, classifier development, and classifier evaluation using R statistical language (14).

### Data Pre-Processing

Data import and normalization of EPIC array data: As outlined in FIG. 6, this stage of analysis includes steps to preprocess the raw Illumina DNA methylation array (IDAT) files generated by the Illumina iScan scanner and output normalized and filtered data. Data preprocessing was performed using the R package Enmix (15). Quality Control (QC) metrics were generated using the ‘QCinfo’ function. Samples that did not pass the QC threshold were excluded from analysis. Using the ‘preprocessENmix’ function, we used the “oob” method for background correction and “RELIC” for dye bias correction. This resulted in signal intensity data on methylated and unmethylated channels of a “MethylSet” class object. The data were quantile normalized using ‘norm.quantile’, separately for Methylated and Unmethylated intensities for Infinium I or Infinium II probes (“quantile1” method). Probe design type bias was corrected using Regression on Correlated Probes (RCP), followed by filtering on QC metrics. Detection P value threshold to identify low quality data points was set to 0.000001. “Impute” parameter was set to “True” (after removing rows or columns with too many missing values, default threshold of >5% missing values). All the steps were combined into a single function which can be applied to either single or multiple IDAT files. Cell types were estimated using ‘estimateCellCounts2’ function and “preprocessNoob” method for EPIC platform in FlowSorted.Blood.EPIC package (16). Methylation age was predicted using “methyAge” function.

Data import and combined normalization of 450K and EPIC array data: IBS and healthy controls were measured on the 450K (N=100 and 31, respectively) and EPIC platforms (IBS N=48 and HCs N=55). Data from the two platforms were preprocessed using the exact same steps and normalized using ‘preprocessNoob function’. The arrays were combined using ‘combineArrays’ (17) function in ‘minfi’ package.

IBS vs HCs analysis was performed using three separate analyses. The first included a larger set of IBS and HCs samples that were run on either the EPIC or 450K methylation arrays. This analysis used the platform type as the batch covariate. The second analysis included a cohort of IBS and HCs from the first analysis that was down-sampled to an equal number of IBS and HCs for a balanced sample to avoid over-fitting. The third analysis included IBS patient and HC samples that were run on the EPIC platform only. This allowed for the identification of differential methylation on probes that were not represented on the 450K platform. All the comparisons discussed in the paper are shown in Table 7.

Probe filtration: From the original 855,790 CpGs measured on the EPIC array, QC-based filtering resulted in 846,790 probes. We further excluded X and Y chromosomal probes (n=18,880), probes with SNPs and repeats (n=25,329), and non-specific or cross-reactive probes (n=29,233) following annotations from the IlluminaHumanMethylation EPICanno.ilm10b2.hg 19 package. After initial filtering, samples were separated into comparison group pairs (described below) and further filtering was applied to exclude probes with low variance (standard deviation <0.02) and probes with consistently high or low methylation beta values (beta <0.1 or >0.9) (18). This resulted in 180,000 to 250,000 probes per pair depending upon the variability between samples.

Making Comparison groups: After normalization and initial filtering, we prepared the data for input into the machine learning models. Table 5 shows all the comparison groups tested and the methylation platforms used to generate the data.

TABLE 7
Comparison groups and DNA methylation platforms

| Comparison groups | Platform | N in Group 1 | N in Group 2 |
|---|---|---|---|
| IBS vs IBD | EPIC | 48 | 43 |
| IBS vs CeD | EPIC | 48 | 34 |
| IBD vs CeD | EPIC | 43 | 34 |
| IBS vs HCs | EPIC only | 48 | 55 |
| | EPIC + 450K | 148 | 86 |
| | Down-sampled | 86 | 86 |
| | EPIC + 450K | | |
| IBD vs HCs | EPIC | 43 | 55 |
| CeD vs HCs | EPIC | 34 | 55 |

IBS, irritable bowel syndrome;
IBD, inflammatory bowel diseases;
CeD, celiac disease;
HCs, healthy controls;
N = number of patients

### Classifier Development and Evaluation

### Covariates

All analyses included age, cell-type proportions (six cell types including CD8−T cells, CD4−T cells, natural killer cells, B-cells, monocytes, neutrophils) and DNA methylation batch as covariates in the models. IBS vs HC analysis for the larger dataset included platform (450K or EPIC) as the batch covariate.

### Generating Training and Test Fold Data

We developed a double cross-validation framework in which we split the data into 10 folds of an outer training set and an outer test set (90:10). We next split the outer training set into inner training and inner test sets (90:10) and performed an internal 10-fold cross validation on these inner training and test sets for choosing model hyperparameters. After the hyperparameters were selected from the inner cross-validation, we trained a model on the entire outer training set, and used it to predict the outer test set. We performed these steps 10 times (once per outer test set) to generate an out of sample predictor.

### Selection of Model Hyperparameters

For each dataset, models on training data were generated using generalized linear models (GLM) via penalized maximum likelihood using glmnet package. Regularization parameter (A) was selected using 10-fold cross-validation on inner training and test data. A model corresponding to A that minimizes the error of cross-validation (Amin) was chosen and prediction was made for each of the 10 outer training-test data splits. Separate models were generated for lasso or elastic net penalties (x=0, 0.1, 0.2, . . . , 1), and the model associated with a resulting in the best performance (based on cross-validated accuracy) was chosen as the final model.

### Evaluation of Model Performance

Various performance metrics were generated based on the 2×2 confusion matrix for each of the comparison, created using caret package and GLMs. These included p-values from linear regression of predicted and true values, accuracy (confidence intervals), accuracy p values, kappa, sensitivity, specificity, positive predictive value (PPV), negative predictive values (NPV), precision, recall, F1, prevalence and balanced accuracy. In these analyses, a model with a low p value (<0.05) and the highest sensitivity and specificity were chosen as the best models. However, all other metrics mentioned above help in addressing different aspects of model performance. Performance metrics were calculated based on the ‘confusion matrix’ which gives the number of true and false positive and negative predictions on test samples.

### REFERENCES

1. Labus J S, et al. Aliment Pharmacol Ther 2004; 20:89-97.
2. Talley N J, et al. Aust N Z J Med 1995; 25:302-8.
3. Schmulson M, et al. Am J Gastroenterol 1999; 94:2929-35.
4. Francis C Y, et al. Aliment Pharmacol Ther 1997; 11:395-402.
5. Zigmond A S, Snaith R P. Acta Psychiatr Scand 1983; 67:361-70.
6. Park S H, et al. Neurogastroenterol Motil 2016; 28:1252-60.
7. Best W R, et al. Gastroenterology 1976; 70:439-44.
8. Thia K, et al. Inflamm Bowel Dis 2011; 17:105-11.
9. Daperno M, et al. Gastrointest Endosc 2004; 60:505-12.
10. Walmsley R S, et al. Gut 1998; 43:29-32.
11. Sutherland L R, et al. Gastroenterology 1987; 92:1894-8.
12. Leffler D A, et al. Clin Gastroenterol Hepatol 2009; 7:530-6, 536 e1-2.
13. Leffler D A, et al. Clin Gastroenterol Hepatol 2009; 7:1328-34, 1334 e1-3.
14. Team R C. R: A language and environment for statistical computing. Vienna, Austria: R Foundation for Statistical Computing, 2020.
15. Xu Z, et al. Nucleic Acids Res 2016; 44: e20.
16. Salas L A, Koestler D C. Illumina EPIC data on immunomagnetic sorted peripheral adult blood cells, 2020.
17. Fortin J P, et al. Bioinformatics 2017; 33:558-560.
18. Mansell G, et al. BMC Genomics 2019; 20:366.

### Supplementary Results

TABLE 8
Medications used by patients and healthy controls (HCs)

| | IBS | IBD | CeD | HCs |
|---|---|---|---|---|
| Drug class | (N = 148) | (N = 46) | (N = 34) | (N = 86) |
| Statins [N (%)] | 2 (1) | 1 (2) | 3 (9) | 3 (3) |
| SSRI or Antidepressants | 11 (7) | 3 (6) | 3 (9) | 0 (0) |
| [N (%)] | | | | |
| Analgesics* [N (%)] | 12 (8) | 1 (2) | 4 (12) | 10 (12) |
| Beta blocker [N (%)] | 0 (0) | 1 (2) | 1 (3) | 0 (0) |
| Benzodiazepine [N (%)] | 3 (2) | 4 (9) | 1 (3) | 0 (0) |

IBS: irritable bowel syndrome; IBD: inflammatory bowel disease; CeD, celiac disease; HCs, Healthy controls; SSRIs: Selective serotonin reuptake inhibitors;
*Analgesics included acetaminophen and non-steroidal anti-inflammatory drugs (NSAIDs) and were taken on an as needed basis.

### Disease Activity Scores for IBS. IBD and CeD Patients

TABLE 9A
Symptom severity scores in IBS patients

| Symptom | IBS N = 148 | | IBS N = 48 | |
|---|---|---|---|---|
| Bowel habit subtype [count (%)] | (450K + Epic) | | (Epic) | |
| IBS-C | 44 | (27) | 13 | (27) |
| IBS-D | 53 | (42) | 20 | (42) |
| IBS-M | 42 | (25) | 12 | (25) |
| IBS-U | 9 | (6) | 3 | (6) |
| Overall severity of GI Symptoms | 9.45 | (4.18) | 9.55 | (4.32) |
| [Mean (SD)] | | | | |
| Abdominal pain score [Mean (SD)] | 8.97 | (4.12) | 9.02 | (3.84) |
| IBS-SSS [Mean (SD)] | 237 | (89.7) | 226 | (86.4) |
| ACE [Mean (SD)] | 1.91 | (1.89) | 1.79 | (2.04) |
| HAD Anxiety [Mean (SD)] | 7.89 | (4.41) | 7.5 | (4.49) |
| HAD Depression [Mean (SD)] | 3.4 | (3.36) | 3.30 | (3.76) |

IBS: irritable bowel syndrome; IBS-C: constipation-predominant IBS; IBS-D, diarrhea-predominant IBS; IBS-M: Mixed IBS; IBS-U: Un-subtyped IBS; UC: ulcerative colitis; HCs, healthy controls; GI, gastrointestinal; Overall Severity of GI Symptoms, 0-20; Abdominal pain score, 0-20; IBS-SSS: IBS symptom severity score, 0-500; ACE: adverse childhood experiences, 0-8; HAD: hospital anxiety and depression scale; HAD Anxiety, 0-21; HAD Depression 0-21; SD: standard deviation.

TABLE 9B
Symptom severity scores in IBD patients
Ulcerative colitis (UC) patients

| | UC UCLA | UC CCF |
|---|---|---|
| | (N = 12) | (N = 10) |
| SCCAI [Mean (SD)] | 6.17 (4.84) | NA |
| UCDAI [Mean (SD)] | NA | 4.29 (1.25) |
| Disease duration [Mean (SD)] | 8.29 (12.57) | 5.11 (7.48) |
| Overall Severity of GI Symptoms [Mean (SD); 0-20] | 7.50 (4.12) | NA |

SCCAI: simple clinical colitis activity index, 0-21, Flare >=5 (past 3 days); UC:
ulcerative colitis; UCDAI: ulcerative colitis disease activity index, score = 0-9: remission (<2),
mild (2-4), moderate (5-7), severe disease (>7); GI, gastrointestinal; Overall Severity of GI
Symptoms, 0-20; SD: standard deviation; UCLA: patients recruited at University of California
Los Angeles; CCF, Crohn's and Colitis Foundation.

| Crohn's disease patients | | |
|---|---|---|
| | Crohn's UCLA | Crohn's CCF |
| | (N = 13) | (N = 12) |

| | | | | |
|---|---|---|---|---|
| SES-CD score [Mean (SD)] | 4.40 | (5.40) | 4.44 | (5.55) |

| | | | |
|---|---|---|---|
| CDAI [Mean (SD)] | 146.36 | (78.10) | NA |

| SCDAI [Mean (SD)] | NA | | 243.82 | (73.77) |
|---|---|---|---|---|
| Disease Location [N (%)]: | | | | |
| Ileal | 5 | (38%) | 6 | (54%) |
| Colonic | 1 | (8%) | 3 | (27%) |
| Ileocolonic | 2 | (15%) | 1 | (9%) |
| Upper GI involvement | 2 | (15%) | 1 | (9%) |
| Not available | 3 | (23%) | 1 | (9%) |
| Disease duration [Years, Mean (SD)] | 6.77 | (7.24) | 4.72 | (4.82) |

| | | | |
|---|---|---|---|
| Overall Severity of GI Symptoms [Mean (SD); 0-20] | 9 | (5.10) | NA |

SES-CD: simple endoscopic score for Crohn's disease score, 0-2 remission, 3-6 mild, 7-15 moderate, >15 severe]; CDAI: Crohn's disease activity index, 0-149 asymptomatic remission; 150-220 mild to moderate; 221-450 moderate to severe; 451-1100 severe to fulminant disease; SCDAI: short CDAI score, 44-150 remission, 150-219 mild, 220-450 moderate, >450 severe; Overall Severity of GI Symptoms, 0-20; GI, gastrointestinal; SD: standard deviation; Samples recruited at UCLA: University of California, Los Angeles and CCF: Crohn's and Colitis Foundation.

TABLE 9C
Symptom severity scores in celiac disease patients

| | Scores |
|---|---|
| CDAT Score [Mean (SD)] | 14.90 (3.94) |
| (7-35 higher score indicates worse gluten-free | |
| diet adherence) | |
| CSI Score [Mean (SD)] | 37.48 (10.76) |
| Overall severity of GI symptoms [Mean (SD)] | 5.90 (4.90) |

CDAT: celiac disease adherence test score, ≤30, remission; 30-45 moderate disease activity; ≥45 indicates relatively poor QoL and worse gluten-free diet adherence; CSI: celiac symptom index, 16-80 and ≤30 indicates high QoL and excellent gluten-free diet adherence, while ≥45 indicates relatively poor QoL and worse gluten-free diet adherence; Overall Severity of GI Symptoms, 0-20; GI, gastrointestinal; SD: standard deviation

TABLE 10A
Differentially Methylated CpG Sites
Associated with Celiac vs Healthy

| | logFC | P.Value | adj.P.Val | UCSC_RefGene_Name |
|---|---|---|---|---|
| cg08854185 | 0.072 | 1.37E−08 | 1.55E−03 | DBNDD1 |
| cg17353680 | −0.128 | 1.51E−07 | 4.07E−03 | KCNK12 |
| cg21523688 | 0.195 | 1.56E−07 | 4.07E−03 | SORD |
| cg20802826 | −0.113 | 3.58E−07 | 4.55E−03 | |
| cg01668008 | 0.028 | 4.72E−07 | 5.01E−03 | |
| cg26840333 | −0.150 | 5.47E−07 | 5.01E−03 | |
| cg14293323 | 0.047 | 6.32E−07 | 5.28E−03 | |
| cg04418355 | −0.104 | 7.25E−07 | 5.84E−03 | HLA-DQB2 |
| cg23580000 | 0.044 | 7.79E−07 | 6.06E−03 | ADCY7 |
| cg24674703 | −0.071 | 8.62E−07 | 6.08E−03 | CD5 |
| cg01395261 | 0.049 | 1.44E−06 | 8.35E−03 | MAST3 |
| cg00902815 | −0.062 | 1.93E−06 | 1.01E−02 | LMO7 |
| cg04990420 | −0.064 | 3.16E−06 | 1.21E−02 | ADORA2A |
| cg22959409 | 0.044 | 3.30E−06 | 1.21E−02 | LRRC33 |
| cg24147428 | 0.060 | 2.86E−06 | 1.21E−02 | SIPA1 |
| cg23966795 | 0.035 | 2.56E−06 | 1.21E−02 | TANC1 |
| cg09704401 | 0.046 | 2.66E−06 | 1.21E−02 | |
| cg14058851 | 0.042 | 4.73E−06 | 1.47E−02 | JARID2 |
| cg07160014 | 0.052 | 4.74E−06 | 1.47E−02 | |
| cg06801857 | −0.060 | 4.96E−06 | 1.49E−02 | GALNT2 |
| cg03334052 | −0.072 | 4.92E−06 | 1.49E−02 | |
| cg20655099 | 0.033 | 5.60E−06 | 1.56E−02 | TNRC18 |
| cg13641591 | 0.034 | 5.61E−06 | 1.56E−02 | UVRAG |
| cg25015416 | 0.037 | 6.16E−06 | 1.66E−02 | CNP |
| cg21493951 | −0.186 | 7.64E−06 | 1.84E−02 | HLA-DQB1 |
| cg18881723 | −0.059 | 8.20E−06 | 1.89E−02 | SLAMF1 |
| cg00903577 | −0.106 | 8.14E−06 | 1.89E−02 | |
| cg03597174 | −0.081 | 8.43E−06 | 1.90E−02 | |
| cg17289597 | 0.029 | 9.57E−06 | 2.06E−02 | ADAMTS14 |
| cg06601266 | 0.040 | 1.08E−05 | 2.06E−02 | C10orf119 |
| cg06679990 | 0.047 | 1.09E−05 | 2.06E−02 | GLI1 |
| cg09259179 | 0.054 | 9.96E−06 | 2.06E−02 | ILDR2 |
| cg19646227 | −0.047 | 1.01E−05 | 2.06E−02 | LOC100130331 |
| cg21593149 | −0.063 | 1.10E−05 | 2.06E−02 | MAEA |
| cg12807330 | 0.046 | 9.58E−06 | 2.06E−02 | PCDH24 |
| cg13630239 | 0.057 | 1.13E−05 | 2.08E−02 | RRP12 |
| cg04628802 | −0.041 | 1.36E−05 | 2.23E−02 | CALB2 |
| cg09837656 | 0.069 | 1.30E−05 | 2.23E−02 | PLEKHO2 |
| cg22335340 | −0.033 | 1.48E−05 | 2.26E−02 | PTPN6 |
| cg14218480 | 0.052 | 1.47E−05 | 2.26E−02 | |
| cg16412914 | 0.029 | 1.54E−05 | 2.28E−02 | AXIN1 |
| cg07839457 | −0.091 | 1.54E−05 | 2.28E−02 | NLRC5 |
| cg21253721 | −0.044 | 1.54E−05 | 2.28E−02 | TUSC5 |
| cg16385865 | −0.090 | 1.57E−05 | 2.29E−02 | |
| cg26653824 | 0.038 | 1.63E−05 | 2.30E−02 | |
| cg05875239 | 0.042 | 1.70E−05 | 2.31E−02 | |
| cg24470880 | 0.041 | 1.72E−05 | 2.32E−02 | |
| cg04747996 | 0.032 | 1.73E−05 | 2.33E−02 | PPM1L |
| cg17177006 | −0.068 | 1.76E−05 | 2.34E−02 | EDEM1 |
| cg03589820 | 0.036 | 1.81E−05 | 2.36E−02 | ATG7 |
| cg15872079 | −0.051 | 1.78E−05 | 2.36E−02 | CMTM7 |
| cg19341652 | −0.101 | 1.79E−05 | 2.36E−02 | DSE |
| cg02192218 | 0.031 | 1.84E−05 | 2.38E−02 | CLN3 |
| cg11598872 | −0.07+ | 2.03E−05 | 2.40E−02 | FLJ45079 |
| cg06117093 | −0.069 | 2.08E−05 | 2.40E−02 | HDAC4 |
| cg24939194 | 0.027 | 2.06E−05 | 2.40E−02 | LFNG |
| cg22514722 | −0.086 | 1.98E−05 | 2.40E−02 | MGLL |
| cg17113856 | 0.024 | 1.95E−05 | 2.40E−02 | PPT2 |
| cg23641597 | −0.045 | 1.95E−05 | 2.40E−02 | |
| cg25256099 | 0.064 | 1.98E−05 | 2.40E−02 | |
| cg26880399 | −0.037 | 2.03E−05 | 2.40E−02 | |
| cg20538986 | −0.026 | 2.04E−05 | 2.40E−02 | |
| cg19149463 | 0.035 | 2.12E−05 | 2.40E−02 | VGLL4 |
| cg25597580 | −0.050 | 2.15E−05 | 2.41E−02 | KIRREL |
| cg04132186 | 0.058 | 2.15E−05 | 2.41E−02 | |
| cg17264271 | 0.059 | 2.16E−05 | 2.41E−02 | ESR1 |
| cg04286697 | 0.021 | 2.21E−05 | 2.44E−02 | B3GNT7 |
| cg11130630 | 0.089 | 2.25E−05 | 2.45E−02 | KCTD10 |
| cg09556700 | 0.035 | 2.24E−05 | 2.45E−02 | SKI |
| cg06376949 | 0.075 | 2.34E−05 | 2.45E−02 | IFIT5 |
| cg15021238 | −0.131 | 2.28E−05 | 2.45E−02 | ITGA6 |
| cg16317457 | 0.063 | 2.51E−05 | 2.55E−02 | |
| cg21500092 | 0.032 | 2.63E−05 | 2.63E−02 | UBE2G1 |
| cg10548978 | 0.045 | 2.67E−05 | 2.64E−02 | C10orf27 |
| cg17884674 | −0.053 | 2.71E−05 | 2.66E−02 | CSNK1G2 |
| cg25887844 | −0.047 | 2.83E−05 | 2.75E−02 | ARHGAP25 |
| cg18247172 | 0.047 | 2.89E−05 | 2.78E−02 | |
| cg27363280 | 0.035 | 2.96E−05 | 2.80E−02 | |
| cg23599820 | 0.056 | 3.02E−05 | 2.82E−02 | KIAA0195 |
| cg13489576 | −0.022 | 3.04E−05 | 2.82E−02 | DLG1 |
| cg04118190 | −0.068 | 3.05E−05 | 2.82E−02 | DUSP2 |
| cg06105699 | 0.061 | 3.12E−05 | 2.82E−02 | ASPSCR1 |
| cg08415156 | 0.039 | 3.23E−05 | 2.82E−02 | CBFA2T3 |
| cg25645491 | 0.023 | 3.30E−05 | 2.82E−02 | HLA-DRA |
| cg24990317 | 0.043 | 3.31E−05 | 2.82E−02 | NCOR2 |
| cg19356389 | 0.036 | 3.30E−05 | 2.82E−02 | RING1 |
| cg01385198 | 0.041 | 3.07E−05 | 2.82E−02 | TPCN2 |
| cg16505704 | 0.043 | 3.15E−05 | 2.82E−02 | |
| cg21855021 | 0.088 | 3.16E−05 | 2.82E−02 | |
| cg10676309 | 0.026 | 3.16E−05 | 2.82E−02 | |
| cg26155674 | −0.055 | 3.21E−05 | 2.82E−02 | |
| cg16214653 | 0.050 | 3.26E−05 | 2.82E−02 | |
| cg19397991 | −0.080 | 3.36E−05 | 2.85E−02 | PASK |
| cg27184903 | 0.053 | 3.40E−05 | 2.87E−02 | APBA2 |
| cg02816525 | 0.062 | 3.63E−05 | 2.99E−02 | ADAM10 |
| cg16650073 | 0.031 | 3.64E−05 | 2.99E−02 | NTHL1 |
| cg02692808 | −0.047 | 3.76E−05 | 3.07E−02 | |
| cg17845617 | 0.029 | 3.83E−05 | 3.12E−02 | PIP5K1C |
| cg18735473 | −0.060 | 3.91E−05 | 3.15E−02 | BRUNOL4 |
| cg21777154 | 0.034 | 4.03E−05 | 3.17E−02 | C1orf21 |
| cg10785537 | 0.048 | 4.05E−05 | 3.17E−02 | DCTN1 |
| cg11963365 | 0.048 | 3.98E−05 | 3.17E−02 | KIAA0141 |
| cg06608359 | −0.048 | 4.06E−05 | 3.17E−02 | |
| cg18496212 | 0.107 | 4.13E−05 | 3.17E−02 | MITF |
| cg19035788 | −0.099 | 4.10E−05 | 3.17E−02 | |
| cg11964207 | 0.021 | 4.39E−05 | 3.18E−02 | DOT1L |
| cg06226386 | −0.097 | 4.38E−05 | 3.18E−02 | FLJ14107 |
| cg05593759 | 0.030 | 4.23E−05 | 3.18E−02 | GRAMD3 |
| cg02417408 | −0.058 | 4.40E−05 | 3.18E−02 | PPM1M |
| cg05266212 | 0.052 | 4.49E−05 | 3.18E−02 | TBX21 |
| cg04995717 | 0.068 | 4.48E−05 | 3.18E−02 | TEK |
| cg04392554 | 0.070 | 4.50E−05 | 3.18E−02 | TTC38 |
| cg14305659 | 0.023 | 4.18E−05 | 3.18E−02 | |
| cg04873169 | 0.042 | 4.56E−05 | 3.19E−02 | GRAMD4 |
| cg14712058 | 0.037 | 4.55E−05 | 3.19E−02 | SIN3B |
| cg02640489 | −0.069 | 4.60E−05 | 3.21E−02 | PNKD |
| cg21097788 | 0.035 | 4.66E−05 | 3.23E−02 | ITPK1 |
| cg22911650 | 0.063 | 4.65E−05 | 3.23E−02 | SIGLEC7 |
| cg14327394 | 0.039 | 4.71E−05 | 3.24E−02 | RGS3 |
| cg04795387 | −0.046 | 4.73E−05 | 3.24E−02 | |
| cg21371809 | −0.073 | 4.79E−05 | 3.25E−02 | FRMD4A |
| cg10422233 | 0.023 | 4.83E−05 | 3.27E−02 | C14orf73 |
| cg12194745 | 0.034 | 4.99E−05 | 3.33E−02 | BAHCC1 |
| cg11773456 | −0.060 | 5.12E−05 | 3.33E−02 | CCDC85C |
| cg24150157 | 0.034 | 5.02E−05 | 3.33E−02 | LIM2 |
| cg04718702 | 0.033 | 5.13E−05 | 3.33E−02 | RIN3 |
| cg18100887 | 0.042 | 5.14E−05 | 3.33E−02 | |
| cg 16042652 | −0.062 | 5.17E−05 | 3.33E−02 | |
| cg24735226 | −0.076 | 5.30E−05 | 3.36E−02 | CACHD1 |
| cg24879939 | 0.041 | 5.30E−05 | 3.36E−02 | PRDM16 |
| cg08149865 | 0.036 | 5.43E−05 | 3.43E−02 | EPB49 |
| cg27168573 | 0.027 | 5.66E−05 | 3.49E−02 | PXN |
| cg26767214 | 0.066 | 5.71E−05 | 3.50E−02 | CHST14 |
| cg27423838 | −0.087 | 5.74E−05 | 3.51E−02 | |
| cg11133963 | 0.035 | 5.83E−05 | 3.53E−02 | ABI3 |
| cg03055021 | 0.035 | 5.95E−05 | 3.53E−02 | FHAD1 |
| cg19910327 | 0.024 | 5.96E−05 | 3.53E−02 | PIPSL |
| cg20516845 | −0.068 | 6.00E−05 | 3.53E−02 | UBASH3A |
| cg22687902 | −0.044 | 5.98E−05 | 3.53E−02 | |
| cg02504211 | 0.035 | 6.07E−05 | 3.57E−02 | C3orf21 |
| cg21201109 | 0.023 | 6.14E−05 | 3.60E−02 | KANK2 |
| cg26852894 | −0.030 | 6.47E−05 | 3.72E−02 | |
| cg22135566 | −0.039 | 6.59E−05 | 3.76E−02 | DNAH10 |
| cg02737268 | −0.067 | 6.83E−05 | 3.79E−02 | CDC25B |
| cg07438421 | 0.031 | 6.83E−05 | 3.79E−02 | SERPINF1 |
| cg00905524 | −0.031 | 6.73E−05 | 3.79E−02 | |
| cg01291375 | 0.037 | 6.78E−05 | 3.79E−02 | |
| cg13247990 | 0.019 | 6.86E−05 | 3.80E−02 | MYLK3 |
| cg00805360 | 0.047 | 6.88E−05 | 3.80E−02 | ADAM8 |
| cg04760448 | −0.099 | 7.12E−05 | 3.86E−02 | COL18A1 |
| cg11930955 | −0.042 | 7.15E−05 | 3.87E−02 | TPCN1 |
| cg00569896 | −0.079 | 7.25E−05 | 3.89E−02 | |
| cg11874323 | 0.033 | 7.36E−05 | 3.91E−02 | C3orf52 |
| cg14473062 | −0.103 | 7.35E−05 | 3.91E−02 | |
| cg11831006 | −0.036 | 7.35E−05 | 3.91E−02 | |
| cg25783892 | −0.055 | 7.48E−05 | 3.95E−02 | GHITM |
| cg18728264 | −0.058 | 7.64E−05 | 3.99E−02 | CXCR5 |
| cg07119168 | 0.040 | 7.66E−05 | 3.99E−02 | SPTB |
| cg00811565 | −0.029 | 7.90E−05 | 4.05E−02 | FAM159B |
| cg02757488 | 0.041 | 7.89E−05 | 4.05E−02 | NUMBL |
| cg26350921 | 0.026 | 7.91E−05 | 4.05E−02 | TBC1D16 |
| cg01191154 | −0.028 | 7.91E−05 | 4.05E−02 | |
| cg11981868 | 0.039 | 7.98E−05 | 4.06E−02 | NOTCH4 |
| cg15903956 | −0.080 | 7.96E−05 | 4.06E−02 | |
| cg08085199 | 0.022 | 8.05E−05 | 4.07E−02 | CIRH1A |
| cg26971042 | −0.047 | 8.09E−05 | 4.08E−02 | TLE3 |
| cg04647790 | −0.046 | 8.40E−05 | 4.10E−02 | FLJ37307 |
| cg22069247 | 0.045 | 8.37E−05 | 4.10E−02 | NMUR1 |
| cg07894334 | 0.071 | 8.28E−05 | 4.10E−02 | SOCS5 |
| cg27206026 | −0.079 | 8.26E−05 | 4.10E−02 | |
| cg17516825 | −0.074 | 8.34E−05 | 4.10E−02 | |
| cg03280235 | −0.059 | 8.54E−05 | 4.12E−02 | PBX2 |
| cg04337403 | 0.027 | 8.53E−05 | 4.12E−02 | TSSC1 |
| cg17113883 | −0.099 | 8.71E−05 | 4.16E−02 | THEMIS |
| cg06430465 | −0.039 | 8.90E−05 | 4.23E−02 | LMF1 |
| cg11254522 | 0.050 | 8.95E−05 | 4.24E−02 | FGR |
| cg20674490 | 0.053 | 9.02E−05 | 4.25E−02 | RUNX3 |
| cg05638439 | 0.064 | 9.04E−05 | 4.25E−02 | WDR90 |
| cg24163242 | 0.074 | 9.18E−05 | 4.29E−02 | |
| cg23526676 | −0.042 | 9.28E−05 | 4.30E−02 | C6orf27 |
| cg27529037 | 0.049 | 9.33E−05 | 4.30E−02 | PCIF1 |
| cg03209332 | −0.057 | 9.29E−05 | 4.30E−02 | |
| cg16775598 | −0.089 | 9.33E−05 | 4.30E−02 | |
| cg16661164 | −0.044 | 9.59E−05 | 4.30E−02 | GOLGA6A |
| cg12436019 | −0.066 | 9.67E−05 | 4.30E−02 | PCCA |
| cg03856153 | −0.041 | 9.65E−05 | 4.30E−02 | PTPRG |
| cg25029657 | 0.034 | 9.86E−05 | 4.31E−02 | ACOT7 |
| cg26802951 | −0.029 | 9.85E−05 | 4.31E−02 | RUFY1 |
| cg01164574 | 0.038 | 9.84E−05 | 4.31E−02 | |
| cg08876481 | 0.039 | 9.91E−05 | 4.32E−02 | LARP4B |
| cg19518672 | 0.080 | 9.92E−05 | 4.32E−02 | SEPTIN8 |
| cg15816464 | 0.051 | 1.01E−04 | 4.38E−02 | SMG6 |
| cg07502661 | −0.041 | 1.01E−04 | 4.38E−02 | |
| cg11970204 | −0.062 | 1.03E−04 | 4.38E−02 | BTRC |
| cg10555106 | 0.026 | 1.02E−04 | 4.38E−02 | LOC388588 |
| cg01914278 | −0.048 | 1.03E−04 | 4.38E−02 | |
| cg14449910 | 0.046 | 1.03E−04 | 4.40E−02 | SLC22A18AS |
| cg07018107 | 0.049 | 1.04E−04 | 4.40E−02 | MIR1208 |
| cg09036531 | −0.045 | 1.04E−04 | 4.40E−02 | |
| cg21507807 | 0.058 | 1.05E−04 | 4.41E−02 | RASA3 |
| cg00772991 | −0.072 | 1.05E−04 | 4.43E−02 | |
| cg09205595 | 0.051 | 1.06E−04 | 4.44E−02 | AGAP3 |
| cg08159594 | −0.069 | 1.06E−04 | 4.44E−02 | NAPEPLD |
| cg06893560 | −0.055 | 1.06E−04 | 4.44E−02 | |
| cg13015534 | −0.088 | 1.07E−04 | 4.45E−02 | ST6GALNAC1 |
| cg23679724 | 0.023 | 1.08E−04 | 4.46E−02 | CTSZ |
| cg03108250 | 0.034 | 1.09E−04 | 4.48E−02 | KIR3DX1 |
| cg05992079 | −0.080 | 1.10E−04 | 4.49E−02 | SIT1 |
| cg14599362 | −0.023 | 1.12E−04 | 4.55E−02 | QSOX2 |
| cg23903129 | −0.061 | 1.14E−04 | 4.63E−02 | GPR133 |
| cg07081759 | 0.055 | 1.15E−04 | 4.63E−02 | FAM53B |
| cg00256485 | 0.036 | 1.17E−04 | 4.68E−02 | LPCAT1 |
| cg02622647 | −0.228 | 1.17E−04 | 4.71E−02 | |
| cg08909156 | 0.044 | 1.20E−04 | 4.77E−02 | TRAPPC3 |
| cg00153395 | 0.038 | 1.20E−04 | 4.78E−02 | JAK1 |
| cg08701429 | 0.032 | 1.21E−04 | 4.78E−02 | |
| cg10821261 | 0.051 | 1.21E−04 | 4.78E−02 | |
| cg02233835 | −0.051 | 1.21E−04 | 4.78E−02 | |
| cg07019638 | −0.049 | 1.22E−04 | 4.78E−02 | EHD1 |
| cg10822495 | −0.032 | 1.22E−04 | 4.78E−02 | SKA2 |
| cg08596595 | −0.051 | 1.25E−04 | 4.82E−02 | ATHL1 |
| cg14592260 | 0.025 | 1.24E−04 | 4.82E−02 | DDX10 |
| cg06858263 | 0.034 | 1.24E−04 | 4.82E−02 | DNAJB6 |
| cg16173109 | −0.075 | 1.24E−04 | 4.82E−02 | |
| cg08401628 | −0.041 | 1.28E−04 | 4.88E−02 | |
| cg26930078 | −0.033 | 1.30E−04 | 4.89E−02 | C17orf53 |
| cg20460514 | 0.038 | 1.30E−04 | 4.89E−02 | LPIN1 |
| cg21444731 | −0.047 | 1.29E−04 | 4.89E−02 | RGL4 |
| cg24735235 | 0.130 | 1.31E−04 | 4.90E−02 | FLJ37453 |
| cg01542019 | 0.044 | 1.31E−04 | 4.90E−02 | TECR |
| cg04607131 | −0.075 | 1.32E−04 | 4.90E−02 | HTR6 |
| cg16278496 | −0.063 | 1.33E−04 | 4.91E−02 | C14orf64 |
| cg12452300 | 0.025 | 1.33E−04 | 4.91E−02 | GRM2 |
| cg24018633 | −0.033 | 1.33E−04 | 4.91E−02 | |
| cg04712122 | −0.116 | 1.35E−04 | 4.91E−02 | BCL11B |
| cg25140190 | 0.048 | 1.34E−04 | 4.91E−02 | C18orf1 |
| cg15278699 | 0.042 | 1.35E−04 | 4.91E−02 | PWWP2B |
| cg21756476 | 0.047 | 1.36E−04 | 4.91E−02 | RORA |
| cg16563370 | 0.050 | 1.36E−04 | 4.91E−02 | SLFN13 |
| cg16773067 | 0.073 | 1.35E−04 | 4.91E−02 | |
| cg19579005 | −0.056 | 1.36E−04 | 4.91E−02 | |
| cg00656410 | 0.032 | 1.37E−04 | 4.91E−02 | SDF4 |
| cg22797297 | 0.077 | 1.37E−04 | 4.91E−02 | |
| cg18651347 | −0.054 | 1.38E−04 | 4.92E−02 | AUTS2 |
| cg20299697 | 0.044 | 1.39E−04 | 4.92E−02 | MRAS |
| cg20292636 | 0.051 | 1.40E−04 | 4.92E−02 | PLEKHA6 |
| cg01449715 | 0.051 | 1.38E−04 | 4.92E−02 | RNASEH2B |
| cg01867764 | −0.063 | 1.38E−04 | 4.92E−02 | |
| cg14106710 | −0.068 | 1.40E−04 | 4.92E−02 | |
| cg27548256 | 0.036 | 1.42E−04 | 4.95E−02 | ACTN4 |
| cg08335767 | −0.028 | 1.42E−04 | 4.96E−02 | HMGA1 |
| cg11115622 | −0.056 | 1.43E−04 | 4.96E−02 | PLEKHG3 |
| cg00875849 | −0.031 | 1.42E−04 | 4.96E−02 | |
| cg04554929 | 0.144 | 1.43E−04 | 4.96E−02 | |
| cg20341836 | 0.047 | 1.46E−04 | 4.99E−02 | HCG4P6 |
| cg08297616 | −0.062 | 1.46E−04 | 4.99E−02 | |
| cg16726039 | −0.094 | 1.46E−04 | 5.00E−02 | |

TABLE 10B
Differentially Methylated CpG Sites Associated with IBD vs Celiac Disease

| | logFC | P.Value | adj.P.Val | UCSC_RefGene_Name | |
|---|---|---|---|---|---|
| cg16455376 | 0.038 | 2.42E−07 | 9.92E−03 | | |
| cg25122699 | 0.050 | 9.33E−08 | 9.92E−03 | CPEB4 | FALSE |
| cg04418355 | 0.102 | 7.04E−07 | 1.59E−02 | HLA-DQB2 | FALSE |
| cg04098052 | −0.094 | 1.94E−06 | 1.88E−02 | | |
| cg08573679 | −0.058 | 1.97E−06 | 1.88E−02 | | |
| cg18181906 | 0.031 | 2.37E−06 | 1.88E−02 | | |
| cg14900154 | −0.034 | 2.31E−06 | 1.88E−02 | ANKFY1 | FALSE |
| cg01613294 | 0.112 | 2.39E−06 | 1.88E−02 | APOL3 | FALSE |
| cg03109660 | 0.099 | 1.46E−06 | 1.88E−02 | RELL1 | FALSE |
| cg10274029 | 0.071 | 3.68E−06 | 2.19E−02 | | |
| cg17492395 | 0.085 | 3.95E−06 | 2.22E−02 | | |
| cg08065963 | 0.058 | 5.27E−06 | 2.44E−02 | | |
| cg13092082 | 0.055 | 6.12E−06 | 2.44E−02 | | |
| cg12113251 | 0.037 | 5.47E−06 | 2.44E−02 | FAM119B | FALSE |
| cg15464148 | 0.068 | 6.07E−06 | 2.44E−02 | LPAR5 | FALSE |
| cg13482010 | 0.059 | 5.87E−06 | 2.44E−02 | SEMASB | FALSE |
| cg21030160 | 0.050 | 6.77E−06 | 2.46E−02 | | |
| cg19668476 | 0.044 | 7.36E−06 | 2.46E−02 | | |
| cg05239811 | 0.054 | 7.08E−06 | 2.46E−02 | C6orf27 | FALSE |
| cg07036035 | 0.051 | 6.96E−06 | 2.46E−02 | TCL6 | FALSE |
| cg11974162 | 0.037 | 8.95E−06 | 2.59E−02 | | |
| cg25214310 | 0.033 | 9.85E−06 | 2.59E−02 | | |
| cg16727774 | 0.051 | 1.02E−05 | 2.59E−02 | CA5A | FALSE |
| cg13620439 | 0.039 | 9.93E−06 | 2.59E−02 | CLU | FALSE |
| cg19672873 | 0.055 | 1.16E−05 | 2.74E−02 | FAM177B | FALSE |
| cg11898431 | 0.095 | 1.20E−05 | 2.75E−02 | | |
| cg05946118 | 0.050 | 1.40E−05 | 2.99E−02 | | |
| cg24364282 | 0.041 | 1.54E−05 | 2.99E−02 | | |
| cg25556035 | 0.033 | 1.47E−05 | 2.99E−02 | NFIX | FALSE |
| cg11035519 | 0.056 | 1.56E−05 | 2.99E−02 | ST14 | FALSE |
| cg03796224 | 0.033 | 1.63E−05 | 3.02E−02 | RBPMS | FALSE |
| cg09073441 | 0.061 | 1.86E−05 | 3.29E−02 | | |
| cg25474070 | 0.044 | 1.93E−05 | 3.37E−02 | IL3 | FALSE |
| cg24540603 | 0.077 | 1.99E−05 | 3.39E−02 | | |
| cg12075776 | 0.050 | 1.98E−05 | 3.39E−02 | DMTF1 | FALSE |
| cg08149865 | −0.047 | 2.00E−05 | 3.39E−02 | EPB49 | FALSE |
| cg03161309 | −0.091 | 2.26E−05 | 3.44E−02 | | |
| cg06183295 | 0.061 | 2.26E−05 | 3.44E−02 | | |
| cg27123273 | 0.049 | 2.38E−05 | 3.44E−02 | | |
| cg06624244 | 0.050 | 2.08E−05 | 3.44E−02 | C1orf64 | FALSE |
| cg08653292 | 0.150 | 2.36E−05 | 3.44E−02 | FAM111A | FALSE |
| cg02376282 | 0.048 | 2.21E−05 | 3.44E−02 | HCCA2 | FALSE |
| cg00328219 | 0.042 | 2.28E−05 | 3.44E−02 | KIF13B | |
| cg08451469 | 0.046 | 2.41E−05 | 3.44E−02 | LOC285830 | FALSE |
| cg02652361 | −0.090 | 2.30E−05 | 3.44E−02 | MALAT1 | FALSE |
| cg23670794 | 0.041 | 2.28E−05 | 3.44E−02 | ZBED2 | FALSE |
| cg07609372 | 0.056 | 3.07E−05 | 3.45E−02 | ASB10 | FALSE |
| cg22792367 | −0.042 | 2.70E−05 | 3.45E−02 | | |
| cg06861560 | 0.025 | 2.73E−05 | 3.45E−02 | | |
| cg02156723 | 0.106 | 2.73E−05 | 3.45E−02 | | |
| cg04337358 | −0.082 | 2.76E−05 | 3.45E−02 | | |
| cg21607649 | 0.056 | 2.91E−05 | 3.45E−02 | | |
| cg18460107 | 0.065 | 3.17E−05 | 3.45E−02 | CLEC4C | FALSE |
| cg12127149 | −0.071 | 2.75E−05 | 3.45E−02 | DYNC1H1 | FALSE |
| cg21638533 | −0.094 | 3.19E−05 | 3.45E−02 | IFFO1 | FALSE |
| cg08120226 | −0.049 | 3.15E−05 | 3.45E−02 | MUC4 | FALSE |
| cg23295629 | 0.089 | 2.50E−05 | 3.45E−02 | PVT1 | FALSE |
| cg16231917 | −0.124 | 2.55E−05 | 3.45E−02 | PVT1 | FALSE |
| cg15415194 | 0.077 | 3.95E−05 | 3.47E−02 | | |
| cg11253913 | 0.060 | 3.77E−05 | 3.47E−02 | CALD1 | FALSE |
| cg18303423 | 0.039 | 3.87E−05 | 3.47E−02 | DIP2C | FALSE |
| cg01047748 | −0.108 | 3.78E−05 | 3.47E−02 | FFAR3 | FALSE |
| cg23971362 | 0.034 | 3.39E−05 | 3.47E−02 | LRRC43 | FALSE |
| cg22798362 | 0.146 | 3.91E−05 | 3.47E−02 | MYO1D | FALSE |
| cg14894368 | 0.039 | 4.09E−05 | 3.47E−02 | NAPSA | FALSE |
| cg01502320 | 0.052 | 3.37E−05 | 3.47E−02 | TNFRSF13B | FALSE |
| cg27652459 | −0.064 | 4.49E−05 | 3.48E−02 | ARHGAP1 | FALSE |
| cg18234547 | 0.040 | 4.33E−05 | 3.48E−02 | | |
| cg01867764 | 0.108 | 4.33E−05 | 3.48E−02 | | |
| cg25364972 | −0.075 | 4.49E−05 | 3.48E−02 | | |
| cg21095280 | 0.079 | 4.52E−05 | 3.48E−02 | CPNE5 | FALSE |
| cg17445097 | 0.055 | 4.27E−05 | 3.48E−02 | PHF12 | FALSE |
| cg17408380 | 0.047 | 4.48E−05 | 3.48E−02 | RPL23A | FALSE |
| og05138138 | 0.073 | 4.46E−05 | 3.48E−02 | UXS1 | FALSE |
| cg17983064 | −0.055 | 4.58E−05 | 3.48E−02 | IL3 | FALSE |
| cg13967697 | 0.047 | 4.62E−05 | 3.49E−02 | EMB | FALSE |
| cg12214295 | 0.046 | 4.75E−05 | 3.50E−02 | | |
| cg14206140 | 0.042 | 5.07E−05 | 3.51E−02 | ADAMTS13 | FALSE |
| cg11354682 | 0.155 | 4.81E−05 | 3.51E−02 | C19orf38 | FALSE |
| cg22790377 | 0.092 | 5.16E−05 | 3.51E−02 | C1orf35 | FALSE |
| cg00792968 | 0.114 | 4.90E−05 | 3.51E−02 | | |
| cg07290761 | 0.072 | 5.50E−05 | 3.51E−02 | | |
| cg05350800 | 0.039 | 5.52E−05 | 3.51E−02 | | |
| cg25107000 | 0.087 | 5.62E−05 | 3.51E−02 | | |
| cg17342247 | −0.054 | 5.50E−05 | 3.51E−02 | CTNS | FALSE |
| cg26207423 | 0.044 | 5.11E−05 | 3.51E−02 | HMBOX1 | FALSE |
| cg03922648 | 0.030 | 5.40E−05 | 3.51E−02 | KLRC1 | FALSE |
| cg14306330 | 0.099 | 5.54E−05 | 3.51E−02 | L3MBTL | FALSE |
| cg13799081 | 0.119 | 4.99E−05 | 3.51E−02 | RBM20 | FALSE |
| cg21022868 | 0.085 | 5.55E−05 | 3.51E−02 | SLAMF9 | FALSE |
| cg18676586 | 0.085 | 4.96E−05 | 3.51E−02 | TNFRSF17 | FALSE |
| cg01727923 | 0.044 | 5.93E−05 | 3.57E−02 | | |
| cg10516886 | 0.084 | 5.93E−05 | 3.57E−02 | CD53 | FALSE |
| cg10584478 | −0.047 | 6.89E−05 | 3.63E−02 | BAHCC1 | FALSE |
| og02873991 | 0.093 | 6.38E−05 | 3.63E−02 | C12orf77 | FALSE |
| cg13400493 | 0.047 | 6.34E−05 | 3.63E−02 | | |
| cg10894274 | 0.042 | 6.35E−05 | 3.63E−02 | | |
| cg22202689 | 0.034 | 6.57E−05 | 3.63E−02 | | |
| cg24428218 | 0.026 | 6.61E−05 | 3.63E−02 | | |
| cg09061684 | −0.038 | 6.66E−05 | 3.63E−02 | | |
| cg14613361 | 0.034 | 6.89E−05 | 3.63E−02 | | |
| cg12240358 | 0.130 | 6.58E−05 | 3.63E−02 | HOMER2 | FALSE |
| cg22796248 | 0.049 | 6.89E−05 | 3.63E−02 | KCNMA1 | FALSE |
| cg08315483 | 0.027 | 6.76E−05 | 3.63E−02 | KIAA0182 | FALSE |
| cg00246547 | 0.028 | 6.31E−05 | 3.63E−02 | LOC90784 | FALSE |
| cg17843665 | 0.101 | 6.49E−05 | 3.63E−02 | MIPEP | FALSE |
| cg23842572 | 0.048 | 6.87E−05 | 3.63E−02 | MPRIP | |
| cg09337121 | −0.047 | 6.38E−05 | 3.63E−02 | PPP1R3E | FALSE |
| cg09521743 | 0.081 | 6.46E−05 | 3.63E−02 | SYNPO2L | FALSE |
| cg05120042 | 0.042 | 6.69E−05 | 3.63E−02 | TOP2A | FALSE |
| cg03339956 | 0.036 | 6.93E−05 | 3.64E−02 | TCF7L2 | FALSE |
| cg06806044 | −0.032 | 7.03E−05 | 3.66E−02 | | |
| cg03550727 | 0.052 | 7.09E−05 | 3.66E−02 | | |
| cg20414082 | 0.041 | 7.09E−05 | 3.66E−02 | | |
| cg13650974 | 0.025 | 7.12E−05 | 3.66E−02 | GLB1L2 | FALSE |
| cg12350474 | −0.037 | 7.41E−05 | 3.70E−02 | | |
| cg11224946 | 0.071 | 7.27E−05 | 3.70E−02 | MEGF6 | FALSE |
| cg24376214 | 0.097 | 7.32E−05 | 3.70E−02 | RASGRP4 | FALSE |
| cg08537367 | −0.073 | 7.28E−05 | 3.70E−02 | ZCCHC14 | FALSE |
| cg10227327 | 0.020 | 7.67E−05 | 3.71E−02 | FLJ43390 | FALSE |
| cg12018809 | 0.048 | 7.65E−05 | 3.71E−02 | LPAR5 | FALSE |
| cg23062006 | 0.032 | 7.59E−05 | 3.71E−02 | LRP4 | FALSE |
| cg09464061 | 0.089 | 7.50E−05 | 3.71E−02 | MGC2752 | FALSE |
| cg07692929 | 0.050 | 7.72E−05 | 3.71E−02 | OLFML1 | FALSE |
| cg15134506 | 0.084 | 7.53E−05 | 3.71E−02 | USP54 | FALSE |
| cg08250081 | −0.073 | 7.83E−05 | 3.72E−02 | | |
| cg00225902 | 0.117 | 7.80E−05 | 3.72E−02 | LRIG1 | FALSE |
| cg09277575 | 0.073 | 8.68E−05 | 3.76E−02 | AKR1D1 | FALSE |
| cg10144598 | 0.033 | 8.07E−05 | 3.76E−02 | ATP2B4 | FALSE |
| cg13630739 | 0.054 | 8.34E−05 | 3.76E−02 | ATP6V0A4 | FALSE |
| cg00898111 | 0.085 | 8.68E−05 | 3.76E−02 | BTBD17 | FALSE |
| cg16643706 | 0.045 | 8.16E−05 | 3.76E−02 | C5orf13 | FALSE |
| cg01285783 | 0.062 | 8.10E−05 | 3.76E−02 | C6orf195 | FALSE |
| cg08342332 | 0.026 | 8.69E−05 | 3.76E−02 | C6orf201 | |
| cg26583584 | 0.068 | 8.10E−05 | 3.76E−02 | | |
| cg15666143 | 0.080 | 8.19E−05 | 3.76E−02 | | |
| cg15529532 | −0.085 | 8.21E−05 | 3.76E−02 | | |
| cg13709640 | 0.038 | 8.30E−05 | 3.76E−02 | | |
| cg27141509 | 0.110 | 8.30E−05 | 3.76E−02 | | |
| cg14685948 | 0.041 | 8.20E−05 | 3.76E−02 | COL13A1 | FALSE |
| cg21711862 | 0.034 | 8.41E−05 | 3.76E−02 | | |
| cg20172258 | 0.033 | 8.61E−05 | 3.76E−02 | | |
| cg23228450 | 0.047 | 8.62E−05 | 3.76E−02 | | |
| cg00937012 | 0.035 | 8.43E−05 | 3.76E−02 | CPEB1 | FALSE |
| cg14249520 | −0.116 | 8.69E−05 | 3.76E−02 | CPT1A | FALSE |
| cg11229005 | 0.028 | 8.75E−05 | 3.76E−02 | | |
| cg04264560 | 0.053 | 8.76E−05 | 3.76E−02 | | |
| cg18817392 | 0.035 | 8.10E−05 | 3.76E−02 | EYA4 | FALSE |
| cg10318258 | 0.037 | 8.80E−05 | 3.76E−02 | RIPK3 | FALSE |
| cg08691422 | 0.069 | 8.51E−05 | 3.76E−02 | TP53 | FALSE |
| cg08287903 | 0.099 | 8.71E−05 | 3.76E−02 | UGT8 | FALSE |
| cg01601926 | 0.025 | 8.35E−05 | 3.76E−02 | USP7 | FALSE |
| cg18559579 | −0.081 | 8.90E−05 | 3.76E−02 | | |
| cg10870160 | −0.032 | 9.10E−05 | 3.77E−02 | RAI1 | FALSE |
| cg14398929 | 0.034 | 9.13E−05 | 3.78E−02 | | |
| cg03799024 | 0.035 | 9.20E−05 | 3.80E−02 | MIR515-2 | FALSE |
| cg15233062 | 0.028 | 9.25E−05 | 3.80E−02 | HOXC4 | FALSE |
| cg05913197 | 0.023 | 9.51E−05 | 3.82E−02 | BRP44L | FALSE |
| cg12561776 | 0.035 | 9.42E−05 | 3.82E−02 | MYO1D | FALSE |
| cg04029168 | −0.029 | 9.41E−05 | 3.82E−02 | PRKCB | FALSE |
| cg02224372 | 0.033 | 9.40E−05 | 3.82E−02 | SHANK2 | FALSE |
| cg25146557 | −0.039 | 9.58E−05 | 3.84E−02 | LOC200726 | FALSE |
| cg20992354 | 0.038 | 9.66E−05 | 3.85E−02 | TDRD5 | FALSE |
| cg01043616 | 0.053 | 1.07E−04 | 3.85E−02 | ACCN2 | FALSE |
| cg13502474 | −0.047 | 1.01E−04 | 3.85E−02 | | |
| cg16974893 | 0.038 | 1.02E−04 | 3.85E−02 | | |
| cg27009439 | 0.044 | 1.02E−04 | 3.85E−02 | | |
| cg24426483 | 0.043 | 1.02E−04 | 3.85E−02 | | |
| cg12449916 | −0.081 | 1.04E−04 | 3.85E−02 | | |
| cg17313709 | −0.082 | 1.05E−04 | 3.85E−02 | | |
| cg25585279 | 0.076 | 1.06E−04 | 3.85E−02 | | |
| cg08128087 | 0.086 | 1.07E−04 | 3.85E−02 | | |
| cg03549563 | 0.067 | 1.07E−04 | 3.85E−02 | | |
| cg06957003 | 0.046 | 1.07E−04 | 3.85E−02 | | |
| cg10585661 | 0.056 | 1.03E−04 | 3.85E−02 | FAM131A | FALSE |
| cg14324502 | −0.102 | 1.08E−04 | 3.85E−02 | ISCA1 | FALSE |
| cg26722342 | 0.022 | 9.71E−05 | 3.85E−02 | ITGA2 | FALSE |
| cg26104475 | −0.051 | 1.03E−04 | 3.85E−02 | LCP2 | FALSE |
| cg21010350 | 0.025 | 1.08E−04 | 3.85E−02 | LOC100128542 | FALSE |
| cg02303324 | −0.107 | 9.89E−05 | 3.85E−02 | MAP7 | FALSE |
| cg17192247 | −0.044 | 1.08E−04 | 3.85E−02 | MAPRE3 | FALSE |
| cg25970363 | 0.032 | 1.03E−04 | 3.85E−02 | PDZK1IP1 | FALSE |
| cg02005758 | −0.026 | 1.01E−04 | 3.85E−02 | RAB7A | FALSE |
| cg06812693 | 0.019 | 1.03E−04 | 3.85E−02 | RBPJ | FALSE |
| cg24137448 | 0.088 | 9.98E−05 | 3.85E−02 | SNX11 | FALSE |
| cg07090356 | −0.066 | 9.87E−05 | 3.85E−02 | SS18 | FALSE |
| cg07173112 | 0.041 | 9.95E−05 | 3.85E−02 | TTLL10 | FALSE |
| cg27644327 | −0.077 | 1.08E−04 | 3.86E−02 | BACH2 | FALSE |
| cg10854767 | 0.039 | 1.08E−04 | 3.86E−02 | PTPRN2 | FALSE |
| cg03219838 | −0.024 | 1.09E−04 | 3.86E−02 | H2AFY | FALSE |
| cg20114528 | 0.047 | 1.11E−04 | 3.88E−02 | ASB8 | FALSE |
| cg11070419 | 0.057 | 1.10E−04 | 3.88E−02 | C4BPA | FALSE |
| cg02127689 | 0.031 | 1.11E−04 | 3.88E−02 | | |
| cg05938409 | 0.051 | 1.11E−04 | 3.88E−02 | | |
| cg07091500 | −0.121 | 1.11E−04 | 3.88E−02 | | |
| cg01646461 | 0.048 | 1.14E−04 | 3.90E−02 | CCR6 | FALSE |
| cg05334190 | 0.022 | 1.15E−04 | 3.90E−02 | | |
| cg19172222 | 0.027 | 1.15E−04 | 3.90E−02 | TBX21 | FALSE |
| cg26280179 | 0.055 | 1.17E−04 | 3.91E−02 | | |
| cg10704395 | −0.035 | 1.18E−04 | 3.93E−02 | CAMK1D | FALSE |
| cg16266809 | 0.097 | 1.18E−04 | 3.93E−02 | | |
| cg13434312 | 0.044 | 1.20E−04 | 3.95E−02 | PTCHD2 | FALSE |
| cg01068906 | −0.072 | 1.20E−04 | 3.96E−02 | NOD2 | FALSE |
| cg08899215 | 0.046 | 1.21E−04 | 3.96E−02 | BAT3 | FALSE |
| cg20849025 | 0.055 | 1.21E−04 | 3.96E−02 | MPEG1 | FALSE |
| cg22512670 | −0.041 | 1.21E−04 | 3.96E−02 | RPS6KA1 | FALSE |
| cg10765387 | 0.046 | 1.24E−04 | 3.98E−02 | COBL | FALSE |
| cg22929787 | −0.058 | 1.22E−04 | 3.98E−02 | | |
| cg21493951 | 0.172 | 1.25E−04 | 3.98E−02 | HLA-DQB1 | FALSE |
| cg11397854 | −0.034 | 1.24E−04 | 3.98E−02 | IQSEC1 | FALSE |
| cg06791592 | 0.085 | 1.25E−04 | 3.98E−02 | PSMB9 | FALSE |
| cg18202741 | 0.060 | 1.26E−04 | 3.98E−02 | AQP6 | FALSE |
| cg14586546 | 0.035 | 1.26E−04 | 3.98E−02 | RALGDS | FALSE |
| cg04352288 | 0.052 | 1.27E−04 | 3.98E−02 | CA5A | FALSE |
| cg19954286 | 0.057 | 1.29E−04 | 3.98E−02 | CCR6 | FALSE |
| cg11650874 | 0.037 | 1.28E−04 | 3.98E−02 | CHID1 | FALSE |
| cg07991229 | −0.042 | 1.29E−04 | 3.98E−02 | | |
| cg03564620 | 0.045 | 1.30E−04 | 3.98E−02 | MYH6 | FALSE |
| cg00754552 | 0.097 | 1.30E−04 | 3.98E−02 | NAPSB | FALSE |
| cg23217386 | 0.094 | 1.28E−04 | 3.98E−02 | TRIM34 | FALSE |
| cg08431718 | 0.037 | 1.31E−04 | 3.99E−02 | | |
| cg24593832 | 0.039 | 1.31E−04 | 3.99E−02 | | |
| cg12079885 | 0.029 | 1.32E−04 | 4.01E−02 | SCARB1 | FALSE |
| cg01942318 | 0.028 | 1.35E−04 | 4.01E−02 | | |
| cg17832704 | −0.037 | 1.35E−04 | 4.01E−02 | SHBG | FALSE |
| cg09012125 | −0.044 | 1.36E−04 | 4.02E−02 | KCNJ5 | FALSE |
| cg09200921 | −0.045 | 1.38E−04 | 4.03E−02 | CDADC1 | FALSE |
| cg04298953 | −0.038 | 1.37E−04 | 4.03E−02 | | |
| cg20547015 | 0.083 | 1.37E−04 | 4.03E−02 | PPP1CC | FALSE |
| cg26878655 | 0.080 | 1.37E−04 | 4.03E−02 | RARA | FALSE |
| cg12459502 | −0.076 | 1.45E−04 | 4.04E−02 | BCL2 | FALSE |
| cg00958560 | −0.074 | 1.42E−04 | 4.04E−02 | C4orf50 | FALSE |
| cg18022036 | 0.090 | 1.39E−04 | 4.04E−02 | | |
| cg17102910 | 0.072 | 1.45E−04 | 4.04E−02 | GPER | FALSE |
| cg11707665 | 0.047 | 1.45E−04 | 4.04E−02 | GPR133 | FALSE |
| cg01005303 | 0.055 | 1.43E−04 | 4.04E−02 | KIAA2013 | FALSE |
| cg21593149 | 0.055 | 1.44E−04 | 4.04E−02 | MAEA | FALSE |
| cg02329430 | −0.082 | 1.45E−04 | 4.04E−02 | NPTN | FALSE |
| cg09620705 | 0.037 | 1.46E−04 | 4.04E−02 | SMOC2 | FALSE |
| cg13858974 | 0.065 | 1.49E−04 | 4.07E−02 | FRMD3 | FALSE |
| cg26788180 | −0.050 | 1.53E−04 | 4.07E−02 | ATF6B | FALSE |
| cg05460965 | −0.042 | 1.53E−04 | 4.07E−02 | CDKN1A | FALSE |
| cg08708790 | 0.066 | 1.51E−04 | 4.07E−02 | | |
| cg02375166 | −0.045 | 1.52E−04 | 4.07E−02 | | |
| cg18555142 | 0.025 | 1.53E−04 | 4.07E−02 | | |
| cg01117384 | 0.083 | 1.54E−04 | 4.07E−02 | PMEPA1 | FALSE |
| cg26192556 | 0.054 | 1.54E−04 | 4.07E−02 | SLCO6A1 | FALSE |
| cg16975095 | 0.028 | 1.56E−04 | 4.08E−02 | | |
| cg00045515 | 0.055 | 1.55E−04 | 4.08E−02 | RBL1 | FALSE |
| cg13864749 | 0.031 | 1.57E−04 | 4.09E−02 | NECAP2 | FALSE |
| cg12496975 | 0.056 | 1.58E−04 | 4.09E−02 | RAPGEF3 | FALSE |
| cg16548911 | −0.078 | 1.59E−04 | 4.09E−02 | ADCY7 | FALSE |
| cg23797615 | 0.053 | 1.61E−04 | 4.09E−02 | CLCF1 | FALSE |
| cg13107839 | 0.061 | 1.60E−04 | 4.09E−02 | | |
| cg18274749 | 0.043 | 1.61E−04 | 4.09E−02 | HSPG2 | FALSE |
| cg20570458 | 0.063 | 1.60E−04 | 4.09E−02 | KALRN | FALSE |
| cg02780029 | 0.071 | 1.60E−04 | 4.09E−02 | RET | FALSE |
| cg15817482 | 0.034 | 1.62E−04 | 4.12E−02 | ASB2 | FALSE |
| cg20769856 | 0.070 | 1.63E−04 | 4.13E−02 | | |
| cg23964739 | 0.020 | 1.64E−04 | 4.13E−02 | CD3EAP | FALSE |
| cg25541259 | 0.059 | 1.68E−04 | 4.14E−02 | DNER | FALSE |
| cg11261261 | 0.055 | 1.68E−04 | 4.14E−02 | DOCK1 | FALSE |
| cg06819445 | 0.023 | 1.69E−04 | 4.14E−02 | DUSP1 | FALSE |
| cg00353330 | 0.066 | 1.66E−04 | 4.14E−02 | | |
| cg10743102 | 0.106 | 1.69E−04 | 4.14E−02 | | |
| cg22851557 | 0.055 | 1.69E−04 | 4.14E−02 | PRDM16 | FALSE |
| cg20721135 | −0.069 | 1.69E−04 | 4.14E−02 | RASA3 | FALSE |
| cg02635449 | 0.047 | 1.68E−04 | 4.14E−02 | RPH3AL | FALSE |
| cg24156393 | 0.048 | 1.70E−04 | 4.14E−02 | | |
| cg22252462 | 0.035 | 1.72E−04 | 4.16E−02 | APOM | FALSE |
| cg00969893 | 0.085 | 1.73E−04 | 4.16E−02 | TLN2 | FALSE |
| cg00085123 | −0.056 | 1.77E−04 | 4.20E−02 | | |
| cg11193767 | −0.092 | 1.77E−04 | 4.20E−02 | LIN54 | FALSE |
| cg20193802 | 0.022 | 1.78E−04 | 4.20E−02 | SEPTIN5 | FALSE |
| cg26736341 | 0.063 | 1.75E−04 | 4.20E−02 | TNF | FALSE |
| cg21097090 | 0.096 | 1.76E−04 | 4.20E−02 | TNFAIP8 | FALSE |
| cg25101396 | −0.087 | 1.79E−04 | 4.20E−02 | ABHD1 | FALSE |
| cg16802439 | −0.099 | 1.79E−04 | 4.20E−02 | GALNS | FALSE |
| cg19360796 | 0.060 | 1.78E−04 | 4.20E−02 | | |
| cg17254229 | 0.051 | 1.80E−04 | 4.21E−02 | | |
| cg08175640 | 0.034 | 1.80E−04 | 4.21E−02 | LTBP1 | FALSE |
| cg02556649 | 0.036 | 1.82E−04 | 4.24E−02 | TNNT3 | FALSE |
| cg20672131 | 0.042 | 1.83E−04 | 4.26E−02 | | |
| cg20893838 | −0.076 | 1.84E−04 | 4.26E−02 | RREB1 | FALSE |
| cg17996401 | −0.040 | 1.84E−04 | 4.26E−02 | | |
| cg13178597 | 0.037 | 1.87E−04 | 4.29E−02 | RGS17 | FALSE |
| cg02770857 | −0.054 | 1.87E−04 | 4.29E−02 | MSI2 | FALSE |
| cg23598010 | 0.073 | 1.89E−04 | 4.30E−02 | | |
| cg07569385 | −0.050 | 1.91E−04 | 4.30E−02 | GJB2 | FALSE |
| cg10356204 | 0.047 | 1.92E−04 | 4.30E−02 | JSRP1 | FALSE |
| cg02124022 | −0.069 | 1.91E−04 | 4.30E−02 | | |
| cg26755793 | 0.033 | 1.91E−04 | 4.30E−02 | KCTD1 | FALSE |
| cg09090048 | 0.022 | 1.91E−04 | 4.30E−02 | VPS26B | FALSE |
| cg08161480 | −0.080 | 1.95E−04 | 4.31E−02 | | |
| cg08861091 | 0.041 | 1.94E−04 | 4.31E−02 | LIM2 | FALSE |
| cg10122294 | −0.060 | 1.95E−04 | 4.32E−02 | TSNAX-DISC1 | FALSE |
| cg12037550 | 0.038 | 1.96E−04 | 4.32E−02 | ADARB2 | FALSE |
| cg13333954 | −0.105 | 1.97E−04 | 4.32E−02 | | |
| cg15386103 | 0.027 | 1.99E−04 | 4.33E−02 | JUP | FALSE |
| cg16424311 | 0.032 | 2.01E−04 | 4.35E−02 | CRISP3 | FALSE |
| cg26557617 | 0.063 | 2.01E−04 | 4.35E−02 | | |
| cg13501907 | −0.033 | 2.04E−04 | 4.35E−02 | | |
| cg23906687 | 0.051 | 2.04E−04 | 4.35E−02 | KLHL30 | FALSE |
| cg00547480 | −0.057 | 2.04E−04 | 4.35E−02 | LDB1 | FALSE |
| cg13804478 | 0.047 | 2.04E−04 | 4.35E−02 | MUC1 | FALSE |
| cg23992426 | 0.040 | 2.08E−04 | 4.39E−02 | NUP160 | FALSE |
| cg14454798 | 0.053 | 2.11E−04 | 4.41E−02 | CPEB4 | FALSE |
| cg00425764 | 0.055 | 2.11E−04 | 4.41E−02 | MYH11 | FALSE |
| cg22637435 | −0.082 | 2.11E−04 | 4.41E−02 | TRAPPC9 | FALSE |
| cg12180319 | 0.063 | 2.13E−04 | 4.43E−02 | FBN1 | FALSE |
| cg16252691 | 0.040 | 2.13E−04 | 4.43E−02 | | |
| cg15357821 | 0.045 | 2.14E−04 | 4.44E−02 | HK3 | FALSE |
| cg02672229 | −0.037 | 2.15E−04 | 4.45E−02 | ABCC2 | FALSE |
| cg15378605 | 0.056 | 2.16E−04 | 4.45E−02 | CHST15 | FALSE |
| cg26382697 | 0.058 | 2.16E−04 | 4.46E−02 | CD81 | FALSE |
| cg16266925 | −0.023 | 2.17E−04 | 4.47E−02 | TXNDC6 | FALSE |
| cg02594183 | 0.034 | 2.18E−04 | 4.47E−02 | ODZ4 | FALSE |
| cg15056348 | −0.032 | 2.19E−04 | 4.48E−02 | INPP5A | FALSE |
| cg06621799 | −0.074 | 2.19E−04 | 4.48E−02 | MIA3 | FALSE |
| cg21025494 | −0.028 | 2.21E−04 | 4.50E−02 | IGF2BP3 | FALSE |
| cg07093324 | −0.080 | 2.23E−04 | 4.51E−02 | ACTR3 | FALSE |
| cg05524038 | 0.034 | 2.23E−04 | 4.51E−02 | CSF1R | FALSE |
| cg07350444 | 0.022 | 2.22E−04 | 4.51E−02 | | |
| cg04494051 | −0.034 | 2.22E−04 | 4.51E−02 | LOC100130987 | FALSE |
| cg18869709 | −0.078 | 2.22E−04 | 4.51E−02 | PACSIN2 | FALSE |
| cg04947533 | 0.039 | 2.25E−04 | 4.51E−02 | DCAF5 | FALSE |
| cg23499632 | 0.032 | 2.25E−04 | 4.51E−02 | | |
| cg17360552 | 0.095 | 2.27E−04 | 4.52E−02 | HLA-DQB2 | FALSE |
| cg00226984 | 0.025 | 2.26E−04 | 4.52E−02 | KLHL30 | FALSE |
| cg20802826 | 0.084 | 2.26E−04 | 4.52E−02 | | |
| cg07180197 | 0.046 | 2.27E−04 | 4.52E−02 | TRIM55 | FALSE |
| cg24850474 | 0.052 | 2.28E−04 | 4.52E−02 | ATP13A1 | FALSE |
| cg03724640 | 0.053 | 2.28E−04 | 4.52E−02 | CD27 | FALSE |
| cg13427748 | −0.034 | 2.28E−04 | 4.52E−02 | | |
| cg23279117 | 0.022 | 2.29E−04 | 4.52E−02 | | |
| cg00232996 | 0.035 | 2.30E−04 | 4.52E−02 | ATF6B | FALSE |
| cg04222159 | 0.032 | 2.30E−04 | 4.52E−02 | NFASC | FALSE |
| cg11906173 | 0.028 | 2.31E−04 | 4.53E−02 | DHX37 | FALSE |
| cg21808287 | 0.031 | 2.31E−04 | 4.53E−02 | TNP1 | FALSE |
| cg14102807 | 0.039 | 2.34E−04 | 4.53E−02 | CD19 | FALSE |
| cg21137557 | 0.036 | 2.34E−04 | 4.53E−02 | LVRN | FALSE |
| cg20315739 | 0.037 | 2.33E−04 | 4.53E−02 | PLA2G4E | FALSE |
| cg25241559 | 0.035 | 2.34E−04 | 4.53E−02 | SNED1 | FALSE |
| cg19743666 | −0.112 | 2.32E−04 | 4.53E−02 | YPEL5 | FALSE |
| cg08413427 | −0.093 | 2.36E−04 | 4.53E−02 | DBF4B | FALSE |
| cg22988581 | 0.032 | 2.35E−04 | 4.53E−02 | SLC17A1 | FALSE |
| cg00372249 | 0.065 | 2.41E−04 | 4.55E−02 | ACSL1 | FALSE |
| cg11561301 | 0.056 | 2.42E−04 | 4.55E−02 | C4orf6 | FALSE |
| cg06639923 | 0.031 | 2.44E−04 | 4.55E−02 | FBXL18 | FALSE |
| cg05365532 | 0.070 | 2.42E−04 | 4.55E−02 | LILRB1 | FALSE |
| cg17920789 | 0.052 | 2.48E−04 | 4.55E−02 | LRCH1 | FALSE |
| cg01973920 | 0.063 | 2.41E−04 | 4.55E−02 | | |
| cg05533001 | 0.022 | 2.39E−04 | 4.55E−02 | MAD1L1 | FALSE |
| cg11906021 | 0.074 | 2.44E−04 | 4.55E−02 | | |
| cg27039898 | −0.081 | 2.46E−04 | 4.55E−02 | | |
| cg00106345 | −0.082 | 2.47E−04 | 4.55E−02 | | |
| cg11405200 | −0.049 | 2.48E−04 | 4.55E−02 | | |
| cg01894985 | 0.112 | 2.40E−04 | 4.55E−02 | MYLK | FALSE |
| cg16814786 | −0.096 | 2.46E−04 | 4.55E−02 | NCOA4 | FALSE |
| cg06965839 | 0.115 | 2.45E−04 | 4.55E−02 | OR1E1 | FALSE |
| cg12399031 | 0.050 | 2.42E−04 | 4.55E−02 | PDLIM1 | FALSE |
| cg07143805 | −0.026 | 2.45E−04 | 4.55E−02 | PMEPA1 | FALSE |
| cg25635916 | 0.028 | 2.38E−04 | 4.55E−02 | SEMA3B | FALSE |
| cg20931972 | −0.080 | 2.50E−04 | 4.55E−02 | CD38 | FALSE |
| cg06238667 | 0.127 | 2.51E−04 | 4.56E−02 | KIF13B | FALSE |
| cg13719443 | −0.042 | 2.51E−04 | 4.56E−02 | LOC285847 | FALSE |
| cg20680284 | 0.040 | 2.52E−04 | 4.56E−02 | | |
| cg19371652 | 0.038 | 2.52E−04 | 4.57E−02 | OAS2 | FALSE |
| cg05394244 | −0.105 | 2.54E−04 | 4.57E−02 | PTP4A1 | FALSE |
| cg09039672 | −0.058 | 2.60E−04 | 4.57E−02 | ACVR1B | FALSE |
| cg21304211 | 0.021 | 2.59E−04 | 4.57E−02 | C1orf183 | FALSE |
| cg21055910 | 0.045 | 2.59E−04 | 4.57E−02 | ELFN2 | FALSE |
| cg20995304 | −0.072 | 2.61E−04 | 4.57E−02 | HDAC7 | FALSE |
| cg00422716 | 0.027 | 2.55E−04 | 4.57E−02 | HYAL4 | FALSE |
| cg00171092 | 0.059 | 2.59E−04 | 4.57E−02 | ITGB5 | FALSE |
| cg04870110 | 0.032 | 2.56E−04 | 4.57E−02 | | |
| cg03403168 | 0.055 | 2.59E−04 | 4.57E−02 | | |
| cg01939741 | 0.023 | 2.61E−04 | 4.57E−02 | | |
| cg12040750 | 0.035 | 2.58E−04 | 4.57E−02 | PRR21 | FALSE |
| cg21853581 | 0.036 | 2.59E−04 | 4.57E−02 | RBPMS | FALSE |
| cg20792206 | 0.045 | 2.57E−04 | 4.57E−02 | SNAPC5 | FALSE |
| cg03884572 | 0.035 | 2.62E−04 | 4.57E−02 | CUX2 | FALSE |
| cg23965590 | 0.071 | 2.63E−04 | 4.58E−02 | CBL | FALSE |
| cg17307811 | 0.030 | 2.63E−04 | 4.58E−02 | UBE2S | FALSE |
| cg18558968 | 0.036 | 2.63E−04 | 4.58E−02 | UST | FALSE |
| cg11741335 | −0.039 | 2.64E−04 | 4.58E−02 | EEPD1 | FALSE |
| cg18648169 | −0.039 | 2.67E−04 | 4.60E−02 | | |
| cg12588917 | 0.072 | 2.68E−04 | 4.60E−02 | HLA-F | FALSE |
| cg05735547 | 0.027 | 2.68E−04 | 4.60E−02 | | |
| cg13994504 | 0.060 | 2.71E−04 | 4.62E−02 | ANKRD2 | FALSE |
| cg00524486 | 0.045 | 2.71E−04 | 4.62E−02 | MUC5B | FALSE |
| cg04551630 | 0.066 | 2.76E−04 | 4.63E−02 | GPR98 | FALSE |
| cg22277567 | 0.074 | 2.74E−04 | 4.63E−02 | HDAC4 | FALSE |
| cg06514003 | 0.070 | 2.76E−04 | 4.63E−02 | KCNH4 | FALSE |
| cg11851792 | 0.062 | 2.72E−04 | 4.63E−02 | | |
| cg03362882 | 0.055 | 2.74E−04 | 4.63E−02 | | |
| cg09390241 | 0.054 | 2.74E−04 | 4.63E−02 | | |
| cg08610076 | −0.053 | 2.73E−04 | 4.63E−02 | RASA3 | FALSE |
| cg07169295 | 0.023 | 2.75E−04 | 4.63E−02 | SYT12 | FALSE |
| cg07345515 | 0.036 | 2.74E−04 | 4.63E−02 | TNXB | FALSE |
| cg18023455 | 0.071 | 2.72E−04 | 4.63E−02 | VWC2 | FALSE |
| cg14833891 | 0.027 | 2.78E−04 | 4.63E−02 | | |
| cg13714407 | 0.097 | 2.78E−04 | 4.63E−02 | RAPGEF1 | FALSE |
| cg16526047 | 0.040 | 2.81E−04 | 4.66E−02 | ISG15 | FALSE |
| cg22201545 | 0.018 | 2.81E−04 | 4.66E−02 | | |
| cg25792439 | −0.059 | 2.82E−04 | 4.67E−02 | CARD14 | FALSE |
| cg15944824 | 0.042 | 2.83E−04 | 4.67E−02 | | |
| cg00625351 | 0.027 | 2.84E−04 | 4.67E−02 | | |
| cg13751548 | 0.046 | 2.84E−04 | 4.67E−02 | PAX5 | FALSE |
| cg24256211 | −0.062 | 2.83E−04 | 4.67E−02 | SAMD4A | FALSE |
| cg02483101 | 0.076 | 2.85E−04 | 4.67E−02 | | |
| cg10006442 | −0.048 | 2.85E−04 | 4.68E−02 | DIDO1 | FALSE |
| cg06978604 | 0.028 | 2.86E−04 | 4.68E−02 | EXOC8 | FALSE |
| cg17685004 | 0.105 | 2.87E−04 | 4.68E−02 | | |
| cg23954655 | 0.088 | 2.87E−04 | 4.68E−02 | STK24 | FALSE |
| cg27258561 | 0.098 | 2.88E−04 | 4.69E−02 | | |
| cg04350675 | −0.085 | 2.89E−04 | 4.69E−02 | | |
| cg21278103 | −0.088 | 2.89E−04 | 4.69E−02 | PTPRA | FALSE |
| cg10399946 | 0.051 | 2.89E−04 | 4.69E−02 | RPS18 | FALSE |
| cg02428506 | 0.033 | 2.90E−04 | 4.69E−02 | ARHGDIG | FALSE |
| cg09366969 | −0.058 | 2.90E−04 | 4.69E−02 | PDE4DIP | FALSE |
| cg11341011 | 0.033 | 2.91E−04 | 4.70E−02 | | |
| cg08972170 | −0.062 | 2.93E−04 | 4.70E−02 | C7orf41 | FALSE |
| cg10126181 | −0.046 | 2.96E−04 | 4.71E−02 | FLJ22536 | FALSE |
| cg14234656 | 0.040 | 2.96E−04 | 4.71E−02 | | |
| cg18690833 | 0.041 | 2.96E−04 | 4.71E−02 | SEL1L2 | FALSE |
| cg05507908 | −0.044 | 3.06E−04 | 4.72E−02 | ANKRD31 | FALSE |
| cg02824029 | −0.067 | 3.04E−04 | 4.72E−02 | GABBR1 | FALSE |
| cg11701022 | 0.021 | 3.05E−04 | 4.72E−02 | IGF2AS | FALSE |
| cg19811144 | 0.050 | 3.06E−04 | 4.72E−02 | KIAA0427 | FALSE |
| cg13861536 | 0.039 | 3.01E−04 | 4.72E−02 | KIAA1614 | FALSE |
| cg14374799 | −0.038 | 2.99E−04 | 4.72E−02 | NPM2 | FALSE |
| cg25104637 | 0.026 | 3.01E−04 | 4.72E−02 | NRXN2 | FALSE |
| cg24435939 | 0.036 | 3.00E−04 | 4.72E−02 | | |
| cg08110488 | −0.060 | 3.00E−04 | 4.72E−02 | | |
| cg01721313 | −0.065 | 3.01E−04 | 4.72E−02 | | |
| cg09978533 | −0.088 | 3.04E−04 | 4.72E−02 | | |
| cg21950037 | 0.025 | 3.06E−04 | 4.72E−02 | | |
| cg06713373 | 0.030 | 2.98E−04 | 4.72E−02 | SLC12A8 | FALSE |
| cg07721005 | 0.020 | 3.04E−04 | 4.72E−02 | TMEM161B | |
| cg20670923 | −0.053 | 3.07E−04 | 4.73E−02 | VAPA | FALSE |
| cg01400750 | 0.089 | 3.09E−04 | 4.73E−02 | ANAPC10 | FALSE |
| cg13718827 | −0.053 | 3.10E−04 | 4.73E−02 | MIR199B | FALSE |
| cg01312828 | 0.040 | 3.09E−04 | 4.73E−02 | | |
| cg14761773 | −0.029 | 3.09E−04 | 4.73E−02 | | |
| cg25573365 | 0.024 | 3.10E−04 | 4.73E−02 | | |
| cg24869834 | 0.053 | 3.09E−04 | 4.73E−02 | SLC7A11 | FALSE |
| cg00849570 | 0.027 | 3.11E−04 | 4.73E−02 | PRDM16 | FALSE |
| cg01046511 | −0.048 | 3.13E−04 | 4.74E−02 | KDM6B | FALSE |
| cg17250082 | 0.179 | 3.14E−04 | 4.74E−02 | | |
| cg02131639 | 0.045 | 3.14E−04 | 4.74E−02 | | |
| cg25954627 | 0.019 | 3.14E−04 | 4.74E−02 | | |
| cg25801292 | −0.032 | 3.15E−04 | 4.74E−02 | KEAP1 | FALSE |
| cg26987928 | 0.056 | 3.15E−04 | 4.74E−02 | | |
| cg11671363 | −0.100 | 3.17E−04 | 4.76E−02 | MIR145 | FALSE |
| cg12833765 | 0.085 | 3.19E−04 | 4.77E−02 | ANTXR1 | FALSE |
| cg15900221 | 0.029 | 3.21E−04 | 4.78E−02 | ARHGEF16 | FALSE |
| cg21105590 | 0.019 | 3.22E−04 | 4.78E−02 | DPPA2 | FALSE |
| cg18087514 | 0.076 | 3.23E−04 | 4.78E−02 | KMO | FALSE |
| cg15528028 | 0.042 | 3.22E−04 | 4.78E−02 | PHC2 | FALSE |
| cg04152616 | 0.026 | 3.21E−04 | 4.78E−02 | | |
| cg01265653 | 0.038 | 3.23E−04 | 4.78E−02 | | |
| cg14544289 | −0.030 | 3.22E−04 | 4.78E−02 | SPTLC2 | FALSE |
| cg03291835 | −0.067 | 3.24E−04 | 4.78E−02 | COL4A1 | FALSE |
| cg19690404 | 0.080 | 3.23E−04 | 4.78E−02 | CPA4 | FALSE |
| cg11849692 | −0.047 | 3.23E−04 | 4.78E−02 | LDB1 | FALSE |
| cg07618110 | 0.050 | 3.24E−04 | 4.78E−02 | MS4A1 | FALSE |
| cg20862309 | 0.051 | 3.25E−04 | 4.78E−02 | ZC3H14 | FALSE |
| cg09959084 | 0.022 | 3.26E−04 | 4.79E−02 | NME1 | FALSE |
| cg03354508 | 0.073 | 3.27E−04 | 4.79E−02 | | |
| cg18792364 | −0.056 | 3.27E−04 | 4.79E−02 | | |
| cg11073381 | 0.056 | 3.43E−04 | 4.80E−02 | ANKFN1 | FALSE |
| cg10089657 | 0.036 | 3.37E−04 | 4.80E−02 | C7orf50 | FALSE |
| cg26886965 | 0.055 | 3.35E−04 | 4.80E−02 | CR1 | FALSE |
| cg09730170 | 0.047 | 3.41E−04 | 4.80E−02 | CST9 | FALSE |
| cg27284034 | 0.045 | 3.47E−04 | 4.80E−02 | CTRB1 | FALSE |
| cg17697873 | 0.031 | 3.40E−04 | 4.80E−02 | DLGAP2 | FALSE |
| cg21163415 | 0.057 | 3.30E−04 | 4.80E−02 | EDN3 | FALSE |
| cg16986578 | 0.026 | 3.42E−04 | 4.80E−02 | ELF3 | FALSE |
| cg25145687 | −0.079 | 3.36E−04 | 4.80E−02 | HCCA2 | FALSE |
| cg14483244 | −0.029 | 3.34E−04 | 4.80E−02 | HDAC5 | FALSE |
| cg11187245 | 0.093 | 3.41E−04 | 4.80E−02 | HLA-B | FALSE |
| cg20052079 | −0.082 | 3.31E−04 | 4.80E−02 | JARID2 | FALSE |
| cg17425484 | 0.045 | 3.34E−04 | 4.80E−02 | KCNMB1 | FALSE |
| cg13057898 | 0.080 | 3.47E−04 | 4.80E−02 | LRRC47 | FALSE |
| cg10723617 | −0.061 | 3.41E−04 | 4.80E−02 | MSI2 | FALSE |
| cg20044211 | 0.018 | 3.33E−04 | 4.80E−02 | NOTCH4 | FALSE |
| cg24180759 | 0.074 | 3.45E−04 | 4.80E−02 | ODZ2 | FALSE |
| cg12630082 | −0.048 | 3.39E−04 | 4.80E−02 | PKDCC | FALSE |
| cg11464160 | −0.092 | 3.31E−04 | 4.80E−02 | PLEKHB2 | |
| cg07582829 | 0.045 | 3.45E−04 | 4.80E−02 | PRKCG | FALSE |
| cg23109530 | 0.049 | 3.29E−04 | 4.80E−02 | | |
| cg19355069 | −0.042 | 3.29E−04 | 4.80E−02 | | |
| cg17534999 | 0.095 | 3.30E−04 | 4.80E−02 | | |
| cg08756898 | 0.045 | 3.34E−04 | 4.80E−02 | | |
| cg17560832 | 0.025 | 3.42E−04 | 4.80E−02 | | |
| cg05373655 | 0.059 | 3.43E−04 | 4.80E−02 | | |
| cg03784113 | 0.044 | 3.43E−04 | 4.80E−02 | | |
| cg12092579 | 0.071 | 3.44E−04 | 4.80E−02 | RASAL2 | FALSE |
| cg24068815 | 0.025 | 3.45E−04 | 4.80E−02 | | |
| cg25315819 | 0.064 | 3.45E−04 | 4.80E−02 | | |
| cg22412536 | 0.020 | 3.38E−04 | 4.80E−02 | RORA | FALSE |
| cg01218206 | 0.088 | 3.47E−04 | 4.80E−02 | SIK3 | FALSE |
| cg16928046 | 0.052 | 3.31E−04 | 4.80E−02 | TBX21 | FALSE |
| cg06233930 | 0.025 | 3.46E−04 | 4.80E−02 | TIMP2 | FALSE |
| cg18152830 | 0.027 | 3.33E−04 | 4.80E−02 | TNFRSF13B | FALSE |
| cg13172153 | 0.039 | 3.32E−04 | 4.80E−02 | TNNI1 | FALSE |
| cg21033855 | −0.030 | 3.45E−04 | 4.80E−02 | TUBGCP4 | FALSE |
| cg24323726 | 0.062 | 3.48E−04 | 4.80E−02 | ZBED2 | FALSE |
| cg13928759 | 0.047 | 3.49E−04 | 4.80E−02 | LGI1 | FALSE |
| cg01245880 | 0.071 | 3.49E−04 | 4.81E−02 | SEC14L4 | FALSE |
| cg09012471 | −0.068 | 3.51E−04 | 4.81E−02 | ARMC8 | FALSE |
| cg10764891 | 0.030 | 3.51E−04 | 4.81E−02 | | |
| cg16422365 | 0.043 | 3.52E−04 | 4.81E−02 | | |
| cg23256882 | 0.063 | 3.54E−04 | 4.82E−02 | | |
| cg03293330 | 0.083 | 3.56E−04 | 4.84E−02 | | |
| cg25247402 | 0.036 | 3.57E−04 | 4.84E−02 | | |
| cg10912634 | 0.021 | 3.60E−04 | 4.86E−02 | SRA1 | FALSE |
| cg20018425 | 0.044 | 3.64E−04 | 4.87E−02 | PANX2 | FALSE |
| cg10316270 | 0.034 | 3.63E−04 | 4.87E−02 | RBM46 | FALSE |
| cg04585787 | 0.042 | 3.62E−04 | 4.87E−02 | | |
| cg00962793 | 0.026 | 3.66E−04 | 4.87E−02 | | |
| cg27025137 | −0.034 | 3.65E−04 | 4.87E−02 | SLC39A11 | FALSE |
| cg03834031 | −0.049 | 3.66E−04 | 4.87E−02 | | |
| cg08879111 | 0.101 | 3.68E−04 | 4.89E−02 | IL1R2 | FALSE |
| cg02278646 | −0.101 | 3.69E−04 | 4.89E−02 | ANO6 | FALSE |
| cg07464859 | 0.024 | 3.70E−04 | 4.89E−02 | C16orf45 | FALSE |
| cg04276765 | 0.056 | 3.70E−04 | 4.89E−02 | | |
| cg04845579 | 0.052 | 3.70E−04 | 4.89E−02 | SULF1 | FALSE |
| cg24219974 | 0.092 | 3.70E−04 | 4.89E−02 | | |
| cg26300517 | 0.071 | 3.71E−04 | 4.89E−02 | | |
| cg03621504 | −0.079 | 3.73E−04 | 4.91E−02 | MED13L | FALSE |
| cg06937882 | −0.100 | 3.76E−04 | 4.92E−02 | C20orf3 | FALSE |
| cg07813254 | 0.031 | 3.75E−04 | 4.92E−02 | FAM43B | FALSE |
| cg09183671 | 0.033 | 3.77E−04 | 4.92E−02 | LOC282997 | FALSE |
| cg06094707 | 0.059 | 3.74E−04 | 4.92E−02 | NPTX1 | FALSE |
| cg26751868 | 0.020 | 3.75E−04 | 4.92E−02 | | |
| cg21878759 | 0.052 | 3.77E−04 | 4.92E−02 | LOXHD1 | FALSE |
| cg10341242 | −0.062 | 3.79E−04 | 4.93E−02 | ADCY7 | FALSE |
| cg20905984 | 0.029 | 3.80E−04 | 4.94E−02 | NCRNA00171 | FALSE |
| cg07352544 | −0.045 | 3.80E−04 | 4.94E−02 | SEPN1 | FALSE |
| cg04981737 | 0.030 | 3.80E−04 | 4.94E−02 | HOXC10 | FALSE |
| cg23838245 | −0.049 | 3.81E−04 | 4.94E−02 | HGF | FALSE |
| cg24048008 | 0.026 | 3.81E−04 | 4.94E−02 | SCARNA2 | FALSE |
| cg03849728 | 0.034 | 3.83E−04 | 4.94E−02 | C16orf53 | FALSE |
| cg15316716 | 0.063 | 3.85E−04 | 4.94E−02 | DOK2 | FALSE |
| cg09380357 | 0.034 | 3.83E−04 | 4.94E−02 | NUB1 | FALSE |
| cg14103123 | 0.032 | 3.82E−04 | 4.94E−02 | RALGDS | FALSE |
| cg21812313 | 0.077 | 3.84E−04 | 4.94E−02 | | |
| cg10017105 | −0.047 | 3.86E−04 | 4.94E−02 | SLC44A4 | FALSE |
| cg26867282 | 0.027 | 3.87E−04 | 4.94E−02 | | |
| cg16879895 | 0.037 | 3.88E−04 | 4.94E−02 | | |
| cg03087880 | 0.065 | 3.90E−04 | 4.95E−02 | ZNF423 | FALSE |
| cg05224770 | 0.097 | 3.91E−04 | 4.95E−02 | IFNG | FALSE |
| cg19076265 | 0.028 | 3.95E−04 | 4.96E−02 | DOCK5 | FALSE |
| cg05033369 | 0.077 | 3.96E−04 | 4.96E−02 | FCRLA | FALSE |
| cg15005421 | 0.035 | 3.95E−04 | 4.96E−02 | FILIP1 | FALSE |
| cg18670076 | 0.060 | 3.93E−04 | 4.96E−02 | LEPREL1 | FALSE |
| cg04358685 | 0.020 | 3.92E−04 | 4.96E−02 | MKRN2 | FALSE |
| cg07167933 | 0.031 | 3.94E−04 | 4.96E−02 | SFTPB | FALSE |
| cg24312874 | 0.036 | 3.94E−04 | 4.96E−02 | | |
| cg08594651 | −0.069 | 3.97E−04 | 4.96E−02 | | |
| cg13615971 | −0.067 | 3.97E−04 | 4.96E−02 | | |
| cg13464004 | 0.042 | 3.94E−04 | 4.96E−02 | TMEM99 | FALSE |
| cg16975985 | 0.019 | 3.93E−04 | 4.96E−02 | TUBA1B | FALSE |
| cg02403412 | 0.021 | 3.96E−04 | 4.96E−02 | TXNRD2 | FALSE |
| cg19517410 | 0.038 | 3.96E−04 | 4.96E−02 | VEPH1 | FALSE |
| cg02158755 | 0.052 | 3.98E−04 | 4.96E−02 | BTNL2 | FALSE |
| cg09305161 | 0.058 | 3.98E−04 | 4.96E−02 | PRDM1 | FALSE |
| cg01618245 | 0.046 | 4.00E−04 | 4.96E−02 | CHRNA4 | FALSE |
| cg06737907 | 0.028 | 4.00E−04 | 4.96E−02 | MEGF11 | FALSE |
| cg06931905 | 0.039 | 4.00E−04 | 4.96E−02 | PLAT | FALSE |
| cg22862005 | −0.070 | 4.01E−04 | 4.96E−02 | SLC26A9 | FALSE |
| cg08846959 | 0.026 | 4.00E−04 | 4.96E−02 | | |
| cg04656451 | −0.053 | 4.02E−04 | 4.96E−02 | | |
| cg13868520 | 0.047 | 4.02E−04 | 4.96E−02 | TNFRSF8 | FALSE |
| cg03883256 | 0.061 | 3.99E−04 | 4.96E−02 | USP2 | FALSE |
| cg12897164 | 0.100 | 4.04E−04 | 4.97E−02 | FBXO32 | FALSE |
| cg07462863 | 0.038 | 4.09E−04 | 4.97E−02 | EVI2A | FALSE |
| cg00633768 | 0.042 | 4.08E−04 | 4.97E−02 | GPR133 | FALSE |
| cg07189401 | −0.031 | 4.08E−04 | 4.97E−02 | LOC100126784 | FALSE |
| cg00530015 | 0.042 | 4.07E−04 | 4.97E−02 | | |
| cg06506598 | 0.018 | 4.10E−04 | 4.97E−02 | FAM151B | FALSE |
| cg03546163 | −0.095 | 4.10E−04 | 4.97E−02 | FKBP5 | FALSE |
| cg16296829 | 0.051 | 4.11E−04 | 4.97E−02 | KCNN3 | FALSE |
| cg00687135 | 0.059 | 4.10E−04 | 4.97E−02 | ZNF385D | FALSE |
| cg07441944 | 0.064 | 4.12E−04 | 4.97E−02 | CD82 | FALSE |
| cg14575739 | 0.036 | 4.13E−04 | 4.97E−02 | LILRA2 | FALSE |
| cg10058885 | 0.040 | 4.13E−04 | 4.97E−02 | | |
| cg22111074 | 0.020 | 4.16E−04 | 4.97E−02 | BSN | FALSE |
| cg04970434 | −0.033 | 4.15E−04 | 4.97E−02 | GP1BA | FALSE |
| cg13583414 | 0.057 | 4.15E−04 | 4.97E−02 | LZTS1 | FALSE |
| cg02892755 | 0.032 | 4.15E−04 | 4.97E−02 | STX12 | FALSE |
| cg25817351 | 0.044 | 4.15E−04 | 4.97E−02 | | |
| cg25264948 | 0.046 | 4.19E−04 | 4.97E−02 | C6orf27 | FALSE |
| cg01466121 | 0.020 | 4.18E−04 | 4.97E−02 | CASP8 | |
| cg21898299 | 0.050 | 4.19E−04 | 4.97E−02 | CORO2B | FALSE |
| cg18835865 | 0.081 | 4.17E−04 | 4.97E−02 | FRMD6 | FALSE |
| cg19222784 | 0.065 | 4.18E−04 | 4.97E−02 | NAV2 | FALSE |
| cg23221723 | −0.077 | 4.19E−04 | 4.97E−02 | TNFRSF10C | FALSE |
| cg07013258 | −0.034 | 4.28E−04 | 4.98E−02 | BCL7C | FALSE |
| cg16846532 | 0.063 | 4.24E−04 | 4.98E−02 | DDC | FALSE |
| cg03531100 | 0.026 | 4.28E−04 | 4.98E−02 | FGF21 | FALSE |
| cg02508664 | −0.040 | 4.26E−04 | 4.98E−02 | HTR1E | FALSE |
| cg24476449 | 0.069 | 4.26E−04 | 4.98E−02 | KSR1 | FALSE |
| cg01311805 | 0.037 | 4.24E−04 | 4.98E−02 | LDLRAD3 | FALSE |
| cg27561099 | 0.060 | 4.27E−04 | 4.98E−02 | MAP3K13 | FALSE |
| cg00564759 | 0.019 | 4.26E−04 | 4.98E−02 | MAZ | FALSE |
| cg01300434 | 0.046 | 4.20E−04 | 4.98E−02 | | |
| cg02133456 | 0.032 | 4.20E−04 | 4.98E−02 | | |
| cg01191154 | 0.035 | 4.22E−04 | 4.98E−02 | | |
| cg24678438 | 0.035 | 4.28E−04 | 4.98E−02 | | |
| cg08428292 | 0.063 | 4.28E−04 | 4.98E−02 | | |
| cg03613822 | 0.097 | 4.31E−04 | 4.98E−02 | DLG4 | FALSE |
| cg13911801 | 0.024 | 4.30E−04 | 4.98E−02 | LOC92973 | FALSE |
| cg02331830 | 0.088 | 4.31E−04 | 4.98E−02 | PLEC1 | FALSE |
| cg26888012 | 0.054 | 4.31E−04 | 4.98E−02 | PSORS1C1 | FALSE |
| cg21496187 | 0.026 | 4.31E−04 | 4.98E−02 | | |
| cg02165330 | −0.085 | 4.32E−04 | 4.98E−02 | SIAE | FALSE |
| cg15924691 | 0.049 | 4.34E−04 | 4.99E−02 | EPHB1 | FALSE |
| cg13572237 | 0.028 | 4.36E−04 | 4.99E−02 | CARD9 | FALSE |
| cg27138949 | 0.047 | 4.36E−04 | 4.99E−02 | CLDN18 | FALSE |
| cg21637761 | 0.034 | 4.35E−04 | 4.99E−02 | CYTSB | FALSE |
| cg05622441 | 0.018 | 4.38E−04 | 4.99E−02 | TERF2 | FALSE |
| cg24758452 | 0.043 | 4.37E−04 | 4.99E−02 | | |
| cg23490246 | 0.068 | 4.38E−04 | 4.99E−02 | HECW2 | FALSE |
| cg15002691 | 0.057 | 4.46E−04 | 4.99E−02 | AKR1D1 | FALSE |
| cg20666917 | −0.041 | 4.41E−04 | 4.99E−02 | C2orf55 | FALSE |
| cg26793227 | −0.059 | 4.47E−04 | 4.99E−02 | EPHA2 | FALSE |
| cg25030266 | 0.028 | 4.44E−04 | 4.99E−02 | ITPRIP | FALSE |
| cg02402436 | 0.118 | 4.44E−04 | 4.99E−02 | LTA | FALSE |
| cg20594465 | 0.037 | 4.45E−04 | 4.99E−02 | PCDH17 | FALSE |
| cg21826978 | 0.041 | 4.41E−04 | 4.99E−02 | PSMB9 | FALSE |
| cg21775570 | −0.073 | 4.46E−04 | 4.99E−02 | SKI | FALSE |
| cg25480132 | 0.022 | 4.43E−04 | 4.99E−02 | STMN3 | FALSE |
| cg24742520 | −0.082 | 4.45E−04 | 4.99E−02 | UBR4 | FALSE |
| cg12975597 | 0.032 | 4.41E−04 | 4.99E−02 | | |
| cg04479464 | −0.022 | 4.41E−04 | 4.99E−02 | | |
| cg27248148 | 0.038 | 4.42E−04 | 4.99E−02 | | |
| cg05612966 | 0.036 | 4.44E−04 | 4.99E−02 | | |
| cg25564535 | 0.049 | 4.42E−04 | 4.99E−02 | ZAP70 | FALSE |
| cg06379435 | −0.070 | 4.47E−04 | 4.99E−02 | | |
| cg08348121 | 0.028 | 4.48E−04 | 5.00E−02 | SKIL | FALSE |

TABLE 10C
Differentially Methylated CpG Sites Associated
with IBD vs Healthy <0.005fdr

| | logFC | P. Value | adj. P. Val | UCSC_RefGene_Name |
|---|---|---|---|---|
| cg03922648 | 0.060 | 2.30E−11 | 1.30E−06 | KLRC1 |
| cg01668008 | 0.041 | 2.31E−11 | 1.30E−06 | |
| cg11906021 | 0.123 | 1.90E−10 | 4.27E−06 | |
| cg11650874 | 0.056 | 3.28E−10 | 6.14E−06 | CHID1 |
| cg11772801 | 0.080 | 8.24E−10 | 8.91E−06 | TMX1 |
| cg03109660 | 0.115 | 9.89E−10 | 9.66E−06 | RELL1 |
| cg21500092 | 0.050 | 9.80E−10 | 9.66E−06 | UBE2G1 |
| cg17313709 | −0.133 | 1.34E−09 | 1.26E−05 | |
| cg06858263 | 0.055 | 2.04E−09 | 1.48E−05 | DNAJB6 |
| cg13719443 | −0.076 | 2.31E−09 | 1.52E−05 | LOC285847 |
| cg14126884 | 0.081 | 3.69E−09 | 2.02E−05 | RAPGEF1 |
| cg22357379 | 0.038 | 5.41E−09 | 2.64E−05 | MIDN |
| cg18274749 | 0.061 | 7.08E−09 | 3.18E−05 | HSPG2 |
| cg13641591 | 0.044 | 1.18E−08 | 4.43E−05 | UVRAG |
| cg15518113 | 0.064 | 1.72E−08 | 5.67E−05 | CD247 |
| cg23966795 | 0.042 | 2.36E−08 | 6.88E−05 | TANC1 |
| cg11978441 | 0.066 | 2.76E−08 | 7.37E−05 | PER3 |
| cg08854185 | 0.067 | 3.88E−08 | 9.21E−05 | DBNDD1 |
| cg07119168 | 0.059 | 4.72E−08 | 1.01E−04 | SPTB |
| cg02991558 | 0.042 | 4.96E−08 | 1.04E−04 | |
| cg01291375 | 0.050 | 5.45E−08 | 1.08E−04 | |
| cg13489576 | −0.034 | 7.48E−08 | 1.33E−04 | DLG1 |
| cg25256099 | 0.092 | 7.92E−08 | 1.36E−04 | |
| cg04787728 | 0.058 | 8.51E−08 | 1.42E−04 | NLRC3 |
| cg21442182 | 0.046 | 9.15E−08 | 1.47E−04 | |
| cg12572295 | 0.058 | 9.65E−08 | 1.49E−04 | GRK4 |
| cg02192218 | 0.043 | 1.30E−07 | 1.74E−04 | CLN3 |
| cg18181906 | 0.036 | 1.40E−07 | 1.78E−04 | |
| cg02652361 | −0.112 | 1.53E−07 | 1.85E−04 | MALAT1 |
| cg01727923 | 0.054 | 1.62E−07 | 1.89E−04 | |
| cg16023434 | 0.065 | 1.66E−07 | 1.90E−04 | |
| cg20114528 | 0.065 | 1.69E−07 | 1.90E−04 | ASB8 |
| cg16815882 | −0.083 | 1.76E−07 | 1.95E−04 | KIAA0319L |
| cg14348967 | −0.073 | 1.87E−07 | 1.98E−04 | |
| cg22013370 | 0.080 | 1.98E−07 | 2.06E−04 | |
| cg21486834 | 0.052 | 2.23E−07 | 2.26E−04 | RHBDF2 |
| cg02816525 | 0.080 | 2.35E−07 | 2.32E−04 | ADAM10 |
| cg24470880 | 0.053 | 2.48E−07 | 2.39E−04 | |
| cg21848117 | 0.040 | 2.59E−07 | 2.44E−04 | SLC38A10 |
| cg03726817 | −0.086 | 2.75E−07 | 2.54E−04 | |
| cg05365532 | 0.100 | 3.05E−07 | 2.72E−04 | LILRB1 |
| cg00809267 | 0.082 | 3.04E−07 | 2.72E−04 | NEDD4L |
| cg14249520 | −0.124 | 3.10E−07 | 2.74E−04 | CPT1A |
| cg18221341 | 0.033 | 3.43E−07 | 2.86E−04 | |
| cg03884572 | 0.047 | 3.52E−07 | 2.90E−04 | CUX2 |
| cg07029002 | 0.085 | 3.62E−07 | 2.93E−04 | ROR1 |
| cg14712058 | 0.047 | 3.70E−07 | 2.94E−04 | SIN3B |
| cg26775087 | 0.036 | 3.93E−07 | 3.06E−04 | SH3BP5 |
| cg11283404 | 0.025 | 4.00E−07 | 3.09E−04 | IKBKB |
| cg25015416 | 0.043 | 4.16E−07 | 3.17E−04 | CNP |
| cg27123273 | 0.051 | 5.03E−07 | 3.57E−04 | |
| cg06861560 | 0.028 | 5.40E−07 | 3.70E−04 | |
| cg23343291 | 0.042 | 6.52E−07 | 4.00E−04 | INF2 |
| cg21046951 | 0.063 | 6.71E−07 | 4.05E−04 | WDR18 |
| cg12559471 | 0.075 | 6.93E−07 | 4.12E−04 | |
| cg00910203 | 0.026 | 7.20E−07 | 4.24E−04 | |
| cg02757488 | 0.056 | 7.80E−07 | 4.49E−04 | NUMBL |
| cg12127149 | −0.077 | 8.32E−07 | 4.75E−04 | DYNC1H1 |
| cg04821520 | 0.048 | 8.82E−07 | 4.93E−04 | |
| cg22732126 | 0.032 | 8.93E−07 | 4.94E−04 | |
| cg02083319 | 0.050 | 9.16E−07 | 4.98E−04 | DOK3 |
| cg04519626 | 0.043 | 9.71E−07 | 5.16E−04 | |
| cg07894334 | 0.091 | 9.88E−07 | 5.21E−04 | SOCS5 |
| cg00923744 | −0.049 | 1.04E−06 | 5.36E−04 | GALNT2 |
| cg23589035 | −0.109 | 1.09E−06 | 5.53E−04 | ASH1L |
| cg06545464 | 0.043 | 1.10E−06 | 5.53E−04 | C6orf10 |
| cg02328326 | −0.137 | 1.11E−06 | 5.56E−04 | MAP2K5 |
| cg03797906 | −0.047 | 1.11E−06 | 5.56E−04 | TRIL |
| cg06679990 | 0.052 | 1.16E−06 | 5.74E−04 | GLI1 |
| cg04861974 | 0.069 | 1.20E−06 | 5.84E−04 | |
| cg06588802 | 0.075 | 1.22E−06 | 5.91E−04 | |
| cg22508930 | 0.030 | 1.24E−06 | 5.99E−04 | RAP1B |
| cg14807945 | 0.064 | 1.26E−06 | 6.05E−04 | ZEB2 |
| cg24611970 | 0.038 | 1.26E−06 | 6.07E−04 | |
| cg12092579 | 0.088 | 1.31E−06 | 6.26E−04 | RASAL2 |
| cg17177006 | −0.083 | 1.35E−06 | 6.31E−04 | EDEM1 |
| cg05839818 | 0.036 | 1.36E−06 | 6.31E−04 | |
| cg25894551 | 0.056 | 1.42E−06 | 6.55E−04 | FXYD2 |
| cg09521743 | 0.083 | 1.44E−06 | 6.60E−04 | SYNPO2L |
| cg11354682 | 0.159 | 1.47E−06 | 6.64E−04 | C19orf38 |
| cg14685948 | 0.046 | 1.49E−06 | 6.70E−04 | COL13A1 |
| cg26104475 | −0.054 | 1.50E−06 | 6.70E−04 | LCP2 |
| cg26102819 | 0.070 | 1.56E−06 | 6.85E−04 | HEG1 |
| cg22202689 | 0.036 | 1.56E−06 | 6.85E−04 | |
| cg16214653 | 0.060 | 1.58E−06 | 6.90E−04 | |
| cg21686632 | 0.063 | 1.65E−06 | 7.09E−04 | VPS13D |
| cg13765961 | 0.109 | 1.68E−06 | 7.16E−04 | MS4A1 |
| cg12102235 | −0.109 | 1.74E−06 | 7.36E−04 | |
| cg27203560 | −0.081 | 1.89E−06 | 7.73E−04 | |
| cg26456304 | 0.047 | 1.90E−06 | 7.76E−04 | |
| cg17579089 | 0.052 | 1.92E−06 | 7.79E−04 | KIF9 |
| cg23109530 | 0.061 | 1.96E−06 | 7.92E−04 | |
| cg01761729 | 0.033 | 2.10E−06 | 8.23E−04 | LIMS2 |
| cg27548256 | 0.044 | 2.16E−06 | 8.35E−04 | ACTN4 |
| cg19910327 | 0.029 | 2.17E−06 | 8.36E−04 | PIPSL |
| cg01504248 | 0.045 | 2.19E−06 | 8.39E−04 | |
| cg25030266 | 0.041 | 2.41E−06 | 8.85E−04 | ITPRIP |
| cg17910470 | 0.053 | 2.43E−06 | 8.91E−04 | USP36 |
| cg13388731 | 0.050 | 2.49E−06 | 9.01E−04 | DNAJB2 |
| cg25801622 | 0.051 | 2.59E−06 | 9.24E−04 | PITPNM2 |
| cg08573679 | −0.057 | 2.71E−06 | 9.48E−04 | |
| cg03413355 | −0.064 | 2.72E−06 | 9.51E−04 | |
| cg14210119 | −0.109 | 2.74E−06 | 9.52E−04 | |
| cg06216065 | 0.043 | 2.78E−06 | 9.57E−04 | ZDHHC18 |
| cg20594465 | 0.050 | 2.83E−06 | 9.66E−04 | PCDH17 |
| cg01400750 | −0.098 | 2.86E−06 | 9.72E−04 | ANAPC10 |
| cg10860364 | 0.037 | 2.89E−06 | 9.79E−04 | ANKRD13B |
| cg09004241 | 0.052 | 2.91E−06 | 9.82E−04 | B3GNT7 |
| cg19850370 | 0.059 | 2.98E−06 | 1.00E−03 | SLC4A4 |
| cg16455376 | 0.032 | 2.99E−06 | 1.00E−03 | |
| cg13373343 | 0.044 | 3.01E−06 | 1.00E−03 | |
| cg17492395 | 0.083 | 3.01E−06 | 1.00E−03 | |
| cg09607915 | 0.061 | 3.15E−06 | 1.04E−03 | FBXO11 |
| cg24235882 | −0.082 | 3.16E−06 | 1.04E−03 | CHIC2 |
| cg07728579 | 0.077 | 3.18E−06 | 1.04E−03 | FSD2 |
| cg14207267 | −0.137 | 3.22E−06 | 1.05E−03 | IQCD |
| cg23249717 | 0.021 | 3.22E−06 | 1.05E−03 | |
| cg10113217 | 0.037 | 3.26E−06 | 1.05E−03 | |
| cg04820773 | 0.029 | 3.29E−06 | 1.06E−03 | |
| cg05435993 | 0.027 | 3.34E−06 | 1.06E−03 | |
| cg13192013 | 0.032 | 3.38E−06 | 1.07E−03 | RAPGEF2 |
| cg16474118 | 0.032 | 3.50E−06 | 1.09E−03 | |
| cg19818308 | 0.077 | 3.51E−06 | 1.09E−03 | |
| cg06676778 | 0.153 | 3.56E−06 | 1.10E−03 | |
| cg12479878 | −0.046 | 3.60E−06 | 1.11E−03 | HLX |
| cg14586546 | 0.048 | 3.73E−06 | 1.13E−03 | RALGDS |
| cg10389454 | −0.054 | 3.80E−06 | 1.15E−03 | CEACAM21 |
| cg18460107 | 0.068 | 3.79E−06 | 1.15E−03 | CLEC4C |
| cg01637125 | 0.090 | 3.80E−06 | 1.15E−03 | |
| cg19465002 | 0.029 | 3.83E−06 | 1.15E−03 | |
| cg25530583 | 0.039 | 3.86E−06 | 1.16E−03 | SLC14A1 |
| cg27655168 | 0.034 | 3.88E−06 | 1.16E−03 | |
| cg08534055 | 0.035 | 3.91E−06 | 1.16E−03 | C16orf72 |
| cg01387036 | 0.038 | 3.94E−06 | 1.16E−03 | CCL1 |
| cg22911650 | 0.077 | 3.93E−06 | 1.16E−03 | SIGLEC7 |
| cg14305659 | 0.025 | 3.95E−06 | 1.16E−03 | |
| cg14111697 | 0.042 | 4.13E−06 | 1.19E−03 | GNAQ |
| cg02287325 | 0.040 | 4.21E−06 | 1.21E−03 | CTRB1 |
| cg18881723 | −0.061 | 4.26E−06 | 1.22E−03 | SLAMF1 |
| cg16210979 | 0.042 | 4.34E−06 | 1.23E−03 | YTHDC2 |
| cg03548857 | −0.081 | 4.38E−06 | 1.23E−03 | FFAR2 |
| cg16412914 | 0.030 | 4.60E−06 | 1.28E−03 | AXIN1 |
| cg01582066 | 0.088 | 4.65E−06 | 1.29E−03 | |
| cg26283496 | 0.106 | 4.74E−06 | 1.31E−03 | |
| cg27443224 | 0.060 | 5.03E−06 | 1.36E−03 | CCL21 |
| cg04162299 | 0.064 | 5.12E−06 | 1.38E−03 | DOCK4 |
| cg27659109 | 0.028 | 5.22E−06 | 1.40E−03 | FZR1 |
| cg11849692 | −0.053 | 5.33E−06 | 1.41E−03 | LDB1 |
| cg03466326 | 0.033 | 5.34E−06 | 1.41E−03 | |
| cg17994788 | 0.074 | 5.52E−06 | 1.44E−03 | C1GALT1 |
| cg24678438 | 0.040 | 5.68E−06 | 1.47E−03 | |
| cg11147278 | −0.101 | 5.75E−06 | 1.48E−03 | |
| cg04150762 | 0.056 | 5.82E−06 | 1.49E−03 | |
| cg08879111 | 0.112 | 5.86E−06 | 1.50E−03 | IL1R2 |
| cg02127689 | 0.037 | 5.86E−06 | 1.50E−03 | |
| cg27161340 | 0.023 | 5.94E−06 | 1.51E−03 | ZCCHC14 |
| cg07091500 | −0.129 | 6.17E−06 | 1.55E−03 | |
| cg09831562 | 0.036 | 6.24E−06 | 1.56E−03 | SOX2OT |
| cg24162801 | 0.056 | 6.25E−06 | 1.56E−03 | C1orf21 |
| cg12062672 | 0.043 | 6.27E−06 | 1.57E−03 | FGF22 |
| cg25020550 | −0.057 | 6.28E−06 | 1.57E−03 | TG |
| cg07729082 | 0.049 | 6.44E−06 | 1.59E−03 | ZC3H18 |
| cg11671363 | −0.104 | 6.49E−06 | 1.59E−03 | MIR145 |
| cg18869709 | −0.080 | 6.53E−06 | 1.60E−03 | PACSIN2 |
| cg23221723 | −0.085 | 6.64E−06 | 1.62E−03 | TNFRSF10C |
| cg07035442 | 0.036 | 6.70E−06 | 1.62E−03 | |
| cg09073441 | 0.052 | 6.73E−06 | 1.62E−03 | |
| cg27117982 | −0.109 | 6.86E−06 | 1.65E−03 | MGST1 |
| cg13084525 | 0.061 | 6.89E−06 | 1.65E−03 | EHF |
| cg21010350 | 0.033 | 6.95E−06 | 1.66E−03 | LOC100128542 |
| cg18247172 | 0.057 | 7.07E−06 | 1.67E−03 | |
| cg18307978 | 0.041 | 7.19E−06 | 1.69E−03 | AQP1 |
| cg26169783 | 0.088 | 7.22E−06 | 1.69E−03 | |
| cg00905524 | −0.039 | 7.28E−06 | 1.70E−03 | |
| cg17426969 | 0.033 | 7.54E−06 | 1.75E−03 | PAN3 |
| cg16662477 | −0.098 | 7.58E−06 | 1.75E−03 | RFFL |
| cg16469419 | 0.032 | 7.63E−06 | 1.76E−03 | MTA2 |
| cg22707397 | 0.058 | 7.71E−06 | 1.77E−03 | |
| cg14632490 | 0.034 | 7.95E−06 | 1.80E−03 | |
| cg05766064 | −0.043 | 8.05E−06 | 1.82E−03 | ABHD3 |
| cg08928189 | 0.064 | 8.13E−06 | 1.82E−03 | |
| cg07338715 | −0.063 | 8.15E−06 | 1.82E−03 | |
| cg19145995 | 0.035 | 8.15E−06 | 1.82E−03 | |
| cg01205058 | 0.037 | 8.38E−06 | 1.86E−03 | |
| cg03478448 | 0.049 | 8.61E−06 | 1.89E−03 | SLC12A3 |
| cg27601906 | 0.029 | 8.80E−06 | 1.93E−03 | GEM |
| cg23948811 | 0.027 | 8.82E−06 | 1.93E−03 | |
| cg04664309 | 0.042 | 8.82E−06 | 1.93E−03 | |
| cg21220247 | 0.031 | 8.92E−06 | 1.94E−03 | |
| cg08439487 | 0.028 | 9.17E−06 | 1.97E−03 | MYLK |
| cg24364282 | 0.042 | 9.17E−06 | 1.97E−03 | |
| cg05875239 | 0.043 | 9.44E−06 | 2.01E−03 | |
| cg27069188 | −0.034 | 9.53E−06 | 2.03E−03 | ITFG3 |
| cg05148180 | 0.039 | 9.61E−06 | 2.04E−03 | |
| cg25887844 | −0.058 | 9.63E−06 | 2.04E−03 | ARHGAP25 |
| cg05235171 | 0.038 | 1.00E−05 | 2.10E−03 | TMPRSS4 |
| cg02375166 | −0.049 | 1.01E−05 | 2.10E−03 | |
| cg13583414 | 0.069 | 1.01E−05 | 2.10E−03 | LZTS1 |
| cg20461912 | −0.020 | 1.02E−05 | 2.12E−03 | FOXL2 |
| cg27658026 | 0.064 | 1.04E−05 | 2.15E−03 | CALHM3 |
| cg07175797 | 0.041 | 1.07E−05 | 2.19E−03 | |
| cg06536806 | 0.064 | 1.08E−05 | 2.20E−03 | |
| cg09502619 | −0.080 | 1.09E−05 | 2.22E−03 | IFT140 |
| cg15244874 | −0.049 | 1.11E−05 | 2.24E−03 | RAB19 |
| cg12749559 | −0.074 | 1.13E−05 | 2.26E−03 | BRP44 |
| cg14718848 | −0.074 | 1.14E−05 | 2.26E−03 | ETS1 |
| cg11358199 | 0.068 | 1.13E−05 | 2.26E−03 | |
| cg05259303 | 0.040 | 1.15E−05 | 2.29E−03 | |
| cg14055655 | 0.060 | 1.16E−05 | 2.30E−03 | PRR12 |
| cg04873169 | 0.044 | 1.16E−05 | 2.30E−03 | GRAMD4 |
| cg00937012 | 0.034 | 1.18E−05 | 2.30E−03 | CPEB1 |
| cg04060561 | 0.072 | 1.18E−05 | 2.30E−03 | ECHDC1 |
| cg12079885 | 0.030 | 1.18E−05 | 2.30E−03 | SCARB1 |
| cg17580616 | 0.043 | 1.19E−05 | 2.31E−03 | MADD |
| cg11304968 | 0.033 | 1.21E−05 | 2.33E−03 | |
| cg15018704 | 0.032 | 1.22E−05 | 2.34E−03 | ADAT3 |
| cg00732432 | −0.030 | 1.25E−05 | 2.38E−03 | CPLX1 |
| cg07692929 | 0.050 | 1.27E−05 | 2.40E−03 | OLFML1 |
| cg08413427 | −0.091 | 1.30E−05 | 2.43E−03 | DBF4B |
| cg01719995 | −0.087 | 1.30E−05 | 2.43E−03 | USP38 |
| cg24119869 | 0.098 | 1.33E−05 | 2.46E−03 | KIR3DX1 |
| cg25029657 | 0.039 | 1.33E−05 | 2.47E−03 | ACOT7 |
| cg00786165 | 0.097 | 1.33E−05 | 2.47E−03 | |
| cg22623033 | 0.035 | 1.36E−05 | 2.49E−03 | PIP5K1C |
| cg10170504 | 0.056 | 1.37E−05 | 2.50E−03 | FLJ43663 |
| cg08753560 | 0.065 | 1.38E−05 | 2.51E−03 | |
| cg17908947 | 0.030 | 1.44E−05 | 2.57E−03 | MAFF |
| cg26341831 | 0.106 | 1.45E−05 | 2.57E−03 | TMEM63A |
| cg22768358 | 0.036 | 1.45E−05 | 2.57E−03 | ZBTB16 |
| cg00360761 | −0.048 | 1.47E−05 | 2.59E−03 | |
| cg02932364 | 0.025 | 1.47E−05 | 2.59E−03 | |
| cg11241750 | 0.057 | 1.48E−05 | 2.61E−03 | GRAMD3 |
| cg20979737 | −0.064 | 1.52E−05 | 2.65E−03 | |
| cg25392995 | 0.094 | 1.55E−05 | 2.67E−03 | TRABD |
| cg22109240 | −0.081 | 1.56E−05 | 2.68E−03 | KCTD18 |
| cg06371582 | −0.047 | 1.56E−05 | 2.68E−03 | TMEM154 |
| cg01049678 | −0.040 | 1.57E−05 | 2.69E−03 | GIMAP8 |
| cg08118034 | 0.029 | 1.59E−05 | 2.71E−03 | GAS2L1 |
| cg13278350 | 0.038 | 1.59E−05 | 2.71E−03 | |
| cg14701962 | 0.044 | 1.59E−05 | 2.71E−03 | C1orf226 |
| cg18926797 | −0.059 | 1.61E−05 | 2.72E−03 | DCAF5 |
| cg08161480 | −0.081 | 1.64E−05 | 2.76E−03 | |
| cg07510346 | 0.038 | 1.70E−05 | 2.82E−03 | MYOM2 |
| cg04453371 | 0.048 | 1.71E−05 | 2.83E−03 | |
| cg17843665 | 0.098 | 1.71E−05 | 2.84E−03 | MIPEP |
| cg07838483 | −0.092 | 1.73E−05 | 2.85E−03 | C19orf12 |
| cg05832823 | 0.035 | 1.73E−05 | 2.85E−03 | TBC1D4 |
| cg01939741 | 0.028 | 1.74E−05 | 2.86E−03 | |
| cg19978416 | 0.041 | 1.75E−05 | 2.86E−03 | |
| cg00699986 | −0.069 | 1.76E−05 | 2.87E−03 | VSX1 |
| cg01822759 | 0.066 | 1.77E−05 | 2.87E−03 | |
| cg09160359 | 0.032 | 1.77E−05 | 2.87E−03 | MESDC1 |
| cg01815912 | 0.037 | 1.80E−05 | 2.89E−03 | NTHL1 |
| cg00221794 | 0.076 | 1.80E−05 | 2.89E−03 | |
| cg25332502 | 0.033 | 1.82E−05 | 2.92E−03 | MBP |
| cg13572237 | 0.033 | 1.84E−05 | 2.94E−03 | CARD9 |
| cg18303397 | −0.086 | 1.85E−05 | 2.94E−03 | IFT122 |
| cg25312939 | 0.041 | 1.87E−05 | 2.96E−03 | |
| cg22798362 | 0.134 | 1.88E−05 | 2.97E−03 | MYO1D |
| cg05422796 | −0.092 | 1.89E−05 | 2.99E−03 | ZFP161 |
| cg19380303 | 0.045 | 1.93E−05 | 3.02E−03 | KIF7 |
| cg11411705 | −0.053 | 1.95E−05 | 3.03E−03 | TSPAN14 |
| cg16501235 | 0.034 | 1.95E−05 | 3.03E−03 | C1orf54 |
| cg19849557 | 0.049 | 1.95E−05 | 3.04E−03 | C6orf136 |
| cg08496123 | 0.021 | 1.96E−05 | 3.04E−03 | HAS1 |
| cg26331247 | 0.074 | 1.99E−05 | 3.06E−03 | LOC284805 |
| cg25474070 | 0.037 | 1.99E−05 | 3.07E−03 | IL3 |
| cg02976539 | 0.050 | 2.03E−05 | 3.10E−03 | SLC9A3R1 |
| cg06214853 | 0.033 | 2.05E−05 | 3.12E−03 | |
| cg19420720 | −0.051 | 2.09E−05 | 3.16E−03 | P4HB |
| cg12860109 | 0.069 | 2.10E−05 | 3.16E−03 | NIPAL2 |
| cg03302822 | 0.058 | 2.13E−05 | 3.19E−03 | C10orf28 |
| cg19103429 | −0.084 | 2.16E−05 | 3.22E−03 | CDK6 |
| cg03596479 | 0.046 | 2.16E−05 | 3.22E−03 | CTSC |
| cg05567236 | −0.043 | 2.16E−05 | 3.22E−03 | SDCCAG8 |
| cg27025137 | −0.035 | 2.17E−05 | 3.22E−03 | SLC39A11 |
| cg24180759 | 0.082 | 2.19E−05 | 3.24E−03 | ODZ2 |
| cg00249522 | 0.029 | 2.20E−05 | 3.25E−03 | |
| cg08823209 | 0.067 | 2.21E−05 | 3.26E−03 | FXYD3 |
| cg00759427 | −0.048 | 2.26E−05 | 3.31E−03 | RGS19 |
| cg08056716 | 0.038 | 2.27E−05 | 3.31E−03 | |
| cg10274029 | 0.057 | 2.28E−05 | 3.32E−03 | |
| cg07935669 | 0.022 | 2.29E−05 | 3.33E−03 | |
| cg19922871 | 0.059 | 2.29E−05 | 3.33E−03 | |
| cg13949137 | 0.046 | 2.33E−05 | 3.38E−03 | |
| cg07998908 | −0.073 | 2.36E−05 | 3.40E−03 | |
| cg22360318 | −0.062 | 2.38E−05 | 3.42E−03 | GAS7 |
| cg03044533 | −0.054 | 2.41E−05 | 3.44E−03 | MAML2 |
| cg18404811 | −0.096 | 2.40E−05 | 3.44E−03 | |
| cg22881265 | −0.036 | 2.42E−05 | 3.46E−03 | |
| cg20460227 | −0.078 | 2.42E−05 | 3.46E−03 | |
| cg02487823 | 0.029 | 2.43E−05 | 3.46E−03 | KIAA0226 |
| cg05027594 | −0.056 | 2.46E−05 | 3.49E−03 | TAPT1 |
| cg05317600 | 0.069 | 2.49E−05 | 3.52E−03 | |
| cg14833891 | 0.031 | 2.55E−05 | 3.57E−03 | |
| cg01603073 | 0.032 | 2.56E−05 | 3.58E−03 | |
| cg13411373 | 0.035 | 2.58E−05 | 3.59E−03 | |
| cg10676309 | 0.025 | 2.58E−05 | 3.59E−03 | |
| cg20460514 | 0.044 | 2.61E−05 | 3.61E−03 | LPIN1 |
| cg03048488 | 0.054 | 2.62E−05 | 3.62E−03 | |
| cg07745671 | 0.055 | 2.63E−05 | 3.62E−03 | |
| cg14283140 | 0.043 | 2.64E−05 | 3.63E−03 | C9orf9 |
| cg27492102 | 0.065 | 2.64E−05 | 3.63E−03 | |
| cg04936009 | −0.070 | 2.66E−05 | 3.64E−03 | |
| cg08640361 | 0.031 | 2.68E−05 | 3.66E−03 | SGK1 |
| cg11647719 | 0.031 | 2.70E−05 | 3.68E−03 | DIDO1 |
| cg04029168 | −0.030 | 2.81E−05 | 3.80E−03 | PRKCB |
| cg23954655 | 0.084 | 2.81E−05 | 3.80E−03 | STK24 |
| cg25885684 | 0.076 | 2.85E−05 | 3.84E−03 | |
| cg11795262 | 0.024 | 2.89E−05 | 3.86E−03 | GATAD2A |
| cg00294940 | 0.041 | 2.92E−05 | 3.89E−03 | SLC5A9 |
| cg27624753 | 0.027 | 2.95E−05 | 3.91E−03 | CRAMP1L |
| cg09434536 | 0.036 | 2.95E−05 | 3.91E−03 | |
| cg07712198 | −0.078 | 3.01E−05 | 3.96E−03 | |
| cg01391071 | 0.024 | 3.09E−05 | 4.02E−03 | NDUFV1 |
| cg24002003 | 0.042 | 3.09E−05 | 4.02E−03 | |
| cg26350921 | 0.033 | 3.12E−05 | 4.05E−03 | TBC1D16 |
| cg13799081 | 0.106 | 3.14E−05 | 4.06E−03 | RBM20 |
| cg00719568 | 0.067 | 3.16E−05 | 4.08E−03 | MOV10 |
| cg03550773 | −0.027 | 3.22E−05 | 4.13E−03 | |
| cg08503994 | 0.046 | 3.23E−05 | 4.13E−03 | C16orf5 |
| cg23780146 | 0.039 | 3.24E−05 | 4.13E−03 | |
| cg19076265 | 0.029 | 3.24E−05 | 4.14E−03 | DOCK5 |
| cg24318076 | 0.064 | 3.25E−05 | 4.14E−03 | ALKBH5 |
| cg12449916 | −0.075 | 3.29E−05 | 4.17E−03 | |
| cg07093324 | −0.096 | 3.34E−05 | 4.21E−03 | ACTR3 |
| cg00771653 | 0.028 | 3.34E−05 | 4.21E−03 | CHCHD10 |
| cg14284394 | 0.044 | 3.37E−05 | 4.23E−03 | |
| cg26793227 | −0.058 | 3.40E−05 | 4.25E−03 | EPHA2 |
| cg15012027 | −0.125 | 3.40E−05 | 4.26E−03 | |
| cg12240358 | 0.118 | 3.43E−05 | 4.28E−03 | HOMER2 |
| cg27262054 | 0.045 | 3.44E−05 | 4.29E−03 | ZFHX3 |
| cg22812413 | −0.109 | 3.47E−05 | 4.31E−03 | |
| cg12063018 | 0.052 | 3.54E−05 | 4.36E−03 | RIN3 |
| cg19668476 | 0.034 | 3.54E−05 | 4.36E−03 | |
| cg06806044 | −0.037 | 3.57E−05 | 4.40E−03 | |
| cg04908088 | 0.043 | 3.58E−05 | 4.41E−03 | |
| cg02639359 | 0.052 | 3.60E−05 | 4.42E−03 | FCHO1 |
| cg03508390 | 0.026 | 3.65E−05 | 4.46E−03 | KALRN |
| cg07834396 | −0.089 | 3.66E−05 | 4.47E−03 | MSRB2 |
| cg08608952 | 0.096 | 3.74E−05 | 4.54E−03 | CDC42 |
| cg16316344 | 0.045 | 3.76E−05 | 4.55E−03 | FNBP4 |
| cg09971646 | −0.099 | 3.77E−05 | 4.56E−03 | DLK1 |
| cg02048220 | 0.040 | 3.79E−05 | 4.58E−03 | TTC7B |
| cg06329316 | 0.036 | 3.84E−05 | 4.62E−03 | MAGI1 |
| cg19423175 | 0.043 | 3.88E−05 | 4.66E−03 | MAP2K2 |
| cg27363280 | 0.035 | 3.90E−05 | 4.67E−03 | |
| cg24646951 | 0.043 | 3.92E−05 | 4.69E−03 | |
| cg06647928 | 0.047 | 3.93E−05 | 4.69E−03 | |
| cg01885559 | −0.053 | 3.96E−05 | 4.70E−03 | KIDINS220 |
| cg26863864 | 0.051 | 4.05E−05 | 4.78E−03 | |
| cg01468567 | 0.079 | 4.08E−05 | 4.80E−03 | TEAD2 |
| cg02304370 | −0.076 | 4.19E−05 | 4.87E−03 | PHRF1 |
| cg27156259 | 0.075 | 4.22E−05 | 4.88E−03 | PXN |
| cg27022326 | 0.072 | 4.24E−05 | 4.89E−03 | PPP2R5C |
| cg08894487 | −0.070 | 4.28E−05 | 4.92E−03 | ARID3A |
| cg01692088 | 0.038 | 4.26E−05 | 4.92E−03 | |
| cg03137209 | 0.040 | 4.27E−05 | 4.92E−03 | |
| cg09676171 | −0.021 | 4.28E−05 | 4.92E−03 | |
| cg13201172 | −0.043 | 4.34E−05 | 4.97E−03 | SCRIB |
| cg08181642 | 0.031 | 4.35E−05 | 4.97E−03 | SLC29A2 |
| cg06892907 | −0.057 | 4.36E−05 | 4.99E−03 | |
| cg23314514 | 0.022 | 4.37E−05 | 4.99E−03 | |
| cg04877737 | 0.061 | 4.38E−05 | 4.99E−03 | |
| cg05938409 | 0.052 | 4.39E−05 | 5.00E−03 | |

TABLE 10D
Differentially Methylated CpG Sites Associated with IBS vs Celiac

| | logFC | P.Value | adj.P.Val | UCSC_RefGene_Name |
|---|---|---|---|---|
| cg07180897 | 0.166 | 2.43E−11 | 5.47E−06 | HLA-DQB2 |
| cg04418355 | 0.121 | 4.02E−10 | 4.52E−05 | HLA-DQB2 |
| cg04795387 | 0.065 | 1.56E−08 | 1.17E−03 | |
| cg21493951 | 0.253 | 3.24E−08 | 1.51E−03 | HLA-DQB1 |
| cg24593918 | −0.299 | 3.35E−08 | 1.51E−03 | HLA-DQB1 |
| cg23464743 | −0.242 | 4.99E−08 | 1.87E−03 | HLA-DQB1 |

TABLE 10E
Differentially Methylated CpG Sites Associated with IBS vs Healthy

| | logFC | P.Value | adj.P.Val | UCSC_RefGene_Name | |
|---|---|---|---|---|---|
| cg25118606 | 1.69E−02 | 3.95E−04 | 1.000 | ACTG2 | FALSE |
| cg14740657 | −1.91E−02 | 3.30E−04 | 1.000 | ADAMTS5 | FALSE |
| cg26328335 | −1.97E−02 | 1.10E−04 | 1.000 | AQP5 | FALSE |
| cg18254586 | −1.37E−02 | 4.38E−04 | 1.000 | ARHGAP26 | FALSE |
| cg21553145 | −2.66E−02 | 3.24E−04 | 1.000 | C8ORFK29 | FALSE |
| cg02981703 | 1.91E−02 | 4.61E−05 | 1.000 | CA6 | FALSE |
| cg04069951 | −1.68E−02 | 1.64E−04 | 1.000 | CD81 | FALSE |
| cg11572548 | −2.54E−02 | 1.39E−04 | 1.000 | CDC42 | FALSE |
| cg00338702 | −1.70E−02 | 1.33E−04 | 1.000 | CHFR | FALSE |
| cg08952029 | −2.49E−02 | 4.68E−04 | 1.000 | CHRDL2 | FALSE |
| cg16277214 | 2.27E−02 | 3.11E−04 | 1.000 | CMTM7 | FALSE |
| cg17732894 | −2.70E−02 | 4.10E−04 | 1.000 | COX19 | FALSE |
| cg27624753 | −1.51E−02 | 4.66E−04 | 1.000 | CRAMP1L | FALSE |
| cg24435844 | −3.40E−02 | 3.18E−04 | 1.000 | GAS2 | |
| cg14299480 | −5.61E−02 | 3.50E−05 | 1.000 | GGN | FALSE |
| cg02523893 | −1.45E−02 | 3.93E−04 | 1.000 | GRK6 | FALSE |
| cg08177681 | −1.39E−02 | 6.28E−05 | 1.000 | HDAC4 | FALSE |
| cg01405445 | −3.85E−02 | 3.27E−04 | 1.000 | HSPB6 | FALSE |
| cg01572696 | −3.53E−02 | 2.57E−04 | 1.000 | IDUA | |
| cg11891591 | −4.57E−02 | 1.42E−04 | 1.000 | LINGO3 | FALSE |
| cg00002033 | −3.77E−02 | 1.63E−04 | 1.000 | LRFN1 | FALSE |
| cg12128164 | −3.20E−02 | 1.61E−04 | 1.000 | LRFN3 | FALSE |
| cg02233750 | −3.61E−02 | 4.00E−04 | 1.000 | MCAT | FALSE |
| cg18271913 | −1.08E−02 | 1.26E−04 | 1.000 | MFSD4 | FALSE |
| cg17843665 | −3.33E−02 | 2.16E−04 | 1.000 | MIPEP | FALSE |
| cg09590897 | −1.19E−02 | 4.59E−04 | 1.000 | MIR637 | FALSE |
| cg08031967 | 2.44E−02 | 2.79E−04 | 1.000 | NXN | FALSE |
| cg22362389 | −3.17E−02 | 2.53E−04 | 1.000 | PDE6G | FALSE |
| cg08424749 | −1.78E−02 | 1.36E−04 | 1.000 | PRRT1 | FALSE |
| cg20790540 | 1.80E−02 | 3.83E−04 | 1.000 | PTCRA | FALSE |
| cg09527123 | −1.37E−02 | 2.03E−04 | 1.000 | RASA3 | FALSE |
| cg07195301 | −1.75E−02 | 3.24E−04 | 1.000 | SNCAIP | FALSE |
| cg18536644 | −1.84E−02 | 1.74E−04 | 1.000 | TSSC4 | FALSE |
| cg11950778 | −2.73E−02 | 3.76E−04 | 1.000 | UGT8 | FALSE |
| cg13610168 | −2.32E−02 | 2.26E−05 | 1.000 | | |
| cg21079030 | −3.68E−02 | 2.89E−05 | 1.000 | | |
| cg08285446 | −2.87E−02 | 1.15E−04 | 1.000 | | |
| cg07511668 | −1.73E−02 | 1.53E−04 | 1.000 | | |
| cg04857403 | −3.23E−02 | 1.59E−04 | 1.000 | | |
| cg23574061 | −1.14E−02 | 1.63E−04 | 1.000 | | |
| cg07534448 | 3.20E−02 | 1.70E−04 | 1.000 | | |
| cg09851545 | −3.95E−02 | 1.83E−04 | 1.000 | | |
| cg03454920 | 3.20E−02 | 2.02E−04 | 1.000 | | |
| cg12400350 | 3.79E−02 | 2.27E−04 | 1.000 | | |
| cg25206280 | −9.13E−03 | 2.29E−04 | 1.000 | | |
| cg12575883 | −7.03E−02 | 4.88E−04 | 1.000 | | |

TABLE 10F
Differentially Methylated CpG Sites Associated with IBS vs IBD

| | logFC | P.Value | adj.P.Val | UCSC_RefGene_Name |
|---|---|---|---|---|
| cg18181906 | −3.54E−02 | 1.51E−08 | 1.69E−03 | |
| cg21848117 | −4.19E−02 | 1.77E−07 | 7.93E−03 | SLC38A10 |
| cg14126884 | −6.03E−02 | 3.03E−07 | 9.68E−03 | RAPGEF1 |
| cg15518113 | −5.13E−02 | 6.62E−07 | 1.35E−02 | CD247 |
| cg04352288 | −6.40E−02 | 9.62E−07 | 1.44E−02 | CA5A |
| cg12572295 | −4.76E−02 | 2.14E−06 | 1.44E−02 | GRK4 |
| cg03922648 | −4.03E−02 | 1.92E−06 | 1.44E−02 | KLRC1 |
| cg06394247 | 7.21E−02 | 1.94E−06 | 1.44E−02 | PAX5 |
| cg06713373 | −4.54E−02 | 1.86E−06 | 1.44E−02 | SLC12A8 |
| cg07119168 | −4.64E−02 | 2.10E−06 | 1.44E−02 | SPTB |
| cg05058976 | −3.05E−02 | 3.35E−06 | 1.63E−02 | BTBD12 |
| cg07728579 | −7.60E−02 | 3.09E−06 | 1.63E−02 | FSD2 |
| cg18274749 | −4.90E−02 | 2.83E−06 | 1.63E−02 | HSPG2 |
| cg18376497 | 9.09E−02 | 4.56E−06 | 1.96E−02 | INPP4B |
| cg09765463 | −4.04E−02 | 4.38E−06 | 1.96E−02 | JAK1 |
| cg11650874 | −4.24E−02 | 6.29E−06 | 2.31E−02 | CHID1 |
| cg23249717 | −2.08E−02 | 6.49E−06 | 2.34E−02 | |
| cg03770523 | −4.04E−02 | 7.53E−06 | 2.35E−02 | KIAA1026 |
| cg27026192 | −3.32E−02 | 7.93E−06 | 2.40E−02 | KIFC3 |
| cg06861560 | −2.35E−02 | 8.55E−06 | 2.46E−02 | |
| cg00719237 | −3.44E−02 | 1.07E−05 | 2.88E−02 | NGFR |
| cg24597774 | −3.47E−02 | 1.10E−05 | 2.88E−02 | SPSB1 |
| cg19563355 | −4.35E−02 | 1.21E−05 | 2.94E−02 | HERC2 |
| cg25104637 | −2.88E−02 | 1.23E−05 | 2.94E−02 | NRXN2 |
| cg04894169 | −3.00E−02 | 1.27E−05 | 2.94E−02 | XPC |
| cg21041100 | −2.82E−02 | 1.33E−05 | 3.00E−02 | RAB5B |
| cg13641591 | −3.25E−02 | 1.37E−05 | 3.03E−02 | UVRAG |
| cg23842572 | −5.40E−02 | 1.60E−05 | 3.31E−02 | MPRIP |
| cg01668008 | −3.10E−02 | 1.71E−05 | 3.43E−02 | |
| cg00754552 | −1.08E−01 | 1.79E−05 | 3.48E−02 | NAPSB |
| cg03109660 | −8.02E−02 | 1.92E−05 | 3.52E−02 | RELL1 |
| cg16618260 | −2.12E−02 | 1.97E−05 | 3.54E−02 | BCL2L12 |
| cg12127149 | 7.39E−02 | 2.02E−05 | 3.55E−02 | DYNC1H1 |
| cg00139496 | −6.77E−02 | 2.19E−05 | 3.72E−02 | GLT8D1 |
| cg00910203 | −2.16E−02 | 2.23E−05 | 3.75E−02 | |
| cg23971362 | −3.18E−02 | 2.25E−05 | 3.77E−02 | LRRC43 |
| cg03108250 | −4.22E−02 | 2.30E−05 | 3.82E−02 | KIR3DX1 |
| cg12478185 | 5.09E−02 | 2.55E−05 | 3.89E−02 | C17orf48 |
| cg12062672 | −4.36E−02 | 2.46E−05 | 3.89E−02 | FGF22 |
| cg23314514 | −2.27E−02 | 2.70E−05 | 3.98E−02 | |
| cg05094429 | −7.20E−02 | 2.90E−05 | 3.99E−02 | CCR6 |
| cg07029002 | −7.09E−02 | 2.85E−05 | 3.99E−02 | ROR1 |
| cg13894747 | −2.21E−02 | 2.97E−05 | 3.99E−02 | |
| cg24721647 | −3.32E−02 | 3.19E−05 | 4.06E−02 | ACSL1 |
| cg03884572 | −3.93E−02 | 3.35E−05 | 4.06E−02 | CUX2 |
| cg11772801 | −5.19E−02 | 3.35E−05 | 4.06E−02 | TMX1 |
| cg25885684 | −8.13E−02 | 3.25E−05 | 4.06E−02 | |
| cg13719443 | 5.25E−02 | 3.45E−05 | 4.15E−02 | LOC285847 |
| cg07808087 | −3.17E−02 | 3.53E−05 | 4.18E−02 | CPEB2 |
| cg01368900 | −5.00E−02 | 3.55E−05 | 4.18E−02 | IQSEC1 |
| cg02053339 | −2.33E−02 | 3.64E−05 | 4.20E−02 | A4GALT |
| cg16231917 | 1.27E−01 | 3.71E−05 | 4.20E−02 | PVT1 |
| cg03796224 | −2.98E−02 | 3.74E−05 | 4.21E−02 | RBPMS |
| cg05166473 | −4.21E−02 | 4.02E−05 | 4.29E−02 | BANP |
| cg15256491 | −3.47E−02 | 4.00E−05 | 4.29E−02 | FOXK1 |
| cg06287140 | −2.31E−02 | 4.22E−05 | 4.41E−02 | BCL7A |
| cg08830672 | 5.10E−02 | 5.14E−05 | 4.88E−02 | ATP6V0D1 |
| cg26838949 | 5.24E−02 | 5.19E−05 | 4.88E−02 | KIF21B |
| cg22350438 | 5.44E−02 | 5.18E−05 | 4.88E−02 | SYNC |
| cg13754665 | 6.36E−02 | 5.02E−05 | 4.88E−02 | VAV2 |
| cg25477163 | −4.84E−02 | 5.18E−05 | 4.88E−02 | ZNF211 |
| cg04786889 | −7.64E−02 | 5.27E−05 | 4.90E−02 | ADAP1 |
| cg17313709 | 9.25E−02 | 5.30E−05 | 4.90E−02 | |
| cg24112097 | −3.40E−02 | 5.63E−05 | 4.92E−02 | BRD1 |
| cg16896205 | −2.29E−02 | 5.50E−05 | 4.92E−02 | CD53 |
| cg18567954 | −3.67E−02 | 5.53E−05 | 4.92E−02 | DTX1 |
| cg02719245 | −5.28E−02 | 5.60E−05 | 4.92E−02 | FLJ45244 |
| cg07169295 | −2.48E−02 | 5.43E−05 | 4.92E−02 | SYT12 |
| cg04829186 | −4.32E−02 | 5.68E−05 | 4.92E−02 | |
| cg09073441 | −4.56E−02 | 5.88E−05 | 4.95E−02 | |
| cg08467887 | −3.47E−02 | 6.57E−05 | 5.24E−02 | EPPK1 |
| cg22707397 | −5.01E−02 | 6.72E−05 | 5.29E−02 | |
| cg22508930 | −2.43E−02 | 6.93E−05 | 5.37E−02 | RAP1B |
| cg20114528 | −5.02E−02 | 7.27E−05 | 5.50E−02 | ASB8 |
| cg15464148 | −6.07E−02 | 7.26E−05 | 5.50E−02 | LPAR5 |
| cg23589035 | 9.20E−02 | 7.53E−05 | 5.56E−02 | ASH1L |
| cg21500092 | −3.24E−02 | 7.54E−05 | 5.56E−02 | UBE2G1 |
| cg16023434 | −4.88E−02 | 7.50E−05 | 5.56E−02 | |
| cg22779330 | −1.85E−02 | 7.86E−05 | 5.61E−02 | MYEOV |
| cg02192117 | 3.17E−02 | 8.70E−05 | 6.00E−02 | TNRC18 |
| cg02667577 | −3.31E−02 | 9.20E−05 | 6.17E−02 | WIPI2 |
| cg20234640 | −4.10E−02 | 9.91E−05 | 6.42E−02 | FCER2 |
| cg13897348 | 9.52E−02 | 9.99E−05 | 6.43E−02 | MIB2 |
| cg15241519 | −3.79E−02 | 1.03E−04 | 6.53E−02 | SORL1 |
| cg06412669 | 3.79E−02 | 1.06E−04 | 6.62E−02 | RPTOR |
| cg13620439 | −3.71E−02 | 1.09E−04 | 6.69E−02 | CLU |
| cg13738327 | −4.23E−02 | 1.10E−04 | 6.69E−02 | LRP5 |
| cg13804478 | −4.01E−02 | 1.09E−04 | 6.69E−02 | MUC1 |
| cg23191354 | −4.72E−02 | 1.10E−04 | 6.69E−02 | OSBPL3 |
| cg03726817 | 6.97E−02 | 1.08E−04 | 6.69E−02 | |
| cg01727923 | −3.86E−02 | 1.08E−04 | 6.69E−02 | |
| cg18904855 | −2.44E−02 | 1.09E−04 | 6.69E−02 | |
| cg14586546 | −3.81E−02 | 1.11E−04 | 6.72E−02 | RALGDS |
| cg10515414 | −2.37E−02 | 1.13E−04 | 6.79E−02 | FHAD1 |
| cg04821520 | −3.81E−02 | 1.15E−04 | 6.88E−02 | |
| cg19910327 | −2.46E−02 | 1.16E−04 | 6.89E−02 | PIPSL |
| cg14940871 | −3.66E−02 | 1.17E−04 | 6.90E−02 | |
| cg25894551 | −4.35E−02 | 1.19E−04 | 6.98E−02 | FXYD2 |
| cg18982073 | 3.87E−02 | 1.21E−04 | 6.99E−02 | ATXN7L3 |
| cg04482712 | −3.97E−02 | 1.24E−04 | 7.07E−02 | SLC9A3R1 |
| cg22212560 | −4.08E−02 | 1.26E−04 | 7.08E−02 | FRMD8 |
| cg27655168 | −2.79E−02 | 1.26E−04 | 7.08E−02 | |
| cg09004241 | −4.36E−02 | 1.31E−04 | 7.10E−02 | B3GNT7 |
| cg14283140 | −4.17E−02 | 1.32E−04 | 7.10E−02 | C9orf9 |
| cg25015416 | −3.17E−02 | 1.31E−04 | 7.10E−02 | CNP |
| cg00937012 | −3.27E−02 | 1.32E−04 | 7.10E−02 | CPEB1 |
| cg06858263 | −3.06E−02 | 1.29E−04 | 7.10E−02 | DNAJB6 |
| cg03126058 | 3.20E−02 | 1.30E−04 | 7.10E−02 | PRDM16 |
| cg24068815 | −2.50E−02 | 1.32E−04 | 7.10E−02 | |
| cg16455376 | −2.44E−02 | 1.36E−04 | 7.12E−02 | |
| cg08879111 | −1.02E−01 | 1.39E−04 | 7.19E−02 | IL1R2 |
| cg07035442 | −3.26E−02 | 1.40E−04 | 7.22E−02 | |
| cg19427746 | 1.09E−01 | 1.42E−04 | 7.23E−02 | CAMTA2 |
| cg08564487 | −3.35E−02 | 1.44E−04 | 7.28E−02 | 45544 |
| cg18912107 | −5.78E−02 | 1.46E−04 | 7.37E−02 | TBPL1 |
| cg01840724 | −3.83E−02 | 1.48E−04 | 7.44E−02 | |
| cg13760683 | −2.15E−02 | 1.53E−04 | 7.60E−02 | LOC284749 |
| cg01881062 | 6.59E−02 | 1.56E−04 | 7.63E−02 | KLHL21 |
| cg13799081 | −1.04E−01 | 1.58E−04 | 7.64E−02 | RBM20 |
| cg07434082 | −4.34E−02 | 1.60E−04 | 7.66E−02 | ARHGEF7 |
| cg14480531 | −3.06E−02 | 1.60E−04 | 7.66E−02 | MSI2 |
| cg02487823 | −2.41E−02 | 1.61E−04 | 7.69E−02 | KIAA0226 |
| cg13765961 | −9.21E−02 | 1.62E−04 | 7.69E−02 | MS4A1 |
| cg01822759 | −5.71E−02 | 1.62E−04 | 7.69E−02 | |
| cg07017331 | 5.77E−02 | 1.62E−04 | 7.70E−02 | |
| cg27295118 | 6.40E−02 | 1.63E−04 | 7.70E−02 | |
| cg24754199 | −4.20E−02 | 1.64E−04 | 7.72E−02 | DIP2C |
| cg09607915 | −5.42E−02 | 1.64E−04 | 7.72E−02 | FBXO11 |
| cg06639923 | −3.20E−02 | 1.66E−04 | 7.76E−02 | FBXL18 |
| cg08687540 | −3.86E−02 | 1.66E−04 | 7.76E−02 | LSP1 |
| cg21480264 | 3.93E−02 | 1.68E−04 | 7.77E−02 | POLN |
| cg15741433 | −9.44E−02 | 1.73E−04 | 7.92E−02 | HIST1H1A |
| cg16771652 | 5.95E−02 | 1.74E−04 | 7.92E−02 | NOD2 |
| cg13076829 | −4.30E−02 | 1.75E−04 | 7.94E−02 | |
| cg22881435 | −7.09E−02 | 1.80E−04 | 8.08E−02 | RAB11FIP1 |
| cg01427313 | −3.18E−02 | 1.81E−04 | 8.09E−02 | ACSF3 |
| cg26102819 | −5.48E−02 | 1.81E−04 | 8.09E−02 | HEG1 |
| cg18696237 | −3.59E−02 | 1.83E−04 | 8.11E−02 | APOBEC3H |
| cg17843376 | 6.71E−02 | 1.82E−04 | 8.11E−02 | DPP6 |
| cg17981790 | −4.17E−02 | 1.84E−04 | 8.12E−02 | FAM49A |
| cg01928820 | −9.63E−02 | 1.83E−04 | 8.12E−02 | PTCRA |
| cg05148180 | −3.50E−02 | 1.88E−04 | 8.20E−02 | |
| cg11199827 | −2.60E−02 | 1.91E−04 | 8.30E−02 | |
| cg14111697 | −3.61E−02 | 1.97E−04 | 8.38E−02 | GNAQ |
| cg11978441 | −4.74E−02 | 1.96E−04 | 8.38E−02 | PER3 |
| cg10775350 | −2.42E−02 | 2.01E−04 | 8.48E−02 | SLC25A41 |
| cg01303420 | −5.63E−02 | 2.05E−04 | 8.51E−02 | C3orf55 |
| cg24137511 | −5.19E−02 | 2.06E−04 | 8.51E−02 | MAST3 |
| cg11080552 | −3.94E−02 | 2.04E−04 | 8.51E−02 | RERE |
| cg06588802 | −5.92E−02 | 2.06E−04 | 8.51E−02 | |
| cg03073429 | −5.57E−02 | 2.08E−04 | 8.59E−02 | CPXM2 |
| cg26382697 | −5.65E−02 | 2.10E−04 | 8.62E−02 | CD81 |
| cg10068330 | −4.40E−02 | 2.10E−04 | 8.62E−02 | |
| cg13707729 | −3.88E−02 | 2.13E−04 | 8.68E−02 | FGFR2 |
| cg07299819 | −2.70E−02 | 2.15E−04 | 8.69E−02 | NPR1 |
| cg07417683 | −5.58E−02 | 2.15E−04 | 8.69E−02 | |
| cg26878318 | −2.97E−02 | 2.17E−04 | 8.74E−02 | CMBL |
| cg06508085 | −2.15E−02 | 2.20E−04 | 8.81E−02 | |
| cg09080120 | −3.75E−02 | 2.26E−04 | 8.86E−02 | B3GALT4 |
| cg08485389 | −5.33E−02 | 2.26E−04 | 8.86E−02 | C16orf71 |
| cg18457597 | 7.42E−02 | 2.24E−04 | 8.86E−02 | C17orf97 |
| cg09199562 | −5.76E−02 | 2.27E−04 | 8.86E−02 | |
| cg18540455 | −3.32E−02 | 2.30E−04 | 8.90E−02 | CLASP2 |
| cg07599366 | 4.04E−02 | 2.35E−04 | 8.96E−02 | CBFA2T3 |
| cg23754918 | −5.28E−02 | 2.35E−04 | 8.96E−02 | IQCE |
| cg02328326 | 1.10E−01 | 2.36E−04 | 8.96E−02 | MAP2K5 |
| cg04597765 | −2.52E−02 | 2.34E−04 | 8.96E−02 | TRIO |
| cg03048488 | −4.38E−02 | 2.36E−04 | 8.96E−02 | |
| cg00041047 | −3.92E−02 | 2.39E−04 | 8.99E−02 | SLC6A16 |
| cg24022651 | −3.58E−02 | 2.41E−04 | 9.02E−02 | FNDC3B |
| cg01637125 | −7.15E−02 | 2.45E−04 | 9.07E−02 | |
| cg13583414 | −6.22E−02 | 2.46E−04 | 9.10E−02 | LZTS1 |
| cg10860364 | −2.92E−02 | 2.47E−04 | 9.10E−02 | ANKRD13B |
| cg27627493 | −3.32E−02 | 2.46E−04 | 9.10E−02 | |
| cg02287325 | −3.17E−02 | 2.51E−04 | 9.16E−02 | CTRB1 |
| cg17908947 | −2.47E−02 | 2.51E−04 | 9.16E−02 | MAFF |
| cg23317857 | −4.18E−02 | 2.52E−04 | 9.17E−02 | TFEB |
| cg21777154 | −2.56E−02 | 2.52E−04 | 9.17E−02 | C1orf21 |
| cg26452434 | −5.64E−02 | 2.62E−04 | 9.25E−02 | GADD45G |
| cg22109240 | 8.78E−02 | 2.61E−04 | 9.25E−02 | KCTD18 |
| cg21010350 | −2.32E−02 | 2.67E−04 | 9.25E−02 | LOC100128542 |
| cg24581462 | −3.19E−02 | 2.67E−04 | 9.25E−02 | PLEKHA2 |
| cg14160419 | −3.35E−02 | 2.68E−04 | 9.25E−02 | RBM47 |
| cg08844849 | −3.85E−02 | 2.66E−04 | 9.25E−02 | SIGLEC10 |
| cg06653632 | −4.56E−02 | 2.56E−04 | 9.25E−02 | SLC15A4 |
| cg05456362 | −3.18E−02 | 2.63E−04 | 9.25E−02 | |
| cg00776427 | −3.69E−02 | 2.65E−04 | 9.25E−02 | |
| cg02539402 | −2.67E−02 | 2.65E−04 | 9.25E−02 | |
| cg11851457 | −2.92E−02 | 2.65E−04 | 9.25E−02 | |
| cg24364282 | −3.16E−02 | 2.81E−04 | 9.41E−02 | |
| cg24462247 | −2.94E−02 | 2.82E−04 | 9.42E−02 | |
| cg15706973 | −2.13E−02 | 2.95E−04 | 9.63E−02 | ACOXL |
| cg09232225 | −1.73E−02 | 2.94E−04 | 9.63E−02 | CES8 |
| cg11938718 | 4.10E−02 | 2.93E−04 | 9.63E−02 | HPCAL1 |
| cg24150157 | −3.04E−02 | 2.97E−04 | 9.64E−02 | LIM2 |
| cg26159905 | −2.83E−02 | 3.03E−04 | 9.66E−02 | ASB10 |
| cg16316344 | −3.84E−02 | 3.02E−04 | 9.66E−02 | FNBP4 |
| cg25117092 | −1.30E−01 | 3.04E−04 | 9.66E−02 | MED12L |
| cg03507593 | −4.40E−02 | 3.01E−04 | 9.66E−02 | NCOR2 |
| cg15828235 | −5.47E−02 | 3.05E−04 | 9.66E−02 | PCP2 |
| cg18697636 | 4.96E−02 | 3.01E−04 | 9.66E−02 | SLC9A3 |
| cg18566313 | −2.86E−02 | 3.05E−04 | 9.66E−02 | SORBS2 |
| cg25480132 | −2.32E−02 | 3.03E−04 | 9.66E−02 | STMN3 |
| cg01719995 | 8.32E−02 | 3.05E−04 | 9.66E−02 | USP38 |
| cg20269046 | 3.44E−02 | 3.04E−04 | 9.66E−02 | ZNF467 |
| cg13489576 | 2.47E−02 | 3.08E−04 | 9.70E−02 | DLG1 |
| cg23534210 | −5.21E−02 | 3.07E−04 | 9.70E−02 | TBCD |
| cg04838847 | −3.46E−02 | 3.14E−04 | 9.79E−02 | GOLSYN |
| cg16238149 | 3.52E−02 | 3.20E−04 | 9.91E−02 | |
| cg13253439 | 2.40E−02 | 3.22E−04 | 9.91E−02 | C6orf136 |
| cg26722342 | −1.77E−02 | 3.22E−04 | 9.91E−02 | ITGA2 |
| cg19215199 | −3.62E−02 | 3.22E−04 | 9.91E−02 | ZMIZ1 |
| cg12988118 | 3.46E−02 | 3.23E−04 | 9.91E−02 | C6orf27 |
| cg25256099 | −6.71E−02 | 3.24E−04 | 9.91E−02 | |
| cg01997884 | −4.12E−02 | 3.24E−04 | 9.91E−02 | |
| cg05979484 | 5.40E−02 | 3.25E−04 | 9.93E−02 | LANCL2 |
| cg20982046 | −9.51E−02 | 3.26E−04 | 9.94E−02 | |
| cg27630771 | −1.71E−02 | 3.30E−04 | 9.98E−02 | ADAMTSL4 |
| cg14405813 | −4.36E−02 | 3.31E−04 | 9.98E−02 | HIPK2 |
| cg18195500 | −2.21E−02 | 3.36E−04 | 1.01E−01 | |
| cg18457731 | −4.08E−02 | 3.37E−04 | 1.01E−01 | COL22A1 |
| cg17350544 | −3.06E−02 | 3.45E−04 | 1.02E−01 | PARD3B |
| cg14321777 | −2.36E−02 | 3.50E−04 | 1.03E−01 | |
| cg05365532 | −7.04E−02 | 3.51E−04 | 1.03E−01 | LILRB1 |
| cg17207690 | −2.04E−02 | 3.52E−04 | 1.03E−01 | NMUR1 |
| cg05885212 | −2.35E−02 | 3.55E−04 | 1.03E−01 | |
| cg01939741 | −2.12E−02 | 3.55E−04 | 1.03E−01 | |
| cg10089657 | −3.48E−02 | 3.56E−04 | 1.04E−01 | C7orf50 |
| cg10906428 | −2.23E−02 | 3.62E−04 | 1.04E−01 | CRIPT |
| cg14019146 | −4.80E−02 | 3.65E−04 | 1.04E−01 | SLC38A3 |
| cg03363417 | −2.23E−02 | 3.66E−04 | 1.04E−01 | SNORD116-24 |
| cg16252691 | −3.53E−02 | 3.64E−04 | 1.04E−01 | |
| cg16931664 | 4.21E−02 | 3.67E−04 | 1.04E−01 | |
| cg15396800 | −2.34E−02 | 3.73E−04 | 1.05E−01 | |
| cg04787728 | −3.75E−02 | 3.75E−04 | 1.05E−01 | NLRC3 |
| cg11479568 | −3.31E−02 | 3.75E−04 | 1.05E−01 | ANK1 |
| cg11615509 | −2.75E−02 | 3.81E−04 | 1.06E−01 | SHROOM3 |
| cg20552236 | −3.71E−02 | 3.89E−04 | 1.07E−01 | CSK |
| cg00314892 | −3.11E−02 | 3.90E−04 | 1.07E−01 | PLCH2 |
| cg10877315 | −1.03E−01 | 3.89E−04 | 1.07E−01 | |
| cg18664667 | 8.21E−02 | 3.92E−04 | 1.07E−01 | ATXN7L1 |
| cg13922442 | −5.11E−02 | 3.93E−04 | 1.07E−01 | TARP |
| cg08991210 | −4.45E−02 | 3.94E−04 | 1.07E−01 | |
| cg13172153 | −3.72E−02 | 4.04E−04 | 1.08E−01 | TNNI1 |
| cg08120226 | 4.61E−02 | 4.05E−04 | 1.08E−01 | MUC4 |
| cg11742472 | −3.18E−02 | 4.08E−04 | 1.09E−01 | ARHGEF10L |
| cg26526397 | −3.05E−02 | 4.10E−04 | 1.09E−01 | |
| cg06920037 | −1.81E−02 | 4.12E−04 | 1.09E−01 | |
| cg26740494 | −6.16E−02 | 4.16E−04 | 1.09E−01 | MMP23A |
| cg08534055 | −2.98E−02 | 4.21E−04 | 1.10E−01 | C16orf72 |
| cg17479280 | −6.58E−02 | 4.21E−04 | 1.10E−01 | MEF2D |
| cg09994445 | −2.95E−02 | 4.20E−04 | 1.10E−01 | ZBTB16 |
| cg21550804 | −1.39E−01 | 4.29E−04 | 1.11E−01 | |
| cg10356204 | −4.08E−02 | 4.31E−04 | 1.11E−01 | JSRP1 |
| cg14363954 | −2.49E−02 | 4.34E−04 | 1.11E−01 | NOTCH1 |
| cg01830256 | −2.77E−02 | 4.39E−04 | 1.12E−01 | PACS2 |
| cg27123273 | −3.73E−02 | 4.42E−04 | 1.13E−01 | |
| cg22558673 | −2.42E−02 | 4.44E−04 | 1.13E−01 | GJA4 |
| cg03048947 | −5.76E−02 | 4.44E−04 | 1.13E−01 | LBR |
| cg14613361 | −2.78E−02 | 4.45E−04 | 1.13E−01 | |
| cg19806398 | −2.81E−02 | 4.46E−04 | 1.13E−01 | CX3CR1 |
| cg14712058 | −3.20E−02 | 4.47E−04 | 1.13E−01 | SIN3B |
| cg00342358 | −4.48E−02 | 4.53E−04 | 1.14E−01 | TGFBR3 |
| cg13482010 | −4.25E−02 | 4.56E−04 | 1.14E−01 | SEMA5B |
| cg13811168 | 1.90E−02 | 4.64E−04 | 1.15E−01 | DUSP8 |
| cg14284211 | 4.91E−02 | 4.63E−04 | 1.15E−01 | FKBP5 |
| cg01205058 | −3.30E−02 | 4.65E−04 | 1.15E−01 | |
| cg24078554 | −2.39E−02 | 4.75E−04 | 1.16E−01 | ZNF697 |
| cg15911493 | −3.81E−02 | 4.76E−04 | 1.16E−01 | AP4S1 |
| cg14348967 | 4.56E−02 | 4.84E−04 | 1.18E−01 | |
| cg07532405 | −4.30E−02 | 4.88E−04 | 1.18E−01 | |
| cg21962791 | −6.14E−02 | 4.90E−04 | 1.18E−01 | PYROXD1 |
| cg01001533 | 4.07E−02 | 4.90E−04 | 1.18E−01 | |
| cg08627089 | −2.62E−02 | 4.97E−04 | 1.19E−01 | FGL1 |
| cg11611244 | −3.05E−02 | 4.96E−04 | 1.19E−01 | HCCA2 |
| cg04398861 | −2.95E−02 | 4.95E−04 | 1.19E−01 | MYL5 |
| cg01235795 | −3.22E−02 | 4.97E−04 | 1.19E−01 | |
| cg06676778 | −1.18E−01 | 4.99E−04 | 1.19E−01 | |

Tables 11A-11F. GO Terms Associated with Differentially Methylated Genes

“#” refers to number of Differentially Methylated (D. M.) Genes

TABLE 11A
IBD vs Healthy p < 0.05

| ONTOLOGY | | TERM | N | # | P. D. M. | FDR |
|---|---|---|---|---|---|---|
| GO:0006955 | BP | immune response | 1577 | 267 | 1.11E−14 | 2.53E−10 |
| GO:0002376 | BP | immune system process | 2455 | 397 | 1.14E−13 | 1.31E−09 |
| GO:0071944 | CC | cell periphery | 5911 | 885 | 7.01E−13 | 5.33E−09 |
| GO:0005886 | CC | plasma membrane | 5427 | 807 | 4.29E−11 | 2.45E−07 |
| GO:0007165 | BP | signal transduction | 5812 | 840 | 9.32E−11 | 4.25E−07 |
| GO:0050896 | BP | response to stimulus | 8581 | 1161 | 2.36E−10 | 8.99E−07 |
| GO:0023052 | BP | signaling | 6295 | 905 | 4.99E−10 | 1.63E−06 |
| GO:0007166 | BP | cell surface receptor | 2675 | 447 | 5.87E−10 | 1.67E−06 |
| | | signaling pathway | | | | |
| GO:0002682 | BF | regulation of immune | 1358 | 227 | 2.59E−09 | 6.01E−06 |
| | | system process | | | | |
| GO:0007154 | BF | cell communication | 6350 | 905 | 2.63E−09 | 6.01E−06 |
| GO:0002684 | BP | positive regulation of | 840 | 153 | 3.58E−09 | 7.43E−06 |
| | | immune system process | | | | |
| GO:0048583 | BF | regulation of response to | 3863 | 599 | 5.29E−09 | 9.50E−06 |
| | | stimulus | | | | |
| GO:0050776 | BF | regulation of immune | 810 | 146 | 5.41E−09 | 9.50E−06 |
| | | response | | | | |
| GO:0001775 | BP | cell activation | 1017 | 182 | 6.55E−09 | 1.07E−05 |
| GO:0045321 | BP | leukocyte activation | 876 | 160 | 8.95E−09 | 1.36E−05 |
| GO:0050778 | BP | positive regulation of | 513 | 101 | 1.20E−08 | 1.71E−05 |
| | | immune response | | | | |
| GO:0002250 | BP | adaptive immune | 438 | 88 | 1.31E−08 | 1.76E−05 |
| | | response | | | | |
| GO:0051716 | BP | cellular response to | 7245 | 994 | 1.55E−08 | 1.97E−05 |
| | | stimulus | | | | |
| GO:0048584 | BP | positive regulation of | 2099 | 342 | 1.88E−08 | 2.26E−05 |
| | | response to stimulus | | | | |
| GO:0023051 | BP | regulation of signaling | 3362 | 544 | 3.00E−08 | 3.42E−05 |
| GO:0009966 | BP | regulation of signal | 2971 | 481 | 4.32E−08 | 4.70E−05 |
| | | transduction | | | | |
| GO:0045087 | BP | innate immune response | 780 | 124 | 9.07E−08 | 9.40E−05 |
| GO:0010646 | BP | regulation of cell | 3346 | 537 | 9.56E−08 | 9.48E−05 |
| | | communication | | | | |
| GO:0002764 | BP | immune response- | 405 | 87 | 1.03E−07 | 9.76E−05 |
| | | regulating signaling | | | | |
| | | pathway | | | | |
| GO:0006952 | BP | defense response | 1644 | 232 | 1.54E−07 | 1.37E−04 |
| GO:0023056 | BP | positive regulation of | 1705 | 291 | 1.59E−07 | 1.37E−04 |
| | | signaling | | | | |
| GO:0002252 | BP | immune effector process | 594 | 107 | 1.62E−07 | 1.37E−04 |
| GO:0002521 | BP | leukocyte differentiation | 551 | 110 | 1.80E−07 | 1.47E−04 |
| GO:0065009 | BP | regulation of molecular | 3048 | 477 | 1.89E−07 | 1.49E−04 |
| | | function | | | | |
| GO:1902533 | BP | positive regulation of | 981 | 177 | 3.02E−07 | 2.27E−04 |
| | | intracellular signal | | | | |
| | | transduction | | | | |
| GO:0009967 | BP | positive regulation of | 1510 | 259 | 3.09E−07 | 2.27E−04 |
| | | signal transduction | | | | |
| GO:0002768 | BP | immune response- | 253 | 61 | 3.27E−07 | 2.29E−04 |
| | | regulating cell surface | | | | |
| | | receptor signaling | | | | |
| | | pathway | | | | |
| GO:0048534 | BP | hematopoietic or | 896 | 164 | 3.32E−07 | 2.29E−04 |
| | | lymphoid organ | | | | |
| | | development | | | | |
| GO:0030097 | BP | hemopoiesis | 856 | 157 | 3.95E−07 | 2.65E−04 |
| GO:0007159 | BP | leukocyte cell-cell | 378 | 78 | 4.44E−07 | 2.89E−04 |
| | | adhesion | | | | |
| GO:0010033 | BP | response to organic | 3016 | 450 | 6.54E−07 | 4.15E−04 |
| | | substance | | | | |
| GO:0010647 | BP | positive regulation of cell | 1701 | 287 | 8.24E−07 | 5.08E−04 |
| | | communication | | | | |
| GO:0045785 | BP | positive regulation of cell | 442 | 97 | 1.34E−06 | 8.03E−04 |
| | | adhesion | | | | |
| GO:0007155 | BP | cell adhesion | 1500 | 270 | 1.37E−06 | 8.03E−04 |
| GO:0098609 | BP | cell-cell adhesion | 920 | 174 | 1.43E−06 | 8.16E−04 |
| GO:0002698 | BP | negative regulation of | 112 | 30 | 1.58E−06 | 8.78E−04 |
| | | immune effector process | | | | |
| GO:0005102 | MF | signaling receptor binding | 1468 | 235 | 1.62E−06 | 8.78E−04 |
| GO:0005576 | CC | extracellular region | 4361 | 557 | 1.82E−06 | 9.64E−04 |
| GO:0022409 | BP | positive regulation of cell- | 288 | 66 | 1.93E−06 | 1.00E−03 |
| | | cell adhesion | | | | |
| GO:0046649 | BP | lymphocyte activation | 717 | 129 | 2.05E−06 | 1.04E−03 |
| GO:0001816 | BP | cytokine production | 820 | 131 | 2.25E−06 | 1.11E−03 |
| GO:0022407 | BP | regulation of cell-cell | 453 | 93 | 2.36E−06 | 1.15E−03 |
| | | adhesion | | | | |
| GO:0006954 | BP | inflammatory response | 821 | 129 | 2.67E−06 | 1.27E−03 |
| GO:0032642 | BP | regulation of chemokine | 97 | 26 | 2.90E−06 | 1.35E−03 |
| | | production | | | | |
| GO:0001819 | BP | positive regulation of | 471 | 87 | 2.95E−06 | 1.35E−03 |
| | | cytokine production | | | | |
| GO:0032602 | BP | chemokine production | 98 | 26 | 3.33E−06 | 1.47E−03 |
| GO:0019221 | BP | cytokine-mediated | 479 | 82 | 3.35E−06 | 1.47E−03 |
| | | signaling pathway | | | | |
| GO:0002523 | BP | leukocyte migration | 19 | 10 | 3.48E−06 | 1.50E−03 |
| | | involved in inflammatory | | | | |
| | | response | | | | |
| GO:0032501 | BP | multicellular organismal | 7364 | 994 | 4.21E−06 | 1.78E−03 |
| | | process | | | | |
| GO:0060326 | BP | cell chemotaxis | 311 | 62 | 4.99E−06 | 2.07E−03 |
| GO:0002253 | BP | activation of immune | 310 | 65 | 5.22E−06 | 2.13E−03 |
| | | response | | | | |
| GO:0002683 | BP | negative regulation of | 424 | 79 | 5.34E−06 | 2.14E−03 |
| | | immune system process | | | | |
| GO:0005615 | CC | extracellular space | 3402 | 445 | 6.13E−06 | 2.39E−03 |
| GO:0071674 | BP | mononuclear cell | 196 | 43 | 6.26E−06 | 2.39E−03 |
| | | migration | | | | |
| GO:0098542 | BP | defense response to | 1019 | 145 | 6.29E−06 | 2.39E−03 |
| | | other organism | | | | |
| GO:1903131 | BP | mononuclear cell | 432 | 85 | 7.67E−06 | 2.87E−03 |
| | | differentiation | | | | |
| GO:0048518 | BP | positive regulation of | 6118 | 866 | 7.93E−06 | 2.92E−03 |
| | | biological process | | | | |
| GO:0002520 | BP | immune system | 955 | 167 | 8.13E−06 | 2.92E−03 |
| | | development | | | | |
| GO:0097529 | BP | myeloid leukocyte | 227 | 47 | 8.18E−06 | 2.92E−03 |
| | | migration | | | | |
| GO:0001817 | BP | regulation of cytokine | 806 | 126 | 8.74E−06 | 3.07E−03 |
| | | production | | | | |
| GO:0042379 | MF | chemokine receptor | 69 | 15 | 9.08E−06 | 3.14E−03 |
| | | binding | | | | |
| GO:1903039 | BP | positive regulation of | 242 | 55 | 9.55E−06 | 3.25E−03 |
| | | leukocyte cell-cell | | | | |
| | | adhesion | | | | |
| GO:0051239 | BP | regulation of multicellular | 2751 | 420 | 1.16E−05 | 3.88E−03 |
| | | organismal process | | | | |
| GO:1903037 | BP | regulation of leukocyte | 34″ | 68 | 1.27E−05 | 4.14E−03 |
| | | cell-cell adhesion | | | | |
| GO:0050865 | BP | regulation of cell | 602 | 108 | 1.27E−05 | 4.14E−03 |
| | | activation | | | | |
| GO:0035864 | BP | response to potassium | 16 | 10 | 1.34E−05 | 4.32E−03 |
| | | ion | | | | |
| GO:0048020 | MF | CCR chemokine receptor | 45 | 11 | 1.36E−05 | 4.32E−03 |
| | | binding | | | | |
| GO:0030155 | BP | regulation of cell | 756 | 146 | 1.45E−05 | 4.52E−03 |
| | | adhesion | | | | |
| GO:0050900 | BP | leukocyte migration | 380 | 72 | 1.53E−05 | 4.72E−03 |
| GO:0044419 | BP | biological process | 1525 | 212 | 1.63E−05 | 4.97E−03 |
| | | involved in interspecies | | | | |
| | | interaction between | | | | |
| | | organisms | | | | |
| GO:0006909 | BP | phagocytosis | 235 | 55 | 1.69E−05 | 5.06E−03 |
| GO:0035556 | BP | intracellular signal | 2640 | 421 | 1.76E−05 | 5.16E−03 |
| | | transduction | | | | |
| GO:0097530 | BP | granulocyte migration | 152 | 33 | 1.76E−05 | 5.16E−03 |
| GO:0043410 | BP | positive regulation of | 490 | 97 | 1.81E−05 | 5.24E−03 |
| | | MAPK cascade | | | | |
| GO:0005126 | MF | cytokine receptor binding | 269 | 47 | 1.93E−05 | 5.46E−03 |
| GO:0050790 | BP | regulation of catalytic | 2325 | 368 | 1.94E−05 | 5.46E−03 |
| | | activity | | | | |
| GO:0071310 | BP | cellular response to | 2398 | 365 | 1.99E−05 | 5.54E−03 |
| | | organic substance | | | | |
| GO:0030098 | BP | lymphocyte differentiation | 381 | 75 | 2.38E−05 | 6.54E−03 |
| GO:0098552 | CC | side of membrane | 574 | 107 | 2.42E−05 | 6.56E−03 |
| GO:0050870 | BP | positive regulation of T | 220 | 50 | 2.50E−05 | 6.72E−03 |
| | | cell activation | | | | |
| GO:0002429 | BP | immune response- | 225 | 52 | 2.64E−05 | 6.93E−03 |
| | | activating cell surface | | | | |
| | | receptor signaling | | | | |
| | | pathway | | | | |
| GO:0002757 | BP | immune response- | 225 | 52 | 2.64E−05 | 6.93E−03 |
| | | activating signal | | | | |
| | | transduction | | | | |
| GO:0045121 | CC | membrane raft | 326 | 75 | 2.73E−05 | 7.07E−03 |
| GO:0098857 | CC | membrane microdomain | 327 | 75 | 2.88E−05 | 7.38E−03 |
| GO:0002460 | BP | adaptive immune | 292 | 57 | 3.05E−05 | 7.73E−03 |
| | | response based on | | | | |
| | | somatic recombination of | | | | |
| | | immune receptors built | | | | |
| | | from immunoglobulin | | | | |
| | | superfamily domains | | | | |
| GO:0002694 | BP | regulation of leukocyte | 550 | 98 | 3.19E−05 | 7.99E−03 |
| | | activation | | | | |
| GO:0008009 | MF | chemokine activity | 49 | 11 | 3.36E−05 | 8.32E−03 |
| GO:0002443 | BP | leukocyte mediated | 378 | 67 | 3.59E−05 | 8.81E−03 |
| | | immunity | | | | |
| GO:0048247 | BP | lymphocyte chemotaxis | 63 | 16 | 3.75E−05 | 9.10E−03 |
| GO:0035865 | BP | cellular response to | 12 | 8 | 4.03E−05 | 9.67E−03 |
| | | potassium ion | | | | |
| GO:0032722 | BP | positive regulation of | 70 | 19 | 4.64E−05 | 1.10E−02 |
| | | chemokine production | | | | |
| GO:0043408 | BP | regulation of MAPK | 685 | 125 | 4.75E−05 | 1.12E−02 |
| | | cascade | | | | |
| GO:0016020 | CC | membrane | 9099 | 1179 | 5.59E−05 | 1.30E−02 |
| GO:0050851 | BP | antigen receptor- | 171 | 42 | 5.86E−05 | 1.35E−02 |
| | | mediated signaling | | | | |
| | | pathway | | | | |
| GO:1905517 | BP | macrophage migration | 55 | 17 | 6.43E−05 | 1.44E−02 |
| GO:0050863 | BP | regulation of T cell | 339 | 66 | 6.43E−05 | 1.44E−02 |
| | | activation | | | | |
| GO:0070887 | BP | cellular response to | 3027 | 440 | 6.48E−05 | 1.44E−02 |
| | | chemical stimulus | | | | |
| GO:0070663 | BP | regulation of leukocyte | 252 | 50 | 6.49E−05 | 1.44E−02 |
| | | proliferation | | | | |
| GO:0002274 | BP | myeloid leukocyte | 230 | 47 | 6.71E−05 | 1.47E−02 |
| | | activation | | | | |
| GO:0031295 | BP | T cell costimulation | 44 | 15 | 7.17E−05 | 1.55E−02 |
| GO:0032103 | BP | positive regulation of | 438 | 75 | 7.21E−05 | 1.55E−02 |
| | | response to external | | | | |
| | | stimulus | | | | |
| GO:0002767 | BP | immune response- | 9 | 6 | 7.37E−05 | 1.57E−02 |
| | | inhibiting cell surface | | | | |
| | | receptor signaling | | | | |
| | | pathway | | | | |
| GO:0050866 | BP | negative regulation of cell | 215 | 45 | 7.56E−05 | 1.60E−02 |
| | | activation | | | | |
| GO:0070374 | BP | positive regulation of | 219 | 46 | 7.68E−05 | 1.61E−02 |
| | | ERK1 and ERK2 cascade | | | | |
| GO:0042127 | BP | regulation of cell | 1716 | 261 | 7.88E−05 | 1.63E−02 |
| | | population proliferation | | | | |
| GO:0019763 | MF | immunoglobulin receptor | 7 | 4 | 7.93E−05 | 1.63E−02 |
| | | activity | | | | |
| GO:0072676 | BP | lymphocyte migration | 116 | 26 | 8.74E−05 | 1.78E−02 |
| GO:0051240 | BP | positive regulation of | 1453 | 238 | 8.79E−05 | 1.78E−02 |
| | | multicellular organismal | | | | |
| | | process | | | | |
| GO:0002695 | BP | negative regulation of | 192 | 40 | 8.93E−05 | 1.79E−02 |
| | | leukocyte activation | | | | |
| GO:0009607 | BP | response to biotic | 1409 | 191 | 9.26E−05 | 1.82E−02 |
| | | stimulus | | | | |
| GO:0051707 | BP | response to other | 1367 | 185 | 9.28E−05 | 1.82E−02 |
| | | organism | | | | |
| GO:0048522 | BP | positive regulation of | 5583 | 793 | 9.43E−05 | 1.84E−02 |
| | | cellular process | | | | |
| GO:0031226 | CC | intrinsic component of | 1718 | 267 | 9.78E−05 | 1.88E−02 |
| | | plasma membrane | | | | |
| GO:0048870 | BP | cell motility | 1739 | 282 | 9.89E−05 | 1.88E−02 |
| GO:0051674 | BP | localization of cell | 1739 | 282 | 9.89E−05 | 1.88E−02 |
| GO:0043207 | BP | response to external | 1370 | 185 | 1.01E−04 | 1.90E−02 |
| | | biotic stimulus | | | | |
| GO:0002699 | BP | positive regulation of | 244 | 47 | 1.04E−04 | 1.94E−02 |
| | | immune effector process | | | | |
| GO:0031294 | BP | lymphocyte costimulation | 46 | 15 | 1.07E−04 | 1.98E−02 |
| GO:1902531 | BP | regulation of intracellular | 1717 | 278 | 1.09E−04 | 2.01E−02 |
| | | signal transduction | | | | |
| GO:0009986 | CC | cell surface | 861 | 146 | 1.11E−04 | 2.02E−02 |
| GO:0034097 | BP | response to cytokine | 907 | 137 | 1.11E−04 | 2.02E−02 |
| GO:0002696 | BP | positive regulation of | 342 | 66 | 1.12E−04 | 2.02E−02 |
| | | leukocyte activation | | | | |
| GO:0002548 | BP | monocyte chemotaxis | 70 | 16 | 1.17E−04 | 2.08E−02 |
| GO:0009605 | BP | response to external | 2661 | 376 | 1.18E−04 | 2.08E−02 |
| | | stimulus | | | | |
| GO:1902107 | BP | positive regulation of | 156 | 38 | 1.25E−04 | 2.18E−02 |
| | | leukocyte differentiation | | | | |
| GO:1903708 | BP | positive regulation of | 156 | 38 | 1.25E−04 | 2.18E−02 |
| | | hemopoiesis | | | | |
| GO:0048519 | BP | negative regulation of | 5710 | 752 | 1.30E−04 | 2.24E−02 |
| | | biological process | | | | |
| GO:0008283 | BP | cell population | 2000 | 299 | 1.34E−04 | 2.30E−02 |
| | | proliferation | | | | |
| GO:0071723 | MF | lipopeptide binding | 10 | 5 | 1.50E−04 | 2.56E−02 |
| GO:0002275 | BP | myeloid cell activation | 92 | 24 | 1.51E−04 | 2.56E−02 |
| | | involved in immune | | | | |
| | | response | | | | |
| GO:0042110 | BP | T cell activation | 503 | 89 | 1.56E−04 | 2.61E−02 |
| GO:0050793 | BP | regulation of | 2436 | 386 | 1.57E−04 | 2.61E−02 |
| | | developmental process | | | | |
| GO:0005887 | CC | integral component of | 1637 | 25: | 1.62E−04 | 2.68E−02 |
| | | plasma membrane | | | | |
| GO:0050867 | BP | positive regulation of cell | 357 | 67 | 1.77E−04 | 2.88E−02 |
| | | activation | | | | |
| GO:0051251 | BP | positive regulation of | 294 | 59 | 1.78E−04 | 2.88E−02 |
| | | lymphocyte activation | | | | |
| GO:0002449 | BP | lymphocyte mediated | 286 | 51 | 1.79E−04 | 2.88E−02 |
| | | immunity | | | | |
| GO:0030595 | BP | leukocyte chemotaxis | 234 | 44 | 1.79E−04 | 2.88E−02 |
| GO:0016477 | BP | cell migration | 1547 | 255 | 1.90E−04 | 3.03E−02 |
| GO:1990266 | BP | neutrophil migration | 126 | 26 | 1.95E−04 | 3.09E−02 |
| GO:0042113 | BP | B cell activation | 262 | 51 | 2.00E−04 | 3.13E−02 |
| GO:0006959 | BP | humoral immune | 236 | 36 | 2.00E−04 | 3.13E−02 |
| | | response | | | | |
| GO:0002697 | BP | regulation of immune | 349 | 60 | 2.02E−04 | 3.13E−02 |
| | | effector process | | | | |
| GO:1903038 | BP | negative regulation of | 143 | 30 | 2.13E−04 | 3.29E−02 |
| | | leukocyte cell-cell | | | | |
| | | adhesion | | | | |
| GO:0097197 | CC | tetraspanin-enriched | 10 | 7 | 2.16E−04 | 3.31E−02 |
| | | microdomain | | | | |
| GO:0048856 | BP | anatomical structure | 5751 | 830 | 2.21E−04 | 3.35E−02 |
| | | development | | | | |
| GO:0051179 | BP | localization | 6104 | 859 | 2.26E−04 | 3.41E−02 |
| GO:0048869 | BP | cellular developmental | 4172 | 619 | 2.34E−04 | 3.51E−02 |
| | | process | | | | |
| GO:0002765 | BP | immune response- | 11 | 6 | 2.41E−04 | 3.60E−02 |
| | | inhibiting signal | | | | |
| | | transduction | | | | |
| GO:0050789 | BP | regulation of biological | 11762 | 1448 | 2.52E−04 | 3.73E−02 |
| | | process | | | | |
| GO:0032944 | BP | regulation of | 232 | 45 | 2.57E−04 | 3.78E−02 |
| | | mononuclear cell | | | | |
| | | proliferation | | | | |
| GO:0030154 | BP | cell differentiation | 4146 | 615 | 2.70E−04 | 3.94E−02 |
| GO:0070302 | BP | regulation of stress- | 200 | 46 | 2.79E−04 | 4.06E−02 |
| | | activated protein kinase | | | | |
| | | signaling cascade | | | | |
| GO:0042221 | BP | response to chemical | 4321 | 566 | 2.91E−04 | 4.20E−02 |
| GO:0044093 | BP | positive regulation of | 1532 | 252 | 3.00E−04 | 4.30E−02 |
| | | molecular function | | | | |
| GO:0000165 | BP | MAPK cascade | 785 | 136 | 3.03E−04 | 4.33E−02 |
| GO:0032879 | BP | regulation of localization | 2793 | 424 | 3.06E−04 | 4.34E−02 |
| GO:0070062 | CC | extracellular exosome | 2144 | 304 | 3.17E−04 | 4.46E−02 |
| GO:0032872 | BP | regulation of stress- | 197 | 45 | 3.35E−04 | 4.66E−02 |
| | | activated MAPK cascade | | | | |
| GO:0046635 | BP | positive regulation of | 68 | 20 | 3.35E−04 | 4.66E−02 |
| | | alpha-beta T cell | | | | |
| | | activation | | | | |
| GO:1903706 | BP | regulation of hemopoiesis | 367 | 69 | 3.38E−04 | 4.67E−02 |
| GO:0031224 | CC | intrinsic component of | 5359 | 670 | 3.53E−04 | 4.86E−02 |
| | | membrane | | | | |
| GO:0046328 | BP | regulation of JNK | 141 | 36 | 3.64E−04 | 4.96E−02 |
| | | cascade | | | | |
| GO:0022408 | BP | negative regulation of | 198 | 41 | 3.67E−04 | 4.96E−02 |
| | | cell-cell adhesion | | | | |
| GO:0002823 | BP | negative regulation of | 54 | 15 | 3.67E−04 | 4.96E−02 |
| | | adaptive immune | | | | |
| | | response based on | | | | |
| | | somatic recombination of | | | | |
| | | immune receptors built | | | | |
| | | from immunoglobulin | | | | |
| | | superfamily domains | | | | |
| GO:0002232 | BP | leukocyte chemotaxis | 8 | 5 | 3.71E−04 | 4.97E−02 |
| | | involved in inflammatory | | | | |
| | | response | | | | |
| GO:0070371 | BP | ERK1 and ERK2 cascade | 334 | 62 | 3.73E−04 | 4.97E−02 |
| GO:0008284 | BP | positive regulation of cell | 946 | 148 | 3.76E−04 | 4.99E−02 |
| | | population proliferation | | | | |
| GO:0051249 | BP | regulation of lymphocyte | 455 | 80 | 3.78E−04 | 4.99E−02 |
| | | activation | | | | |

TABLE 11B
IBS vs Healthy p < 0.05

| ONTOLOGY | | TERM | N | DE | P.DE | FDR |
|---|---|---|---|---|---|---|
| GO:0031224 | CC | intrinsic component of | 5359 | 31 | 0.00027638 | 1 |
| | | membrane | | | | |
| GO:0042113 | BP | B cell activation | 262 | 5 | 0.002028412 | 1 |
| GO:0016021 | CC | integral component of | 5194 | 28 | 0.00204853 | 1 |
| | | membrane | | | | |
| GO:0061812 | MF | cyclic ADP-ribose hydrolase | 1 | 1 | 0.003578322 | 1 |
| GO:0042044 | BP | fluid transport | 31 | 2 | 0.004495101 | 1 |
| GO:0006029 | BP | proteoglycan metabolic | 80 | 3 | 0.004722518 | 1 |
| | | process | | | | |
| GO:0007186 | BP | G protein-coupled receptor | 1204 | 8 | 0.006175726 | 1 |
| | | signaling pathway | | | | |
| GO:0008418 | MF | protein-N-terminal asparagine | 2 | 1 | 0.007303176 | 1 |
| | | amidohydrolase activity | | | | |
| GO:0071402 | BP | cellular response to lipoprotein | 36 | 2 | 0.007539902 | 1 |
| | | particle stimulus | | | | |
| GO:0005217 | MF | intracellular ligand-gated ion | 30 | 2 | 0.00831452 | 1 |
| | | channel activity | | | | |
| GO:0090162 | BP | establishment of epithelial cell | 31 | 2 | 0.008430724 | 1 |
| | | polarity | | | | |
| GO:0030545 | MF | signaling receptor regulator | 534 | 5 | 0.012249445 | 1 |
| | | activity | | | | |
| GO:0014902 | BP | myotube differentiation | 120 | 3 | 0.012475759 | 1 |
| GO:0099634 | CC | postsynaptic specialization | 116 | 3 | 0.013314047 | 1 |
| | | membrane | | | | |
| GO:0030225 | BP | macrophage differentiation | 49 | 2 | 0.013975475 | 1 |
| GO:0018212 | BP | peptidyl-tyrosine modification | 374 | 5 | 0.015310247 | 1 |
| GO:0008277 | BP | regulation of G protein-coupled | 137 | 3 | 0.015649037 | 1 |
| | | receptor signaling pathway | | | | |
| GO:0070665 | BP | positive regulation of leukocyte | 157 | 3 | 0.016031144 | 1 |
| | | proliferation | | | | |
| GO:0098794 | CC | postsynapse | 607 | 7 | 0.016455647 | 1 |
| GO:0005125 | MF | cytokine activity | 235 | 3 | 0.016626729 | 1 |
| GO:0099179 | BP | regulation of synaptic | 4 | 1 | 0.017620978 | 1 |
| | | membrane adhesion | | | | |
| GO:0009988 | BP | cell-cell recognition | 69 | 2 | 0.017715414 | 1 |
| GO:0006826 | BP | iron ion transport | 58 | 2 | 0.017973039 | 1 |
| GO:0001923 | BP | B-1 B cell differentiation | 5 | 1 | 0.020889894 | 1 |
| GO:0070661 | BP | leukocyte proliferation | 328 | 4 | 0.021382206 | 1 |
| GO:0030166 | BP | proteoglycan biosynthetic | 56 | 2 | 0.022519592 | 1 |
| | | process | | | | |
| GO:0001537 | MF | N-acetylgalactosamine 4-O- | 5 | 1 | 0.022697309 | 1 |
| | | sulfotransferase activity | | | | |
| GO:0042446 | BP | hormone biosynthetic process | 64 | 2 | 0.023172951 | 1 |
| GO:0050854 | BP | regulation of antigen receptor- | 66 | 2 | 0.023208539 | 1 |
| | | mediated signaling pathway | | | | |
| GO:0006949 | BP | syncytium formation | 61 | 2 | 0.023841121 | 1 |
| GO:0045178 | CC | basal part of cell | 268 | 4 | 0.024227296 | 1 |
| GO:0007386 | BP | compartment pattern | 5 | 1 | 0.025303982 | 1 |
| | | specification | | | | |
| GO:0016811 | MF | hydrolase activity, acting on | 69 | 2 | 0.025467608 | 1 |
| | | carbon-nitrogen (but not | | | | |
| | | peptide) bonds, in linear | | | | |
| | | amides | | | | |
| GO:0005102 | MF | signaling receptor binding | 1468 | 10 | 0.027792606 | 1 |
| GO:0050731 | BP | positive regulation of peptidyl- | 188 | 3 | 0.028320399 | 1 |
| | | tyrosine phosphorylation | | | | |
| GO:0005178 | MF | integrin binding | 156 | 3 | 0.028620053 | 1 |
| GO:1904951 | BP | positive regulation of | 306 | 4 | 0.031132232 | 1 |
| | | establishment of protein | | | | |
| | | localization | | | | |
| GO:0044691 | BP | tooth eruption | 7 | 1 | 0.031568116 | 1 |
| GO:0050871 | BP | positive regulation of B cell | 79 | 2 | 0.03216027 | 1 |
| | | activation | | | | |
| GO:0008237 | MF | metallopeptidase activity | 185 | 3 | 0.032518718 | 1 |
| GO:0060485 | BP | mesenchyme development | 299 | 4 | 0.032532112 | 1 |
| GO:0001547 | BP | antral ovarian follicle growth | 9 | 1 | 0.032586818 | 1 |
| GO:0043550 | BP | regulation of lipid kinase | 68 | 2 | 0.033056524 | 1 |
| | | activity | | | | |
| GO:0004703 | MF | G protein-coupled receptor | 7 | 1 | 0.033492222 | 1 |
| | | kinase activity | | | | |
| GO:0007610 | BP | behavior | 605 | 6 | 0.0335959 | 1 |
| GO:0036128 | CC | CatSper complex | 13 | 1 | 0.03418808 | 1 |
| GO:0010324 | BP | membrane invagination | 71 | 2 | 0.034339001 | 1 |
| GO:0032782 | BP | bile acid secretion | 12 | 1 | 0.034946738 | 1 |
| GO:0042310 | BP | vasoconstriction | 82 | 2 | 0.036123849 | 1 |
| GO:0050900 | BP | leukocyte migration | 380 | 4 | 0.036239543 | 1 |
| GO:0050655 | BP | dermatan sulfate proteoglycan | 9 | 1 | 0.037965036 | 1 |
| | | metabolic process | | | | |
| GO:0042445 | BP | hormone metabolic process | 229 | 3 | 0.038089733 | 1 |
| GO:0030546 | MF | signaling receptor activator | 492 | 4 | 0.03911146 | 1 |
| | | activity | | | | |
| GO:0033572 | BP | transferrin transport | 10 | 1 | 0.040245082 | 1 |
| GO:0005883 | CC | neurofilament | 9 | 1 | 0.040555722 | 1 |
| GO:0044771 | BP | meiotic cell cycle phase | 12 | 1 | 0.040590394 | 1 |
| | | transition | | | | |
| GO:0032501 | BP | multicellular organismal | 7364 | 33 | 0.040812079 | 1 |
| | | process | | | | |
| GO:0008344 | BP | adult locomotory behavior | 76 | 2 | 0.040938513 | 1 |
| GO:0070243 | BP | regulation of thymocyte | 11 | 1 | 0.041611832 | 1 |
| | | apoptotic process | | | | |
| GO:0004312 | MF | fatty acid synthase activity | 12 | 1 | 0.041676566 | 1 |
| GO:0061043 | BP | regulation of vascular wound | 14 | 1 | 0.042585207 | 1 |
| | | healing | | | | |
| GO:0099612 | BP | protein localization to axon | 9 | 1 | 0.042643927 | 1 |
| GO:0009111 | BP | vitamin catabolic process | 15 | 1 | 0.042834486 | 1 |
| GO:0031226 | CC | intrinsic component of plasma | 1718 | 11 | 0.042871358 | 1 |
| | | membrane | | | | |
| GO:0032944 | BP | regulation of mononuclear cell | 232 | 3 | 0.042878333 | 1 |
| | | proliferation | | | | |
| GO:0015669 | BP | gas transport | 21 | 1 | 0.043680801 | 1 |
| GO:0031235 | CC | intrinsic component of the | 10 | 1 | 0.043786132 | 1 |
| | | cytoplasmic side of the plasma | | | | |
| | | membrane | | | | |
| GO:0002573 | BP | myeloid leukocyte | 210 | 3 | 0.04417326 | 1 |
| | | differentiation | | | | |
| GO:0034982 | BP | mitochondrial protein | 13 | 1 | 0.044455419 | 1 |
| | | processing | | | | |
| GO:0098948 | CC | intrinsic component of | 80 | 2 | 0.045017797 | 1 |
| | | postsynaptic specialization | | | | |
| | | membrane | | | | |
| GO:0005869 | CC | dynactin complex | 16 | 1 | 0.045077075 | 1 |
| GO:0021932 | BP | hindbrain radial glia guided cell | 9 | 1 | 0.045188863 | 1 |
| | | migration | | | | |
| GO:0009966 | BP | regulation of signal | 2971 | 17 | 0.046224293 | 1 |
| | | transduction | | | | |
| GO:0016668 | MF | oxidoreductase activity, acting | 13 | 1 | 0.046538503 | 1 |
| | | on a sulfur group of donors, | | | | |
| | | NAD(P) as acceptor | | | | |
| GO:0034650 | BP | cortisol metabolic process | 12 | 1 | 0.046659445 | 1 |
| GO:0051262 | BP | protein tetramerization | 88 | 2 | 0.046723368 | 1 |
| GO:0071421 | BP | manganese ion | 10 | 1 | 0.046776877 | 1 |
| | | transmembrane transport | | | | |
| GO:0045202 | CC | synapse | 1302 | 10 | 0.046871939 | 1 |
| GO:0045187 | BP | regulation of circadian | 15 | 1 | 0.047600902 | 1 |
| | | sleep/wake cycle, sleep | | | | |
| GO:0002521 | BP | leukocyte differentiation | 551 | 5 | 0.048177625 | 1 |
| GO:0048548 | BP | regulation of pinocytosis | 12 | 1 | 0.049218607 | 1 |
| GO:0030100 | BP | regulation of endocytosis | 208 | 3 | 0.049290129 | 1 |
| GO:0030955 | MF | potassium ion binding | 12 | 1 | 0.049480737 | 1 |
| GO:0050913 | BP | sensory perception of bitter | 41 | 1 | 0.049515502 | 1 |
| | | taste | | | | |
| GO:0042976 | BP | activation of Janus kinase | 15 | 1 | 0.049548899 | 1 |
| | | activity | | | | |
| GO:0021860 | BP | pyramidal neuron development | 11 | 1 | 0.049795415 | 1 |
| GO:0070863 | BP | positive regulation of protein | 13 | 1 | 0.049881621 | 1 |
| | | exit from endoplasmic | | | | |
| | | reticulum | | | | |

TABLE 11C
IBS vs IBD p < 0.05

| ONTOLOGY | | TERM | N | DE | P.DE | FDR |
|---|---|---|---|---|---|---|
| GO:0006959 | BP | humoral immune response | 236 | 9 | 5.93396728328521e−05 | 1 |
| GO:0002250 | BP | adaptive immune response | 438 | 14 | 0.000193365 | 1 |
| GO:0002455 | BP | humoral immune response | 49 | 4 | 0.000805573 | 1 |
| | | mediated by circulating Ig | | | | |
| GO:0023023 | MF | MHC protein complex binding | 34 | 4 | 0.000912125 | 1 |
| GO:1903555 | BP | regulation of tumor necrosis factor | 186 | 7 | 0.001782103 | 1 |
| | | superfamily cytokine production | | | | |
| GO:0021626 | BP | central nervous system maturation | 7 | 2 | 0.002060378 | 1 |
| GO:0019724 | BP | B cell mediated immunity | 143 | 6 | 0.002398658 | 1 |
| GO:1903556 | BP | negative regulation of TNF | 78 | 4 | 0.002506275 | 1 |
| | | superfamily cytokine production | | | | |
| GO:0097356 | CC | perinucleolar compartment | 5 | 2 | 0.002774315 | 1 |
| GO:0008378 | MF | galactosyltransferase activity | 32 | 3 | 0.002896286 | 1 |
| GO:0001818 | BP | negative regulation of cytokine | 362 | 9 | 0.002897151 | 1 |
| | | production | | | | |
| GO:1900016 | BP | negative regulation of cytokine | 39 | 3 | 0.00290315 | 1 |
| | | production involved in inflammatory | | | | |
| | | response | | | | |
| GO:0003713 | MF | transcription coactivator activity | 264 | 10 | 0.003244951 | 1 |
| GO:0003712 | MF | transcription coregulator activity | 489 | 15 | 0.003340449 | 1 |
| GO:0002460 | BP | adaptive immune response based | 292 | 9 | 0.003644504 | 1 |
| | | on somatic recombination of | | | | |
| | | immune receptors built from lg | | | | |
| | | superfamily domains | | | | |
| GO:0015026 | MF | coreceptor activity | 46 | 4 | 0.003793799 | 1 |
| GO:0070841 | BP | inclusion body assembly | 24 | 3 | 0.004263027 | 1 |
| GO:0043296 | CC | apical junction complex | 148 | 7 | 0.005928583 | 1 |
| GO:0004113 | MF | 2′,3′-cyclic-nucleotide 3′- | 1 | 1 | 0.006643455 | 1 |
| | | phosphodiesterase activity | | | | |
| GO:0002449 | BP | lymphocyte mediated immunity | 286 | 8 | 0.006716964 | 1 |
| GO:0002291 | BP | T cell activation via T cell receptor | 10 | 2 | 0.006985691 | 1 |
| | | contact with antigen bound to MHC | | | | |
| | | molecule on APC | | | | |
| GO:1990822 | BP | basic aa transmembrane transport | 10 | 2 | 0.006998714 | 1 |
| GO:0140375 | MF | immune receptor activity | 140 | 5 | 0.007356828 | 1 |
| GO:0035748 | CC | myelin sheath abaxonal region | 6 | 2 | 0.007836502 | 1 |
| GO:0048730 | BP | epidermis morphogenesis | 34 | 3 | 0.00784594 | 1 |
| GO:0061245 | BP | establishment or maintenance of | 50 | 4 | 0.007919531 | 1 |
| | | bipolar cell polarity | | | | |
| GO:0044877 | MF | protein-containing complex binding | 1267 | 27 | 0.00794488 | 1 |
| GO:0005112 | MF | Notch binding | 24 | 3 | 0.008087761 | 1 |
| GO:0006968 | BP | cellular defense response | 53 | 3 | 0.008296711 | 1 |
| GO:0001540 | MF | amyloid-beta binding | 81 | 5 | 0.008654882 | 1 |
| GO:0048531 | MF | beta-1,3-galactosyltransferase | 17 | 2 | 0.008776578 | 1 |
| | | activity | | | | |
| GO:0070161 | CC | anchoring junction | 834 | 22 | 0.009260642 | 1 |
| GO:0002703 | BP | regulation of leukocyte mediated | 233 | 7 | 0.009292159 | 1 |
| | | immunity | | | | |
| GO:0019865 | MF | immunoglobulin binding | 24 | 2 | 0.009624687 | 1 |
| GO:0003158 | BP | endothelium development | 133 | 6 | 0.009769046 | 1 |
| GO:0002532 | BP | production of molecular mediator | 96 | 4 | 0.009774377 | 1 |
| | | involved in inflammatory response | | | | |
| GO:0009986 | CC | cell surface | 861 | 19 | 0.009994826 | 1 |
| GO:0035914 | BP | skeletal muscle cell differentiation | 64 | 4 | 0.010058813 | 1 |
| GO:0071819 | CC | DUBm complex | 2 | 1 | 0.010202551 | 1 |
| GO:0050680 | BP | negative regulation of epithelial cell | 163 | 6 | 0.010354253 | 1 |
| | | proliferation | | | | |
| GO:0090084 | BP | negative regulation of inclusion | 11 | 2 | 0.010408578 | 1 |
| | | body assembly | | | | |
| GO:0031122 | BP | cytoplasmic microtubule | 57 | 4 | 0.010998628 | 1 |
| | | organization | | | | |
| GO:0097119 | BP | postsynaptic density protein 95 | 8 | 2 | 0.01108439 | 1 |
| | | clustering | | | | |
| GO:0042924 | MF | neuromedin U binding | 2 | 1 | 0.01129335 | 1 |
| GO:0032486 | BP | Rap protein signal transduction | 13 | 2 | 0.011685076 | 1 |
| GO:0060419 | BP | heart growth | 96 | 5 | 0.012026418 | 1 |
| GO:0002819 | BP | regulation of adaptive immune | 185 | 6 | 0.012174249 | 1 |
| | | response | | | | |
| GO:0030165 | MF | PDZ domain binding | 82 | 5 | 0.012264734 | 1 |
| GO:0060627 | BP | regulation of vesicle-mediated | 527 | 14 | 0.01226622 | 1 |
| | | transport | | | | |
| GO:0002443 | BP | leukocyte mediated immunity | 378 | 9 | 0.01229636 | 1 |
| GO:0048792 | BP | spontaneous exocytosis of | 1 | 1 | 0.012365956 | 1 |
| | | neurotransmitter | | | | |
| GO:0060525 | BP | prostate glandular acinus | 9 | 2 | 0.012736728 | 1 |
| | | development | | | | |
| GO:0031341 | BP | regulation of cell killing | 100 | 4 | 0.012759783 | 1 |
| GO:1905957 | BP | regulation of cellular response to | 1 | 1 | 0.013829156 | 1 |
| | | alcohol | | | | |
| GO:0035853 | BP | chromosome passenger complex | 1 | 1 | 0.014180505 | 1 |
| | | localization to spindle midzone | | | | |
| GO:0098792 | BP | xenophagy | 13 | 2 | 0.015024183 | 1 |
| GO:0042772 | BP | DNA damage response, signal | 18 | 2 | 0.015157416 | 1 |
| | | transduction resulting in | | | | |
| | | transcription | | | | |
| GO:0004888 | MF | transmembrane signaling receptor | 1228 | 18 | 0.015169838 | 1 |
| | | activity | | | | |
| GO:0031333 | BP | negative regulation of protein- | 143 | 6 | 0.015300209 | 1 |
| | | containing complex assembly | | | | |
| GO:0051660 | BP | establishment of centrosome | 10 | 2 | 0.015341237 | 1 |
| | | localization | | | | |
| GO:0005881 | CC | cytoplasmic microtubule | 75 | 4 | 0.01562827 | 1 |
| GO:0071936 | MF | coreceptor activity involved in Wnt | 9 | 2 | 0.015710919 | 1 |
| | | signaling pathway | | | | |
| GO:0014067 | BP | negative regulation of phosphatidyl- | 19 | 2 | 0.015763381 | 1 |
| | | inositol 3-kinase signaling | | | | |
| GO:0050678 | BP | regulation of epithelial cell | 382 | 10 | 0.016079289 | 1 |
| | | proliferation | | | | |
| GO:0050673 | BP | epithelial cell proliferation | 443 | 11 | 0.016568849 | 1 |
| GO:0060253 | BP | negative regulation of glial cell | 13 | 2 | 0.016804304 | 1 |
| | | proliferation | | | | |
| GO:2000107 | BP | negative regulation of leukocyte | 49 | 3 | 0.017017632 | 1 |
| | | apoptotic process | | | | |
| GO:0001885 | BP | endothelial cell development | 64 | 4 | 0.017036687 | 1 |
| GO:0014009 | BP | glial cell proliferation | 44 | 3 | 0.01736427 | 1 |
| GO:0048755 | BP | branching morphogenesis of a | 10 | 2 | 0.017364408 | 1 |
| | | nerve | | | | |
| GO:0046974 | MF | histone methyltransferase activity | 10 | 2 | 0.017394991 | 1 |
| | | (H3-K9 specific) | | | | |
| GO:0005085 | MF | guanyl-nucleotide exchange factor | 223 | 9 | 0.017447532 | 1 |
| | | activity | | | | |
| GO:0010839 | BP | negative regulation of keratinocyte | 15 | 2 | 0.017453717 | 1 |
| | | proliferation | | | | |
| GO:0016553 | BP | base conversion or substitution | 20 | 2 | 0.017743893 | 1 |
| | | editing | | | | |
| GO:0014731 | CC | spectrin-associated cytoskeleton | 9 | 2 | 0.017966875 | 1 |
| GO:0002820 | BP | negative regulation of adaptive | 58 | 3 | 0.018101739 | 1 |
| | | immune response | | | | |
| GO:0009897 | CC | external side of plasma membrane | 367 | 9 | 0.018554807 | 1 |
| GO:0015801 | BP | aromatic amino acid transport | 13 | 2 | 0.019282259 | 1 |
| GO:0030849 | CC | autosome | 2 | 1 | 0.019306081 | 1 |
| GO:1901096 | BP | regulation of autophagosome | 16 | 2 | 0.019325722 | 1 |
| | | maturation | | | | |
| GO:0106015 | BP | negative regulation of inflammatory | 3 | 1 | 0.019346371 | 1 |
| | | response to wounding | | | | |
| GO:0005911 | CC | cell-cell junction | 494 | 14 | 0.019793634 | 1 |
| GO:0002889 | BP | regulation of immunoglobulin | 61 | 3 | 0.019846032 | 1 |
| | | mediated immune response | | | | |
| GO:0051645 | BP | Golgi localization | 14 | 2 | 0.019861439 | 1 |
| GO:0001886 | BP | endothelial cell morphogenesis | 11 | 2 | 0.019962078 | 1 |
| GO:0021859 | BP | pyramidal neuron differentiation | 13 | 2 | 0.019994956 | 1 |
| GO:0043550 | BP | regulation of lipid kinase activity | 68 | 4 | 0.020072578 | 1 |
| GO:0098739 | BP | import across plasma membrane | 188 | 7 | 0.020303963 | 1 |
| GO:0015182 | MF | L-asparagine transmembrane | 3 | 1 | 0.020526682 | 1 |
| | | transporter activity | | | | |
| GO:0045446 | BP | endothelial cell differentiation | 117 | 5 | 0.020529456 | 1 |
| GO:0019956 | MF | chemokine binding | 33 | 2 | 0.020895216 | 1 |
| GO:0002698 | BP | negative regulation of immune | 112 | 4 | 0.021275648 | 1 |
| | | effector process | | | | |
| GO:0061077 | BP | chaperone-mediated protein folding | 70 | 3 | 0.021413521 | 1 |
| GO:0001771 | BP | immunological synapse formation | 14 | 2 | 0.021702736 | 1 |
| GO:0002716 | BP | negative regulation of natural killer | 20 | 2 | 0.021780978 | 1 |
| | | cell mediated immunity | | | | |
| GO:0031115 | BP | negative regulation of microtubule | 14 | 2 | 0.021825472 | 1 |
| | | polymerization | | | | |
| GO:0033002 | BP | muscle cell proliferation | 232 | 7 | 0.021954983 | 1 |
| GO:1990544 | BP | mitochondrial ATP transmembrane | 4 | 1 | 0.021957493 | 1 |
| | | transport | | | | |
| GO:0034497 | BP | protein localization to phagophore | 17 | 2 | 0.022672239 | 1 |
| | | assembly site | | | | |
| GO:0006898 | BP | receptor-mediated endocytosis | 245 | 8 | 0.02273113 | 1 |
| GO:0099084 | BP | postsynaptic specialization | 33 | 3 | 0.022798726 | 1 |
| | | organization | | | | |
| GO:0017158 | BP | regulation of calcium ion-dependent | 40 | 3 | 0.023026998 | 1 |
| | | exocytosis | | | | |
| GO:0006857 | BP | oligopeptide transport | 16 | 2 | 0.023156239 | 1 |
| GO:0050613 | MF | delta14-sterol reductase activity | 2 | 1 | 0.023379435 | 1 |
| GO:1990001 | BP | inhibition of cysteine-type | 3 | 1 | 0.023560098 | 1 |
| | | endopeptidase activity involved in | | | | |
| | | apoptotic process | | | | |
| GO:0016324 | CC | apical plasma membrane | 356 | 10 | 0.023570952 | 1 |
| GO:0032102 | BP | negative regulation of response to | 429 | 10 | 0.023626064 | 1 |
| | | external stimulus | | | | |
| GO:0042982 | BP | amyloid precursor protein metabolic | 95 | 4 | 0.024111005 | 1 |
| | | process | | | | |
| GO:0047915 | MF | ganglioside galactosyltransferase | 1 | 1 | 0.024208808 | 1 |
| | | activity | | | | |
| GO:0090218 | BP | positive regulation of lipid kinase | 37 | 3 | 0.02442202 | 1 |
| | | activity | | | | |
| GO:0001570 | BP | vasculogenesis | 80 | 4 | 0.024467851 | 1 |
| GO:0061462 | BP | protein localization to lysosome | 46 | 3 | 0.024764052 | 1 |
| GO:1903318 | BP | negative regulation of protein | 28 | 2 | 0.025072722 | 1 |
| | | maturation | | | | |
| GO:0030277 | BP | maintenance of gastrointestinal | 22 | 2 | 0.025081237 | 1 |
| | | epithelium | | | | |
| GO:0016598 | BP | protein arginylation | 2 | 1 | 0.025159316 | 1 |
| GO:0042923 | MF | neuropeptide binding | 32 | 2 | 0.025200493 | 1 |
| GO:0019863 | MF | IgE binding | 5 | 1 | 0.025370829 | 1 |
| GO:1903709 | BP | uterine gland development | 1 | 1 | 0.025398899 | 1 |
| GO:0002830 | BP | positive regulation of type 2 immune | 17 | 2 | 0.0256565 | 1 |
| | | response | | | | |
| GO:0002376 | BP | immune system process | 2455 | 37 | 0.025676115 | 1 |
| GO:0033181 | CC | plasma membrane proton- | 2 | 1 | 0.025965834 | 1 |
| | | transporting V-type ATPase | | | | |
| | | complex | | | | |
| GO:1903306 | BP | negative regulation of regulated | 24 | 2 | 0.02654216 | 1 |
| | | secretory pathway | | | | |
| GO:0098552 | CC | side of membrane | 574 | 13 | 0.026734138 | 1 |
| GO:0001772 | CC | immunological synapse | 44 | 3 | 0.026861914 | 1 |
| GO:0090051 | BP | negative regulation of cell migration | 42 | 2 | 0.026876467 | 1 |
| | | involved in sprouting angiogenesis | | | | |
| GO:0042488 | BP | positive regulation of odontogenesis | 2 | 1 | 0.027108676 | 1 |
| | | of dentin-containing tooth | | | | |
| GO:0006955 | BP | immune response | 1577 | 24 | 0.027222717 | 1 |
| GO:0030124 | CC | AP-4 adaptor complex | 4 | 1 | 0.027251182 | 1 |
| GO:0051546 | BP | keratinocyte migration | 19 | 2 | 0.027537238 | 1 |
| GO:0032495 | BP | response to muramyl dipeptide | 20 | 2 | 0.02774257 | 1 |
| GO:0007603 | BP | phototransduction, visible light | 19 | 2 | 0.02797378 | 1 |
| GO:0030100 | BP | regulation of endocytosis | 208 | 7 | 0.028146564 | 1 |
| GO:0032692 | BP | negative regulation of interleukin-1 | 50 | 2 | 0.028153242 | 1 |
| | | production | | | | |
| GO:0019902 | MF | phosphatase binding | 193 | 7 | 0.028207934 | 1 |
| GO:0007159 | BP | leukocyte cell-cell adhesion | 378 | 9 | 0.028702811 | 1 |
| GO:0022407 | BP | regulation of cell-cell adhesion | 453 | 11 | 0.028747792 | 1 |
| GO:0045806 | BP | negative regulation of endocytosis | 56 | 3 | 0.02901504 | 1 |
| GO:0002456 | BP | T cell mediated immunity | 109 | 4 | 0.029177137 | 1 |
| GO:0008283 | BP | cell population proliferation | 2000 | 33 | 0.029204012 | 1 |
| GO:0070861 | BP | regulation of protein exit from | 25 | 2 | 0.02934141 | 1 |
| | | endoplasmic reticulum | | | | |
| GO:0031800 | MF | type 3 metabotropic glutamate | 3 | 1 | 0.029584359 | 1 |
| | | receptor binding | | | | |
| GO:0045747 | BP | positive regulation of Notch | 46 | 3 | 0.029616058 | 1 |
| | | signaling pathway | | | | |
| GO:0044062 | BP | regulation of excretion | 24 | 2 | 0.029745651 | 1 |
| GO:0035872 | BP | nucleotide-binding domain, leucine | 26 | 2 | 0.030089055 | 1 |
| | | rich repeat containing receptor | | | | |
| | | signaling pathway | | | | |
| GO:0031111 | BP | negative regulation of microtubule | 40 | 3 | 0.03019507 | 1 |
| | | polymerization or depolymerization | | | | |
| GO:1901201 | BP | regulation of extracellular matrix | 16 | 2 | 0.030885922 | 1 |
| | | assembly | | | | |
| GO:0034330 | BP | cell junction organization | 686 | 18 | 0.030964375 | 1 |
| GO:2000301 | BP | negative regulation of synaptic | 4 | 1 | 0.031028846 | 1 |
| | | vesicle exocytosis | | | | |
| GO:0150146 | BP | cell junction disassembly | 21 | 2 | 0.031061491 | 1 |
| GO:0050881 | BP | musculoskeletal movement | 54 | 3 | 0.031539666 | 1 |
| GO:0006622 | BP | protein targeting to lysosome | 23 | 2 | 0.031557806 | 1 |
| GO:0072382 | BP | minus-end-directed vesicle | 1 | 1 | 0.031720772 | 1 |
| | | transport along microtubule | | | | |
| GO:0042277 | MF | peptide binding | 303 | 8 | 0.031999443 | 1 |
| GO:1902897 | BP | regulation of postsynaptic density | 2 | 1 | 0.032028011 | 1 |
| | | protein 95 clustering | | | | |
| GO:0030011 | BP | maintenance of cell polarity | 19 | 2 | 0.032166236 | 1 |
| GO:0048514 | BP | blood vessel morphogenesis | 700 | 15 | 0.032589052 | 1 |
| GO:0061383 | BP | trabecula morphogenesis | 46 | 3 | 0.032592377 | 1 |
| GO:0005874 | CC | microtubule | 433 | 11 | 0.032622776 | 1 |
| GO:1905833 | BP | negative regulation of microtubule | 1 | 1 | 0.032940853 | 1 |
| | | nucleation | | | | |
| GO:0090410 | BP | malonate catabolic process | 1 | 1 | 0.033024863 | 1 |
| GO:1902600 | BP | proton transmembrane transport | 124 | 4 | 0.033103551 | 1 |
| GO:0008532 | MF | N-acetyllactosaminide beta-1,3-N- | 5 | 1 | 0.033126954 | 1 |
| | | acetylglucosaminyltransferase | | | | |
| | | activity | | | | |
| GO:0046622 | BP | positive regulation of organ growth | 52 | 3 | 0.033550552 | 1 |
| GO:0045824 | BP | negative regulation of innate | 73 | 3 | 0.033554493 | 1 |
| | | immune response | | | | |
| GO:0035338 | BP | long-chain fatty-acyl-CoA | 19 | 2 | 0.033631244 | 1 |
| | | biosynthetic process | | | | |
| GO:0042110 | BP | T cell activation | 503 | 11 | 0.034091977 | 1 |
| GO:0061647 | BP | histone H3-K9 modification | 50 | 3 | 0.034148773 | 1 |
| GO:0050254 | MF | rhodopsin kinase activity | 3 | 1 | 0.034483349 | 1 |
| GO:0009582 | BP | detection of abiotic stimulus | 137 | 5 | 0.034672536 | 1 |
| GO:1900248 | BP | negative regulation of cytoplasmic | 2 | 1 | 0.034820394 | 1 |
| | | translational elongation | | | | |
| GO:0007219 | BP | Notch signaling pathway | 180 | 6 | 0.035379261 | 1 |
| GO:1990573 | BP | potassium ion import across plasma | 50 | 3 | 0.035805654 | 1 |
| | | membrane | | | | |
| GO:0003281 | BP | ventricular septum development | 73 | 4 | 0.035808221 | 1 |
| GO:0045216 | BP | cell-cell junction organization | 205 | 7 | 0.03595016 | 1 |
| GO:0036297 | BP | interstrand cross-link repair | 31 | 2 | 0.036186934 | 1 |
| GO:0060977 | BP | coronary vasculature | 19 | 2 | 0.036630036 | 1 |
| | | morphogenesis | | | | |
| GO:0030155 | BP | regulation of cell adhesion | 756 | 17 | 0.036983212 | 1 |
| GO:0016241 | BP | regulation of macroautophagy | 158 | 5 | 0.037004582 | 1 |
| GO:0002064 | BP | epithelial cell development | 203 | 7 | 0.037021714 | 1 |
| GO:0090101 | BP | negative regulation of | 135 | 5 | 0.037068438 | 1 |
| | | transmembrane receptor protein | | | | |
| | | serine/threonine kinase signaling | | | | |
| | | pathway | | | | |
| GO:0051338 | BP | regulation of transferase activity | 876 | 18 | 0.037330026 | 1 |
| GO:0031110 | BP | regulation of microtubule | 88 | 4 | 0.037605687 | 1 |
| | | polymerization or depolymerization | | | | |
| GO:0001816 | BP | cytokine production | 820 | 14 | 0.037714043 | 1 |
| GO:0098728 | BP | germline stem cell asymmetric | 2 | 1 | 0.03796328 | 1 |
| | | division | | | | |
| GO:0009583 | BP | detection of light stimulus | 65 | 3 | 0.03830637 | 1 |
| GO:0005938 | CC | cell cortex | 309 | 10 | 0.038343836 | 1 |
| GO:0032689 | BP | negative regulation of interferon- | 41 | 2 | 0.038751295 | 1 |
| | | gamma production | | | | |
| GO:0033218 | MF | amide binding | 381 | 9 | 0.038939403 | 1 |
| GO:0042997 | BP | negative regulation of Golgi to | 5 | 1 | 0.039314073 | 1 |
| | | plasma membrane protein transport | | | | |
| GO:0070160 | CC | tight junction | 132 | 5 | 0.039611972 | 1 |
| GO:0003333 | BP | amino acid transmembrane | 100 | 4 | 0.039711058 | 1 |
| | | transport | | | | |
| GO:1903408 | BP | positive regulation of P-type | 3 | 1 | 0.039725299 | 1 |
| | | sodium:potassium-exchanging | | | | |
| | | transporter activity | | | | |
| GO:0099081 | CC | supramolecular polymer | 985 | 19 | 0.039826427 | 1 |
| GO:0051548 | BP | negative regulation of keratinocyte | 2 | 1 | 0.039912398 | 1 |
| | | migration | | | | |
| GO:0002052 | BP | positive regulation of neuroblast | 24 | 2 | 0.039932665 | 1 |
| | | proliferation | | | | |
| GO:0001653 | MF | peptide receptor activity | 151 | 4 | 0.04068086 | 1 |
| GO:0007517 | BP | muscle organ development | 332 | 9 | 0.04074387 | 1 |
| GO:0019904 | MF | protein domain specific binding | 68 | 16 | 0.040870547 | 1 |
| GO:0001910 | BP | regulation of leukocyte mediated | 85 | 3 | 0.040984281 | 1 |
| | | cytotoxicity | | | | |
| GO:0036409 | CC | histone H3-K14 acetyltransferase | 2 | 1 | 0.041267418 | 1 |
| | | complex | | | | |
| GO:0016247 | MF | channel regulator activity | 141 | 5 | 0.041367959 | 1 |
| GO:0060648 | BP | mammary gland bud | 3 | 1 | 0.041609409 | 1 |
| | | morphogenesis | | | | |
| GO:0050750 | MF | low-density lipoprotein particle | 24 | 2 | 0.041646684 | 1 |
| | | receptor binding | | | | |
| GO:0002768 | BP | immune response-regulating cell | 253 | 7 | 0.041829159 | 1 |
| | | surface receptor signaling pathway | | | | |
| GO:0007202 | BP | activation of phospholipase C | 30 | 2 | 0.041852108 | 1 |
| | | activity | | | | |
| GO:0002720 | BP | positive regulation of cytokine | 67 | 3 | 0.041896863 | 1 |
| | | production involved in immune | | | | |
| | | response | | | | |
| GO:0035783 | BP | CD4-positive, alpha-beta T cell | 3 | 1 | 0.042024434 | 1 |
| | | costimulation | | | | |
| GO:0000706 | BP | meiotic DNA double-strand break | 4 | 1 | 0.042305226 | 1 |
| | | processing | | | | |
| GO:1902230 | BP | negative regulation of intrinsic | 30 | 2 | 0.042340468 | 1 |
| | | apoptotic signaling pathway in | | | | |
| | | response to DNA damage | | | | |
| GO:0034329 | BP | cell junction assembly | 420 | 12 | 0.042598548 | 1 |
| GO:0060713 | BP | labyrinthine layer morphogenesis | 22 | 2 | 0.04260272 | 1 |
| GO:0060193 | BP | positive regulation of lipase activity | 68 | 3 | 0.042829842 | 1 |
| GO:0099080 | CC | supramolecular complex | 1316 | 23 | 0.042837997 | 1 |
| GO:0060039 | BP | pericardium development | 19 | 2 | 0.04299296 | 1 |
| GO:0005672 | CC | transcription factor TFIIA complex | 5 | 1 | 0.043048086 | 1 |
| GO:0002684 | BP | positive regulation of immune | 840 | 15 | 0.043070066 | 1 |
| | | system process | | | | |
| GO:0030197 | MF | extracellular matrix constituent, | 4 | 1 | 0.043216266 | 1 |
| | | lubricant activity | | | | |
| GO:0003231 | BP | cardiac ventricle development | 126 | 5 | 0.04379506 | 1 |
| GO:0050790 | BP | regulation of catalytic activity | 2325 | 39 | 0.044128756 | 1 |
| GO:0042379 | MF | chemokine receptor binding | 69 | 2 | 0.044210429 | 1 |
| GO:0023024 | MF | MHC class I protein complex | 6 | 1 | 0.044326322 | 1 |
| | | binding | | | | |
| GO:0036092 | BP | phosphatidylinositol-3-phosphate | 21 | 2 | 0.044346057 | 1 |
| | | biosynthetic process | | | | |
| GO:0036003 | BP | positive regulation of transcription | 24 | 2 | 0.044508219 | 1 |
| | | from RNA polymerase II promoter in | | | | |
| | | response to stress | | | | |
| GO:0050863 | BP | regulation of T cell activation | 339 | 8 | 0.044540506 | 1 |
| GO:0003017 | BP | lymph circulation | 2 | 1 | 0.044623994 | 1 |
| GO:1900226 | BP | negative regulation of NLRP3 | 7 | 1 | 0.044686109 | 1 |
| | | inflammasore complex assembly | | | | |
| GO:0045861 | BP | negative regulation of proteolysis | 346 | 7 | 0.044862316 | 1 |
| GO:0070383 | BP | DNA cytosine deamination | 9 | 1 | 0.044877628 | 1 |
| GO:0140677 | MF | molecular function activator activity | 34 | 2 | 0.045137542 | 1 |
| GO:0022612 | BP | gland morphogenesis | 123 | 5 | 0.04517429 | 1 |
| GO:0031348 | BP | negative regulation of defense | 268 | 6 | 0.045223123 | 1 |
| | | response | | | | |
| GO:0033591 | BP | response to L-ascorbic acid | 5 | 1 | 0.045618498 | 1 |
| GO:0110135 | BP | Norrin signaling pathway | 3 | 1 | 0.045696794 | 1 |
| GO:0045178 | CC | basal part of cell | 268 | 8 | 0.045794717 | 1 |
| GO:0001568 | BP | blood vessel development | 782 | 16 | 0.045931506 | 1 |
| GO:0021699 | BP | cerebellar cortex maturation | 3 | 1 | 0.046001474 | 1 |
| GO:0006865 | BP | amino acid transport | 145 | 5 | 0.046087047 | 1 |
| GO:0002434 | BP | immune complex clearance | 5 | 1 | 0.046148909 | 1 |
| GO:0097225 | CC | sperm midpiece | 38 | 2 | 0.046201346 | 1 |
| GO:0097038 | CC | perinuclear endoplasmic reticulum | 21 | 2 | 0.046554734 | 1 |
| GO:0006007 | BP | glucose catabolic process | 20 | 2 | 0.046707759 | 1 |
| GO:0017134 | MF | fibroblast growth factor binding | 23 | 2 | 0.046732727 | 1 |
| GO:0002786 | BP | regulation of antibacterial peptide | 6 | 1 | 0.04684374 | 1 |
| | | production | | | | |
| GO:0030371 | MF | translation repressor activity | 287 | 3 | 0.04685252 | 1 |
| GO:0042987 | BP | amyloid precursor protein catabolic | 68 | 3 | 0.0469205 | 1 |
| | | process | | | | |
| GO:1902963 | BP | negative regulation of | 2 | 1 | 0.046938739 | 1 |
| | | metalloendopeptidase activity | | | | |
| | | involved in amyloid precursor | | | | |
| | | protein catabolic process | | | | |
| GO:0048659 | BP | smooth muscle cell proliferation | 172 | 5 | 0.047303425 | 1 |
| GO:0006950 | BP | response to stress | 3735 | 51 | 0.047318886 | 1 |
| GO:2000765 | BP | regulation of cytoplasmic translation | 28 | 2 | 0.047440932 | 1 |
| GO:0002520 | BP | immune system development | 955 | 18 | 0.047496047 | 1 |
| GO:2000342 | BP | negative regulation of chemokine | 6 | 1 | 0.047618504 | 1 |
| | | (C-X-C motif) ligand 2 production | | | | |
| GO:2000660 | BP | negative regulation of interleukin-1- | 7 | 1 | 0.047639513 | 1 |
| | | mediated signaling pathway | | | | |
| GO:0023026 | MF | MHC class II protein complex | 25 | 2 | 0.047689518 | 1 |
| | | binding | | | | |
| GO:0019960 | MF | C-X3-C chemokine binding | 5 | 1 | 0.047901685 | 1 |
| GO:0043154 | BP | negative regulation of cysteine-type | 76 | 3 | 0.047917118 | 1 |
| | | endopeptidase activity involved in | | | | |
| | | apoptotic process | | | | |
| GO:0016941 | MF | natriuretic peptide receptor activity | 4 | 1 | 0.047998626 | 1 |
| GO:0010747 | BP | positive regulation of long-chain | 2 | 1 | 0.048003448 | 1 |
| | | fatty acid import across plasma | | | | |
| | | membrane | | | | |
| GO:0015179 | MF | L-amino acid transmembrane | 61 | 3 | 0.048119686 | 1 |
| | | transporter activity | | | | |
| GO:0070482 | BP | response to oxygen levels | 320 | 8 | 0.048257172 | 1 |
| GO:0004530 | MF | deoxyribonuclease I activity | 5 | 1 | 0.048276252 | 1 |
| GO:0042325 | BP | regulation of phosphorylation | 1242 | 23 | 0.048289512 | 1 |
| GO:0001576 | BP | globoside biosynthetic process | 2 | 1 | 0.048573217 | 1 |
| GO:0042105 | CC | alpha-beta T cell receptor complex | 5 | 1 | 0.048817003 | 1 |
| GO:0008593 | BP | regulation of Notch signaling | 95 | 4 | 0.048878217 | 1 |
| | | pathway | | | | |
| GO:0003150 | BP | muscular septum morphogenesis | 4 | 1 | 0.048904496 | 1 |
| GO:0050778 | BP | positive regulation of immune | 513 | 10 | 0.049110175 | 1 |
| | | response | | | | |
| GO:0035176 | BP | social behavior | 52 | 3 | 0.049322027 | 1 |
| GO:1903849 | BP | positive regulation of aorta | 2 | 1 | 0.049352817 | 1 |
| | | morphogenesis | | | | |
| GO:1905039 | BP | carboxylic acid transmembrane | 152 | 5 | 0.049592728 | 1 |
| | | transport | | | | |
| GO:0002920 | BP | regulation of humoral immune | 45 | 2 | 0.049615091 | 1 |
| | | response | | | | |
| GO:0032480 | BP | negative regulation of type I | 32 | 2 | 0.049671349 | 1 |
| | | interferon production | | | | |
| GO:0032396 | MF | inhibitory MHC class I receptor | 11 | 1 | 0.049713917 | 1 |
| | | activity | | | | |
| GO:0072679 | BP | thymocyte migration | 6 | 1 | 0.04975941 | 1 |
| GO:0007017 | BP | microtubule-based process | 887 | 17 | 0.049896362 | 1 |
| GO:0034157 | BP | positive regulation of toll-like | 6 | 1 | 0.049938322 | 1 |
| | | receptor 7 signaling pathway | | | | |

TABLE 11D
GO terms associated with differentially methylated genes

| ONTOLOGY | | TERM | N | # | P. D. M. | FDR |
|---|---|---|---|---|---|---|
| GO:0002376 | BP | immune system process | 2455 | 24 | 4.68252933408645e−05 | 0.213673179 |
| GO:0042110 | BP | T cell activation | 503 | 9 | 0.000399939 | 0.531925478 |
| GO:0001775 | BP | cell activation | 1017 | 13 | 0.000483576 | 0.53287668 |
| GO:0002520 | BP | immune system development | 955 | 13 | 0.000512374 | 0.53287668 |
| GO:0030097 | BP | hemopoiesis | 856 | 12 | 0.000659675 | 0.557449552 |
| GO:0050867 | BP | positive regulation of cell | 357 | 7 | 0.001057763 | 0.67038665 |
| | | activation | | | | |
| GO:0045321 | BP | leukocyte activation | 876 | 11 | 0.001490143 | 0.777732604 |
| GO:0070938 | CC | contractile ring | 10 | 2 | 0.001495011 | 0.777732604 |
| GO:0002250 | BP | adaptive immune response | 438 | 7 | 0.001841986 | 0.840535019 |
| GO:0045785 | BP | positive regulation of cell | 442 | 8 | 0.002167197 | 0.932957783 |
| | | adhesion | | | | |
| GO:0003334 | BP | keratinocyte development | 13 | 2 | 0.002292678 | 0.938214099 |
| GO:0009187 | BP | cyclic nucleotide metabolic | 39 | 3 | 0.002441434 | 0.938214099 |
| | | process | | | | |
| GO:0060135 | BP | maternal process involved in | 58 | 3 | 0.002552618 | 0.938214099 |
| | | female pregnancy | | | | |
| GO:0030135 | CC | coated vesicle | 286 | 6 | 0.002814543 | 0.938214099 |
| GO:0002696 | BP | positive regulation of leukocyte | 342 | 6 | 0.004425743 | 1 |
| | | activation | | | | |
| GO:0008270 | MF | zinc ion binding | 807 | 10 | 0.00453604 | 1 |
| GO:0030865 | BP | cortical cytoskeleton organization | 61 | 3 | 0.004770302 | 1 |
| GO:0042924 | MF | neuromedin U binding | 2 | 1 | 0.004955973 | 1 |
| GO:0050865 | BP | regulation of cell activation | 602 | 8 | 0.005105031 | 1 |
| GO:0048663 | BP | neuron fate commitment | 63 | 3 | 0.005183146 | 1 |
| GO:0046631 | BP | alpha-beta T cell activation | 156 | 4 | 0.006794479 | 1 |
| GO:0002460 | BP | adaptive immune response based | 292 | 5 | 0.007005652 | 1 |
| | | on somatic recombination of | | | | |
| | | immune receptors built from lg | | | | |
| | | superfamily domains | | | | |
| GO:0046914 | MF | transition metal ion binding | 1077 | 11 | 0.008155437 | 1 |
| GO:0030217 | BP | T cell differentiation | 263 | 5 | 0.008225708 | 1 |
| GO:0071944 | CC | cell periphery | 5911 | 38 | 0.008455538 | 1 |
| GO:0031224 | CC | intrinsic component of membrane | 5359 | 32 | 0.008583897 | 1 |
| GO:0031227 | CC | intrinsic component of endoplasmic | 170 | 4 | 0.008722349 | 1 |
| | | reticulum membrane | | | | |
| GO:0002262 | BP | myeloid cell homeostasis | 160 | 4 | 0.009274888 | 1 |
| GO:0097110 | MF | scaffold protein binding | 67 | 3 | 0.009278876 | 1 |
| GO:0002252 | BP | immune effector process | 594 | 7 | 0.009788319 | 1 |
| GO:0002694 | BP | regulation of leukocyte activation | 550 | 7 | 0.010389583 | 1 |
| GO:0048872 | BP | homeostasis of number of cells | 282 | 5 | 0.010805866 | 1 |
| GO:0022803 | MF | passive transmembrane transporter | 485 | 7 | 0.011139522 | 1 |
| | | activity | | | | |
| GO:0003401 | BP | axis elongation | 27 | 2 | 0.01118224 | 1 |
| GO:0071168 | BP | protein localization to chromatin | 32 | 2 | 0.011252785 | 1 |
| GO:0044706 | BP | multi-multicellular organism process | 213 | 4 | 0.011735944 | 1 |
| GO:0005886 | CC | plasma membrane | 5427 | 35 | 0.01174312 | 1 |
| GO:0048821 | BP | erythrocyte development | 37 | 2 | 0.011956529 | 1 |
| GO:0030099 | BP | myeloid cell differentiation | 388 | 6 | 0.012391967 | 1 |
| GO:0042726 | BP | flavin-containing compound | 6 | 1 | 0.012834856 | 1 |
| | | metabolic process | | | | |
| GO:0010043 | BP | response to zinc ion | 53 | 2 | 0.013335411 | 1 |
| GO:0048513 | BP | animal organ development | 3452 | 26 | 0.013587033 | 1 |
| GO:0005201 | MF | extracellular matrix structural | 171 | 4 | 0.013861085 | 1 |
| | | constituent | | | | |
| GO:0046058 | BP | cAMP metabolic process | 22 | 2 | 0.014062199 | 1 |
| GO:0045165 | BP | cell fate commitment | 266 | 5 | 0.014878462 | 1 |
| GO:0002443 | BP | leukocyte mediated immunity | 378 | 5 | 0.015718819 | 1 |
| GO:0001913 | BP | T cell mediated cytotoxicity | 48 | 2 | 0.016073738 | 1 |
| GO:0018406 | BP | protein C-linked glycosylation via 2′- | 5 | 1 | 0.016719317 | 1 |
| | | alpha-mannosyl-L-tryptophan | | | | |
| GO:0030133 | CC | transport vesicle | 397 | 6 | 0.017239659 | 1 |
| GO:0019722 | BP | calcium-mediated signaling | 202 | 4 | 0.017381358 | 1 |
| GO:0030155 | BP | regulation of cell adhesion | 756 | 9 | 0.017463605 | 1 |
| GO:0019724 | BP | B cell mediated immunity | 143 | 3 | 0.017595245 | 1 |
| GO:0006955 | BP | immune response | 1577 | 12 | 0.017630148 | 1 |
| GO:0098552 | CC | side of membrane | 574 | 7 | 0.018135486 | 1 |
| GO:0019932 | BP | second-messenger-mediated | 302 | 5 | 0.018238486 | 1 |
| | | signaling | | | | |
| GO:0032182 | MF | ubiquitin-like protein binding | 116 | 3 | 0.018706386 | 1 |
| GO:0050776 | BP | regulation of immune response | 810 | 8 | 0.01881816 | 1 |
| GO:0009986 | CC | cell surface | 861 | 9 | 0.018825937 | 1 |
| GO:0031428 | CC | box C/D RNP complex | 7 | 1 | 0.018854773 | 1 |
| GO:0043266 | BP | regulation of potassium ion | 109 | 3 | 0.018918254 | 1 |
| | | transport | | | | |
| GO:0030666 | CC | endocytic vesicle membrane | 190 | 4 | 0.019345552 | 1 |
| GO:0070004 | MF | cysteine-type exopeptidase activity | 5 | 1 | 0.019391363 | 1 |
| GO:0019900 | MF | kinase binding | 758 | 9 | 0.019801137 | 1 |
| GO:0032355 | BP | response to estradiol | 123 | 3 | 0.022472109 | 1 |
| GO:0010563 | BP | negative regulation of phosphorus | 439 | 6 | 0.022532888 | 1 |
| | | metabolic process | | | | |
| GO:0045912 | BP | negative regulation of carbohydrate | 51 | 2 | 0.022749441 | 1 |
| | | metabolic process | | | | |
| GO:0140352 | BP | export from cell | 845 | 9 | 0.022833346 | 1 |
| GO:0003012 | BP | muscle system process | 446 | 6 | 0.023121799 | 1 |
| GO:0002449 | BP | lymphocyte mediated immunity | 286 | 4 | 0.023559745 | 1 |
| GO:0048538 | BP | thymus development | 45 | 2 | 0.023988583 | 1 |
| GO:0034762 | BP | regulation of transmembrane | 569 | 1 | 0.024398238 | 1 |
| | | transport | | | | |
| GO:0005768 | CC | endosome | 986 | 10 | 0.025220492 | 1 |
| GO:0043209 | CC | myelin sheath | 44 | 2 | 0.02557631 | 1 |
| GO:1902041 | BP | regulation of extrinsic apoptotic | 50 | 2 | 0.026136698 | 1 |
| | | signaling pathway via death domain | | | | |
| | | receptors | | | | |
| GO:0030658 | CC | transport vesicle membrane | 200 | 4 | 0.026534412 | 1 |
| GO:0031226 | CC | intrinsic component of plasma | 1718 | 14 | 0.026606587 | 1 |
| | | membrane | | | | |
| GO:0035616 | BP | histone H2B conserved C-terminal | 4 | 1 | 0.026642735 | 1 |
| | | lysine deubiquitination | | | | |
| GO:0001541 | BP | ovarian follicle development | 57 | 2 | 0.026744399 | 1 |
| GO:0019899 | MF | enzyme binding | 2048 | 17 | 0.026977576 | 1 |
| GO:0040029 | BP | regulation of gene expression, | 141 | 3 | 0.027401263 | 1 |
| | | epigenetic | | | | |
| GO:0071692 | BP | protein localization to extracellular | 355 | 5 | 0.027411309 | 1 |
| | | region | | | | |
| GO:0002682 | BP | regulation of immune system | 1358 | 11 | 0.027661749 | 1 |
| | | process | | | | |
| GO:0010572 | BP | positive regulation of platelet | 9 | 1 | 0.027867608 | 1 |
| | | activation | | | | |
| GO:0015318 | MF | inorganic molecular entity | 716 | 8 | 0.02804362 | 1 |
| | | transmembrane transporter activity | | | | |
| GO:0016020 | CC | membrane | 9099 | 49 | 0.028117099 | 1 |
| GO:0003823 | MF | antigen binding | 59 | 2 | 0.028218739 | 1 |
| GO:2000354 | BP | regulation of ovarian follicle | 6 | 1 | 0.029293124 | 1 |
| | | development | | | | |
| GO:0030139 | CC | endocytic vesicle | 338 | 5 | 0.029377409 | 1 |
| GO:0051018 | MF | protein kinase A binding | 50 | 2 | 0.029576823 | 1 |
| GO:0045058 | BP | T cell selection | 49 | 2 | 0.030614959 | 1 |
| GO:0002064 | BP | epithelial cell development | 203 | 4 | 0.030861778 | 1 |
| GO:0007195 | BP | adenylate cyclase-inhibiting | 6 | 1 | 0.031512883 | 1 |
| | | dopamine receptor signaling | | | | |
| | | pathway | | | | |
| GO:0043647 | BP | inositol phosphate metabolic | 55 | 2 | 0.031601368 | 1 |
| | | process | | | | |
| GO:0002263 | BP | cell activation involved in immune | 287 | 4 | 0.032051468 | 1 |
| | | response | | | | |
| GO:0097708 | CC | intracellular vesicle | 2433 | 18 | 0.03207883 | 1 |
| GO:0001221 | MF | transcription coregulator binding | 108 | 3 | 0.032163383 | 1 |
| GO:0001222 | MF | transcription corepressor binding | 44 | 2 | 0.032565067 | 1 |
| GO:0012506 | CC | vesicle membrane | 1180 | 11 | 0.032964249 | 1 |
| GO:0001665 | MF | alpha-N-acetylgalactosaminide | 6 | 1 | 0.033257661 | 1 |
| | | alpha-2,6-sialyltransferase activity | | | | |
| GO:0048863 | BP | stem cell differentiation | 210 | 4 | 0.033511889 | 1 |
| GO:0021764 | BP | amygdala development | 5 | 1 | 0.034050707 | 1 |
| GO:0042476 | BP | odontogenesis | 130 | 3 | 0.034204502 | 1 |
| GO:0050732 | BP | negative regulation of peptidyl- | 58 | 2 | 0.034313101 | 1 |
| | | tyrosine phosphorylation | | | | |
| GO:0031080 | CC | nuclear pore outer ring | 9 | 1 | 0.03434151 | 1 |
| GO:0050878 | BP | regulation of body fluid levels | 379 | 5 | 0.034602775 | 1 |
| GO:0001099 | MF | basal RNA polymerase II | 72 | 2 | 0.034659647 | 1 |
| | | transcription machinery binding | | | | |
| GO:0034765 | BP | regulation of ion transmembrane | 472 | 6 | 0.034710384 | 1 |
| | | transport | | | | |
| GO:2000241 | BP | regulation of reproductive process | 175 | 3 | 0.034954723 | 1 |
| GO:0046501 | BP | protoporphyrinogen IX metabolic | 11 | 1 | 0.035156371 | 1 |
| | | process | | | | |
| GO:0045580 | BP | regulation of T cell differentiation | 151 | 3 | 0.037612497 | 1 |
| GO:0032940 | BP | secretion by cell | 780 | 8 | 0.037994395 | 1 |
| GO:0098791 | CC | Golgi apparatus subcompartment | 377 | 5 | 0.038008734 | 1 |
| GO:0034703 | CC | cation channel complex | 220 | 4 | 0.038867631 | 1 |
| GO:0021872 | BP | forebrain generation of neurons | 50 | 2 | 0.039450273 | 1 |
| GO:0010517 | BP | regulation of phospholipase activity | 66 | 2 | 0.039815481 | 1 |
| GO:0005261 | MF | cation channel activity | 342 | 5 | 0.039834107 | 1 |
| GO:1904026 | BP | regulation of collagen fibril | 8 | 1 | 0.039940164 | 1 |
| | | organization | | | | |
| GO:0086002 | BP | cardiac muscle cell action potential | 53 | 2 | 0.040473185 | 1 |
| | | involved in contraction | | | | |
| GO:0007343 | BP | egg activation | 10 | 1 | 0.040494469 | 1 |
| GO:0010467 | BP | gene expression | 6619 | 33 | 0.040536231 | 1 |
| GO:0002819 | BP | regulation of adaptive immune | 185 | 3 | 0.040613384 | 1 |
| | | response | | | | |
| GO:0035098 | CC | ESC/E(Z) complex | 9 | 1 | 0.040848796 | 1 |
| GO:0043353 | BP | enucleate erythrocyte differentiation | 9 | 1 | 0.040869865 | 1 |
| GO:1903764 | BP | regulation of potassium ion export | 8 | 1 | 0.04121234 | 1 |
| | | across plasma membrane | | | | |
| GO:0005770 | CC | late endosome | 277 | 4 | 0.041345122 | 1 |
| GO:0006893 | BP | Golgi to plasma membrane | 59 | 2 | 0.041435784 | 1 |
| | | transport | | | | |
| GO:0001833 | BP | inner cell mass cell proliferation | 13 | 1 | 0.041533115 | 1 |
| GO:0007548 | BP | sex differentiation | 279 | 4 | 0.041550009 | 1 |
| GO:0033093 | CC | Weibel-Palade body | 6 | 1 | 0.042659167 | 1 |
| GO:0002521 | BP | leukocyte differentiation | 551 | 6 | 0.04266427 | 1 |
| GO:0046661 | BP | male sex differentiation | 163 | 3 | 0.042965249 | 1 |
| GO:2000649 | BP | regulation of sodium ion | 57 | 2 | 0.042988863 | 1 |
| | | transmembrane transporter activity | | | | |
| GO:0036500 | BP | ATF6-mediated unfolded protein | 7 | 1 | 0.043051885 | 1 |
| | | response | | | | |
| GO:0044262 | BP | cellular carbohydrate metabolic | 284 | 4 | 0.043121073 | 1 |
| | | process | | | | |
| GO:0052658 | MF | inositol-1,4,5-trisphosphate 5- | 7 | 1 | 0.043173362 | 1 |
| | | phosphatase activity | | | | |
| GO:0005351 | MF | carbohydrate:proton symporter | 7 | 1 | 0.04337399 | 1 |
| | | activity | | | | |
| GO:0043299 | BP | leukocyte degranulation | 75 | 2 | 0.044189824 | 1 |
| GO:0002292 | BP | T cell differentiation involved in | 76 | 2 | 0.044373682 | 1 |
| | | immune response | | | | |
| GO:0042698 | BP | ovulation cycle | 70 | 2 | 0.044426656 | 1 |
| GO:1902105 | BP | regulation of leukocyte | 282 | 4 | 0.044427367 | 1 |
| | | differentiation | | | | |
| GO:0016840 | MF | carbon-nitrogen lyase activity | 13 | 1 | 0.044628255 | 1 |
| GO:0043433 | BP | negative regulation of DNA-binding | 188 | 3 | 0.044649758 | 1 |
| | | transcription factor activity | | | | |
| GO:0051179 | BP | localization | 6104 | 37 | 0.044963022 | 1 |
| GO:0098609 | BP | cell-cell adhesion | 920 | 9 | 0.044998482 | 1 |
| GO:0075294 | BP | positive regulation by symbiont of | 11 | 1 | 0.045238166 | 1 |
| | | entry into host | | | | |
| GO:0098796 | CC | membrane protein complex | 1155 | 10 | 0.045560798 | 1 |
| GO:0002285 | BP | lymphocyte activation involved in | 199 | 3 | 0.046075462 | 1 |
| | | immune response | | | | |
| GO:0010008 | CC | endosome membrane | 518 | 6 | 0.046087958 | 1 |
| GO:2001187 | BP | positive regulation of CD8-positive, | 9 | 1 | 0.046306087 | 1 |
| | | alpha-beta T cell activation | | | | |
| GO:0048016 | BP | inositol phosphate-mediated | 56 | 2 | 0.046891226 | 1 |
| | | signaling | | | | |
| GO:0007032 | BP | endosome organization | 85 | 2 | 0.047024829 | 1 |
| GO:0030853 | BP | negative regulation of granulocyte | 8 | 1 | 0.047342439 | 1 |
| | | differentiation | | | | |
| GO:0030199 | BP | collagen fibril organization | 61 | 2 | 0.047406948 | 1 |
| GO:0010959 | BP | regulation of metal ion transport | 400 | 5 | 0.047529171 | 1 |
| GO:0061133 | MF | endopeptidase activator activity | 13 | 1 | 0.04806015 | 1 |
| GO:0007155 | BP | cell adhesion | 1500 | 13 | 0.048144492 | 1 |
| GO:0002468 | BP | dendritic cell antigen processing | 14 | 1 | 0.048240077 | 1 |
| | | and presentation | | | | |
| GO:0035748 | CC | myelin sheath abaxonal region | 6 | 1 | 0.048267756 | 1 |
| GO:0035701 | BP | hematopoietic stem cell migration | 8 | 1 | 0.048525252 | 1 |
| GO:0047144 | MF | 2-acylglycerol-3-phosphate O- | 9 | 1 | 0.048672096 | 1 |
| | | acyltransferase activity | | | | |
| GO:0043627 | BP | response to estrogen | 68 | 2 | 0.048801205 | 1 |
| GO:0005764 | CC | lysosome | 720 | 7 | 0.048807065 | 1 |
| GO:0072665 | BP | protein localization to vacuole | 68 | 2 | 0.048865681 | 1 |
| GO:0097550 | CC | transcription preinitiation complex | 10 | 1 | 0.04904877 | 1 |
| GO:0031996 | MF | thioesterase binding | 11 | 1 | 0.049185309 | 1 |
| GO:0014870 | BP | response to muscle inactivity | 9 | 1 | 0.049472448 | 1 |
| GO:0019865 | MF | immunoglobulin binding | 24 | 1 | 0.049750907 | 1 |
| GO:0008499 | MF | UDP-galactose:beta-N- | 14 | 1 | 0.049754114 | 1 |
| | | acetylglucosamine beta-1,3- | | | | |
| | | galactosyltransferase activity | | | | |
| GO:0030056 | CC | hemidesmosome | 6 | 1 | 0.049810758 | 1 |
| GO:0070294 | BP | renal sodium ion absorption | 8 | 1 | 0.049858896 | 1 |

TABLE 11E
GO terms associated with differentially methylated genes

| ONTOLOGY | | TERM | N | DE | P.DE | FDR |
|---|---|---|---|---|---|---|
| GO:0002682 | BP | regulation of immune | 1358 | 58 | 1.232982014191e−07 | 0.001627418 |
| | | system process | | | | |
| GO:0045321 | BP | leukocyte activation | 876 | 44 | 2.14035477092829e−07 | 0.001627418 |
| GO:0002376 | BP | immune system process | 2455 | 87 | 2.42485668202827e−07 | 0.001627418 |
| GO:0046649 | BP | lymphocyte activation | 717 | 39 | 2.8531176545826e−07 | 0.001627418 |
| GO:0050865 | BP | regulation of cell | 602 | 34 | 5.46431081998438e−07 | 0.002493474 |
| | | activation | | | | |
| GO:0050863 | BP | regulation of T cell | 339 | 24 | 6.67455998109249e−07 | 0.002538113 |
| | | activation | | | | |
| GO:0042110 | BP | T cell activation | 503 | 30 | 1.19303322286559e−06 | 0.003485074 |
| GO:0051249 | BP | regulation of lymphocyte | 455 | 28 | 1.22197530730965e−06 | 0.003485074 |
| | | activation | | | | |
| GO:0002694 | BP | regulation of leukocyte | 550 | 31 | 1.4733816303661e−06 | 0.003735186 |
| | | activation | | | | |
| GO:0022407 | BP | regulation of cell-cell | 453 | 29 | 2.14144102847023e−06 | 0.004885912 |
| | | adhesion | | | | |
| GO:0001775 | BP | cell activation | 1017 | 46 | 2.92152428663093e−06 | 0.006059773 |
| GO:1903037 | BP | regulation of leukocyte cell- | 341 | 22 | 6.39609461938174e−06 | 0.012161108 |
| | | cell adhesion | | | | |
| GO:0002250 | BP | adaptive immune response | 438 | 24 | 8.01119462441313e−06 | 0.014060263 |
| GO:0007159 | BP | leukocyte cell-cell | 378 | 23 | 1.06473373848136e−05 | 0.017352118 |
| | | adhesion | | | | |
| GO:0002684 | BP | positive regulation of | 840 | 37 | 1.86945982892847e−05 | 0.02843573 |
| | | immune system process | | | | |
| GO:0002366 | BP | leukocyte activation | 283 | 18 | 2.43823329847659e−05 | 0.034769207 |
| | | involved in immune | | | | |
| | | response | | | | |
| GO:0002263 | BP | cell activation involved in | 287 | 18 | 3.49425569737905e−05 | 0.046897022 |
| | | immune response | | | | |

TABLE 11F
GO terms associated with differentially methylated genes

| ONTOLOGY | | TERM | N | DE | P.DE | FDR |
|---|---|---|---|---|---|---|
| GO:0002250 | BP | adaptive immune response | 438 | 31 | 6.21E−09 | 8.49E−05 |
| GO:0002376 | BP | immune system process | 2455 | 97 | 7.45E−09 | 8.49E−05 |
| GO:0051239 | BP | regulation of multicellular organismal | 2751 | 113 | 1.66E−08 | 1.26E−04 |
| | | process | | | | |
| GO:0016064 | BP | Ig mediated immune response | 140 | 15 | 1.51E−07 | 8.59E−04 |
| GO:0019724 | BP | B cell mediated immunity | 143 | 15 | 2.11E−07 | 9.65E−04 |
| GO:0002449 | BF | lymphocyte mediated immunity | 286 | 21 | 4.35E−07 | 1.59E−03 |
| GO:0002460 | BP | adaptive immune response based on | 292 | 22 | 4.89E−07 | 1.59E−03 |
| | | somatic recombination of immune | | | | |
| | | receptors built from Ig superfamily domains | | | | |
| GO:0006955 | BP | immune response | 1577 | 162 | 7.58E−07 | 1.96E−03 |
| GO:0002682 | BP | regulation of immune system process | 1358 | 59 | 7.71E−07 | 1.96E−03 |
| GO:0001775 | BP | cell activation | 1017 | 49 | 1.34E−06 | 3.06E−03 |
| GO:0001816 | BP | cytokine production | 820 | 39 | 1.57E−06 | 3.25E−03 |
| GO:0002707 | BP | negative regulation of lymphocyte mediated | 55 | 9 | 2.05E−06 | 3.76E−03 |
| | | immunity | | | | |
| GO:0001817 | BP | regulation of cytokine production | 806 | 38 | 2.14E−06 | 3.76E−03 |
| GO:0042113 | BP | B cell activation | 262 | 20 | 2.66E−06 | 4.34E−03 |
| GO:0006959 | BP | humoral immune response | 236 | 15 | 3.91E−06 | 5.73E−03 |
| GO:0002443 | BP | leukocyte mediated immunity | 378 | 23 | 4.02E−06 | 5.73E−03 |
| GO:0045321 | BP | leukocyte activation | 876 | 42 | 7.70E−06 | 9.84E−03 |
| GO:0048534 | BP | hematopoietic or lymphoid organ | 896 | 45 | 7.76E−06 | 9.84E−03 |
| | | development | | | | |
| GO:0002704 | BP | negative regulation of leukocyte mediated | 67 | 9 | 1.14E−05 | 1.33E−02 |
| | | immunity | | | | |
| GO:0009986 | CC | cell surface | 861 | 43 | 1.17E−05 | 1.33E−02 |
| GO:0002819 | BP | regulation of adaptive immune response | 185 | 15 | 1.24E−05 | 1.35E−02 |
| GO:0002706 | BP | regulation of lymphocyte mediated | 173 | 14 | 1.42E−05 | 1.47E−02 |
| | | immunity | | | | |
| GO:0002822 | BP | regulation of adaptive immune response | 171 | 14 | 1.67E−05 | 1.66E−02 |
| | | based on somatic recombination of immune | | | | |
| | | receptors built from lg superfamily domains | | | | |
| GO:0032501 | BP | multicellular organismal process | 7364 | 218 | 1.92E−05 | 1.82E−02 |
| GO:0001818 | BP | negative regulation of cytokine production | 362 | 19 | 2.14E−05 | 1.94E−02 |
| GO:0002923 | BP | regulation of humoral immune response | 18 | 5 | 2.22E−05 | 1.94E−02 |
| | | mediated by circulating immunoglobulin | | | | |
| GO:0002455 | BP | humoral immune response mediated by | 49 | 7 | 2.98E−05 | 2.51E−02 |
| | | circulating immunoglobulin | | | | |
| GO:0002520 | BP | immune system development | 955 | 45 | 3.36E−05 | 2.73E−02 |
| GO:0002684 | BP | positive regulation of immune system | 840 | 38 | 3.74E−05 | 2.82E−02 |
| | | process | | | | |
| GO:0002252 | BP | immune effector process | 594 | 29 | 3.79E−05 | 2.82E−02 |
| GO:0050896 | BP | response to stimulus | 8581 | 241 | 3.84E−05 | 2.82E−02 |
| GO:0046649 | BP | lymphocyte activation | 717 | 35 | 4.58E−05 | 3.26E−02 |
| GO:0030097 | BP | hemopoiesis | 856 | 41 | 5.40E−05 | 3.73E−02 |
| GO:0002701 | BP | negative regulation of production of | 38 | 6 | 6.15E−05 | 4.13E−02 |
| | | molecular mediator of immune response | | | | |
| GO:0050778 | BP | positive regulation of immune response | 513 | 26 | 7.52E−05 | 4.90E−02 |

TABLE 12
Classifier performance for IBS bowel habit subtypes

| | IBS-D vs | IBS-C vs | IBS-D vs | IBS-D vs | IBS-D vs | IBS-C vs | IBS-C vs | IBS-M vs | IBS-M vs |
|---|---|---|---|---|---|---|---|---|---|
| | HC | HC | IBS-C | IBD | celiac | IBD | Celiac | IBD | Celiac |
| AUC | 0.65 | 0.67 | 0.67 | 0.85 | 0.77 | 0.70 | 0.64 | 0.77 | 0.77 |
| Accuracy | 0.60 | 0.58 | 0.58 | 0.69 | 0.62 | 0.50 | 0.51 | 0.65 | 0.74 |
| Lower CI | | | | | | | | | |
| Accuracy | 0.76 | 0.74 | 0.77 | 0.89 | 0.87 | 0.75 | 0.79 | 0.88 | 0.95 |
| Upper CI | | | | | | | | | |
| Sensitivity | 0.50 | 0.42 | 0.71 | 0.7 | 0.60 | 0.77 | 0.31 | 0.08 | 0.58 |
| Specificity | 0.80 | 0.79 | 0.65 | 0.85 | 0.85 | 0.60 | 0.79 | 0.96 | 0.97 |
| F1 | 0.55 | 0.46 | 0.67 | 0.68 | 0.65 | 0.48 | 0.33 | 0.13 | 0.70 |
| Accuracy | 0.05 | 0.47 | 5.35E−03 | 0.04 | 3.07E−02 | 1.00 | 0.87 | 0.69 | 0.03 |
| P-value | | | | | | | | | |

IBS: irritable bowel syndrome; IBD: inflammatory bowel diseases; HCs: healthy controls; AUC, area under receiver operating characteristic (ROC) curve; CI: confidence interval

TABLE 13
Sensitivity analysis for assessing classifier performance
after excluding patients using medications

| | IBS (n = 40) vs IBD (n = 39) | IBS (n = 40) vs celiac (n = 26) |
|---|---|---|
| AUC | 0.81 | 0.79 |
| Accuracy | 0.76 (0.65-0.85) | 0.77 (0.65-0.87) |
| Accuracy Lower CI | 0.65 | 0.65 |
| Accuracy Upper CI | 0.85 | 0.87 |
| Sensitivity | 0.83 | 0.8 |
| Specificity | 0.69 | 0.73 |
| F1 | 0.78 | 0.81 |
| Accuracy P-value | 3.54E−06 | 3.20E−03 |

IBS: irritable bowel syndrome; IBD: inflammatory bowel diseases; HCs: healthy controls; AUC, area under receiver operating characteristic (ROC) curve; CI: confidence interval

TABLE 14A
Classifier-Associated CpGs for IBS IBD

| | | | | | |
|---|---|---|---|---|---|
| cg17420885 | cg24688963 | cg02063695 | cg13754665 | cg18581491 | cg13343932 |
| cg04352288 | cg17030488 | cg15431103 | cg19241279 | cg10621606 | cg25312694 |
| cg14578009 | cg20075528 | cg08596837 | cg07812827 | cg08306178 | cg02719245 |
| cg20982046 | cg10189135 | cg16503797 | cg18796948 | cg13760845 | cg26678425 |
| cg27107076 | cg01579216 | cg23534210 | cg06778637 | cg00454549 | cg04771433 |
| cg24597774 | cg23774349 | cg13683550 | cg18265608 | cg04547002 | cg20240860 |
| cg18728950 | cg17486396 | cg23971362 | cg09372538 | cg04648490 | cg00680446 |
| cg02983950 | cg12098511 | cg16618260 | cg11965685 | cg00139496 | cg23428387 |
| cg23249717 | cg25876879 | cg23139982 | cg13496999 | cg26793147 | cg21550804 |
| cg21848117 | cg09819656 | cg05872500 | cg19246761 | cg13173255 | cg11983196 |
| cg19503640 | cg07503286 | cg08526401 | cg04894169 | cg14019146 | cg27507681 |
| cg03346936 | cg26367552 | cg19977871 | cg18860133 | cg04340874 | cg01001533 |
| cg07624539 | cg07699771 | cg11093123 | cg15741433 | cg17775965 | cg09723524 |
| cg25144893 | cg16608407 | cg27437304 | cg18181906 | cg26372517 | cg01409259 |
| cg21184415 | cg09893703 | cg25453556 | cg07709959 | cg03738915 | cg17313709 |
| cg06934654 | cg09063262 | cg17377463 | cg03363417 | cg07017331 | cg17055322 |
| cg23191354 | cg16231917 | cg14799392 | cg17176470 | cg12871059 | cg18539608 |
| cg21041100 | cg18660468 | cg14363954 | cg12520615 | cg06242807 | cg21951526 |
| cg27393730 | cg17821372 | cg01229567 | cg10205527 | cg25297453 | cg07299819 |
| cg13994504 | cg13480937 | cg16931664 | cg23937295 | cg09341463 | cg15711011 |
| cg20464175 | cg27026192 | cg12478185 | cg01655226 | cg25057682 | IBD_batch |
| cg11726250 | cg24465561 | cg11255208 | cg13061170 | cg22591916 | |
| cg23499033 | cg01591592 | cg05275595 | cg26452434 | cg04795387 | |

TABLE 14B
Classifier-Associated CpGs for IBS vs Celiac Disease

| | | | | | |
|---|---|---|---|---|---|
| cg20748533 | cg18105749 | cg22280258 | cg26710563 | cg10979474 | cg04795387 |
| cg04418355 | cg07056602 | cg26680399 | cg11786848 | cg01198434 | cg24441440 |
| cg04908625 | cg17486396 | cg14898366 | cg01958356 | cg07713642 | cg00680446 |
| cg18834259 | cg21297282 | cg03091070 | cg14269096 | cg08827024 | cg07909178 |
| cg04870110 | cg07180897 | cg15240619 | cg25595439 | cg26372517 | cg15449914 |
| cg 12458875 | cg14417067 | cg12403132 | cg16376794 | cg10289901 | cg17264271 |

TABLE 14C
Classifier-Associated CpGs for IBD vs Celiac Disease

| | | | | | |
|---|---|---|---|---|---|
| cg25042346 | cg02780029 | cg21057699 | cg11742472 | cg07285331 | cg16455376 |
| cg05058672 | cg11898431 | cg11587926 | cg03616118 | cg01047748 | cg13929657 |
| cg26948907 | cg26867282 | cg03806328 | cg25106332 | cg09760564 | cg25214310 |
| cg13187827 | cg00250571 | cg10122294 | cg03760101 | cg17803876 | cg06668703 |
| cg23842572 | cg12833015 | cg01367324 | cg18268310 | cg26163234 | cg25642099 |
| cg13502474 | cg09730524 | cg10905078 | cg05869631 | cg11635702 | cg04337358 |
| cg09965384 | cg03636197 | cg24465561 | cg17534999 | cg26099730 | cg09940832 |
| cg04286438 | cg14900154 | cg14112935 | cg21163415 | cg14373277 | cg05855948 |
| cg22790377 | cg26052321 | cg03531326 | cg14321936 | cg19465345 | cg23116704 |
| cg13777717 | cg17832704 | cg24899138 | cg07185006 | cg05779272 | cg22698657 |
| cg08839808 | cg09521743 | cg21030160 | cg25107000 | cg01045132 | cg24048008 |
| cg13568106 | cg19694398 | cg23534210 | cg05144928 | cg05491977 | cg07702689 |
| cg05239811 | cg16761000 | cg18376497 | cg04707013 | cg18527739 | cg04845579 |
| cg27469738 | cg07699771 | cg17342247 | cg13098432 | cg13141890 | cg00631797 |
| cg18792364 | cg05328326 | cg23971362 | cg11977443 | cg03301058 | cg25713705 |
| cg02382016 | cg04098052 | cg20172258 | cg00226984 | cg18440480 | cg24654681 |
| cg14058476 | cg25264948 | cg04720592 | cg26914237 | cg26726006 | cg07514400 |
| cg03531100 | cg08573679 | cg15745662 | cg18181906 | cg02652361 | cg03372407 |
| cg23062006 | cg22666627 | cg13967697 | cg12523211 | cg16925459 | cg17313709 |
| cg00898880 | cg17360552 | cg23526676 | cg09426924 | cg21030548 | cg17055322 |
| cg03346936 | cg16231917 | cg11364301 | cg11701022 | cg23212215 | cg17250082 |
| cg23279117 | cg12802310 | cg01086075 | cg01502930 | cg15218522 | cg26364491 |
| cg03613822 | cg16262933 | cg24909101 | cg27071793 | cg27123273 | cg09067378 |
| cg13768953 | cg07180897 | cg21438681 | cg01613294 | cg10289901 | cg21210642 |
| cg22792367 | cg03842309 | cg03549563 | cg22510814 | cg15941598 | cg25419339 |
| cg04418355 | cg15235494 | cg21717353 | cg23937295 | cg22387578 | cg16854130 |
| cg19619387 | cg24045841 | cg14398929 | cg02911802 | cg08110488 | cg23992051 |
| cg08451469 | cg25122699 | cg13501907 | cg10896498 | cg14263063 | cg14480531 |
| cg19593741 | cg23710218 | cg05275595 | cg08296433 | cg27258561 | cg04222466 |
| cg13994504 | cg10144598 | cg10585661 | cg06388338 | cg08450021 | cg00875152 |

TABLE 14D
Classifier-Associated CpGs for IBS vs Healthy Control

| | | | | | |
|---|---|---|---|---|---|
| cg02624129 | cg00195749 | cg21943444 | cg09513276 | cg08295857 | cg25690897 |
| cg06263372 | cg16191389 | cg05952367 | cg04214626 | cg01001422 | cg04106948 |
| cg16335098 | cg09634154 | cg18706888 | cg11044476 | cg09819732 | cg17503389 |
| cg19725516 | cg26903210 | cg23200342 | cg02418535 | cg13459801 | cg08631528 |
| cg14612068 | cg13099417 | cg02563346 | cg15388309 | cg24586554 | cg14619716 |
| cg26056498 | cg08734931 | cg03783925 | cg16621833 | cg10373866 | cg01263077 |
| cg01650137 | cg22744090 | cg17865872 | cg01450228 | cg04612503 | cg14607642 |
| cg24193659 | cg08325814 | cg22322277 | cg00538381 | cg25354551 | cg17814781 |
| cg08057038 | cg09391799 | cg01011550 | cg07314988 | cg01839492 | cg16096531 |
| cg04574346 | cg23331303 | cg13807886 | cg24426044 | cg04310180 | cg17643330 |
| cg11462099 | cg11202686 | cg10230831 | cg06358534 | cg13795252 | cg23650249 |
| cg01242196 | cg25243766 | cg04358463 | cg17412351 | cg10522979 | cg03015952 |
| cg11850943 | cg26866348 | cg25028849 | cg13176070 | cg06251395 | cg08400962 |
| cg16204066 | cg03588855 | cg16921950 | cg20044118 | cg19192520 | cg18536644 |
| cg18297027 | cg07663278 | cg17618987 | cg13859764 | cg04628393 | cg03736826 |
| cg17974166 | cg24881973 | cg06874063 | cg25003047 | cg26120813 | cg07530656 |
| cg20059140 | cg24755503 | cg20519373 | cg26575637 | cg00242965 | cg26666292 |
| cg22990158 | cg08242859 | cg05607391 | cg05088382 | cg09422291 | cg12231227 |
| cg04554272 | cg01191262 | cg07957690 | cg24552587 | cg10879867 | cg15319693 |
| cg00038736 | cg18517369 | cg04404246 | cg08219773 | cg20713092 | cg08958748 |
| cg04825924 | cg15822321 | cg07161397 | cg06611109 | cg02701080 | cg01507147 |
| cg25006249 | cg18516067 | cg09001440 | cg27209722 | cg20845050 | cg01647314 |
| cg16577651 | cg23142004 | cg20738895 | cg00347369 | cg05769317 | cg11736690 |
| cg14578009 | cg18049228 | cg12331517 | cg00228931 | cg18480328 | cg25220765 |
| cg10714960 | cg14727987 | cg03851401 | cg02284273 | cg01954600 | cg04285822 |
| cg18343290 | cg01061047 | cg13099046 | cg18309140 | cg17402294 | cg14451730 |
| cg26980034 | cg18105749 | cg05491836 | cg09975804 | cg12464374 | cg08120071 |
| cg24274117 | cg27315287 | cg13299780 | cg00256709 | cg07007312 | cg23304165 |
| cg03645783 | cg03617023 | cg22124136 | cg07411382 | cg27601906 | cg17369140 |
| cg17743250 | cg08449403 | cg14078639 | cg02515725 | cg21829166 | cg09440270 |
| cg04316233 | cg14200127 | cg22181644 | cg19128731 | cg16841498 | cg21300460 |
| cg02519751 | cg14036981 | cg02809149 | cg22699443 | cg07086770 | cg10572337 |
| cg13764778 | cg01599397 | cg02503117 | cg00961616 | cg02026608 | cg00680446 |
| cg23322676 | cg11754222 | cg09722057 | cg03558873 | cg04857403 | cg15001728 |
| cg26638005 | cg09204545 | cg14194192 | cg09805196 | cg16104831 | cg02181349 |
| cg03743720 | cg27541226 | cg15197125 | cg22792461 | cg02950427 | cg08665903 |
| cg15683497 | cg02798614 | cg13158379 | cg11179610 | cg22374486 | cg07764113 |
| cg04494562 | cg06960220 | cg07727640 | cg10437320 | cg07713642 | cg00041585 |
| cg25753693 | cg07441122 | cg07634830 | cg12990614 | cg16145774 | cg16357582 |
| cg03701575 | cg19496587 | cg03613937 | cg03179435 | cg08129953 | cg13906078 |
| cg23634134 | cg20945232 | cg16843862 | cg08192793 | cg06229024 | cg02234048 |
| cg05017199 | cg04690110 | cg12553150 | cg07812827 | cg04088932 | cg21550804 |
| cg11515089 | cg05242561 | cg04464671 | cg09598431 | cg08846589 | cg08187479 |
| cg01426303 | cg13393785 | cg27139156 | cg12054190 | cg21118414 | cg23029597 |
| cg07939626 | cg13530673 | cg06331242 | cg00797839 | cg13030572 | cg04320476 |
| cg10411146 | cg15767271 | cg24977055 | cg08022717 | cg21173351 | cg06722483 |
| cg26788501 | cg26358374 | cg01479530 | cg07753203 | cg15742445 | cg11356485 |
| cg20982046 | cg06393340 | cg13440073 | cg03115308 | cg11629812 | cg01292375 |
| cg12102320 | cg07997370 | cg11956655 | cg04876703 | cg01284619 | cg03762366 |
| cg02676175 | cg04596558 | cg20966357 | cg11183001 | cg07879533 | cg01434025 |
| cg26328335 | cg01814130 | cg01960569 | cg23471274 | cg12128164 | cg11296268 |
| cg11254426 | cg05662369 | cg25264804 | cg10843030 | cg06651119 | cg11836949 |
| cg11970204 | cg13263947 | cg01826884 | cg13188283 | cg04110585 | cg03126799 |
| cg04947136 | cg05552010 | cg05230207 | cg03570920 | cg02264319 | cg11877105 |
| cg16609260 | cg03560957 | cg19650697 | cg13177778 | cg04504559 | cg10386420 |
| cg20938507 | cg02635949 | cg23534210 | cg20347377 | cg16755025 | cg09549987 |
| cg08251704 | cg09676171 | cg22253931 | cg10769180 | cg18315870 | cg03337492 |
| cg16911275 | cg11278212 | cg18923006 | cg10928044 | cg13131115 | cg05275618 |
| cg13581422 | cg04633204 | cg27624753 | cg07832722 | cg21953146 | cg24646951 |
| cg11245990 | cg14157514 | cg24435844 | cg17009616 | cg09699787 | cg10045903 |
| cg15006627 | cg21961499 | cg02199333 | cg26843807 | cg13573611 | cg21167985 |
| cg17842918 | cg16468389 | cg13497082 | cg02685867 | cg07545526 | cg04628802 |
| cg06744585 | cg08114852 | cg16111277 | cg24696500 | cg16649771 | cg26922546 |
| cg13081331 | cg08410888 | cg13617812 | cg03299636 | cg11392231 | cg25818609 |
| cg16049751 | cg25518066 | cg04264070 | cg13260217 | cg24522010 | cg06641416 |
| cg11964207 | cg17208781 | cg08757828 | cg13755830 | cg06128161 | cg15251675 |
| cg09469667 | cg14118679 | cg17859326 | cg22851561 | cg19480425 | cg05752943 |
| cg18645906 | cg22433759 | cg00652274 | cg00555420 | cg14791008 | cg03698571 |
| cg02449103 | cg18554826 | cg09851545 | cg05446587 | cg09101205 | cg05820281 |
| cg07136023 | cg07680530 | Cg06726820 | cg26256126 | cg09291824 | cg22012327 |
| cg12162424 | cg09303139 | cg06871323 | cg01853346 | cg26697654 | cg02022344 |
| cg23936215 | cg07302841 | cg10116893 | cg00607051 | cg17404981 | cg01768308 |
| cg20482390 | cg04936009 | cg21982246 | cg06257936 | cg01273232 | cg25541156 |
| cg00919702 | cg03343013 | cg04712314 | cg23997185 | cg22026062 | cg26981703 |
| cg13390867 | cg27487344 | cg16138024 | cg12331932 | cg25261265 | cg21371809 |
| cg16379462 | cg24093642 | cg05992598 | cg17594757 | cg24661245 | cg05820007 |
| cg23911912 | cg09555706 | cg20498291 | cg12047815 | cg16326814 | cg22357379 |
| cg05865911 | cg24928106 | cg14265502 | cg06966912 | cg06168473 | cg11983196 |
| cg02783799 | cg13600897 | cg24719521 | cg14694712 | cg20563666 | cg06748804 |
| cg08017326 | cg02402368 | cg20978864 | cg13374658 | cg02002551 | cg06028808 |
| cg21164975 | cg08712970 | cg12234708 | cg12842251 | cg05633888 | cg24210711 |
| cg10801328 | cg09113046 | cg16720947 | cg03815480 | cg25714432 | cg10075976 |
| cg13819127 | cg20506843 | cg20506632 | cg17055154 | cg24821307 | cg24601344 |
| cg17781710 | cg17784808 | cg11999255 | cg09146328 | cg24134672 | cg11230031 |
| cg02098786 | cg10882484 | cg07052546 | cg03824347 | cg22781632 | cg00690544 |
| cg02614024 | cg25936380 | cg03054026 | cg13787932 | cg14566103 | cg16237800 |
| cg19858576 | cg00962903 | cg04476394 | cg22308813 | cg12694063 | cg01742568 |
| cg07141605 | cg01651710 | cg15685749 | cg12666403 | cg04100322 | cg24737201 |
| cg17915676 | cg17935173 | cg15088324 | cg17700392 | cg17068631 | cg07989895 |
| cg07258910 | cg01995986 | cg15858512 | cg06068935 | cg08109808 | cg20322977 |
| cg26910511 | cg01142721 | cg15403517 | cg09114261 | cg17172683 | cg22229142 |
| cg15215251 | cg23626798 | cg10436120 | cg23243982 | cg04171117 | cg21243351 |
| cg26564280 | cg26933632 | cg05002165 | cg16631283 | cg04343893 | cg15121445 |
| cg00002033 | cg21242079 | cg24099956 | cg19611364 | cg05648752 | cg01050273 |
| cg18527352 | cg17102495 | cg07000567 | cg05730537 | cg11965365 | cg24555250 |
| cg14299480 | cg02428178 | cg24375215 | cg05078833 | cg20017856 | cg15922611 |
| cg21877005 | cg19824055 | cg16426475 | cg22382805 | cg19256458 | cg14249990 |
| cg10081626 | cg13353999 | cg04311466 | cg13238479 | cg00588430 | cg09406856 |
| cg12126857 | cg19091715 | cg10860199 | cg12711961 | cg16064424 | cg13932290 |
| cg05581851 | cg09674169 | cg01342572 | cg05951984 | cg00935887 | cg26936966 |
| cg07763662 | cg19434640 | cg18767846 | cg07709959 | cg04383212 | cg15123104 |
| cg02447304 | cg18993757 | cg00466402 | cg03363417 | cg08531298 | cg16277843 |
| cg24463509 | cg04759104 | cg19257111 | cg09096031 | cg20043244 | cg01450725 |
| cg17907729 | cg14846617 | cg08350092 | cg20744229 | cg13759632 | cg06604589 |
| cg24460126 | cg20708141 | cg03182908 | cg22616525 | cg08853304 | cg27472353 |
| cg07970752 | cg15813011 | cg17459182 | cg26049040 | cg05979484 | cg21583440 |
| cg04053101 | cg14040696 | cg10552127 | cg02480144 | cg01579244 | cg18469436 |
| cg01572696 | cg26561907 | cg09966375 | cg10439113 | cg13408214 | cg16495154 |
| cg27404186 | cg11496569 | cg01227572 | cg18896979 | cg09057405 | cg05204532 |
| cg06245229 | cg08548089 | cg23022828 | cg08344551 | cg01572718 | cg14041855 |
| cg20669292 | cg04565065 | cg07716408 | cg20468415 | cg23758309 | cg24018665 |
| cg16886828 | cg15406387 | cg08526401 | cg19978674 | cg05626790 | cg16318442 |
| cg18271913 | cg13343392 | cg13192836 | cg25616547 | cg03073429 | cg24978725 |
| cg17102205 | cg08762472 | cg17378969 | cg12476754 | cg01171887 | cg13750905 |
| cg20744217 | cg05485853 | cg19090360 | cg25459623 | cg08031967 | cg16810339 |
| cg22705016 | cg00481382 | cg08627089 | cg27012920 | cg21582240 | cg02188862 |
| cg27126872 | cg01841825 | cg06983508 | cg16872106 | cg00670942 | cg10991836 |
| cg07244326 | cg05447875 | cg08950410 | cg25349314 | cg23212215 | cg14931118 |
| cg05063952 | cg25741609 | cg25652742 | cg11059493 | cg26870771 | cg09681594 |
| cg04488153 | cg09911480 | cg01690390 | cg20953364 | cg13868361 | cg20540428 |
| cg15351009 | cg22314031 | cg01381617 | cg01090161 | cg00875400 | cg27319658 |
| cg16834823 | cg09782171 | cg11552903 | cg04304417 | cg20924006 | cg06090684 |
| cg15574461 | cg01056004 | cg13613073 | cg12728061 | cg18340953 | cg08697116 |
| cg10220895 | cg03250393 | cg24169812 | cg03005851 | cg21670003 | cg11450055 |
| cg19997218 | cg16379698 | cg15352421 | cg14820271 | cg10270155 | cg12041830 |
| cg10953317 | cg16997101 | cg06394333 | cg23749637 | cg06731236 | cg16127496 |
| cg06928741 | cg17207545 | cg04186487 | cg06364996 | cg06524264 | cg02207386 |
| cg13769283 | cg05202042 | cg10439678 | cg08706575 | cg06879998 | cg22954052 |
| cg09826306 | cg25858471 | cg13543375 | cg27519373 | cg04482072 | cg02698708 |
| cg00691874 | cg23763117 | cg11595575 | cg19907661 | cg14321658 | cg08390696 |
| cg04908625 | cg19928450 | cg24718699 | cg25880958 | cg05286501 | cg05160449 |
| cg25460807 | cg05356319 | cg00085123 | cg22910862 | cg24867298 | cg04021197 |
| cg25820703 | cg18440960 | cg15764645 | cg11008866 | cg11685510 | cg06057319 |
| cg13007784 | cg04202774 | cg14181940 | cg25163248 | cg15315372 | cg16178091 |
| cg00172812 | cg02618039 | cg23117497 | cg07226201 | cg06778853 | cg14684678 |
| cg12313476 | cg16904686 | cg13019673 | cg14085543 | cg05557583 | cg19222569 |
| cg03024212 | cg09517141 | cg14608505 | cg12060669 | cg11572548 | cg09263164 |
| cg14327228 | cg18224942 | cg08825109 | cg05647993 | cg09889154 | cg18444763 |
| cg05922723 | cg25547830 | cg18735473 | cg06138439 | cg10357888 | cg01224715 |
| cg10612452 | cg08596595 | cg02104409 | cg26133337 | cg03356492 | cg12875254 |
| cg22453529 | cg21805940 | cg18115575 | cg10441094 | cg13463492 | cg19452802 |
| cg11115431 | cg26363053 | cg04692870 | cg09418601 | cg16201418 | |
| cg26984593 | cg20589883 | cg17339882 | cg16656316 | cg10189456 | |
| cg16037983 | cg07442408 | cg03740761 | cg09138512 | cg22111536 | |
| cg14003978 | cg26787256 | cg13895830 | cg05030030 | cg04763273 | |

TABLE 14E
Classifier-Associated CpGs for IBD vs Healthy Control

| | | | | | |
|---|---|---|---|---|---|
| cg10302973 | cg14816899 | cg27635859 | cg05085844 | cg00667467 | cg16038868 |
| cg14743534 | cg03922648 | cg18706888 | cg20522437 | cg13760845 | cg15319693 |
| cg05787209 | cg22256784 | cg06207791 | cg15436480 | cg00454549 | cg23966795 |
| cg19725516 | cg06247806 | cg03783925 | cg22620356 | cg11746684 | cg17109563 |
| cg19579217 | cg13866747 | cg15635962 | cg24171640 | cg13526474 | cg11613272 |
| cg03897095 | cg09998591 | cg17865872 | cg16510709 | cg01668008 | cg11383809 |
| cg17119907 | cg13574820 | cg21057699 | cg19241279 | cg27601906 | cg26463496 |
| cg05687710 | cg06984666 | cg06697074 | cg00306311 | cg15319704 | cg19145240 |
| cg25006249 | cg17459778 | cg06874063 | cg02825765 | cg13489576 | cg16767274 |
| cg01147352 | cg13328862 | cg10122294 | cg26118221 | cg01939741 | cg13878023 |
| cg18040354 | cg00790177 | cg19007410 | cg15105517 | cg07390791 | cg00140754 |
| cg25260865 | cg19035215 | cg03347353 | cg06920022 | cg03458265 | cg04932658 |
| cg05396397 | cg13984188 | cg06784095 | cg10193422 | cg11800019 | cg17884064 |
| cg23715045 | cg08642198 | cg13099046 | cg24570159 | cg17803876 | cg07910479 |
| cg01843946 | cg12610070 | cg01909818 | cg24126941 | cg09337043 | cg01731495 |
| cg20409636 | cg01225565 | cg08612384 | cg01664441 | cg21523688 | cg00413728 |
| cg10426581 | cg00711090 | cg07330997 | cg09350362 | cg14016257 | cg07245356 |
| cg12201399 | cg05698846 | Cg04464671 | cg08789486 | cg01380261 | cg25719685 |
| cg21908235 | cg20696345 | cg11906021 | cg07468305 | cg17582074 | cg23780614 |
| cg09328979 | cg05766064 | cg07251446 | cg13177778 | cg07810881 | cg25248588 |
| cg04829186 | cg13201172 | cg05294930 | cg01870519 | cg01445725 | cg00269725 |
| cg04494562 | cg19403377 | cg26557617 | cg25772418 | cg08252745 | cg22076945 |
| cg17953300 | cg10852431 | cg03645661 | cg02770097 | cg11635702 | cg21550107 |
| cg08439487 | cg24803346 | cg11277907 | cg01689239 | cg23319696 | cg03546163 |
| cg20988098 | cg15612485 | cg27586596 | cg21046951 | cg12751142 | cg08684314 |
| cg09087363 | cg17588577 | cg13064346 | cg17705647 | cg15324288 | cg20506944 |
| cg26328335 | cg10740486 | cg11116818 | cg00880157 | cg14373277 | cg22056336 |
| cg05082557 | cg04558084 | cg05230207 | cg04689398 | cg13085024 | cg11814956 |
| cg21479860 | cg03057481 | cg01656470 | cg17213476 | cg19465345 | cg12467933 |
| cg11970204 | cg25687744 | cg14606694 | cg15888865 | cg21702966 | cg04114982 |
| cg07978099 | cg23774349 | cg15537651 | cg26184673 | cg20959703 | cg12943115 |
| cg08839808 | cg06964816 | cg10769442 | cg18553358 | cg22874598 | cg27616543 |
| cg02206767 | cg01429657 | cg00135500 | cg06615754 | cg22395811 | Cg06127003 |
| cg14972139 | cg13334727 | cg19805274 | cg13496999 | cg11772801 | cg24646951 |
| cg10800833 | cg27295118 | cg03325664 | cg09420728 | cg09757099 | cg02920129 |
| cg02001279 | cg17208781 | cg21714266 | cg04894169 | cg25138693 | cg20586895 |
| cg08832624 | cg22433759 | cg21013431 | cg20588045 | cg22508930 | cg16412914 |
| cg18728950 | cg12898496 | cg04819618 | cg01305291 | cg27154217 | cg08098550 |
| cg01600222 | cg09521743 | cg24311634 | cg12888861 | cg27331871 | cg05586573 |
| cg06915202 | cg05997779 | cg20172258 | cg14465701 | cg06419546 | cg06641416 |
| cg13919358 | cg15920453 | cg20461812 | cg08140458 | cg13702733 | cg06687923 |
| cg15992284 | cg02386293 | cg09831562 | cg09036531 | cg06386482 | cg04367545 |
| cg14927855 | cg11821532 | cg02667577 | cg18860133 | cg02673002 | cg20474755 |
| cg18792364 | cg11203771 | cg14632490 | cg11977443 | cg22467301 | cg02804617 |
| cg06093039 | cg23412850 | cg24668440 | cg07245367 | cg00574183 | cg01768308 |
| cg23776628 | cg06763280 | cg10160570 | cg16102930 | cg15531562 | cg09038078 |
| cg20269046 | cg07503286 | cg23483316 | cg18181906 | cg22994555 | cg22357379 |
| cg22196260 | cg13931396 | cg00082540 | cg01530498 | cg12598169 | cg17187183 |
| cg07233159 | cg21379099 | cg07549924 | cg07808087 | cg11047325 | cg03993991 |
| cg02948862 | cg26243277 | cg22281120 | cg20883720 | cg19019944 | cg09994445 |
| cg05029822 | cg26415535 | cg13967697 | cg03221779 | cg14348967 | cg08638481 |
| cg14058476 | cg18829411 | cg01507442 | cg01143853 | cg25297453 | cg24968203 |
| cg18598117 | cg06861560 | cg09526234 | cg07167143 | cg04820773 | cg08679405 |
| cg12479878 | cg11987645 | cg27041049 | cg06035456 | cg27117982 | cg27161340 |
| cg08854185 | cg08573679 | cg07035517 | cg11587439 | cg17161468 | cg08682385 |
| cg21486834 | cg03671431 | cg06364604 | cg09555097 | cg02652361 | cg23096476 |
| cg12500951 | cg13817905 | cg03578689 | cg09103311 | cg20895669 | cg24895637 |
| cg15750303 | cg06718625 | cg20979737 | cg20468415 | cg01413141 | cg24771104 |
| cg11650874 | cg15600051 | cg13641591 | cg07935669 | cg00147160 | cg21150347 |
| cg12634306 | cg10023613 | cg21500092 | cg12476754 | cg11283404 | cg06299093 |
| cg06220449 | cg03725573 | cg12559471 | cg00914726 | cg14318940 | Cg07407950 |
| cg06582706 | cg11958060 | cg21090298 | cg20209548 | cg22492847 | cg03372407 |
| cg03321553 | cg00254017 | cg20655099 | cg03172058 | cg21030548 | cg17313709 |
| cg16188984 | cg11618577 | cg27309858 | cg07876788 | cg23358629 | cg00106685 |
| cg09387867 | cg26719720 | cg12592723 | cg15357934 | cg15218522 | cg04378886 |
| cg00425918 | cg15176487 | cg23490796 | cg16908208 | cg26838949 | cg21954465 |
| cg01493297 | cg06474923 | cg05501761 | cg27519373 | cg13868361 | cg02908707 |
| cg17158414 | cg14596140 | cg24238356 | Cg23825301 | cg14474319 | cg14050060 |
| cg08214808 | cg02433148 | cg07448986 | cg23562858 | cg04845466 | cg05350409 |
| cg13768953 | cg19028983 | cg13902335 | cg23937295 | cg14969100 | cg13586425 |
| cg06934654 | cg01637125 | cg19602308 | cg19907661 | cg07097098 | cg02484776 |
| cg18540455 | cg12547439 | cg24998464 | cg26599989 | cg10861005 | cg16731079 |
| cg19619387 | cg15897209 | cg03878190 | cg26939341 | cg25057682 | cg11738543 |
| cg21248554 | cg06588028 | cg23262340 | cg01655226 | cg07767033 | cg11182600 |
| cg06684987 | cg21419800 | cg15764645 | cg21934311 | cg04001676 | cg16669388 |
| cg25241559 | cg18811498 | cg25941083 | cg12575162 | cg06731236 | cg01738251 |
| cg12648201 | cg20345446 | cg21498511 | cg20158050 | cg03797906 | cg22163395 |
| cg08847173 | cg17207545 | cg08798305 | cg09197432 | cg11685510 | cg21951526 |
| cg09826306 | cg08254831 | cg14363954 | cg06809476 | cg01368737 | cg25419339 |
| cg00172812 | cg14318858 | cg11293814 | cg15059558 | cg13343932 | cg07150906 |
| cg23754918 | cg11602997 | cg08667706 | cg21253459 | cg00021325 | cg06448156 |
| cg00034101 | cg10503232 | cg04787728 | cg08622043 | cg24295653 | cg25063737 |
| cg02144874 | cg21682295 | cg12833011 | cg14107424 | cg02779288 | cg11932260 |
| cg14010405 | cg18544564 | cg02113536 | cg18581491 | cg09889154 | cg09916663 |
| cg08894487 | cg14416269 | cg24990989 | cg10061567 | cg00543315 | cg24887111 |
| cg12863693 | cg15235494 | cg04378708 | cg20461912 | cg14263063 | cg11598806 |
| cg17369032 | cg01772700 | cg12945409 | cg20174102 | cg09130447 | cg23992051 |
| cg13256645 | cg27068143 | cg16931664 | cg14853964 | cg22053732 | cg14480531 |
| cg05880582 | cg19328475 | cg07467736 | cg04746287 | cg25690897 | cg26668276 |
| cg24668990 | cg17821372 | cg22206167 | cg05280794 | cg00875541 | IBD_batch |
| cg25653341 | cg26621918 | cg09017815 | cg14317608 | cg20528093 | |
| cg01365568 | cg19945708 | cg21996068 | cg08558340 | cg03966582 | |

TABLE 14F
Classifier-Associated CpGs for Celiac Disease vs Healthy Control

| | | | | | |
|---|---|---|---|---|---|
| cg08230215 | cg16299091 | cg01022365 | cg14174367 | cg01668008 | cg14599091 |
| cg10244976 | cg27121980 | cg26512468 | cg03451788 | cg21523688 | cg15519048 |
| cg13187827 | cg13984188 | cg02230855 | cg17955729 | cg08993331 | cg17818632 |
| cg02722214 | cg18357676 | cg23667124 | cg16621833 | cg01380261 | cg02931642 |
| cg25231832 | cg01225565 | cg27586596 | cg12098187 | cg08827024 | cg09019137 |
| cg01630199 | cg14813923 | cg23534210 | cg20868217 | cg26840333 | cg04628802 |
| cg15755348 | cg17749578 | cg18450626 | cg03856153 | cg02980621 | cg02484210 |
| cg24680632 | cg27304042 | cg08139565 | cg20471125 | cg00365524 | cg01863392 |
| cg24147428 | cg23774349 | cg09851545 | cg17205324 | cg10935889 | cg17144547 |
| cg04494562 | cg01787798 | cg06726820 | cg05480055 | cg20802826 | cg06178073 |
| cg22379213 | cg07302841 | cg18848419 | cg17353680 | cg18161748 | cg01768308 |
| cg00590324 | cg20914572 | cg09474319 | cg08789486 | cg12648819 | cg13894462 |
| cg11970204 | cg18407168 | cg08909806 | cg11272074 | cg03301058 | cg16981703 |
| cg21493951 | cg05266212 | cg16349662 | cg19851529 | cg22110654 | cg00256485 |
| cg04865726 | cg06587659 | cg20978864 | cg20113744 | cg15531562 | cg22832883 |
| cg05239811 | cg04634427 | cg04476394 | cg12047815 | cg23903129 | cg18816397 |
| cg15974673 | cg06091535 | cg04286697 | cg08289744 | cg02755938 | cg07407950 |
| cg07856470 | cg08159594 | cg05029456 | cg03183354 | cg11241750 | cg13420277 |
| cg01567751 | cg06376949 | cg00082540 | cg09096031 | cg05871802 | cg27622679 |
| cg12387154 | cg22666627 | cg01507442 | cg10407113 | cg26460320 | cg08470863 |
| cg26352088 | cg14040696 | cg14126466 | cg00914726 | cg23992426 | cg14050060 |
| cg08854185 | cg21297282 | cg13641591 | cg10338066 | cg19038327 | cg24793351 |
| cg06026468 | cg22151017 | cg16602850 | cg00595754 | cg06731236 | cg16669388 |
| cg06105699 | cg07180897 | cg23526676 | cg01887469 | cg00191853 | cg10215507 |
| cg01011367 | cg08574479 | cg14592260 | cg25645491 | cg03356492 | cg24978725 |
| cg05618683 | cg21682295 | cg04948915 | cg08459440 | cg24973907 | cg00811565 |
| cg23384886 | cg12666336 | cg17378969 | cg25397029 | cg01091620 | cg18736141 |
| cg08214808 | cg16763513 | cg09886037 | cg06522206 | cg03597174 | cg17113856 |
| cg04418355 | cg20589883 | cg22334188 | cg03513867 | cg00875541 | cg08018572 |
| cg25604594 | cg18767636 | cg25146575 | cg16253639 | cg10434392 | cg03276785 |
| cg05764839 | cg00971695 | cg00875849 | cg07007382 | cg06835821 | cg10519437 |
| cg06327674 | cg04711042 | cg02411293 | cg01974213 | cg13532822 | cg03478739 |
| cg23641597 | cg01533183 | cg26880399 | cg27206026 | cg19145240 | |
| cg25121721 | cg12749087 | cg18077418 | cg02692808 | cg19646227 | |

Annotation of probes using IlluminaHumanMethylationEPICanno.ilm10b2.hg19 package can be used to obtain exact information on probe sequences using the CpG ID. IlluminaHumanMethylationEPICanno.ilm10b2.hg19.

Throughout this application various publications are referenced. The disclosures of these publications in their entireties are hereby incorporated by reference into this application in order to describe more fully the state of the art to which this invention pertains.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

1. A method of classifying biological samples based on gastrointestinal disease, the method comprising:
(a) measuring the amount of DNA methylation at CpG sites in DNA in a biological sample obtained from the subject to generate a genome-wide DNA CpG methylation profile of the subject, wherein the measuring comprises probing at least 200,000 CpG sites;
(b) performing a normalization of technical noise and bias correction on the profile;
(c) filtering the profile to retain data from a first set of probed CpG sites that is the same as a first set of CpG sites from a first training data set trained on a first pairwise comparison between biological samples, wherein the first pairwise comparison is selected from:
(i) irritable bowel syndrome (IBS) and inflammatory bowel disease (IBD);
(ii) IBS and celiac disease (CeD);
(iii) IBD and CeD;
(iv) IBS and healthy control;
(v) IBD and healthy control; and
(vi) CeD and healthy control;
(d) classifying the CpG methylation profile of the biological samples as IBS, IBD, CeD, or healthy based on a probability cutoff derived from the first training data set.

2. The method of claim 1, wherein the filtering of step (c) is repeated to retain data from a second set of probes that is the same as a second set of probes from a second training data set trained on a second pairwise comparison between biological samples, wherein the second pairwise comparison is selected from (i) to (vi), and wherein the second pairwise comparison differs from the first pairwise comparison.

3. The method of claim 2, wherein the filtering of step (c) is repeated to retain data from subsequent sets of probes corresponding to subsequent training data sets trained on subsequent pairwise comparisons between biological samples, wherein each of the remaining pairwise comparisons of (i) to (vi) is performed.

4. The method of claim 1, wherein the probing comprises contacting the biological samples with a methylation microarray.

5. The method of claim 1, wherein the biological sample comprises blood, plasma, serum, or mucosal tissue.

6. The method of claim 5, wherein the sample is peripheral blood mononuclear cells (PBMCs), peripheral blood lymphocytes (PBL), or whole blood.

7. The method of claim 1, further comprising measuring the amount of fecal calprotectin and/or blood C-reactive protein (CRP) in the biological sample.

8. The method of claim 1, wherein the training data set is trained using GLMNET algorithm.

9. The method of claim 1, wherein the training data set is trained using regularization parameter value and elastic net mixing parameter.

10. The method of claim 1, wherein the classifying is implemented by a computer.

11. A method of screening for irritable bowel syndrome (IBS), inflammatory bowel disease (IBD) and celiac disease (CeD) in a subject, the method comprising performing the method of classifying of claim 1 on a biological sample obtained from the subject, and identifying the subject as having IBS, IBD, or CeD based on the classifying of step (d).

12. The method of claim 11, further comprising treating the subject for IBS, IBD, or CeD.

13. The method of claim 12, wherein the treatment for IBS comprises administration of one or more of fiber supplements, laxatives, anti-diarrheal medication, loperamide, a bile acid binder, cholestyramine, colestipol, colesevelam, anticholinergic medications, dicyclomine or hysocyamine, tricyclic antidepressants, imipramine, desipramine, nortriptyline, selective serotonin reuptake inhibitors (SSRI) such as fluoxetine, paroxetine, serotonin and norepinephrine reuptake inhibitor (SNRI), such as duloxetine, tetracyclic antidepressant, such as mirtazapine, pregabalin, gabapentin, Alosetron, Eluxadoline, Rifaximin, Lubiprostone, Plecanatide, Tenapanor or Linaclotide.

14. The method of claim 12, wherein the treatment for IBD comprises administration of one or more of anti-inflammatory drugs, ude aminosalicylates, mesalamine, balsalazide, olsalazine, immune system suppressors, corticosteroids, azathioprine, mercaptopurine, methotrexate, tofacitinib, Upadacitinib, ozanimod, infliximab, adalimumab, golimumab, certolizumab, vedolizumab, ustekinumab, Risankizumab, antibiotics, anti-diarrheal medications, psyllium powder, methylcellulose, or loperamide.

15. The method of claim 12, wherein the treatment for CeD comprises one or more of gluten-free diet, vitamin and mineral supplements, steroids, azathioprine, or budesonide.

16. A method of treating a subject suffering from gastrointestinal disease, the method comprising:
(a) measuring the amount of DNA methylation at CpG sites in DNA in a biological sample obtained from the subject to generate a genome-wide DNA CpG methylation profile of the subject, wherein the measuring comprises probing at least 200,000 CpG sites;
(b) performing a normalization of technical noise and bias correction on the profile;
(c) filtering the profile to retain data from a first set of probed CpG sites that is the same as a first set of CpG sites from a first training data set trained on a first pairwise comparison between biological samples, wherein the first pairwise comparison is selected from:
(i) irritable bowel syndrome (IBS) and inflammatory bowel disease (IBD);
(ii) IBS and celiac disease (CeD);
(iii) IBD and CeD;
(iv) IBS and healthy control;
(v) IBD and healthy control; and
(vi) CeD and healthy control;
(d) classifying the CpG methylation profile of the biological samples as IBS, IBD, CeD, or healthy based on a probability cutoff derived from the first training data set; and
(e) treating the subject with administration of one or more of fiber supplements, laxatives, anti-diarrheal medication, loperamide, a bile acid binder, cholestyramine, colestipol, colesevelam, anticholinergic medications, dicyclomine or hysocyamine, tricyclic antidepressants, imipramine, desipramine, nortriptyline, selective serotonin reuptake inhibitors (SSRI) such as fluoxetine, paroxetine, serotonin and norepinephrine reuptake inhibitor (SNRI), such as duloxetine, tetracyclic antidepressant, such as mirtazapine, pregabalin, gabapentin, Alosetron, Eluxadoline, Rifaximin, Lubiprostone, Plecanatide, Tenapanor or Linaclotide when the profile is classified as IBS in step (d);
treating the subject with administration of one or more of anti-inflammatory drugs, ude aminosalicylates, mesalamine, balsalazide, olsalazine, immune system suppressors, corticosteroids, azathioprine, mercaptopurine, methotrexate, tofacitinib, Upadacitinib, ozanimod, infliximab, adalimumab, golimumab, certolizumab, vedolizumab, ustekinumab, Risankizumab, antibiotics, anti-diarrheal medications, psyllium powder, methylcellulose, or loperamide when the profile is classified as IBD in step (d); and
treating the subject with administration of one or more of gluten-free diet, vitamin and mineral supplements, steroids, azathioprine, or budesonide when the profile is classified as CeD in step (d).
